SEQUENCE LISTING (89 sequences)

SEQ ID NO: 1 - DNA, 2372 bp, synthetic construct
  source 1..2372: mol_type = other DNA
gacaagtagcaaacttttaggcaattttccaatcagagaatattattgtccattgcaatttgaaaaatagaaacggattcaagtaaaaacttacataataacaactaaaataaagaagcctaagacaatgaccttaattgcttcgaactatccacggccttggtgcgatgacactagaaattttcaaaacgaatttaaattgttaaattaacttcaaaattgaaaatatgaattcattaactaaaaaaccaaaaaatatcttttattcaattacgattttcatatcaaaattgaaccaaaccatatcatttataatattattatatttttataaaagtgaccttgaaaagaaatgtcatttcattataattaaggatttaaggatgtttattttttaaaagttgttcaaggaatataatatcaaaatcttacctttatactgaataaacaaactcatccttataaacaactactttcatatatttaagtaatgtaaaaagtaaactaattaattatcaaagaagctaaatatttgaggaaaatataggaaaaaaatagtttgactaaagaaaaaatatacaaaagtgaaattatgtaattttaaaagtattgacctgttaatgtaacattttaaataaatatagtaaaataatagttaattaaaaaaagaatcacaataatataaataaatcataattttttaaaaattcaaataaaatattatttgttgaaaattcaatccgaattcacacttcaagaaggttacccgatatatcatcctgttataaaaaagtaagaaacacgttataacacacattttcatgcacgtgaaaaataaaaataaaatatttttttgataaatattaattattaatctgttaattttattaatatgagaaattaaacacataatcttttacttatttattttttcttaaccctcaatcaatcttatatcccttatgataaatcaaacacactaataagtaattcattatttggttaaatactatctcaaaatttgtgcacaacaagttaaatgataaacctaacatgcaccgaatcacccaactattggtgtgtctgtattcctgtttaagtacatttatcgttaatccaaaggcgagagagaacagaaagacggaatgttctattcacgtaaacatgcataaaaagataacccaaaaaatgttgttgggtttaatacatttgtttgattatgaatagtgtatttgtattagtaattttattaatttttgtctgtgaaattacttttgtacaaataatatttttttaaaaaaattacaacaaaaaagtagacagagaatagaaaatatataataaaatagatgatataataataaaactcttaaatataagatgaaaaataatttataattaattgaagatttaataacttttacactaacattccacgtatattaaacttttaaacaataagacttgactcaatttgtttttggtttcatagctaaagttttttaaactaccaaacaattaaaagttatcattaataatacttttaattattataaaaattaataaatttatcatatataatgatatgtgattgaataatattataaaattattttacactaaaactgatttacctaaatgttaaagaattttttaactagtaaaaaaaatataaaaattaactcaaatgtatttgccaaataagttgggccatttaataacattctctcttattttcttcttgtttttaaaagaataagttggaccaaatatatttcttaaaaaactttaagaaaggaatataaaaataatgcatggagattatatatttttaataaataatatgtgcatttgattgtataaagtttttctatgtattttcattcggtcacaattattatatttactatctttgttgattttataacaattaccttaaaaatcttatgaaagatactttttataagttgacaatgtaagtatgtaactgatttgagttttttaaagtgtcatcgaaatatcagttgttaatattatcctttaccttacattttttaattttaaacttgtatgattgactaattaaatgtctaaaaactttagatttttcataatttcataatgaaatattaataacattatataatttcaattactaaaatattaatagttaactcttccagttctagtttaacttccccaaacctttgtagactctagctattattaaaaaaatgaaaaaacaaaaggcataacaaaacgctgtagcaggagtaggatgtggacctaattattgttgtattgtaacgaagctcaacgttagagaggcagcttttgctttataaaaaccagagattgaacaaccaaagagc

SEQ ID NO: 2 - DNA, 2849 bp, synthetic construct
  source 1..2849: mol_type = other DNA
tcagtgtggcagtttggtcgttgagtgagtgagtgaggctggtttcactttcacattgcctcttcaaaatgactttctccttttccttcttcaccatcaccactctgctttctctgttctctctaattctgcttcatgccaatgccttcccagtccccatgcatcgccaccctcgctttgccactcataactacagagatgctctcactaaatccattctcttctttgaaggccagaggtcagggaagctccctcctaaccagagaatgtcttggaggagagactctggcctctctgatggctcagccatgcacgtatgcatataccaactctcacattaccatgttttctggtttttgtttttggtttgcttatttatgtgtgaatgtgcaatgcaggttgatttagttggagggtactatgatgctggggacaatgtaaaatttggttttcccatggccttcaccaccaccatgctttcatggagcgttattgagtttggtgggctaatgaaaggtgagttgcagaatgccagagaggccattcgctggggcactgattatcttctcaaagccactgcacatccaaacaccatttatgttcaggtcagttaagacaacaacactcttgctaaaatcaacttatacccttttgaaagtaactatggaaaagttttttttttcaaatatggggtaagtcttctttagattctgatatttgtttgtttgtttgttatttggctttgcaggtgggagacgctaagaaggaccatgcttgttgggagagaccagaggacatggacacactaagaagcgtgtttaaaatagatgcaaacacacctggttcagaagttgccgcggaaactgctgcagctcttgcagctgcttctcttgtttttagaagaagtgaccccacatactccaaagttttagtgaggagagccatcagggtaagtaaacctttggttcaatttttgctaattattatttttaatagaccaaactgctcccaactaaatcttaaaagagtattttgtttctgagtgtaggtcttccagtttgctgataagtacaggggatcctacagcaatgccttgaaaccttatgtgtgccccttctattgctcttactctggttatcaggtaaagtttcaactgttccatatgagtgatgttgttcagtttagttggatgagacataatcattactcaaattcttttgatgaataattattatggtaggatgagctgttgtggggtgctgcctggctgcacaaggctaccaggaatccaatgtacctaaactacatcaaagttaatggccagatccttggggctgcagagtttgacaacacctttgggtgggataacaagcatgctggagcaagaatacttctttccaaggtacaaattactcatccatcacatgtattctgctgtatatatattgcaaaactagatagaaagtaaaaactaataatggtttgaaatttacatttttgtgttccatggttttaggaattcctggttcaaagggtacaatccctccatgactacaagggtcatgcggacaatttcgtctgttctctaattcctggaacctctttttcttccactcaatataccccaggtgaagtcccttttccttcttaattcttttacttatcctctctcccagtggaaactgcgcgtgtgtgtggtccagtttttgtaacggttacttgcttgttactaggtgggcttcttttcaagatgagtgatagcaacatgcagtatgtcacatccacctccttccttctgctaacatatgccaaatacttgacccaatcccatatgcttgttaactgtggtggaatcacagtaaccccaaggagactccggacaatagctaagaaacaggttacaaaatgtgtctattatatgttgaataagttttaatcaattaaaatactttctatatgattagttaaaaattatttaaaataattaattttgataagttttatttcttaatcaacataactaaatcaggagcagaaaagagaaaattattagatgagaaataaaacaaacaaaaaaaaaattctaaccaattatgaagaacttcttaaacaaagaatgtaaaagagtgccaccagaaaaagtgttgttagatttcctattcttgttagttgaacttgattttttttttaatttttgtaattttaggtggattacttgcttggagacaacccgttgaagatgtcgtacatggtggggtatggtccacggtacccacgaaggatacaccataggggatcatctctaccgtcgattgctgtgcacccgggaaagatccagtgttccgcagggttcagtgtgatgaattcacaatctcccaacccaaacattctagtgggggccattgttggtggaccggatgagcatgataggttcccagatcaacggtcagattatgagcaatcagagccagctacatacattaactcaccccttgtaggagcactggcctatcttgcacactcattcggtcaactctaggaaccaaccatatcgtgtatcccatgttttattttactagtttcgttagttacttacttgtgtctcctagaattatgcacacactcatttgagaccagtacggcagtaccacaccactaagccttataaacttgcttacaagcttaactttactactatgtgtaacttcatattgccgccatgcttttgtaagtattatgatttcggaaagtgttatatatgtatctcccgtgatgtagatgtgtttgattttgcatgttatttgggtgtaaaaaatggattaagtttggaatagctagggtatcggttc

SEQ ID NO: 3 - DNA, 1518 bp, synthetic construct
  source 1..1518: mol_type = other DNA
atgactttctccttttccttcttcaccatcaccactctgctttctctgttctctctaattctgcttcatgccaatgccttcccagtccccatgcatcgccaccctcgctttgccactcataactacagagatgctctcactaaatccattctcttctttgaaggccagaggtcagggaagctccctcctaaccagagaatgtcttggaggagagactctggcctctctgatggctcagccatgcacgttgatttagttggagggtactatgatgctggggacaatgtaaaatttggttttcccatggccttcaccaccaccatgctttcatggagcgttattgagtttggtgggctaatgaaaggtgagttgcagaatgccagagaggccattcgctggggcactgattatcttctcaaagccactgcacatccaaacaccatttatgttcaggtgggagacgctaagaaggaccatgcttgttgggagagaccagaggacatggacacactaagaagcgtgtttaaaatagatgcaaacacacctggttcagaagttgccgcggaaactgctgcagctcttgcagctgcttctcttgtttttagaagaagtgaccccacatactccaaagttttagtgaggagagccatcagggtcttccagtttgctgataagtacaggggatcctacagcaatgccttgaaaccttatgtgtgccccttctattgctcttactctggttatcaggatgagctgttgtggggtgctgcctggctgcacaaggctaccaggaatccaatgtacctaaactacatcaaagttaatggccagatccttggggctgcagagtttgacaacacctttgggtgggataacaagcatgctggagcaagaatacttctttccaaggaattcctggttcaaagggtacaatccctccatgactacaagggtcatgcggacaatttcgtctgttctctaattcctggaacctctttttcttccactcaatataccccaggtgggcttcttttcaagatgagtgatagcaacatgcagtatgtcacatccacctccttccttctgctaacatatgccaaatacttgacccaatcccatatgcttgttaactgtggtggaatcacagtaaccccaaggagactccggacaatagctaagaaacaggtggattacttgcttggagacaacccgttgaagatgtcgtacatggtggggtatggtccacggtacccacgaaggatacaccataggggatcatctctaccgtcgattgctgtgcacccgggaaagatccagtgttccgcagggttcagtgtgatgaattcacaatctcccaacccaaacattctagtgggggccattgttggtggaccggatgagcatgataggttcccagatcaacggtcagattatgagcaatcagagccagctacatacattaactcaccccttgtaggagcactggcctatcttgcacactcattcggtcaactctag

SEQ ID NO: 4 - DNA, 2385 bp, synthetic construct
  source 1..2385: mol_type = other DNA
gacaagtagcaaacttttaggcaattttccaatcatagaatattattgtccattgcaaattgaaaaatagaaacggattcaagtaaaaacttacataataacaactaaaataaaataaaataaagaagcctaagacaatgaccttaaatgcttcgaactatccacggccttggtgcgatgacactagaaattttcaaaacgaatttaaattgttaaattaacttcaaaattgaaaatatgaattcattaactaaaaaaccaaaaaatatcttttattcaattacgattttcatatcaaaattgaaccaaaccatatcatttataatattattatatttttataaaagaaatgtcatttcattataattaaggatttaaggatgtttattttttaaaagttgttcaaggaatataatatcaaaatcttacctttatactgaataaacaaactcatccttataaacaactactttcatatatttaagtaatgtaaaaagtaaactaattaattatcaaagaagctaaatatttgaggaaaatataggaaaaaaatagtttgactaaagaaaaaatatacagaagtgaaattatgtaattttaaaagtattgacctgttaatgtaacattttaaataaatatagtaaaataatagttaattaataaaagaatcacaataatataaataaatcataattttttaaaaattcaaataaaatattatttgttgaaaattcaatccgaattcacacttcaagaaggttacccgatatatcatcctgttataaaaaaataagaaacacgttataacacacactttcatgcacgtgaaaaataaaaataaaatatttttttggtaaatattaattattaatctgttaattttattaatatgagaaattcaacacataatcttttacttgtttattttttcttaaccctcaatcaatcttatatcccttatgataaatcaaacacactaataagtaattcattatttggttaaatactatctcaaaatttgtgcacaacaagttaaatgataaacctaacatgcaccgaatcacccaactattggtgtgtctgtattcctctttaagtacatttatcgttaatccaaaggcgagagagaacagaaagacggaatgttctattcacgtaaacatgcataaaaagataacccaaaaaatgttgttgggtttaatacatttgtttgattatgaatagtgtatttgtattagtaattttattaacttttgtctgtgaaattacttttgtacaaataatatttttaaaaaaaaattacaacaaaaaagtagaaagagaatagaaaatatataataaaatagatgatataataataaaactcttaaatataagatgaaaaataatttataattaattgaagatttaataacttttacactaacattccacgtatattaaacttttaaacaataagacttgactcaatttgtttttggtttcatagctatatattattagtgtaaagttttttaaactaccaaacaattaaaagttatcattaataacacttttaattattataaaaattaataaatttatcatatataataatatgtgattgaataatattataaaattattttacactaaaactgatttacctaaatgttaaagaattttttaactagtaaaaaaaatataaaaattaacttaaatgtactgccaaataagttgggccatttaataacattctctcttattttcttattgtttttacaagaataagttggaccaaatatatttcttaaaaaactttaagaaaggaatataaaaataatgcatggagattatatttttttttaataaataatatgtgcatttgattgtataaagtttttctatgtattttcattcggtcacaattattatatttgctatctttgttgattttataacaattaccttaaaaatcttatgaaagatactttttataagttgacaatgtaagtatgtaactgatttgagttttttaaagtgtcatcgaaatatcagttgttaatattatcccttaccttacattttttaattttaaacttgtatgattgactaattaaatgtctaaaaactttagatttttcttaatttcataatgaaatattaataacattatataatttcaattactaaaatattaatagttaactcttccagttctagtttaacttccccaaacctttgtagactctagctattattaaaaaaatgaaaaaacaaaagtcataacaaaacgctgtagcaggagtaggatgtggacctaattattgttgtattgtaacgaagctcaacgttagagaggcagcttttgctttataaaaaccagagattgaacaaccaaagagc

SEQ ID NO: 5 - DNA, 2790 bp, synthetic construct
  source 1..2790: mol_type = other DNA
tcagtgtggcagtttggtcgttgagtgagtgagtgaggctggtttcactttcacattgcctcttcaaaatgactttctccttttccttcttcaccatcaccactctgctttctctgttctctctaattctgcttcatgccaatgccttcccagtccccatacatcgccaccctcgctttgcagggaagctccctcctaaccagagaatgtcttggaggagagactctggcctctctgatggctcagccatgcacgtatgcatataccaactctcacattaccatgtttttttgtttttgtttttggtttgcttatttatgtgtgaatgtgcaatgcaggttgatttggttggagggtactatgatgctggggacaatgtgaaatttggttttcccatggccttcaccatcaccatgctttcatggagcgttattgagtttggtgggctaatgaaaggtgagttgcagaatgccagagaggccattcgctggggcactgattatcttctcaaagccactgcacatccaaacaccatttatgttcaggtcagttaagacaacaacactcttgctaaaatcaacttatacccttttgaaagtaactatggaaaagtttttattttcaaatatggggtaagtcttctttagattctgatatttgtttgtttgtttgttatttggctttgcaggtgggagacgctaagaaggaccatgcttgttgggagagaccagaggacatggacacactaagaagcgcgtttaaaatagatgcaaacacacctggttcagaagttgccgcggaaactgctgcagctcttgcagctgcttctcttgtttttagaagaagtgaccccacatactccaaagttttagtgaggagagccatcagggtaagtaaacctttggttcaatttttgctaattattatttttaatagaccaaactgctcccaactaaatctcaaaagactattttgtttctgagtgtaggtcttccagtttgctgataagtacaggggatcctacagcaatgccttgaaaccttatgtgtgccccttctattgctcttactctggttatcaggtaaagtttcaactgttccatatgagtgatgttgttcagtttagttggatgagacataatcattactcaaattcttttgatgaataattattatggtaggatgagctgttgtggggtgctgcctggctgcacaaggctaccaggaatccaatgtacctaaactacatcaaagttaatggccagatccttggggctgcagagtttgacaacacctttgggtgggataacaagcatgctggagcaagaatacttctttccaaggtacaaattactcatccatcacatgtattctgctgtatatatattgcaaaactagatagaaagtaaaaactaataatggtttgaaatttacatttttgtgttccatggttttaggaattcctggttcaaagggtacaatccctccatgactacaagggtcatgcggacaatttcgtctgttctctaattcctggaacctctttttcttccactcaatataccccaggtgaagtcccttttccttcttaattcttttacttatcctctctcccagtggaaactgcgcgtgtgtgtggtccagtttttgtaacggttacttgcttgttactaggtgggcttcttttcaagatgagtgatagcaacatgcagtatgtcacatccacctccttcctactgctaacatatgccaaatacttgacccaatcccatatgcttgttaactgtggtggaatcacagtaaccccaaggagactccggacaatagctaagaaacaggttacaaaatgtgtctattatatgttgaataagttttaatcaattaaaatactttctatatgattagttaaaaattatttaaaataattaattttgataagttttatttcttattcaacataactaaatcaggagcagaaaagagaaaattattagatgagaaataaaacaaacaaaaaaaaaattctaaccaattatgaagaacttcttaaacaaagaatgtaagagagtgccaccagaaaaagtgttgttagatttcctattcttgttagttgaacttgatttttttttttaatttttgtaattttaggtggattacttgcttggagacaacccgttgaagatgtcgtacatggtggggtatggtccacggtacccacgaaggatacaccataggggatcatctctaccgtcgattgctgtgcacccgggaaagatccagtgttccgcagggttcagtgtgatgaattcacaatctcccaacccaaacattctagtgggggccattgttggtggaccggatgagcatgataggttcccagatcaacggtcagattatgagcaatcagagccagctacatacattaactcaccccttgtaggagcactggcctatcttgcacactcattcggtcaactctaggaaccaaccatatcgtgtatccaatgttttattttactagtttcgttagttacttacttgtgtctcctagaattatgcacacactcatttgagaccagtacggcagtaccacaccactaagccttataaacttgcttacaagcttaactttactactatgtgtaacttcatattgccgccatgcttttgtaagtattatgatttcggaaagtgttatatatgtatctcccgtgatgtagatgtgtttgattttgcatgttatttgggtgtaaaaaatggattaagtttggaatagctagggtatcggttc

SEQ ID NO: 6 - DNA, 1518 bp, synthetic construct
  source 1..1518: mol_type = other DNA
atgactttctccttttccttcttcaccatcaccactctgctttctctgttctctctaattctgcttcatgccaatgccttcccagtccccatacatcgccaccctcgctttgccactcataactacagagatgctctcactaaatccattctcttctttgaaggccagaggtcagggaagctccctcctaaccagagaatgtcttggaggagagactctggcctctctgatggctcagccatgcacgttgatttggttggagggtactatgatgctggggacaatgtgaaatttggttttcccatggccttcaccatcaccatgctttcatggagcgttattgagtttggtgggctaatgaaaggtgagttgcagaatgccagagaggccattcgctggggcactgattatcttctcaaagccactgcacatccaaacaccatttatgttcaggtgggagacgctaagaaggaccatgcttgttgggagagaccagaggacatggacacactaagaagcgcgtttaaaatagatgcaaacacacctggttcagaagttgccgcggaaactgctgcagctcttgcagctgcttctcttgtttttagaagaagtgaccccacatactccaaagttttagtgaggagagccatcagggtcttccagtttgctgataagtacaggggatcctacagcaatgccttgaaaccttatgtgtgccccttctattgctcttactctggttatcaggatgagctgttgtggggtgctgcctggctgcacaaggctaccaggaatccaatgtacctaaactacatcaaagttaatggccagatccttggggctgcagagtttgacaacacctttgggtgggataacaagcatgctggagcaagaatacttctttccaaggaattcctggttcaaagggtacaatccctccatgactacaagggtcatgcggacaatttcgtctgttctctaattcctggaacctctttttcttccactcaatataccccaggtgggcttcttttcaagatgagtgatagcaacatgcagtatgtcacatccacctccttcctactgctaacatatgccaaatacttgacccaatcccatatgcttgttaactgtggtggaatcacagtaaccccaaggagactccggacaatagctaagaaacaggtggattacttgcttggagacaacccgttgaagatgtcgtacatggtggggtatggtccacggtacccacgaaggatacaccataggggatcatctctaccgtcgattgctgtgcacccgggaaagatccagtgttccgcagggttcagtgtgatgaattcacaatctcccaacccaaacattctagtgggggccattgttggtggaccggatgagcatgataggttcccagatcaacggtcagattatgagcaatcagagccagctacatacattaactcaccccttgtaggagcactggcctatcttgcacactcattcggtcaactctag

SEQ ID NO: 7 - protein, 505 aa, synthetic construct
MTFSFSFFTITTLLSLFSLILLHANAFPVPMHRHPRFATHNYRDALTKSILFFEGQRSGKLPPNQRMSWRRDSGLSDGSAMHVDLVGGYYDAGDNVKFGFPMAFTTTMLSWSVIEFGGLMKGELQNAREAIRWGTDYLLKATAHPNTIYVQVGDAKKDHACWERPEDMDTLRSVFKIDANTPGSEVAAETAAALAAASLVFRRSDPTYSKVLVRRAIRVFQFADKYRGSYSNALKPYVCPFYCSYSGYQDELLWGAAWLHKATRNPMYLNYIKVNGQILGAAEFDNTFGWDNKHAGARILLSKEFLVQRVQSLHDYKGHADNFVCSLIPGTSFSSTQYTPGGLLFKMSDSNMQYVTSTSFLLLTYAKYLTQSHMLVNCGGITVTPRRLRTIAKKQVDYLLGDNPLKMSYMVGYGPRYPRRIHHRGSSLPSIAVHPGKIQCSAGFSVMNSQSPNPNILVGAIVGGPDEHDRFPDQRSDYEQSEPATYINSPLVGALAYLAHSFGQL

SEQ ID NO: 8 - protein, 505 aa, synthetic construct
MTFSFSFFTITTLLSLFSLILLHANAFPVPIHRHPRFATHNYRDALTKSILFFEGQRSGKLPPNQRMSWRRDSGLSDGSAMHVDLVGGYYDAGDNVKFGFPMAFTITMLSWSVIEFGGLMKGELQNAREAIRWGTDYLLKATAHPNTIYVQVGDAKKDHACWERPEDMDTLRSAFKIDANTPGSEVAAETAAALAAASLVFRRSDPTYSKVLVRRAIRVFQFADKYRGSYSNALKPYVCPFYCSYSGYQDELLWGAAWLHKATRNPMYLNYIKVNGQILGAAEFDNTFGWDNKHAGARILLSKEFLVQRVQSLHDYKGHADNFVCSLIPGTSFSSTQYTPGGLLFKMSDSNMQYVTSTSFLLLTYAKYLTQSHMLVNCGGITVTPRRLRTIAKKQVDYLLGDNPLKMSYMVGYGPRYPRRIHHRGSSLPSIAVHPGKIQCSAGFSVMNSQSPNPNILVGAIVGGPDEHDRFPDQRSDYEQSEPATYINSPLVGALAYLAHSFGQL

SEQ ID NO: 9 - DNA, 1713 bp, synthetic construct
  source 1..1713: mol_type = other DNA
gcacccaaccctttatgaacaaagccaatgtgacattgtgaagcttcaagtcatcagagaaagatgaggacaactcattcttcgctcttgcgtctgaactctgtaacccatttaaacatgtttgttggttagtcaaaacggcactgagataggtttcaaaatctttagcttcagaagtgtgaagaaaaccactaacttggttagcagtggcatagatgtttgataagtattctagactttgttcaacaacgaattgccaatcttcaagagcaccaagtgagtattgagacaaagacaagctaccttgaagatatgagtccactaagttcaagaacttacgagcttgggacaaggactttcgaaaagaaatacgaccatagtcgaagatgtttccattttgattagcaagcatggttttgcaataagtagggtctagggtggactcacaaatagtttcgggtggaacaatggaggataaactggtatatgctatggacaatgaggccaagaccgagaaggaaagcatttaggaaacaaacaacttaaggtcaaagatatacttgacagtcattggttttttttttttatgaaggatttagggttgttgttactacggatgaacaaataaggtgcatgactagtttgtcttatatagcggacgaagattttgtgttattataaggattagtgggaatgaactatgggagctttgtcagttagaagtcaacatccaaaaggaatttgtcttggtgtgtttgaagttgttgccatggctaccacgaatgcagtcccaaccggccattttgaaattttttattaggagcttaggaggaacatacattaagtttactataaacgtccatgtatgcaaatattccttgggattatatttattcgtttgaattgatattaaggtttgttttggtacatggtttattaccttgtgacatatatttaatttaattttagtttgtcaacgcgggattggtatccaacaaaaaaacatgtatgtatatattgctggtcatgcttctatttcttatattaataatatttctcttggatattgtattggctcaggaggccctgccccgctattgaggaaattaagatatattcagaatattatcaagtcagacgtaacacatagtcacataactgaaattgaagatcaaatattcatatatgctttgggagcaaagcttttagatttttatgttacttgattttacacatcaatttcacatttcaggttaattttgtatatatctttctataaaactagcccttccaaaagaacaagaaataaactgcaaaaggaattaccatttcagccaaacagaattagcctgaagcaatatatttaaagctttcctaccagaaaggcaggtatgaaataattaagagagtggaaaagggaattgaagctccagtacttgatgcaggagctaaaatgattaaggatctcacgcataacaacacgttgcatactcgagagttcaattaatcgaggtggcatatttaacctagtcagtatgaggtccatgtcacattatgactaaagctagctcatttttttttcaaaaaataaaaataaaaataaaaaaataaaaaataaaatatatatatatatatatatatatatatatatatatatatatatatatatagtttcacagtgtcaacgtgaaacaccccactccatgttacaat

SEQ ID NO: 10 - DNA, 4600 bp, synthetic construct
  source 1..4600: mol_type = other DNA
ccaaccactctatttatccacagcacgtaccctgctagctagttacacatacacagtaatcctaacccacctcctaagtcctatcacatcatgaacaacctcacacttgcatccattctgaccgtgatttcttctctactattctttggaacaactcatttaaccaacacccaaacaacaagagtcccagatcaaaaacataaacacctccatttccaaaagcacatacaagtagtagcccaatccacatgcgaaggaacactctacccagacctatgtgtcttaacactagccacattcccagatctcacaacaaaatctgtcccacaagtgatatcctcagtggtcaaccataccatgtacgaggtaagatcaacgtcctacaactgcagcggcctcaaaaagatgctcaaaaacctcaacccactcgaccagagagccctcgacgactgtctcaaactgtttgaagacaccagcgtcgagctcaaagccaccatcgacgatctctccatcaagagcaccatagggtctaaactgcaccatgacttgcagactctgctgagtggagcaatgaccaacttgtacacgtgcctcgatggctttgcgtacagcaaagggcgcgtgggggacagaatcgagaagaagctgcttcaaatatcgcatcacgtgagcaactcgttggccatgctgaacaaagtgcctggagttgagaaattaacaacttcttcggaatctgatgaggtgtttccagaatatggaaagatgcaaaaggggttcccttcgtgggtgtcctctaaagaccgaaagcttcttcaagctaaagtgaatgagaccaagttcaatcttgttgttgccaaagatggcactggcaacttcaccactataggggaagcactgtctgtggctcccaactcaagcacaactaggtacataacaaaatactttatttcagaccaatgtgattgattataatttacactagtataaatgaattacatagtaaaaacgtagtaccaatgggatacaaattaagagaaaagagaaaggtgttagtagagtgtcaatttttttttaaagatttttaataatgccatgtattcaattggaaatgtatacactatatacagtgagctttttttttttttatgtataagtgaaaagaataaatattaagcattagtcaaaataaaatacagaagtcatatgagttatttaaaaatcacgttaagaaactcattctcacattatatctatgtataattatatatgacttagatgtactgttttcaatctgatgtaagaaactcattcttacattatacctctcctatttgattgtaagaaatatttcttcattgggttgtaaaattaaatgagtgtaagtgataacaggtttgtgatacacgtaacggcgggggcatacttcgagaatgtggaagtgataaggaagaagacgaatctaatgtttgttggagacggtattggaaagacagtagtgaaaggcagtaggaatgttgaggacgggtggaccatttttcaatctgctactgttggtaagtattaaatttagagtgcagcaatgcattcggaaggtccccttaaaaatcggttcgtcagcattcatcgggcagaatatggctctgataccatattaaatttatagtgtagcaaagacattcgaaaaattcccttaaaaaccggtttataaggggtggcctaccgaactatataagtacttatcaaaatttgctaaacatccaatgtaggactattttcaacagtaaatatgtctttcttcttcccaccttctgctactatttttaattagttatgtttttttcttggtttgatattccaactttgaaaaataaaaataggtatgatatatagaatggtcataagtgagatttgattagtagctcggtcacttaataaagttttataaatgctctactttgaggctggcgttgtgattgttaagtcaaagacagagaaaaaaaatcattagaaaaagttagaacaagacaacgaaaatggcaacttgatgcacagcccacatgttggtttaaaaattattaataaactaattttgctaggtgattttgtagttaaaatacataaagtagatgtgggaagatcattttaaatggtttggacagacatggactacttgggtttgtcaagacatcgtgcagttcctgcattgcattgaaaaatagcgttgtagaggttgatgcatgtaattatcttttgttttgttttgtgtgtgtacactagtaaaatcctacctcttgtgagaataagcacaacttagttttgaatgtttggaataaaaccaaggaaagttcaaaagataaaaggctttgtcattttaaaatttttgaggattctgctttccgtgattgaacataatgctgaaaaaagataggtgatgagtgcatgtatatatgtttaaagactattttcaaaggcatcactgcaaatttaaactaccccagctatcaatgacgtatgcatgaaatataaatagtgtcaaagtgatgctagtattattggttacaatgctgactagctagattccaaaatgcttgttttttttttgtgatcattgcccgtcgtcaactattcctgttatgggaataaattttgaagactctaattttgtgttttcagtgttctgtttggtttttccagttatattgcaatccagtaatatcaattggacttctccatcagacaacgggtctcaatcacaatgacattagcaaatatgtattgtaaataaatcaaatttcttatttcttgttaaaggtagcagtaataaacctagatttgggatctcctgcccgaaagcatgttttctttattaatattttccgagctatgagtggtgacaccattttttggaaagaaaatgacgtgagtaatcttggtgaaagatagaaaaagaacagagaaaaaaaacataaccggtgtggtctaatgagaagaaataaaaatagaattggaaactcttttataagacaaaaaaaaaaaaaaatacatgcaggcagaggtaggactatctttggccttagatacatgataattttatttatattttcgcgaaaagtagagttacctctatggttggtcaaagtaaagggtccacatgcctatccacgcccacctaaagatctgcataattctcatcacaaccaaactactttttcagtgccattaggtccactttgcttgttttttcttctatgcttccccttttacttttctgcttttgctcttaacaaataaaaatgctttaatttcctcttttgggataacatggataaaagaaagtgaattttatgatctttattattattcatgtgacaaataatgtgcagtacaaagttaacagacttttagttctctttcttcgtttcaatgatacctttatgttgagtttgtgtctcccagcaaagtttgctctttattgttcatttatgaataatgatccaaaatctaatcctaacagcgttgacaataatggtatgttgcaactattcagttgtaactcagttggcaacacatcttaagtttatgaaaaaaagatctgtgttcaatccttacaagaaaaaggatgaataattttaaatgtaattaagtctcgaacagaatgattatgattgttgttaataaaggaaaatggtatgttgcagctgttgtaggagcaggattcatagcaaagggtataacatttgagaagtcagcaggacccgacaaacaccaagctgtggcactgagaagtggtgtgctgacttctcagctttctaccaatgcagtttcgttggctaccaggacactctctacgtccattccctgcgccaattctaccgtgaacgtgacatttatggcactgtagacttcattttcggcaatgcagctgtggtattccaaaactgcaacttatacgcacgcaagccaaacgaaaatcagaagaacttgttcatggcacaaggcagagaggaccctaaccaaaacactggcatatccatcttgaactgcaagattgcagctgctgcagatttgatccctgtgaaatcctcgttcaagagctacctaggacgtccttggaaaatgtactctatgactgttgtgttaaaatcttacgtggatatagacccagcagggtggttggaatggaatgaaacatttgcattggatacgttgtattatggggagtacatgaataggggtccatgttcaaacacaagtggtagggttacgtggccaggttatagggtcattaacagctccattgaggcaagccaattcacagttggacagtttattcaagacaatgattggttgaacaacactggcatcccattcttctctggtttgagttgagaatatttttaggttgagctgaacctaggagatcacgtgatttgtaaggttacattacattacattcctgtgtattacttcattggagttcctatagtatagggctattgtggctatagcagtcacgtgatgactatacccaaaaattagtcttgtgagggattatatattttagtgctctaataaaagcaactaaccccaccagagatat

SEQ ID NO: 11 - DNA, 1200 bp, synthetic construct
  source 1..1200: mol_type = other DNA
atgaacaacctcacacttgcatccattctgaccgtgatttcttctctactattctttggaacaactcatttaaccaacacccaaacaacaagagtcccagatcaaaaacataaacacctccatttccaaaagcacatacaagtagtagcccaatccacatgcgaaggaacactctacccagacctatgtgtcttaacactagccacattcccagatctcacaacaaaatctgtcccacaagtgatatcctcagtggtcaaccataccatgtacgaggtaagatcaacgtcctacaactgcagcggcctcaaaaagatgctcaaaaacctcaacccactcgaccagagagccctcgacgactgtctcaaactgtttgaagacaccagcgtcgagctcaaagccaccatcgacgatctctccatcaagagcaccatagggtctaaactgcaccatgacttgcagactctgctgagtggagcaatgaccaacttgtacacgtgcctcgatggctttgcgtacagcaaagggcgcgtgggggacagaatcgagaagaagctgcttcaaatatcgcatcacgtgagcaactcgttggccatgctgaacaaagtgcctggagttgagaaattaacaacttcttcggaatctgatgaggtgtttccagaatatggaaagatgcaaaaggggttcccttcgtgggtgtcctctaaagaccgaaagcttcttcaagctaaagtgaatgagaccaagttcaatcttgttgttgccaaagatggcactggcaacttcaccactataggggaagcactgtctgtggctcccaactcaagcacaactaggtttgtgatacacgtaacggcgggggcatacttcgagaatgtggaagtgataaggaagaagacgaatctaatgtttgttggagacggtattggaaagacagtagtgaaaggcagtaggaatgttgaggacgggtggaccatttttcaatctgctactgttgctgttgtaggagcaggattcatagcaaagggtataacatttgagaagtcagcaggacccgacaaacaccaagctgtggcactgagaagtggtgtgctgacttctcagctttctaccaatgcagtttcgttggctaccaggacactctctacgtccattccctgcgccaattctaccgtgaacgtgacatttatggcactgtag

SEQ ID NO: 12 - DNA, 2638 bp, synthetic construct
  source 1..2638: mol_type = other DNA
gcacccaaccctttatgaacaaagccaatgtgacattgtgaagcttcaagtcatcagagaaagatgaggacaactcattcttcgctcttgcgtctgaactctgtaacccatttaaacatgtttgttggttagtcaaaacggcactgagataggtttcaaaatctttagcttctgaagtgtgaagaaaaccactaactcggttagcagtggcatagatgtttgataagtattctagactttgttcaacaacgaattgccaatcttcaagagcaccaagtgagtattgagacaaagacaagctaccttgaagatatgagtccactaagttcaagaacttacgagcttgggacaaggactttcgaaaagaaatacgaccatagtcgaagatgtttccattttgattagcaagcatggttttgcaataagtagggtctagggtggactcacaaatagtttcgggtggaacaatggaggataaactggtatatgctatggacaatgagaccaagaccgagaaggaaagcatttaggaaaaaaacaacttaaggtcaaagatatacttgacagtcattagttttctcttttgtttttcttcgtatgcttgtaaggaacccatgagttgctcaatagtcatggtctttaaatccttgttttcttcaatgttggtaacaatgaagtcaaaacttggatttaaagttcgaagtattttttccatgaccttcacctcatcaacattttcaccatttcttttaagttgattgactacggccaatactcgagaaaaataatcagaaattgactcggactcctccataaacaaacgctcaaagtcacctctaagagtttgaagacgaatcttttttacctgctcaactcctttgttgcaagtttgaagcttatcccatgcttctttggccgtcgttgcgttggatatcttctcaaatgtatcttcatccaccgattgataaatgagaaagagagctttcttgtctctctttcttgactccttcaacgtctcctttacaccttggcttagcgagacttcatcttgctcctcgaagccattctctacgatatcccacacatcttgagctcctagtagcgccttcatcttgatactccaattatcatagttgttctttgtgagcatcggcatttggaaatgaaaacctccattcgccatcttttgaggatcttgaagctctgataccactttgttggaaataaggctttttatgtttaggaaaagtgtttaggaatattggagactttgaatagaaacttgataggaaggagaattctttatggaggagagaactttgtatttttgcttgatacaaatgtgtaggattacatctctatttatactactctaaggagaactctagacacactaattctagagagttctcaactctagagatccaaagagtattctagagaatattaaaaccataagaaatatctagacattccaaacactacaagaattctctagaaacatgacccataattacttaagcccaaaataactaagtccaagaaaccaaataattaatttgggcccaaatcaagtttatatttcaacacaaataaggtgcatgactagtttgtcttatatagcggacgaagattttgtgttattataaggattagtgggaatgaactatgggagctttgtcagttagaagtcaacatccaaaaggaatttgtcttgtttgtttgaagttgttgccatggctaccacgaatgcagtcccaaccggccattttgaaattttttattaggagcttaggaggaacatacattaagtttactataaacgtccatgtatgcaaatattccttgggattatatttattcgtttgaattgatattaaggtttgttttggtacatggtttattaccttgtgacatatatttaatttaattttagtttgtcaacgcgggattggtatccaacaaaaaaacatgtatgtatatattgctggtcatgcttctatttcttatattaataatatttctcttggatattgtattggctcaggaggccctgccccgctattgaggaaattaagatatattcagaatattatcaagtcagacgtaacacatagtcacataactgaaattgaagatcaaatattcatatatgctttgggagcaaagcttttagatttttatgttacttgattttacacatcaatttcacatttcaggttaattttgtatatatctttctataaaactagcccttccaaaagaacaagaaataaactgcaaaaggaattaccatttcagccaaacagaattagcctgaagcaatatatttaaagctttcctaccagaaaggcaggtatgaaataattaagagagtggaaaagggaattgaagctccagtacttgatgcaggagctaaaatgattaaggatctcacgcataacaacacgttgcatactcgagagttcaattaatcgaggtggcatatttaacctagtcagtatgaggtccatgtcacattatgactaaagctagctcattttttttcaaaaaaaaaaaaatatatatatatatatatatatatatatatatatagtttcacagtgtcaacgtgaaacaccccactccatgttacaat

SEQ ID NO: 13 - DNA, 4331 bp, synthetic construct
  source 1..4331: mol_type = other DNA
ccaaccactctatttatccacagcacgtaccctgctagctagttacacatacacagtaatcctaacccacctcctaagtcctatcacatcatgaacaacctcacacttgcatccattctgaccgtgatttcttctctactattctttggaacaactcatttaaccaacacccaaacaacaagagtcccagatcaaaaacataaacacctccatttccaaaagcatatacaagtagtagcccaatccacatgcgaaggaacactctacccagacctatgtgtcttaacactagccacattcccagatctcacaacaaaatctgtcccacaagtgatatcctcagtggtcaaccataccatgtacgagataagatcaacgtcctacaactgcagcggcctcaaaaagatgctcaaaaacctcaacccactcgaccagagagccctcgactactgtctcaaactgtttgaagacaccagcgtcgagctcaaagccaccatcgacgatctctccatcaagagcaccatagggtcgaaacggcatcatgacttgcagactctgctgagtggagcaatgaccaacttgtacacgtacctcgatggctttgcgtacagcaaagggcgcgtgggggacagaatcgagaagaagctgcttcaaatatcgcatcacgtgagcaactcgttggccatgctgaacaaagtgcctggagttgagaaattaacaacttcttcggattctgatgaggtgtttccagaatatggaaagatgcaaaaggggttcccttcgtgggtgtcctccaaagaccgaaagcttctttaagctaaagtgaatgagaccaagttcaatcttgttgttgccaaagatggcactggcaacttcaccactataggggaagcactgtctgtggctcccaactcaagcacaactaggtacataacaaaatactttatttcagaccaatgtgattgattataatttactctagtataaatgaattacatagtaaaaacgtagtaccaatgggatacaaattaagagaaaagagaaaggtgttagtagagtgtcaatttttttttaaagatttttaataatgccatgtattcaattggaaatgtatacactatatacagtgagctttttttttttttttttatgtataagtgaaaagaataaatattaagcattagtcaaaataaaatacagaagtcatatgagttatttaaaaatcacgttaagaaactcattctcacattatatctatgtataattatatatgacttagatgtactgttttcaatccgatgtaagaaactcattcttacattatacctctcctatttgattgtaagaaatatttcttcattgggttgtaaaattaaatgagtgtaagtgataacaggtttgtgatacacgtaacggcgggggcatacttcgagaatgtggaagtgataaggaagaagacgaatctaatgtttgttggagacggtattggaaagacagtagtgaaaggcagtaggaatgttgaggacgggtggacaatttttcaatctgctactgttggtaagtatgtctttcttcttcccaccttctgctactatttttaattagttatgtttttttcttggtttgatattccaactttgaaaaataaaaataggtatgatatatagaatggtcataagtgagatttgattagtagctcggtcacttaataaagttttataaatgctctactttgaggctggcgttgtgattgttaagtcaaagacagaaaaaaaatcattagaaaaagttagaacaagacaacgaaaatggcaacttgatgcacagcccacatgttggtttaaaaattattaataaactaattttgctaggtgattttgtagttaaaatacataatgtagatgtgggaagatcattttaaatggtttggacagacatggactacttgggtttgtcaagacatcgtgcagttcctgcattgcattgaaaaatagtgttgtagaggttgatgcatgtaattattttttttttgttttgtgtgtgtacactagtaaaatcctacctctggtgagaataagcacaacttagttttgaatgtttggaataaaaccaaggaaagttcaaaagataaaaggctttgccattttaaagactaggtgatgagtacatgtatatatgtttaaagactattttcaaaggcatcactgcaaatttaaactaccccagctatcaatgacgtatgcatgaaatataaatagtgtcaaagtgatgctagtattattggttacaatgctgactagctagattccaaaatggttgttttttttttttatcattgcccgttgtcaactattcctgttatgggaataaattttgaggactctaattttgtgttttcagtgttctgtttggtttttccagttatattgcaatccagtaatatcaattggacttctccatcagacaacgcgtctcaatcacaatgacattagcaaatatatattgtaaataaatcaaatttcttatttcttgttaaagttagcagtaataaacctagatttgggatctcctgcccgaaagcatgttttctttattaatattttccgagctatgagtggtgacaccattttttggaaagaaaatgacgtgagtaatcttggtgaaagatagaaaaagaacagagaaaaaaaacataaccggtgtggtctaatgagaagaaataaaaatagaattagaaactcttttataagaccaaaaaaaaaaaaaatacatgcaggcaggcagaggaggtaggactatctctggccttagatacatgataattttatttatattttcgcgaaaagtagagttacctctatggttggtcaaagtaaagggtccacatgcctatccacgcccacctaaagatctgcataattctcatcacaaccaaactactttttcagtgccattaggtccactttgcttgttttttcttctatgcttccccttttacttttctgcttttgcttttaacaaataaaaatgctttaatttcctcttttgggataacatggataaaagaaagtgaattttatgatctttattattcatgtgacaaataatgtgcagtacaaagttaacagacttttagttttctttcttcgtttcaatgatacctttatgttgagtttgtgtctctcagcaaagtttgctctttattgttcatttatgaataatgatccaaaatctaatcctaacagcgttgacaataatggtatgttgcaactattcagttgtaactcagttggcaacagatcttaagtttatggaaaaaaaatctgtgttcaatccttacaagaaaaaggatgaataatttaaaatgtaattaagtctcgaacagaatgattatgattgttgttaataaaagaaaatggtatgttgcagctgttgtaggagcaggattcatagcaaagggtataacatttgagaagtcagcaggacccgacaaacaccaagctgtggcactgagaagtggtgtgctgacttctcagctttctaccaatgcagtttcgttggctaccaggacactctctacgtccattccctgcgtcaattctaccgtgaacgtgacatttatggcactgtagacttcattttcggcaatgcagctgtggtattccaaaactgcaacttatacgcacgcaagccaaacgaaaatcagaagaacttgttcatggcacaaggcagagaggaccctaaccaaaacactggcatatccatcttgaactgcaagattgcagctgctgcagatttgatccctgtgaaatcctcgttcaagagctacctaggacgtccttggaaaatgtactctatgactgttgtgttaaaatcttacgtggatatagacccagcagggtggttggaatggaatgaaacatttgcattggatacgttgtattatggggagtacatgaataggggtccatgttcaaacacaagtggtagggttacgtggccaggttatagggtcattaacagctccattgaggcaagccaattcacagttggacagtttattcaagacaatgattggttgaacaacactggcatcccattcttctctggtttgagttgagaatatttttaggttgagctgaacctaggagatcacgtgatttgtaaggttacattacattacattcctgtgtattacttcattggagttcctatagtatagggctattgtggctatagcagtcacgtgatgactatacccaaaaattagtcttgtgagggattatatattttagtgctctaataaaagcaactaaccccaccagagatat

SEQ ID NO: 14 - DNA, 1200 bp, synthetic construct
  source 1..1200: mol_type = other DNA
atgaacaacctcacacttgcatccattctgaccgtgatttcttctctactattctttggaacaactcatttaaccaacacccaaacaacaagagtcccagatcaaaaacataaacacctccatttccaaaagcatatacaagtagtagcccaatccacatgcgaaggaacactctacccagacctatgtgtcttaacactagccacattcccagatctcacaacaaaatctgtcccacaagtgatatcctcagtggtcaaccataccatgtacgagataagatcaacgtcctacaactgcagcggcctcaaaaagatgctcaaaaacctcaacccactcgaccagagagccctcgactactgtctcaaactgtttgaagacaccagcgtcgagctcaaagccaccatcgacgatctctccatcaagagcaccatagggtcgaaacggcatcatgacttgcagactctgctgagtggagcaatgaccaacttgtacacgtacctcgatggctttgcgtacagcaaagggcgcgtgggggacagaatcgagaagaagctgcttcaaatatcgcatcacgtgagcaactcgttggccatgctgaacaaagtgcctggagttgagaaattaacaacttcttcggattctgatgaggtgtttccagaatatggaaagatgcaaaaggggttcccttcgtgggtgtcctccaaagaccgaaagcttctttaagctaaagtgaatgagaccaagttcaatcttgttgttgccaaagatggcactggcaacttcaccactataggggaagcactgtctgtggctcccaactcaagcacaactaggtttgtgatacacgtaacggcgggggcatacttcgagaatgtggaagtgataaggaagaagacgaatctaatgtttgttggagacggtattggaaagacagtagtgaaaggcagtaggaatgttgaggacgggtggacaatttttcaatctgctactgttgctgttgtaggagcaggattcatagcaaagggtataacatttgagaagtcagcaggacccgacaaacaccaagctgtggcactgagaagtggtgtgctgacttctcagctttctaccaatgcagtttcgttggctaccaggacactctctacgtccattccctgcgtcaattctaccgtgaacgtgacatttatggcactgtag

SEQ ID NO: 15 - protein, 399 aa, synthetic construct
MNNLTLASILTVISSLLFFGTTHLTNTQTTRVPDQKHKHLHFQKHIQVVAQSTCEGTLYPDLCVLTLATFPDLTTKSVPQVISSVVNHTMYEVRSTSYNCSGLKKMLKNLNPLDQRALDDCLKLFEDTSVELKATIDDLSIKSTIGSKLHHDLQTLLSGAMTNLYTCLDGFAYSKGRVGDRIEKKLLQISHHVSNSLAMLNKVPGVEKLTTSSESDEVFPEYGKMQKGFPSWVSSKDRKLLQAKVNETKFNLVVAKDGTGNFTTIGEALSVAPNSSTTRFVIHVTAGAYFENVEVIRKKTNLMFVGDGIGKTVVKGSRNVEDGWTIFQSATVAVVGAGFIAKGITFEKSAGPDKHQAVALRSGVLTSQLSTNAVSLATRTLSTSIPCANSTVNVTFMAL

SEQ ID NO: 16 - protein, 241 aa, synthetic construct
MNNLTLASILTVISSLLFFGTTHLTNTQTTRVPDQKHKHLHFQKHIQVVAQSTCEGTLYPDLCVLTLATFPDLTTKSVPQVISSVVNHTMYEIRSTSYNCSGLKKMLKNLNPLDQRALDYCLKLFEDTSVELKATIDDLSIKSTIGSKRHHDLQTLLSGAMTNLYTYLDGFAYSKGRVGDRIEKKLLQISHHVSNSLAMLNKVPGVEKLTTSSDSDEVFPEYGKMQKGFPSWVSSKDRKLL

SEQ ID NO: 17 - DNA, 116 bp, synthetic construct
  source 1..116: mol_type = other DNA
gaaggtgaccaagttcatgctttgtcttccagatccgagtcgattggaaggtcggagtcaacggatttgtcttccagatccgagtcgatagatgaaattatacaccgtggggcctt

SEQ ID NO: 18 - DNA, 112 bp, synthetic construct
  source 1..112: mol_type = other DNA
gaaggtgaccaagttcatgctggtgtcggtgatgcggtgaattgaaggtcggagtcaacggattggtgtcggtgatgcggtgaatagatgaaattatacaccgtggggcctt

SEQ ID NO: 19 - DNA, 119 bp, synthetic construct
  source 1..119: mol_type = other DNA
gaaggtgaccaagttcatgcttaagtggtgtcgttaataatgattttcgaaggtcggagtcaacggatttaagtggtgtcgttaataatgatttttaccatcgtcaaccaactaatgtt

SEQ ID NO: 20 - DNA, 110 bp, synthetic construct
  source 1..110: mol_type = other DNA
gaaggtgaccaagttcatgctgctccatgaaagcatggtgggaaggtcggagtcaacggattgctccatgaaagcatggtgacgtatgcatataccaactctcacattac

SEQ ID NO: 21 - DNA, 121 bp, synthetic construct
  source 1..121: mol_type = other DNA
gaaggtgaccaagttcatgctgaacttggtctcattcactttagcttagaaggtcggagtcaacggattaacttggtctcattcactttagcttgtgtcctcyaaagaccgaaagcttctt

SEQ ID NO: 22 - DNA, 100 bp, synthetic construct
  source 1..100: mol_type = other DNA
gaaggtgaccaagttcatgctcgctcaagatgtcgagcaggaaggtcggagtcaacggattcgctcaagatgtcgagcacgatggtggtgaagttcccgt

SEQ ID NO: 23 - DNA, 109 bp, synthetic construct
  source 1..109: mol_type = other DNA
gaaggtgaccaagttcatgcttatcattggcggacctacatgtgaaggtcggagtcaacggatttatcattggcggacctacatgcaattagtaacggacccaagatcc

SEQ ID NO: 24 - DNA, 106 bp, synthetic construct
  source 1..106: mol_type = other DNA
gaaggtgaccaagttcatgctccaaccaaccttatatctccgtgaaggtcggagtcaacggattccaaccaaccttatatctccgcttggcaccaaacttcccatc

SEQ ID NO: 25 - DNA, 21 bp, synthetic construct
  source 1..21: mol_type = other DNA
atgactttctccttttccttc

SEQ ID NO: 26 - DNA, 19 bp, synthetic construct
  source 1..19: mol_type = other DNA
ctagagttgaccgaatgag

SEQ ID NO: 27 - DNA, 22 bp, synthetic construct
  source 1..22: mol_type = other DNA
atgaacaacctcacacttgcat

SEQ ID NO: 28 - DNA, 24 bp, synthetic construct
  source 1..24: mol_type = other DNA
ataaatgtcacgttcacggtagaa

SEQ ID NO: 29 - DNA, 21 bp, synthetic construct
  source 1..21: mol_type = other DNA
cccgttcatttcaatcacccc

SEQ ID NO: 30 - DNA, 22 bp, synthetic construct
  source 1..22: mol_type = other DNA
ccctccaaccaaatcaacatgc

SEQ ID NO: 31 - DNA, 19 bp, synthetic construct
  source 1..19: mol_type = other DNA
aatgccttcccagtcccca

SEQ ID NO: 32 - DNA, 22 bp, synthetic construct
  source 1..22: mol_type = other DNA
ccctccaactaaatcaacgtgc

SEQ ID NO: 33 - DNA, 21 bp, synthetic construct
  source 1..21: mol_type = other DNA
tcggaatctgatgaggtgttt

SEQ ID NO: 34 - DNA, 22 bp, synthetic construct
  source 1..22: mol_type = other DNA
ttggcaacaacaagattgaact

SEQ ID NO: 35 - DNA, 21 bp, synthetic construct
  source 1..21: mol_type = other DNA
atgaatgacggttcccatgta

SEQ ID NO: 36 - DNA, 21 bp, synthetic construct
  source 1..21: mol_type = other DNA
ggcattaaggcagctcactct

SEQ ID NO: 37 - DNA, 20 bp, synthetic construct
  source 1..20: mol_type = other DNA
gaccttcttcgtttctcgca

SEQ ID NO: 38 - DNA, 20 bp, synthetic construct
  source 1..20: mol_type = other DNA
cgaacctctcaatcacacgc

SEQ ID NO: 39 - DNA, 21 bp, synthetic construct
  source 1..21: mol_type = other DNA
gtctttctccatcgtcctcac

SEQ ID NO: 40 - DNA, 21 bp, synthetic construct
  source 1..21: mol_type = other DNA
tgacccaatcccatatgcttg

SEQ ID NO: 41 - DNA, 21 bp, synthetic construct
  source 1..21: mol_type = other DNA
aagataggccagtgctcctac

SEQ ID NO: 42 - DNA, 21 bp, synthetic construct
  source 1..21: mol_type = other DNA
tgtaggtcttccagtttgctg

SEQ ID NO: 43 - DNA, 21 bp, synthetic construct
  source 1..21: mol_type = other DNA
gagaacagacgaaattgtccg

SEQ ID NO: 44 - DNA, 21 bp, synthetic construct
  source 1..21: mol_type = other DNA
aaaacgctgtagcaggagtag

SEQ ID NO: 45 - DNA, 20 bp, synthetic construct
  source 1..20: mol_type = other DNA
ttcatcctgactaacaaccg

SEQ ID NO: 46 - DNA, 20 bp, synthetic construct
  source 1..20: mol_type = other DNA
tgtgagatctgggaatgtgg

SEQ ID NO: 47 - DNA, 20 bp, synthetic construct
  source 1..20: mol_type = other DNA
cacagcacgtaccctgctag

SEQ ID NO: 48 - DNA, 22 bp, synthetic construct
  source 1..22: mol_type = other DNA
gcagaaggtgggaagaagaaag

SEQ ID NO: 49 - DNA, 20 bp, synthetic construct
  source 1..20: mol_type = other DNA
cggttcgtcagcattcatcg

SEQ ID NO: 50 - DNA, 22 bp, synthetic construct
  source 1..22: mol_type = other DNA
gtctgttaactttgtactgcac

SEQ ID NO: 51 - DNA, 55 bp, synthetic construct
  source 1..55: mol_type = other DNA
ggggacaagtttgtacaaaaaagcaggcttagacaagtagcaaacttttaggcaa

SEQ ID NO: 52 - DNA, 52 bp, synthetic construct
  source 1..52: mol_type = other DNA
ggggacaagtttgtacaaaaaagcaggcttaacctaacatgcaccgaatcac

SEQ ID NO: 53 - DNA, 54 bp, synthetic construct
  source 1..54: mol_type = other DNA
ggggaccactttgtacaagaaagctgggtagctctttggttgttcaatctctgg

SEQ ID NO: 54 - DNA, 36 bp, synthetic construct
  source 1..36: mol_type = other DNA
tgcttagggcccgacaagtagcaaacttttaggcaa

SEQ ID NO: 55 - DNA, 41 bp, synthetic construct
  source 1..41: mol_type = other DNA
tgcttaggcgcgcctttgaagaggcaatgtgaaagtgaaac

SEQ ID NO: 56 - DNA, 35 bp, synthetic construct
  source 1..35: mol_type = other DNA
tgcttaggcgcgccatgactttctccttttccttc

SEQ ID NO: 57 - DNA, 31 bp, synthetic construct
  source 1..31: mol_type = other DNA
tgcttacctaggctagagttgaccgaatgag

SEQ ID NO: 58 - DNA, 25 bp, synthetic construct
  source 1..25: mol_type = other DNA
gattgacacacgcgcagtttccact

SEQ ID NO: 59 - DNA, 25 bp, synthetic construct
  source 1..25: mol_type = other DNA
aaacagtggaaactgcgcgtgtgtc

SEQ ID NO: 60 - DNA, 25 bp, synthetic construct
  source 1..25: mol_type = other DNA
gattgacctggttcagaagttgccg

SEQ ID NO: 61 - DNA, 25 bp, synthetic construct
  source 1..25: mol_type = other DNA
aaaccggcaacttctgaaccaggtc

SEQ ID NO: 62 - DNA, 25 bp, synthetic construct
  source 1..25: mol_type = other DNA
tggggtaagtcttctttagattctg

SEQ ID NO: 63 - DNA, 21 bp, synthetic construct
  source 1..21: mol_type = other DNA
tgacccaatcccatatgcttg

SEQ ID NO: 64 - DNA, 21 bp, synthetic construct
  source 1..21: mol_type = other DNA
aagataggccagtgctcctac

SEQ ID NO: 65 - DNA, 21 bp, synthetic construct
  source 1..21: mol_type = other DNA
gtctttctccatcgtcctcac

SEQ ID NO: 66 - DNA, 21 bp, synthetic construct
  source 1..21: mol_type = other DNA
gagaacagacgaaattgtccg

SEQ ID NO: 67 - DNA, 18 bp, synthetic construct
  source 1..18: mol_type = other DNA
tttgccaaataagttggg

SEQ ID NO: 68 - DNA, 11 bp, synthetic construct
  source 1..11: mol_type = other DNA
ttattaatttt

SEQ ID NO: 69 - DNA, 13 bp, synthetic construct
  source 1..13: mol_type = other DNA
aataacactttta

SEQ ID NO: 70 - DNA, 21 bp, synthetic construct
  source 1..21: mol_type = other DNA
acaacaaaaaagtagaaagag

SEQ ID NO: 71 - DNA, 11 bp, synthetic construct
  source 1..11: mol_type = other DNA
tatttttttgg

SEQ ID NO: 72 - DNA, 11 bp, synthetic construct
  source 1..11: mol_type = other DNA
ttaattaataa

SEQ ID NO: 73 - DNA, 13 bp, synthetic construct
  source 1..13: mol_type = other DNA
ctatatattatta

SEQ ID NO: 74 - DNA, 30 bp, synthetic construct
  source 1..30: mol_type = other DNA
cacacctggttcagaagttgccgcggaaac

SEQ ID NO: 75 - DNA, 30 bp, synthetic construct
  source 1..30: mol_type = other DNA
cacacctggttcnnnnnnnnnnnnnnnnnn

SEQ ID NO: 76 - DNA, 11 bp, synthetic construct
  source 1..11: mol_type = other DNA
cacacctggtt

SEQ ID NO: 77 - DNA, 10 bp, synthetic construct
  source 1..10: mol_type = other DNA
ccgcggaaac

SEQ ID NO: 78 - DNA, 23 bp, synthetic construct
  source 1..23: mol_type = other DNA
cacacctggttcagaagttcccc

SEQ ID NO: 79 - DNA, 17 bp, synthetic construct
  source 1..17: mol_type = other DNA
cacacctggttcagaag

SEQ ID NO: 80 - DNA, 10 bp, synthetic construct
  source 1..10: mol_type = other DNA
ccgctcagcc

SEQ ID NO: 81 - DNA, 30 bp, synthetic construct
  source 1..30: mol_type = other DNA
cacacctggttcagaagttgnnnnnnnnnn

SEQ ID NO: 82 - DNA, 16 bp, synthetic construct
  source 1..16: mol_type = other DNA
cacacctggttcagaa

SEQ ID NO: 83 - DNA, 30 bp, synthetic construct
  source 1..30: mol_type = other DNA
cacacctggttcagaagttgactgggagag

SEQ ID NO: 84 - DNA, 18 bp, synthetic construct
  source 1..18: mol_type = other DNA
cacacctggttcagaagt

SEQ ID NO: 85 - DNA, 19 bp, synthetic construct
  source 1..19: mol_type = other DNA
cacacctggttcagaagtt

SEQ ID NO: 86 - DNA, 30 bp, synthetic construct
  source 1..30: mol_type = other DNA
cacacctggttcagaannnnnnnnnnnnnn

SEQ ID NO: 87 - DNA, 30 bp, synthetic construct
  source 1..30: mol_type = other DNA
cacacctggttcagaagttgccnnnnnnnn

SEQ ID NO: 88 - DNA, 19 bp, synthetic construct
  source 1..19: mol_type = other DNA
ctcacctggttcagaagtt

SEQ ID NO: 89 - DNA, 30 bp, synthetic construct
  source 1..30: mol_type = other DNA
cacacctggttcagaagtnnnnnnnnnnnn

(19) United States
(12) Patent Application Publication
(10) Pub. No.: US 2026/0132416 A1
(43) Pub. Date: May 14, 2026

(54) Compositions And Methods For Increasing Resistance To Nematodes In Plants

(71) Applicant: University of Georgia Research Foundation, Inc., Athens, GA (US)

(72) Inventors: Melissa G. Mitchum, Athens, GA (US); Kelly Goode, Athens, GA (US); Zenglu Li, Athens, GA (US); Wayne Parrott, Athens, GA (US)

(21) Appl. No.: 19/385,846

(22) Filed: Nov. 11, 2025

Related U.S. Application Data
(60) Provisional application No. 63/719,027, filed on Nov. 11, 2024.

(51) Int. Cl. C12N 15/82; C12N 9/22; C12N 15/11

(52) U.S. Cl. CPC C12N 15/8285; C12N 9/222; C12N 15/111; C12N 15/8201; C12N 15/8218; C12N 2310/20; C12Y 302/01004

(57) ABSTRACT

Root-knot nematodes (RKN) are pathogens affecting soybean production worldwide. Rmi1 (Resistance to M. incognita 1) is an additive gene for M. incognita resistance identified in soybean cv. Forrest, which resides in a major QTL on Chromosome 10. It has been established that Rmi1 is associated with multiple polymorphisms in the promoter and sequences of genes encoding β-1,4-endoglucancase (EG) and pectin methylesterase (PME1), both of which exhibit higher expression in susceptible than in Rmi1 resistant plants. EG expression is correlated with RKN feeding cell formation, indicating EG functions as a susceptibility factor for soybean resistance to M. incognita. Plants, including genetically modified and transgenic plants, cells and seeds thereof that lack expression or functional gene products of EG and/or PME1 are provided. Compositions and methods for reducing or removing EG and/or PME1 expression in plants are also described.

### CROSS-REFERENCE TO RELATED APPLICATIONS

This application claims benefit of U.S. Provisional Application No. 63/719,027, filed Nov. 11, 2024, is hereby incorporated herein by reference in its entirety.

### STATEMENT REGARDING FEDERALLY SPONSORED RESEARCH OR DEVELOPMENT

This invention was made with Government Support under 1842396 awarded by the National Science Foundation. The government has certain rights in the invention.

### REFERENCE TO SEQUENCE LISTING

The Sequence Listing XML submitted as a file named “UGA_2025-098-02_US_ST26.xml,” created on Nov. 10, 2025, and having a size of 110,857 bytes is hereby incorporated by reference pursuant to 37 C.F.R. § 1.834(c)(1).

### FIELD OF THE INVENTION

The field of the invention is generally related to compositions and methods for increasing nematode resistance in plants.

### BACKGROUND OF THE INVENTION

Root-knot nematodes (RKN) are the most economically dangerous pathogen threat to soybean production in the southeast US, accounting for more than an estimated $165 million yield loss (2.4%) in 2023 (Crop Protection Network 2024). A 2021 soil survey of Georgia soybean fields identified RKN in 49% of tested samples (Mitchum et al. 2023). Meloidogyne incognita was by far the most prevalent RKN species identified, being found in 80% of RKN+ samples.

The primary methods for combatting RKN are crop rotation, nematicides, and genetic resistance. The wide host range of RKN, especially that of M. incognita, makes crop rotation unlikely to successfully combat RKN. Of the three most reported crops to be rotated with soybean in Georgia, cotton, corn, and peanut, both cotton and corn are compatible hosts for M. incognita (Mitchum et al. 2023). Nematicides are available and effective, but are less desirable due to their high toxicity, and the regulation of available nematicides makes them problematic as a standard defense against RKN. In addition, the extensive host range of RKN makes devising a crop rotation pattern that gives sufficient time between compatible hosts difficult.

Plant breeding programs have attempted the introgression of host plant resistance in elite cultivars. From an agricultural perspective, host plant resistance is found in individual genotypes that suffer less damage from certain nematodes than other genotypes of that crop. However, these efforts have been hampered by the lack of functional understanding of the genetic basis of resistance to most parasites, and by the diverse ways in which parasites cause damage to plants. Genetic resistance is available, effective, and environmentally friendly making it the best option for RKN control.

The first locus for resistance to RKN in soybean was identified from a cross between the susceptible cv. Bossier and the moderately resistant cv. Forrest (Luzzi et al. 1994). Forrest was selected for the study because, at the time, it represented the highest level of RKN resistance commercially available. Screening of F2 plants showed a 1:2:1 segregation ratio of susceptible:intermediate:resistant responses to M. incognita, indicating a single additive gene conferred RKN resistance in Forrest, named Resistance to M. incognita-1 (Rmi1). Pedigree analysis of forty-eight soybean lines showed that Forrest and its relatives initially inherited resistance from the line Palmetto (Ha et al. 2004).

Additional germplasm screening later identified PI 96354, a better resistant parent for use in breeding. PI 96354 was found to have fewer galls and eggs per root system than Forrest after M. incognita infection (Luzzi et al. 1987). The value of PI 96354 was further confirmed in a field microplot study, showing that PI 96354 had <1% yield suppression when exposed to M. incognita, with Bossier and Forrest showing 97% and 12% yield suppression, respectively (Herman et al. 1990).

An investigation into the specific mechanisms of M. incognita resistance showed that Rmi1 resistance functioned primarily in the emigration of J2s from soybean roots in the days following penetration (Herman et al. 1991). The more resistant PI 96354 had additional resistance stemming from delayed development of the nematodes remaining in the root and decreased fecundity of the few female nematodes making it to egg production.

RFLP mapping of M. incognita resistance used 101 F2:3 Bossier x PI 96354 lines, identifying a major additive QTL on chromosome 10 and a minor dominant QTL on chromosome 18 (Tamulonis et al. 1997). Since the chromosome 10 QTL was predicted to have additive action, it was hypothesized that this region correlated to Rmi1 from Forrest but was never independently verified. This study was hampered by a lack of polymorphic markers in the mapped region. Li et al. (2001) utilized the same mapping population to more finely map the region once SSR markers were available. This study identified a major QTL between markers Satt358 and Satt492.

After the sequencing of the soybean genome, new avenues for narrowing the candidate region were available. A population of 188 F5:6 RILs from a Bossier x PI 96354 cross were evaluated for M. incognita resistance and genotyped using twenty-eight SSR markers between Satt358 and Satt492. This identified a 235 kb region bordered by BARCSOYSSR10-0090 and BARCSOYSSR10-0105, an interval containing thirty annotated genes (Pham et al. 2013). However, in spite of the significant advances in the field, the precise bases for genetic nematode resistance remains elusive.

Accordingly, it is an object of the invention to provide compositions and methods for improving a plant's resistance to nematode parasites, preferably while maintaining a high level of agronomic performance.

### BRIEF SUMMARY OF THE INVENTION

Compositions and methods for increasing pest resistance in a plant are disclosed. It has been discovered that altered expression of the protein encoded by the β-1,4-endoglucancase (EG) and/or pectin methylesterase (PME1) genes in soybean increases pest resistance in plants.

A plant or plant cell including a genetic modification that reduces, inhibits or silences expression or translation of a target polynucleotide having a nucleic acid sequence of
(i) any one of SEQ ID NOs:1-3, or a complement thereof, and/or a nucleic acid encoding the polypeptide of SEQ ID NO:7 or 8, or a complement thereof; and/or
(ii) any one of SEQ ID NOs:9-11, or a complement thereof, and/or a nucleic acid encoding the polypeptide of SEQ ID NO:15 or 16, or a complement thereof is provided. Typically, the plant is a transgenic or non-transgenic plant. In some forms, the genetic modification is induced by gene editing technology optionally selected from CRISPR/Cas, TALENs, and Zinc Finger Nucleases.

A transgenic plant or transgenic plant cell, including a polynucleotide including an expression control sequence operably linked to a nucleic acid sequence encoding an antisense nucleic acid that reduces, inhibits or silences expression or translation of a target polynucleotide having a nucleic acid sequence of
(i) any one of SEQ ID NOs:1-3, or a complement thereof, and/or a nucleic acid encoding the polypeptide of SEQ ID NO:7 or 8, or a complement thereof; and/or
(ii) any one of SEQ ID NOs:9-11, or a complement thereof, and/or a nucleic acid encoding the polypeptide of SEQ ID NO:15 or 16, or a complement thereof is also provided. In some forms, the nucleic acid alters, reduces, inhibits, or silences expression or translation of the target polynucleotide by RNAi, dsRNA, miRNA, siRNA, or transacting small-interfering RNAs (tasiRNA). In some forms, the plant or cell includes a second polynucleotide including a promoter sequence including SEQ ID NO:4, or a fragment thereof including 50, 100, 150, 250, 500, 750, 1,000, 1,250, 1,500, or 2,000 or more nucleotides of SEQ ID NO:4 operably linked to one or more nucleic acid sequence(s) encoding one or more nematode susceptibility genes.

A plant or plant cell including a polynucleotide including a promoter sequence of SEQ ID NO:4, or a fragment thereof including 50, 100, 150, 250, 500, 750, 1,000, 1,250, 1,500, or 2,000 or more nucleotides of SEQ ID NO:4 operably linked to one or more nucleic acid sequence(s) encoding one or more nematode susceptibility genes is also described.

A plant or plant cell including a polynucleotide including a nucleic acid sequence of SEQ ID NO:5 or 6 is also provided, optionally wherein the plant or cell expresses a polypeptide having an amino acid sequence of SEQ ID NO:8.

A plant or plant cell including a polynucleotide including a promoter sequence of SEQ ID NO:4, or a fragment thereof including 50, 100, 150, 250, 500, 750, 1,000, 1,250, 1,500, or 2,000 or more nucleotides of SEQ ID NO:4 operably linked to a polynucleotide including a nucleic acid sequence of SEQ ID NO:5 is also described. In some forms, the plant or cell exhibits reduced expression of one or more nematode susceptibility genes, or has a reduced amount of one or more proteins encoded by one or more nematode susceptibility genes, relative to a wild-type control plant. In some forms, the plant or cell does not express one or more nematode susceptibility genes, or does not express one or more proteins encoded by one or more nematode susceptibility genes, relative to a wild-type control plant. In some forms, one nematode susceptibility gene includes a gene encoding a β-1,4-endoglucancase enzyme, optionally wherein the optionally wherein the β-1,4-endoglucancase enzyme includes an amino acid sequence of SEQ ID NO:7. In some forms, one nematode susceptibility gene includes Glyma.10G017000, optionally wherein the nematode susceptibility gene includes SEQ ID NO:2. In some forms, the plant or cell further includes a polynucleotide including a promoter sequence including SEQ ID NO:12, or a fragment thereof including 50, 100, 150, 250, 500, 750, 1,000, 1,250, 1,500, or 2,000 or more nucleotides of SEQ ID NO:12 operably linked to one or more nucleic acid sequence(s) encoding one or more nematode susceptibility genes.

A plant or plant cell including a polynucleotide including a promoter sequence of SEQ ID NO:12, or a fragment thereof including 50, 100, 150, 250, 500, 750, 1,000, 1,250, 1,500, or 2,000 or more nucleotides of SEQ ID NO:12 operably linked to one or more nucleic acid sequence(s) encoding one or more nematode susceptibility genes is also provided.

A plant or plant cell including a polynucleotide including a nucleic acid sequence of SEQ ID NO:13 or 14, optionally wherein the plant or cell expresses a polypeptide having an amino acid sequence of SEQ ID NO:16, is also described.

A plant or plant cell including a polynucleotide including a promoter sequence of SEQ ID NO:12, or a fragment thereof including 50, 100, 150, 250, 500, 750, 1,000, 1,250, 1,500, or 2,000 or more nucleotides of SEQ ID NO:12 operably linked to a polynucleotide including a nucleic acid sequence of SEQ ID NO:13 is also provided. In some forms, the plant or cell exhibits reduced expression of one or more nematode susceptibility genes, or has a reduced amount of one or more proteins encoded by one or more nematode susceptibility genes, relative to a wild-type control plant. In some forms, the plant or cell does not express of one or more nematode susceptibility genes, or does not express one or more proteins encoded by one or more nematode susceptibility genes, relative to a wild-type control plant.

In some forms, one nematode susceptibility gene includes a gene encoding a pectin methylesterase inhibitor, optionally wherein the optionally wherein the pectin methylesterase inhibitor includes an amino acid sequence of SEQ ID NO:15. In some forms, one nematode susceptibility gene includes Glyma.10G017100, optionally wherein the nematode susceptibility gene includes SEQ ID NO:10.

A plant or plant cell including: (i) a polynucleotide including a promoter sequence of SEQ ID NO:1, or a fragment thereof including 50, 100, 150, 250, 500, 750, 1,000, 1,250, 1,500, or 2,000 or more nucleotides of SEQ ID NO: 1; and/or (ii) a polynucleotide including a promoter sequence of SEQ ID NO:9, or a fragment thereof including 50, 100, 150, 250, 500, 750, 1,000, 1,250, 1,500, or 2,000 or more nucleotides of SEQ ID NO:9; operably linked to one or more nucleic acid sequence(s) encoding one or more heterogenous transgenes, optionally wherein the transgene(s) includes pest resistance gene optionally a nematode resistance gene is also provided. In some forms, the plant includes a soybean plant optionally, wherein the soybean plant includes a Glycine max plant, optionally wherein the Glycine max is a cultivar selected from the group including Jack, Resnik, Williams 82, Corsoy, Crawford, Hutcheson, Benning, Woodruff, Kunitz and Champ. In some forms, the plant includes a cereal crop plant, such as wheat, oat, barley, or rice; or a forage plant such as bahiagrass, dallisgrass, kleingrass, guineagrass, reed canarygrass, orchardgrass, ricegrass, foxtail, or vetch; or a legume such as lentil, or chickpea; or an oilseed such as canola; or a vegetable such as onion or carrot; or a specialty crop such as caraway, hemp, or sesame. In some forms, the plant is resistant to one or more species of nematode relative to a non-plant of the same species. In some forms, the nematode genus is selected from the group including Heterodera, and Meloidogyne. In some forms, the nematode is selected from Heterodera glycines, Heterodera schachtii, and Meloidogyne incognita. In some forms, the nematode is Meloidogyne incognita. In some forms, the resistance is conferred through reduction of nematode colonization, reduction of nematode reproduction, reduction of syncytium formation, tolerance, or combinations thereof. In some forms, the plant further includes a pest resistance QTL.

Seeds and/or progeny of any of the described plants are also provided.

A foodstuff including a plant part from any of the described plants is also provided

Methods for increasing pest resistance in a plant including altering or reducing expression of Glyma.10G017000 β-1,4-endoglucancase, or its ortholog in another plant species by an amount effective to increase pest resistance in the plant, are also described. In some forms, expression of the Glyma.10G017000 β-1,4-endoglucancase is altered or reduced by reducing expression or translation of a target polynucleotide having a nucleic acid sequence according to SEQ ID NO:2 or 3, or a complement thereof, or a nucleic acid encoding the polypeptide of SEQ ID NO:7, or a complement thereof.

Methods for increasing pest resistance in a plant including altering or reducing expression of Glyma.10G017100 pectin methylesterase inhibitor, or its ortholog in another plant species by an amount effective to increase pest resistance in the plant are also described. In some forms, expression of the Glyma.10G017100 pectin methylesterase inhibitor is altered or reduced by reducing expression or translation of a target polynucleotide having a nucleic acid sequence according to SEQ ID NO:10 or 11, or a complement thereof, or a nucleic acid encoding the polypeptide of SEQ ID NO:16, or a complement thereof. In some forms, the pest includes a nematode, optionally wherein the nematode genus is selected from the group including Heterodera, and Meloidogyne, optionally wherein the nematode is selected from Heterodera glycines, Heterodera schachtii, and Meloidogyne incognita.

Methods of increasing nematode resistance including reducing expression of Glyma.10G017000 β-1,4-endoglucancase or its ortholog in another plant species by an amount effective to increase nematode resistance in the plant are also provided. In some forms, expression of the Glyma.10G017000 β-1,4-endoglucancase, or its ortholog in another plant species, is reduced by reducing expression or inhibiting translation of a target polynucleotide having a nucleic acid sequence according to any one of SEQ ID NO:1, 2, 3, or a complement thereof, or a nucleic acid encoding the polypeptide of SEQ ID NO:7 or 8, or a complement thereof.

A method of increasing nematode resistance including reducing expression of Glyma.10G017100 pectin methylesterase inhibitor or its ortholog in another plant species by an amount effective to increase nematode resistance in the plant is also provided. In some forms, expression of the Glyma.10G017000 β-1,4-endoglucancase, or its ortholog in another plant species, is reduced by reducing expression or inhibiting translation of a target polynucleotide having a nucleic acid sequence according to any one of SEQ ID NO:9, 10, 11, or a complement thereof, or a nucleic acid encoding the polypeptide of SEQ ID NO:15 or 16, or a complement thereof. In some forms, the plant includes a polynucleotide including any one of SEQ ID NOs:4-6 and/or 12-14. In some forms, the plant is a soybean plant, optionally wherein the soybean plant includes a Glycine max plant, optionally wherein the Glycine max is a cultivar selected from the group including Jack, Resnik, Williams 82, Corsoy, Crawford, Hutcheson, Benning, Woodruff, Kunitz and Champ.

### BRIEF DESCRIPTION OF THE DRAWINGS

FIGS. 1A-IF are a schematic representation of the genomic region of the major QTL, including (A) Gm10 region identified by Tamulonis et al. 1997 and Li et al. 2001, (B) Markers used by Pham et al. 2013 to fine map M. incognita resistance in PI 96354, (C) 235-kb region identified by Pham et al. 2013. The Wm82.a6.v1 reference genome (displayed) annotates 32 genes in this region. Corresponding mapping studies are overlayed. The box indicates the 30-kb region identified by Xu et al. 2013. The green box indicates genes identified by Passianotto et al. 2017. (D) Markers used for fine-mapping of candidate region, (E) KASP genotyping results of recombinant F5 lines with corresponding phenotype data. Root systems were scored 1-5, with 1=resistant and 5=susceptible. Gall scores shown are the mean of all replicates. (F) The fine-mapped region between markers G10.168 and G10.172 contains two genes—Glyma.10G017000 (EG) and Glyma.10G017100 (PME1).

FIGS. 2A-2B are graphs depicting distribution of galls scores among (A) F2:3 plants, in an initial screen of 173 F2:3 lines and (B) F2:3 plants in a screen of lines found to segregate at GSM039. Bars are colored by KASP genotyping results. The resistant parent, Forrest, is TT at GSM039. The susceptible parent, Bossier, is AA at GSM039. Root systems were scored 1-5, with 1 being the level of galling on Forrest and 5 being the level of galling on Bossier.

FIGS. 3A-3H are histograms showing expression analysis of EG and PME1 among RNAseq samples for (A-D) normalized read counts when comparing (A) infected to non-infected ExF67 (susceptible) tissue at 2 dpi; (B) infected to non-infected ExF63 (resistant) tissue at 2 dpi; (C) infected to non-infected ExF67 tissue at 4 dpi and (D) infected to non-infected ExF63 tissue at 4 dpi. Since PME1 was excluded from edgeR analysis due to lack of reads, there are no significance bars for those comparisons; (E-H) qPCR when comparing (E) infected to non-infected ExF67 tissue at 2 dpi; (F) infected to non-infected tissue at 2 dpi, G) infected to non-infected ExF67 tissue at 4 dpi; and (H) infected to non-infected ExF63 tissue at 4 dpi

FIGS. 4A-4H are histograms showing expression analysis of EG and PME1 among RNAseq samples for (A-D) normalized read counts when comparing (A) infected ExF67 (susceptible) tissue to infected ExF63 (resistant) tissue at 2 dpi, (B) non-infected ExF67 tissue to non-infected ExF63 tissue at 2 dpi, (C) infected ExF67 tissue to infected ExF63 tissue at 4 dpi, (D) non-infected ExF67 tissue to non-infected ExF63 tissue at 4 dpi. Since PME1 was excluded from edgeR analysis due to lack of reads, there are no significance bars for those comparisons. (E-H) qPCR when comparing (E) infected ExF67 tissue to infected ExF63 tissue at 2 dpi, (F) non-infected ExF67 tissue to non-infected ExF63 tissue at 2 dpi, (G) infected ExF67 tissue to infected ExF63 tissue at 4 dpi, and (H) non-infected ExF67 tissue to non-infected ExF63 tissue at 4 dpi

FIG. 5 is a histogram showing EG expression in root tips collected from three susceptible lines (Williams 82, Essex, ExF67) and three resistant lines (PI 96354, Forrest, ExF63). There is a 3× increase in expression among susceptible lines when compared to the resistant lines.

FIG. 6 is a histogram showing expression of GmCel7 in non-infected ExF63 (resistant) and ExF67 (susceptible) root tips.

FIGS. 7A-7C are schematics depicting the polymorphisms present in (A) the EG promoter, with indels indicated by the size of the insertion or deletions in the resistant allele; (B) the EG gene sequence, showing SNPs resulting in an amino acid change are marked; and (C) the EG protein sequence. Domains are shown as predicted by InterPro (Paysan-Lafosse et al. 2023). All polymorphisms are present in both Forrest and PI 96354 when comparing the susceptible lines Williams 82, Bossier, and Essex. The figures illustrate the difference in the resistant alleles against the Wm82.a6.v1 reference annotation (Espina et al. 2024).

FIGS. 8A-8C are schematics depicting the polymorphisms present in (A) the PME1 promoter, with indels indicated by the size of the insertion or deletions in the resistant allele; (B) the PME1 gene sequence, showing SNPs resulting in an amino acid change; and (C) the PME1 protein sequence Domains are shown as predicted by InterPro (Paysan-Lafosse et al. 2023). All polymorphisms are present in both Forrest and PI 96354 when comparing the susceptible lines Williams 82, Bossier, and Essex. The figures illustrate the difference in the resistant alleles against the Wm82.a6.v1 reference annotation (Espina et al. 2024).

FIG. 9 is a schematic, showing a model of the EG genomic sequence. Exons are marked in gray. Sites for forward primers are shown at −2.4 kb and −1.3 kb. Primers used for confirmation of CRISPR edits are marked with names given in Table 5.3. PAM sites for CRISPR gRNA targets are indicated.

FIG. 10 is a schematic, showing vectors for overexpression of EG (Glyma.10G017000). EV=empty pG2RNAi2 vector, Gm:R=CDS of the resistant Forrest under the Gumbi promoter, Gm:S=CDS of the susceptible Essex under the Gmubi promoter, Na:R=CDS of the resistant Forrest under the native Forrest promoter, Na:S=CDS of the susceptible Essex under the native Essex promoter.

FIGS. 11A-11B are a schematic, showing transcription factor binding sites unique to either the susceptible or resistant EG promoter as determined by (A) PlantPAN4.0 (Chow et al. 2024); and (B) PLACE (Higo et al. 1999) FIG. 12 is a schematic, showing the relationships between EGase amino acid sequences from Arabidopsis, tobacco, tomato, and soybean. The evolutionary relationships among plant EGases were calculated using Geneious Prime 2024.0.5. EG (Glyma.10G017000) and its ortholog are indicated with a box.

FIGS. 13A-13D are histograms, showing qPCR expression analysis at 6 dpi between (A) infected galls to non-infected roots in ExF67 (susceptible, B) infected galls to non-infected roots in ExF63 (resistant); (C) non-infected tissue of ExF67 and ExF63; and (D) gall-enriched tissue of ExF67 and ExF63.

FIGS. 14A-14E show (A) Composite soybean roots showing GFP fluorescence seen under fluorescent light; (B) Amplified fragments of DNA extracted from hairy root replicates amplified by primers F1/R1, the length of an uncut fragment is 1,827 bp; (C) Amplification of ExF67 #7 with F2/R2 and F1/R3. The expected band for F2/R2 is 1,346 bp; the expected band size for F1/R3 is 898 bp; (D) Sequences of knockout lines showing a single band at 1,827 bp; SEQ ID NO:74 is CACACCTGGTTCAGAAGTTGCCGCGGAAAC, SEQ ID NO:75 is CACACCTGGTTCNNNNNNNNNNNNNNNNNN, SEQ ID NO:76 is CACACCTGGTT, SEQ ID NO:77 is CCGCGGAAAC, SEQ ID NO:78 is CACACCTGGTTCAGAAGTTCCCC, SEQ ID NO:79 is CACACCTGGTTCAGAAG, SEQ ID NO:80 is CCGCTCAGCC, and SEQ ID NO:81 is CACACCTGGTTCAGAAGTTGNNNNNNNNNN (E) Dot plots showing the total number of nematodes, the number of swollen nematodes, and the percentage of nematodes appearing swollen per root system of each genotype. Data was included from CRISPR lines only if an edit was confirmed. Mean±s.d. are shown; letters indicate significant differences FIGS. 15A-15E show (A) Composite soybean roots showing GFP fluorescence seen under fluorescent light; (B) Amplified fragments of DNA extracted from hairy root replicates amplified by primers F1/R1. The length of an uncut fragment is 1,827 bp; (C) Amplification of ExF67 #6 and #10 with F2/R2 and F1/R3. The expected band for F2/R2 is 1,346 bp; the expected band size for F1/R3 is 898 bp; (D) Sequences of knockout lines showing a single band at 1,827 bp; SEQ ID NO:82 is CACACCTGGTTCAGAA, SEQ ID NO:83 is CACACCTGGTTCAGAAGTTGACTGGGAGAG, SEQ ID NO:84 is CACACCTGGTTCAGAAGT, SEQ ID NO:85 is CACACCTGGTTCAGAAGTT, SEQ ID NO:86 is CACACCTGGTTCAGAANNNNNNNNNNNNNN, SEQ ID NO:87 is CACACCTGGTTCAGAAGTTGCCNNNNNNNN, SEQ ID NO:88 is CTCACCTGGTTCAGAAGTT, and SEQ ID NO:89 is CACACCTGGTTCAGAAGTNNNNNNNNNNNN (E) Dot plots showing the total number of nematodes, the number of swollen nematodes, and the percentage of nematodes appearing swollen per root system of each genotype. Data was included from CRISPR lines only if an edit was confirmed. Mean±s.d. are shown; letters indicate significant differences.

FIGS. 16A-16D are histograms, showing expression of EG (A) in ExF63 (resistant) roots transformed with either EV (pG2RNAi2) or Gm:S; (B) ExF67 (susceptible) roots transformed with either EV (pG2RNAi2) or Gm:R; (C) ExF63 roots transformed with either EV (pG2RNAi2) or Na:S; and (D) ExF67 roots transformed with either EV (pG2RNAi2) or Na:R.

FIGS. 17A-17D show overexpression of susceptible and resistant coding sequences. The susceptible EG was expressed under Gmubi in the resistant genotype; the resistant EG was expressed under Gmubi in the susceptible genotype; (A) Composite soybean roots showing GFP fluorescence seen under fluorescent light; (B) counts of nematodes per root system of each genotype/construct condition; (C) counts of swollen nematodes of each genotype/construct condition; (D) the percentage of nematodes appearing swollen per root system of each genotype. Mean±s.d. are shown; letters indicate significant differences.

FIGS. 18A-18D show overexpression of susceptible EG under Gmubi or the susceptible native promoter in ExF63 (resistant); (A) Composite soybean roots showing GFP fluorescence seen under fluorescent light; (B) counts of nematodes per root system of each genotype/construct condition; (C) counts of swollen nematodes of each genotype/construct condition; (D) the percentage of nematodes appearing swollen per root system of each genotype. Mean±s.d. are shown; letters indicate significant differences.

FIGS. 19A-19D show overexpression of susceptible or resistant EG under the corresponding native promoter in ExF67 (susceptible); (A) Composite soybean roots showing GFP fluorescence seen under fluorescent light; (B) counts of nematodes per root system of each genotype/construct condition; (C) counts of swollen nematodes of each genotype/construct condition; (D) the percentage of nematodes appearing swollen per root system of each genotype. Mean±s.d. are shown; letters indicate significant differences.

### DETAILED DESCRIPTION OF THE INVENTION

### I. Definitions

Unless otherwise indicated, the disclosure encompasses conventional techniques of plant breeding, microbiology, cell biology and recombinant DNA, which are within the skill of the art. See, e.g., Sambrook, et al., Molecular Cloning: A Laboratory Manual, 3rd edition (2001); Current Protocols In Molecular Biology [(F. M. Ausubel, et al. eds., (1987)]; Plant Breeding: Principles and Prospects (Plant Breeding, Vol 1) M. D. Hayward, N. O. Bosemark, I. Romagosa; Chapman & Hall, (1993); Coligan, Dunn, Ploegh, Speicher and Wingfeld, eds. (1995) Current Protocols in Protein Science (John Wiley & Sons, Inc.); the series Methods in Enzymology (Academic Press, Inc.): PCR 2: A Practical Approach (M. J. MacPherson, B. D. Hames and G. R. Taylor eds. (1995)].

Unless otherwise noted, technical terms are used according to conventional usage. Definitions of common terms in molecular biology may be found in Lewin, Genes VII, published by Oxford University Press, 2000; Kendrew et al. (eds.), The Encyclopedia of Molecular Biology, published by Wiley-Interscience, 1999; and Robert A. Meyers (ed.), Molecular Biology and Biotechnology, a Comprehensive Desk Reference, published by VCH Publishers, Inc., 1995; Sambrook, et al., Molecular Cloning: A Laboratory Manual 3rd. edition (2001), Cold Spring Harbor Laboratory Press.

To facilitate understanding of the disclosure, the following definitions are provided:

As used herein, the term “pest” or “plant pest” refers to a destructive nematode or animal, such as nematodes, that attacks crops.

As used herein, the term “pest resistance” or “pest resistant plant” refers to the consequence of heritable plant qualities that result in a plant being relatively less damaged by a pest than a plant without the qualities. A pest resistant plant is typically one that yields more than a susceptible plant when confronted with pest invasion. Resistance of plants is relative and is based on comparison with plants lacking the resistance characters, i.e., susceptible plants. Pest resistant plants typically suppress pest abundance or elevate the damage tolerance level of the plants. A pest resistant plant can alter the relationship a pest has with its plant host. For example, the pest resistance can be antibiosis, antixenosis (non-preference), or tolerance. In some forms, antibiosis and/or antixenosis result in reduction of nematode colonization, reduction of nematode reproduction, reduction of syncytium formation, or combinations thereof.

As used herein, the term “nematode resistance” or “nematode resistant plant” refers to pest resistance or pest resistant plant wherein the pest is a nematode.

As used herein, the term “pest susceptibility” or “pest susceptible plant” refers to a plant that is not pest resistant.

As used herein, the term “nematode susceptibility” or “nematode susceptible plant” refers to pest susceptibility or a pest susceptible plant wherein the pest is a nematode or nematodes.

As used herein, the term “antixenosis” refers to a property of a plant that makes it unattractive to parasitic pests. Antixenosis typically affects the biology of the parasite or plant so pest abundance and subsequent damage is reduced compared to that which would have occurred if the parasite was on a susceptible crop variety. Antibiosis resistance can result in increased mortality or reduced longevity and reproduction of the parasite. Antixenosis resistance can cause parasite response when the parasite attempts to use the resistant plant for food, oviposition, or shelter. Methods of measuring antixenosis are known in the art.

As used herein, the term “antibiosis” refers to an association between a plant and a parasite, such as a nematode, that is detrimental to the parasite, or an antagonistic association between a parasite and a metabolic substance produced by a plant. Antibiosis affects the behavior of parasite, such as a nematode, and usually is expressed as non-preference of the parasite for a resistant plant compared with a susceptible plant. Antibiosis resistance can cause a parasite response when the parasite, such as a nematode, attempts to use the resistant plant for food, oviposition, or shelter. Methods of measuring antibiosis are known in the art.

As used herein, the term “tolerance” refers to a property in which a plant is able to withstand or recover from damage caused by a parasite, such as a nematode, abundance equal to that damaging a plant without resistance characteristics (susceptible). Tolerance is a plant response to a parasite, such as a nematode. Thus, tolerance resistance differs from antibiosis and antixenosis resistance in how it affects the parasite-plant relationship. Tolerant plants can be damaged, but this damage does not affect the plant's ability to survive and reproduce.

The term, “herbivory,” as used herein, refers to the process whereby an organism, such as a parasite, feeds on a plant or a plant-like organism.

The term “plant” is used in its broadest sense. It includes, but is not limited to, any species of woody, ornamental or decorative crop or cereal, and fruit or vegetable plant. It also refers to a plurality of plant cells that are largely differentiated into a structure that is present at any stage of a plant's development. Such structures include, but are not limited to, a fruit, shoot, stem, leaf, flower petal, etc.

The term “non-naturally occurring plant” refers to a plant that does not occur in nature without human intervention. Non-naturally occurring plants include transgenic plants and plants produced by non-transgenic means such as plant breeding.

The term “plant tissue” includes differentiated and undifferentiated tissues of plants including those present in roots, shoots, leaves, pollen, seeds and tumors, as well as cells in culture (e.g., single cells, protoplasts, embryos, callus, etc.). Plant tissue may be in planta, in organ culture, tissue culture, or cell culture. The term “plant part” as used herein refers to a plant structure, a plant organ, or a plant tissue.

The term “plant material” refers to leaves, stems, roots, flowers or flower parts, fruits, pollen, egg cells, zygotes, seeds, cuttings, cell or tissue cultures, or any other part or product of a plant.

The term “plant organ” refers to a distinct and visibly structured and differentiated part of a plant such as a root, stem, leaf, flower bud, or embryo.

The term “plant cell” refers to a structural and physiological unit of a plant, including a protoplast and a cell wall. The plant cell may be in form of an isolated single cell or a cultured cell, or as a part of higher organized unit such as, for example, a plant tissue, a plant organ, or a whole plant.

The term “plant cell culture” refers to cultures of plant units such as, for example, protoplasts, cell culture cells, cells in plant tissues, pollen, pollen tubes, ovules, embryo sacs, zygotes and embryos at various stages of development.

The term “transgenic plant” refers to a plant or tree that contains recombinant genetic material not normally found in plants or trees of this type and which has been introduced into the plant in question (or into progenitors of the plant) by human manipulation. Thus, a plant that is grown from a plant cell into which recombinant DNA is introduced by transformation is a transgenic plant, as are all offspring of that plant that contain the introduced transgene (whether produced sexually or asexually). It is understood that the term transgenic plant encompasses the entire plant or tree and parts of the plant or tree, for instance grains, seeds, flowers, leaves, roots, fruit, pollen, stems etc.

The term “construct” refers to a recombinant genetic molecule having one or more isolated polynucleotide sequences. Genetic constructs used for transgene expression in a host organism include in the 5′-3′ direction, a promoter sequence; a sequence encoding a gene of interest; and a termination sequence. The construct may also include selectable marker gene(s) and other regulatory elements for expression.

The term “gene” refers to a DNA sequence that encodes through its template or messenger RNA a sequence of amino acids characteristic of a specific peptide, polypeptide, or protein. The term “gene” also refers to a DNA sequence that encodes an RNA product.

The term gene as used herein with reference to genomic DNA includes intervening, non-coding regions as well as regulatory regions and can include 5′ and 3′ ends.

The term “orthologous genes” or “orthologs” refer to genes that have a similar nucleic acid sequence because they were separated by a speciation event.

As used herein, “polypeptide” refers generally to peptides and proteins having more than about ten amino acids. The polypeptides can be “exogenous,” meaning that they are “heterologous,” i.e., foreign to the host cell being utilized, such as human polypeptide produced by a bacterial cell.

The term “isolated” is meant to describe a compound of interest (e.g., nucleic acids) that is in an environment different from that in which the compound naturally occurs, e.g., separated from its natural milieu such as by concentrating a peptide to a concentration at which it is not found in nature. “Isolated” is meant to include compounds that are within samples that are substantially enriched for the compound of interest and/or in which the compound of interest is partially or substantially purified. Isolated nucleic acids are at least 60% free, preferably 75% free, and most preferably 90% free from other associated components. An “isolated” nucleic acid molecule or polynucleotide is a nucleic acid molecule that is identified and separated from at least one contaminant nucleic acid molecule with which it is ordinarily associated in the natural source. The isolated nucleic can be, for example, free of association with all components with which it is naturally associated. An isolated nucleic acid molecule is other than in the form or setting in which it is found in nature.

As used herein, the term “locus” refers to a specific position along a chromosome or DNA sequence. Depending upon context, a locus could be a gene, a marker, a chromosomal band or a specific sequence of one or more nucleotides.

The term “vector” refers to a replicon, such as a plasmid, phage, or cosmid, into which another DNA segment may be inserted so as to bring about the replication of the inserted segment. The vectors can be expression vectors.

The term “expression vector” refers to a vector that includes one or more expression control sequences.

The term “expression control sequence” refers to a DNA sequence that controls and regulates the transcription and/or translation of another DNA sequence. Control sequences that are suitable for prokaryotes, for example, include a promoter, optionally an operator sequence, a ribosome binding site, and the like. Eukaryotic cells are known to utilize promoters, polyadenylation signals, and enhancers.

The term “promoter” refers to a regulatory nucleic acid sequence, typically located upstream (5′) of a gene or protein coding sequence that, in conjunction with various elements, is responsible for regulating the expression of the gene or protein coding sequence. The promoters suitable for use in the constructs of this disclosure are functional in plants and in host organisms used for expressing the disclosed polynucleotides. Many plant promoters are publicly known. These include constitutive promoters, inducible promoters, tissue- and cell-specific promoters and developmentally-regulated promoters. Exemplary promoters and fusion promoters are described, e.g., in U.S. Pat. No. 6,717,034, which is herein incorporated by reference in its entirety.

A nucleic acid sequence or polynucleotide is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contiguous and in reading frame. Linking can be accomplished by ligation at convenient restriction sites. If such sites do not exist, synthetic oligonucleotide adaptors or linkers are used in accordance with conventional practice.

“Transformed,” “transgenic,” “transfected” and “recombinant” refer to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed,” “non-transgenic,” or “non-recombinant” host refers to a wild-type organism, e.g., a bacterium or plant, which does not contain the heterologous nucleic acid molecule.

The term “endogenous” with regard to a nucleic acid refers to nucleic acids normally present in the host.

The term “heterologous” refers to elements occurring where they are not normally found. For example, a promoter may be linked to a heterologous nucleic acid sequence, e.g., a sequence that is not normally found operably linked to the promoter. When used herein to describe a promoter element, heterologous means a promoter element that differs from that normally found in the native promoter, either in sequence, species, or number. For example, a heterologous control element in a promoter sequence may be a control/regulatory element of a different promoter added to enhance promoter control, or an additional control element of the same promoter. The term “heterologous” thus can also encompasses “exogenous” and “non-native” elements.

As used herein, “homologous” means derived from the same species. For example, a homologous trait is any characteristic of organisms that is derived from a common ancestor. Homologous sequences can be orthologous or paralogous. Homologous sequences are orthologous if they were separated by a speciation event: when a species diverges into two separate species, the divergent copies of a single gene in the resulting species are said to be orthologous. Orthologs, or orthologous genes, are genes in different species that are similar to each other because they originated from a common ancestor. Homologous sequences are paralogous if they were separated by a gene duplication event: if a gene in an organism is duplicated to occupy two different positions in the same genome, then the two copies are paralogous.

The term “percent (%) sequence identity” is defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods. For purposes herein, the % sequence identity of a given nucleotides or amino acids sequence C to, with, or against a given nucleic acid sequence D (which can alternatively be phrased as a given sequence C that has or includes a certain % sequence identity to, with, or against a given sequence D) is calculated as follows:

$$100 times the fraction W / Z ,$$

where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C.

As used herein, “polypeptide” refers generally to peptides and proteins having more than about ten amino acids. The polypeptides can be “exogenous,” meaning that they are “heterologous,” i.e., foreign to the host cell being utilized, such as human polypeptide produced by a bacterial cell.

The term “stringent hybridization conditions” as used herein mean that hybridization will generally occur if there is at least 95% and preferably at least 97% sequence identity between the probe and the target sequence. Examples of stringent hybridization conditions are overnight incubation in a solution including 50% formamide, 5×SSC (150 mM NaCl, 15 mM trisodium citrate), 50 mM sodium phosphate (pH 7.6), 5×Denhardt's solution, 10% dextran sulfate, and 20 μg/ml denatured, sheared carrier DNA such as salmon sperm DNA, followed by washing the hybridization support in 0.1×SSC at approximately 65° C. Other hybridization and wash conditions are well known and are exemplified in Sambrook, et al., Molecular Cloning: A Laboratory Manual, Third Edition, Cold Spring Harbor, N.Y. (2000).

As used herein, a “cultivar” refers to a cultivated variety.

As used herein, “germplasm” refers to one or more phenotypic characteristics, or one or more genes encoding said one or more phenotypic characteristics, capable of being transmitted between generations.

As used herein, the term “progenitor” refers to any of the species, varieties, cultivars, or germplasm, from which a plant is derived.

As used herein, the term “derivative species, germplasm or variety” refers to any plant species, germplasm or variety that is produced using a stated species, variety, cultivar, or germplasm, using standard procedures of sexual hybridization, recombinant DNA technology, tissue culture, mutagenesis, or a combination of any one or more said procedures.

As used herein, the terms “introgression”, “introgressed” and “introgressing” refer to both a natural and artificial process whereby genes of one species, variety or cultivar are moved into the genome of another species, variety or cultivar, by crossing those species. The process may optionally be completed by backcrossing to the recurrent parent.

As used herein, “plant part” or “part of a plant” can include, but is not limited to cuttings, cells, protoplasts, cell tissue cultures, callus (calli), cell clumps, embryos, stamens, pollen, anthers, pistils, ovules, flowers, seed, petals, leaves, stems, and roots.

As used herein, “quantitative trait loci (QTL)” refers a region of DNA that is closely linked to a specific phenotypic trait. Typically, QTLs underlie continuous traits (those traits that vary continuously) as opposed to discrete traits.

As used herein, a “hybrid” is typically derived from one or more crosses between different varieties, germplasms, populations, breeds or cultivars within a single species, between different subspecies within a species, or between different species within a genus. Typically, hybrids between subspecies are referred to as “intra-specific hybrids” and hybrids between different species within a genus are referred to as “interspecific hybrids.”

As used herein, unless otherwise defined, to “have one or more QTLs” or to “have one or more desirable QTLs” means to have at least one allele of the superior genotype of a particular QTL.

As used herein “superior genotype” is the genotype of the species associated with the desired trait or desired quality of the trait between two plants.

The term “CRISPR/Cas” or “clustered regularly interspaced short palindromic Repeats” or “CRISPR” refers to DNA loci containing short repetitions of base sequences followed by short segments of spacer DNA from previous exposures to a virus or plasmid. Bacteria and archaea have evolved adaptive immune defenses termed CRISPR/CRISPR associated (Cas) systems that use short RNA to direct degradation of foreign nucleic acids. In bacteria, the CRISPR system provides acquired immunity against invading foreign DNA via RNA-guided DNA cleavage. The “CRISPR/Cas” system or “CRISPR/Cas-mediated gene editing” refers to a CRISPR/Cas system that has been modified for genome editing/engineering. For a type II CRISPR/Cas system, it is typically composed of a “guide” RNA (gRNA) and a non-specific CRISPR-associated endonuclease (Cas9). “Guide RNA (gRNA)” is used interchangeably herein with “short guide RNA (sgRNA)” or “single guide RNi” (sgRNA). The sgRNA is a short synthetic RNA composed of a “scaffold” sequence necessary for Cas9-binding and a user-defined, ˜20 nucleotide “spacer” or “targeting” sequence which defines the genomic target to be modified. The genomic target of Cas9 can be modified by changing the targeting sequence present in the sgRNA.

The term “cleavage” refers to the breakage of covalent bonds, such as in the backbone of a nucleic acid molecule or the hydrolysis of peptide bonds. Cleavage can be initiated by a variety of methods, including, but not limited to, enzymatic or chemical hydrolysis of a phosphodiester bond. Both single-stranded cleavage and double-stranded cleavage are possible. Double-stranded cleavage can occur as a result of two distinct single-stranded cleavage events. DNA cleavage can result in the production of either blunt ends or staggered ends. In certain embodiments, fusion polypeptides can be used for targeting cleaved double stranded DNA.

The term “knockdown” refers to a decrease in gene expression of one or more genes. The term “knockout”, or “KO” refers to the ablation of gene expression of one or more genes.

### II. Compositions for Increasing Nematode Resistance

Plant resistance to nematodes, such as Southern Root-Knot nematodes reduces the need for pesticide applications, therefore diminishing production costs and pesticide concerns. Yield loss occurs because the nematode establishes an obligate biotrophic interaction with its host by modifying selected root cells into a feeding site (syncytium) that enables sequestration of nutrients required to complete its 25- to 30-day life cycle. Consequently, SCN can undergo multiple generations in a single growing season leading to rapid increases in field population densities. Syncytium formation is mediated by stylet-secreted effectors (Mitchum et al., 2013) produced in the esophageal glands and delivered to the plant through its mouthpart (stylet). In order for the SCN to successfully parasitize soybean, the formation and long-term maintenance of a feeding site (syncytium) is essential; a process mediated by stylet-secreted effectors (SSEs) that originate from three esophageal gland cells, one dorsal and two sub-ventral (Hussey & Mims, 1990; Hussey, 1989; Mitchum et al., 2013). In soybean, resistance to a broad range of parasites such as southern root-knot nematode Meloidogyne incognita (Rmi1) is found in PI 96354 and other soybean genotypes with Rmi1 (e.g. Forrest). Its resistance is conferred by a major quantitative trait loci (QTL), which reduces loss of soybean plant yield. The major QTL is on chromosome 10 (Chr10), and has a major effect in reduction of nematode colonization and syncytium formation.

It has been discovered that each of two genes located within the major QTL on Chr10 confer Rmi1. A first gene associated with Rmi1 encodes a varied or non-wild-type form of a β-1,4-endoglucancase (EG) enzyme, that has altered or reduced activity, or is inactive relative to the wild-type form of the gene. A second gene associated with Rmi1 encodes a varied or non-wild-type form of a pectin methylesterase (PME1) that has altered or reduced activity, or is inactive relative to the wild-type form of the gene. Both of the EG and PME1 genes on the major QTL contain multiple polymorphisms in the promoter sequences and in the gene sequences, including three missense SNPs in EG and six missense SNPs in PMEL.

It has been demonstrated that the EG gene is more highly-expressed in susceptible soybean plant lines than in Rmi1 resistant soybean plant lines, regardless of infection status. Accordingly, it has been established that expression of the wild-type EG plays a role in host susceptibility, and that a varied or non-wild-type form of a β-1,4-endoglucancase (EG) enzyme, that has altered or reduced activity, or is inactive relative to the wild-type form of EG, confers Rmi1. Therefore, modified plants, such as soybean plants, that encode the varied or non-wild-type form of EG that has altered or reduced activity, or is inactive are provided. Seeds and offspring of the modified plants are also provided. Typically, the plants are resistant to nematodes, such as southern root-knot nematode Meloidogyne incognita relative to plants that encode the varied or non-wild-type form of EG.

It has been demonstrated that the PME1 gene is more highly-expressed in susceptible soybean plant lines than in Rmi1 resistant soybean plant lines, regardless of infection status. Accordingly, it has been established that expression of the wild-type PME1 plays a role in host susceptibility, and that a varied or non-wild-type form of a pectin methylesterase enzyme, that has altered or reduced activity, or is inactive relative to the wild-type form of PME1, confers Rmi1. Therefore, modified plants, such as soybean plants, that encode the varied or non-wild-type form of PME1 that has altered or reduced activity, or is inactive are provided. Seeds and offspring of the modified plants are also provided. Typically, the plants are resistant to nematodes, such as southern root-knot nematode Meloidogyne incognita relative to plants that encode the varied or non-wild-type form of PME1.

Compositions for modifying nematode resistance in plants, such as soybean plants, are also provided. In some forms, the compositions reduce or inhibit the activity of an endogenous β-1,4-endoglucancase (EG) enzyme, or control sequences thereof responsible for nematode susceptibility in the plant. In some forms, the compositions reduce or inhibit the activity of an endogenous pectin methylesterase inhibitor (PME1), or control sequences thereof responsible for nematode susceptibility in the plant.

In some forms, the compositions include compositions for introducing into the plant one or more heterologous genes, or control sequences that promote, increase or activate nematode resistance, such as Rmi1. In some forms, the compositions include one or more functional nucleic acids, such as silencing RNA. In some forms, a heterologous gene that confers nematode resistance is under the control of a promoter sequence that is activated or increased in response to herbivory. Transgenic and hybrid plants, such as soybean plants, with increased resistance to one or more species of nematodes, such as southern root-knot nematode Meloidogyne incognita, compared to a non-transgenic plant of the same species or cultivar are also disclosed.

### A. β-1,4-Endoglucancase

In some forms, gene(s) associated with susceptibility to parasites, such as nematodes, include genetic material associated with or encoding a β-1,4-endoglucanses (also known as “EG”, or “EGases”, or “cellulases”).

β-1,4-endoglucanses hydrolyze bonds in the β-1,4-glucan backbone of cellulose, a compound accounting for 30% of cell wall mass (Ochoa-Villarreal et al. 2012). The first line of defense for a potential host plant when confronted by a nematode pathogen is the root epidermal cell wall. The plant cell wall is primarily composed of cellulose, which can account for up to 40% of the total cell wall mass. In addition to cellulose, the cell wall contains hemicelluloses, xyloglucan, pectin, and various proteoglycans

It has been discovered that a β-1,4-endoglucanse is responsible for pest susceptibility in plants. Accordingly, altering, reducing or inhibiting the expression of a functional a β-1,4-endoglucanse in a plant can increase the pest resistance of the plant. In other pathogen systems, EGases can act as susceptibility factors. SlCel1 and SlCel2 are needed for tomato susceptibility to Botrytis cinerea (Flors et al. 2007). In Arabidopsis, plants lacking the EGase KOR1 were more susceptible to P. syringae (López-Cruz et al. 2014). Expression of several plant EGases has been identified in giant cells, including the tobacco NtCel7 and NtCel8 and the Arabidopsis AtCel1 (Goellner et al. 2001, Sukno et al. 2006). Knockouts of an Arabidopsis EGase (At4g16260) increased susceptibility to H. schachtii, while overexpression of a different Arabidopsis EGase (AtCel6) in soybean decreased susceptibility to both Heterodera glycines (soybean cyst nematode; SCN) and M. incognita (Hamamouch et al. 2012, Woo et al. 2014). RNAi knockouts of GmCel7 decreased susceptibility of soybean to SCN (Woo et al. 2014). Therefore, in some forms, compositions reduce, prevent or decrease expression of the functional gene product of one or more β-1,4-endoglucanse genes in a plant, such as a soybean plant.

### 1. Glyma.10G017000

In some forms, the β-1,4-endoglucanses is a soybean Glyma.10G017000. Polynucleotides having a Glyma.10G017000 gene from a soybean plant are disclosed. The term “Glyma.10G017000^{susceptible}” refers to a gene that encodes a functional β-1,4-endoglucanses in soybean. The term “Glyma.10G017000^{resistant}” refers to a gene that encodes altered β-1,4-endoglucanses, such as a gene that includes one or more missense mutation(s), or which encodes a truncated or alternative splice variant β-1,4-endoglucanse protein, in soybean. Plants that express a functional β-1,4-endoglucanses, such as the Glyma.10G017000^{susceptible} disclosed herein, are susceptible to herbivory, while plants that express an altered gene that encodes an altered β-1,4-endoglucanse, such as a gene that includes one or more missense mutation(s), or which encodes a truncated or alternative splice variant β-1,4-endoglucanse protein, such as the Glyma.10G017000^{resistant} disclosed herein, are resistant to herbivory.

It is understood that where coding sequences for a Glyma.10G017000 gene is provided, also provided are the non-coding sequences that are known or can be identified to correspond to the coding sequence that is provided. For example, where a Glyma.10G017000 gene is provided, also provided for use in the disclosed compositions and methods is the 5′ untranslated region (UTR), which contains the endogenous promoter for the Glyma.10G017000 gene. It is understood that the skilled artisan can identify these sequences with routine skill and experimentation based on the sequences that are provided.

i. Nucleic Acids
a. Glyma.10G017000^{susceptible}

In some forms, the Glyma.10G017000^{susceptible} gene has a promoter sequence as set forth in SEQ ID NO:1, below:

GACAAGTAGCAAACTTTTAGGCAATTTTCCAATCAGAGAATATTATTGTCCATTGCAATTTG
AAAAATAGAAACGGATTCAAGTAAAAACTTACATAATAACAACTAAAATAAAGAAGCCTAAG
ACAATGACCTTAATTGCTTCGAACTATCCACGGCCTTGGTGCGATGACACTAGAAATTTTCA
AAACGAATTTAAATTGTTAAATTAACTTCAAAATTGAAAATATGAATTCATTAACTAAAAAA
CCAAAAAATATCTTTTATTCAATTACGATTTTCATATCAAAATTGAACCAAACCATATCATT
TATAATATTATTATATTTTTATAAAAGTGACCTTGAAAAGAAATGTCATTTCATTATAATTA
AGGATTTAAGGATGTTTATTTTTTAAAAGTTGTTCAAGGAATATAATATCAAAATCTTACCT
TTATACTGAATAAACAAACTCATCCTTATAAACAACTACTTTCATATATTTAAGTAATGTAA
AAAGTAAACTAATTAATTATCAAAGAAGCTAAATATTTGAGGAAAATATAGGAAAAAAATAG
TTTGACTAAAGAAAAAATATACAAAAGTGAAATTATGTAATTTTAAAAGTATTGACCTGTTA
ATGTAACATTTTAAATAAATATAGTAAAATAATAGTTAATTAAAAAAAGAATCACAATAATA
TAAATAAATCATAATTTTTTAAAAATTCAAATAAAATATTATTTGTTGAAAATTCAATCCGA
ATTCACACTTCAAGAAGGTTACCCGATATATCATCCTGTTATAAAAAAGTAAGAAACACGTT
ATAACACACATTTTCATGCACGTGAAAAATAAAAATAAAATATTTTTTTGATAAATATTAAT
TATTAATCTGTTAATTTTATTAATATGAGAAATTAAACACATAATCTTTTACTTATTTATTT
TTTCTTAACCCTCAATCAATCTTATATCCCTTATGATAAATCAAACACACTAATAAGTAATT
CATTATTTGGTTAAATACTATCTCAAAATTTGTGCACAACAAGTTAAATGATAAACCTAACA
TGCACCGAATCACCCAACTATTGGTGTGTCTGTATTCCTGTTTAAGTACATTTATCGTTAAT
CCAAAGGCGAGAGAGAACAGAAAGACGGAATGTTCTATTCACGTAAACATGCATAAAAAGAT
AACCCAAAAAATGTTGTTGGGTTTAATACATTTGTTTGATTATGAATAGTGTATTTGTATTA
GTAATTTTATTAATTTTTGTCTGTGAAATTACTTTTGTACAAATAATATTTTTTTAAAAAAA
TTACAACAAAAAAGTAGACAGAGAATAGAAAATATATAATAAAATAGATGATATAATAATAA
AACTCTTAAATATAAGATGAAAAATAATTTATAATTAATTGAAGATTTAATAACTTTTACAC
TAACATTCCACGTATATTAAACTTTTAAACAATAAGACTTGACTCAATTTGTTTTTGGTTTC
ATAGCTAAAGTTTTTTAAACTACCAAACAATTAAAAGTTATCATTAATAATACTTTTAATTA
TTATAAAAATTAATAAATTTATCATATATAATGATATGTGATTGAATAATATTATAAAATTA
TTTTACACTAAAACTGATTTACCTAAATGTTAAAGAATTTTTTAACTAGTAAAAAAAATATA
AAAATTAACTCAAATGTATTTGCCAAATAAGTTGGGCCATTTAATAACATTCTCTCTTATTT
TCTTCTTGTTTTTAAAAGAATAAGTTGGACCAAATATATTTCTTAAAAAACTTTAAGAAAGG
AATATAAAAATAATGCATGGAGATTATATATTTTTAATAAATAATATGTGCATTTGATTGTA
TAAAGTTTTTCTATGTATTTTCATTCGGTCACAATTATTATATTTACTATCTTTGTTGATTT
TATAACAATTACCTTAAAAATCTTATGAAAGATACTTTTTATAAGTTGACAATGTAAGTATG
TAACTGATTTGAGTTTTTTAAAGTGTCATCGAAATATCAGTTGTTAATATTATCCTTTACCT
TACATTTTTTAATTTTAAACTTGTATGATTGACTAATTAAATGTCTAAAAACTTTAGATTTT
TCATAATTTCATAATGAAATATTAATAACATTATATAATTTCAATTACTAAAATATTAATAG
TTAACTCTTCCAGTTCTAGTTTAACTTCCCCAAACCTTTGTAGACTCTAGCTATTATTAAAA
AAATGAAAAAACAAAAGGCATAACAAAACGCTGTAGCAGGAGTAGGATGTGGACCTAATTAT
TGTTGTATTGTAACGAAGCTCAACGTTAGAGAGGCAGCTTTTGCTTTATAAAAACCAGAGAT
TGAACAACCAAAGAGC

(SEQ ID NO:1; Glyma.10G017000^{susceptible}, Williams 82) or a variant thereof having at least 90%, 95%, or more sequence identity to SEQ ID NO: 1.

In other forms, the Glyma.10G017000^{susceptible} gene has a sequence as set forth in SEQ ID NO:2, below:

TCAGTGTGGCAGTTTGGTCGTTGAGTGAGTGAGTGAGGCTGGTTTCACTTTCACATTGCCTC
TTCAAAATGACTTTCTCCTTTTCCTTCTTCACCATCACCACTCTGCTTTCTCTGTTCTCTCT
AATTCTGCTTCATGCCAATGCCTTCCCAGTCCCCATGCATCGCCACCCTCGCTTTGCCACTC
ATAACTACAGAGATGCTCTCACTAAATCCATTCTCTTCTTTGAAGGCCAGAGGTCAGGGAAG
CTCCCTCCTAACCAGAGAATGTCTTGGAGGAGAGACTCTGGCCTCTCTGATGGCTCAGCCAT
GCACGTATGCATATACCAACTCTCACATTACCATGTTTTCTGGTTTTTGTTTTTGGTTTGCT
TATTTATGTGTGAATGTGCAATGCAGGTTGATTTAGTTGGAGGGTACTATGATGCTGGGGAC
AATGTAAAATTTGGTTTTCCCATGGCCTTCACCACCACCATGCTTTCATGGAGCGTTATTGA
GTTTGGTGGGCTAATGAAAGGTGAGTTGCAGAATGCCAGAGAGGCCATTCGCTGGGGCACTG
ATTATCTTCTCAAAGCCACTGCACATCCAAACACCATTTATGTTCAGGTCAGTTAAGACAAC
AACACTCTTGCTAAAATCAACTTATACCCTTTTGAAAGTAACTATGGAAAAGTTTTTTTTTT
CAAATATGGGGTAAGTCTTCTTTAGATTCTGATATTTGTTTGTTTGTTTGTTATTTGGCTTT
GCAGGTGGGAGACGCTAAGAAGGACCATGCTTGTTGGGAGAGACCAGAGGACATGGACACAC
TAAGAAGCGTGTTTAAAATAGATGCAAACACACCTGGTTCAGAAGTTGCCGCGGAAACTGCT
GCAGCTCTTGCAGCTGCTTCTCTTGTTTTTAGAAGAAGTGACCCCACATACTCCAAAGTTTT
AGTGAGGAGAGCCATCAGGGTAAGTAAACCTTTGGTTCAATTTTTGCTAATTATTATTTTTA
ATAGACCAAACTGCTCCCAACTAAATCTTAAAAGAGTATTTTGTTTCTGAGTGTAGGTCTTC
CAGTTTGCTGATAAGTACAGGGGATCCTACAGCAATGCCTTGAAACCTTATGTGTGCCCCTT
CTATTGCTCTTACTCTGGTTATCAGGTAAAGTTTCAACTGTTCCATATGAGTGATGTTGTTC
AGTTTAGTTGGATGAGACATAATCATTACTCAAATTCTTTTGATGAATAATTATTATGGTAG
GATGAGCTGTTGTGGGGTGCTGCCTGGCTGCACAAGGCTACCAGGAATCCAATGTACCTAAA
CTACATCAAAGTTAATGGCCAGATCCTTGGGGCTGCAGAGTTTGACAACACCTTTGGGTGGG
ATAACAAGCATGCTGGAGCAAGAATACTTCTTTCCAAGGTACAAATTACTCATCCATCACAT
GTATTCTGCTGTATATATATTGCAAAACTAGATAGAAAGTAAAAACTAATAATGGTTTGAAA
TTTACATTTTTGTGTTCCATGGTTTTAGGAATTCCTGGTTCAAAGGGTACAATCCCTCCATG
ACTACAAGGGTCATGCGGACAATTTCGTCTGTTCTCTAATTCCTGGAACCTCTTTTTCTTCC
ACTCAATATACCCCAGGTGAAGTCCCTTTTCCTTCTTAATTCTTTTACTTATCCTCTCTCCC
AGTGGAAACTGCGCGTGTGTGTGGTCCAGTTTTTGTAACGGTTACTTGCTTGTTACTAGGTG
GGCTTCTTTTCAAGATGAGTGATAGCAACATGCAGTATGTCACATCCACCTCCTTCCTTCTG
CTAACATATGCCAAATACTTGACCCAATCCCATATGCTTGTTAACTGTGGTGGAATCACAGT
AACCCCAAGGAGACTCCGGACAATAGCTAAGAAACAGGTTACAAAATGTGTCTATTATATGT
TGAATAAGTTTTAATCAATTAAAATACTTTCTATATGATTAGTTAAAAATTATTTAAAATAA
TTAATTTTGATAAGTTTTATTTCTTAATCAACATAACTAAATCAGGAGCAGAAAAGAGAAAA
TTATTAGATGAGAAATAAAACAAACAAAAAAAAAATTCTAACCAATTATGAAGAACTTCTTA
AACAAAGAATGTAAAAGAGTGCCACCAGAAAAAGTGTTGTTAGATTTCCTATTCTTGTTAGT
TGAACTTGATTTTTTTTTTAATTTTTGTAATTTTAGGTGGATTACTTGCTTGGAGACAACCC
GTTGAAGATGTCGTACATGGTGGGGTATGGTCCACGGTACCCACGAAGGATACACCATAGGG
GATCATCTCTACCGTCGATTGCTGTGCACCCGGGAAAGATCCAGTGTTCCGCAGGGTTCAGT
GTGATGAATTCACAATCTCCCAACCCAAACATTCTAGTGGGGGCCATTGTTGGTGGACCGGA
TGAGCATGATAGGTTCCCAGATCAACGGTCAGATTATGAGCAATCAGAGCCAGCTACATACA
TTAACTCACCCCTTGTAGGAGCACTGGCCTATCTTGCACACTCATTCGGTCAACTCTAGGAA
CCAACCATATCGTGTATCCCATGTTTTATTTTACTAGTTTCGTTAGTTACTTACTTGTGTCT
CCTAGAATTATGCACACACTCATTTGAGACCAGTACGGCAGTACCACACCACTAAGCCTTAT
AAACTTGCTTACAAGCTTAACTTTACTACTATGTGTAACTTCATATTGCCGCCATGCTTTTG
TAAGTATTATGATTTCGGAAAGTGTTATATATGTATCTCCCGTGATGTAGATGTGTTTGATT
TTGCATGTTATTTGGGTGTAAAAAATGGATTAAGTTTGGAATAGCTAGGGTATCGGTTC

(SEQ ID NO:2, Glyma.10G017000^{susceptible}, Williams 82) or a variant thereof having at least 90%, 95%, or more sequence identity to SEQ ID NO:2.

In other forms, the Glyma.10G017000^{susceptible} gene has a CDS sequence as set forth in SEQ ID NO:3, below:

ATGACTTTCTCCTTTTCCTTCTTCACCATCACCACTCTGCTTTCTCTGTTCTCTCTAATTCT
GCTTCATGCCAATGCCTTCCCAGTCCCCATGCATCGCCACCCTCGCTTTGCCACTCATAACT
ACAGAGATGCTCTCACTAAATCCATTCTCTTCTTTGAAGGCCAGAGGTCAGGGAAGCTCCCT
CCTAACCAGAGAATGTCTTGGAGGAGAGACTCTGGCCTCTCTGATGGCTCAGCCATGCACGT
TGATTTAGTTGGAGGGTACTATGATGCTGGGGACAATGTAAAATTTGGTTTTCCCATGGCCT
TCACCACCACCATGCTTTCATGGAGCGTTATTGAGTTTGGTGGGCTAATGAAAGGTGAGTTG
CAGAATGCCAGAGAGGCCATTCGCTGGGGCACTGATTATCTTCTCAAAGCCACTGCACATCC
AAACACCATTTATGTTCAGGTGGGAGACGCTAAGAAGGACCATGCTTGTTGGGAGAGACCAG
AGGACATGGACACACTAAGAAGCGTGTTTAAAATAGATGCAAACACACCTGGTTCAGAAGTT
GCCGCGGAAACTGCTGCAGCTCTTGCAGCTGCTTCTCTTGTTTTTAGAAGAAGTGACCCCAC
ATACTCCAAAGTTTTAGTGAGGAGAGCCATCAGGGTCTTCCAGTTTGCTGATAAGTACAGGG
GATCCTACAGCAATGCCTTGAAACCTTATGTGTGCCCCTTCTATTGCTCTTACTCTGGTTAT
CAGGATGAGCTGTTGTGGGGTGCTGCCTGGCTGCACAAGGCTACCAGGAATCCAATGTACCT
AAACTACATCAAAGTTAATGGCCAGATCCTTGGGGCTGCAGAGTTTGACAACACCTTTGGGT
GGGATAACAAGCATGCTGGAGCAAGAATACTTCTTTCCAAGGAATTCCTGGTTCAAAGGGTA
CAATCCCTCCATGACTACAAGGGTCATGCGGACAATTTCGTCTGTTCTCTAATTCCTGGAAC
CTCTTTTTCTTCCACTCAATATACCCCAGGTGGGCTTCTTTTCAAGATGAGTGATAGCAACA
TGCAGTATGTCACATCCACCTCCTTCCTTCTGCTAACATATGCCAAATACTTGACCCAATCC
CATATGCTTGTTAACTGTGGTGGAATCACAGTAACCCCAAGGAGACTCCGGACAATAGCTAA
GAAACAGGTGGATTACTTGCTTGGAGACAACCCGTTGAAGATGTCGTACATGGTGGGGTATG
GTCCACGGTACCCACGAAGGATACACCATAGGGGATCATCTCTACCGTCGATTGCTGTGCAC
CCGGGAAAGATCCAGTGTTCCGCAGGGTTCAGTGTGATGAATTCACAATCTCCCAACCCAAA
CATTCTAGTGGGGGCCATTGTTGGTGGACCGGATGAGCATGATAGGTTCCCAGATCAACGGT
CAGATTATGAGCAATCAGAGCCAGCTACATACATTAACTCACCCCTTGTAGGAGCACTGGCC
TATCTTGCACACTCATTCGGTCAACTCTAG

(SEQ ID NO:3, Glyma.10G017000^{susceptible}, Essex) or a variant thereof having at least 90%, 95%, or more sequence identity to SEQ ID NO:3.

Therefore, a polynucleotide is disclosed having a nucleic acid sequence SEQ ID NO: 1, or 2, or 3, or a fragment or variant thereof. Also disclosed is a fragment or variant of Glyma.10G017000^{susceptible} coding sequence having a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO:1, or 2, or 3. A fragment can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 50, 75, 100, or more nucleotides shorter than SEQ ID NO:1, or 2, or 3.

Also disclosed is a polynucleotide that hybridizes under stringent conditions to a polynucleotide having the nucleic acid sequence SEQ ID NO: 1, or 2, or 3, or a fragment or variant thereof.

ii. Glyma.10G017000^{resistant}

In some forms, the resistant form of Glyma.10G017000 is derived from a QTL derived from the Forrest soybean line.

Forrest is a soybean line with moderate resistance to M. incognita (Herman et al. 1990). It possesses a single additive gene conferring this resistance, named Resistance to M. incognita-1, or Rmi1 (Luzzi et al. 1994). This resistance manifests primarily through emigration of second-stage juveniles (J2s) before the initiation of a feeding site, with 53% fewer nematodes remaining in the roots after sixteen days than in roots of the susceptible cv. Bossier (Herman et al. 1991). In the more resistant PI 96354, resistance is further demonstrated through delayed nematode development and decreased fecundity (Moura et al. 1993). PI 96354 possesses an additional resistance QTL with dominant action (Tamulonis et al. 1997, Li et al. 2001). Since a major QTL on chromosome 10 has been identified in every M. incognita resistance mapping study in soybean, it is likely deployed in a majority of soybean fields, though precise records are not kept (Fourie et al. 2008, Xu et al. 2013, Jiao et al. 2015, Passianotto et al. 2017).

Soybean plants containing a major QTL, which express Glyma.10G017000^{resistant}, do not express a functional Glyma.10G017000 protein. Glyma.10G017000^{resistant} contains a SNP unique to nematode-resistant soybeans, which produces an altered Glyma.10G017000 protein that may be non-expressed, or a truncated or alternative splice variant of the wildtype protein. The protein encoded by Glyma.10G017000^{resistant} may have altered function or be non-functional.

It has also been discovered that expression of Glyma10G017000 in soybean is induced by herbivory. Accordingly, the control/promoter sequences that control expression of Glyma10G017000 can be used to promote herbivory-induced expression of a gene of interest. In some embodiments, the control sequences include genomic untranslated regions 5′ of Glyma10G017000, 3′ of Glyma10G017000, or combinations thereof.

In other embodiments, the control/promoter sequence that controls expression of Glyma10G017000 include 50, 100, 150, 250, 500, 750, 1,000, 1,250, 1,500, 2,500, 5,000 nucleotides 5′ of Glyma10G017000. In some embodiments, the control sequence ends immediately before the ATG start codon of Glyma10G017000. For example, the control sequence that controls expression of Glyma10G017000 can include 50, 100, 150, 250, 500, 750, 1,000, 1,250, 1,500, 2,500, 5,000 nucleotides of the nucleic acid sequence

In some forms, the Glyma.10G017000^{resistant} gene has a promoter sequence as set forth in SEQ ID NO:4, below:

GACAAGTAGCAAACTTTTAGGCAATTTTCCAATCATAGAATATTATTGTCCATTGCAAATTG
AAAAATAGAAACGGATTCAAGTAAAAACTTACATAATAACAACTAAAATAAAATAAAATAAA
GAAGCCTAAGACAATGACCTTAAATGCTTCGAACTATCCACGGCCTTGGTGCGATGACACTA
GAAATTTTCAAAACGAATTTAAATTGTTAAATTAACTTCAAAATTGAAAATATGAATTCATT
AACTAAAAAACCAAAAAATATCTTTTATTCAATTACGATTTTCATATCAAAATTGAACCAAA
CCATATCATTTATAATATTATTATATTTTTATAAAAGAAATGTCATTTCATTATAATTAAGG
ATTTAAGGATGTTTATTTTTTAAAAGTTGTTCAAGGAATATAATATCAAAATCTTACCTTTA
TACTGAATAAACAAACTCATCCTTATAAACAACTACTTTCATATATTTAAGTAATGTAAAAA
GTAAACTAATTAATTATCAAAGAAGCTAAATATTTGAGGAAAATATAGGAAAAAAATAGTTT
GACTAAAGAAAAAATATACAGAAGTGAAATTATGTAATTTTAAAAGTATTGACCTGTTAATG
TAACATTTTAAATAAATATAGTAAAATAATAGTTAATTAATAAAAGAATCACAATAATATAA
ATAAATCATAATTTTTTAAAAATTCAAATAAAATATTATTTGTTGAAAATTCAATCCGAATT
CACACTTCAAGAAGGTTACCCGATATATCATCCTGTTATAAAAAAATAAGAAACACGTTATA
ACACACACTTTCATGCACGTGAAAAATAAAAATAAAATATTTTTTTGGTAAATATTAATTAT
TAATCTGTTAATTTTATTAATATGAGAAATTCAACACATAATCTTTTACTTGTTTATTTTTT
CTTAACCCTCAATCAATCTTATATCCCTTATGATAAATCAAACACACTAATAAGTAATTCAT
TATTTGGTTAAATACTATCTCAAAATTTGTGCACAACAAGTTAAATGATAAACCTAACATGC
ACCGAATCACCCAACTATTGGTGTGTCTGTATTCCTCTTTAAGTACATTTATCGTTAATCCA
AAGGCGAGAGAGAACAGAAAGACGGAATGTTCTATTCACGTAAACATGCATAAAAAGATAAC
CCAAAAAATGTTGTTGGGTTTAATACATTTGTTTGATTATGAATAGTGTATTTGTATTAGTA
ATTTTATTAACTTTTGTCTGTGAAATTACTTTTGTACAAATAATATTTTTAAAAAAAAATTA
CAACAAAAAAGTAGAAAGAGAATAGAAAATATATAATAAAATAGATGATATAATAATAAAAC
TCTTAAATATAAGATGAAAAATAATTTATAATTAATTGAAGATTTAATAACTTTTACACTAA
CATTCCACGTATATTAAACTTTTAAACAATAAGACTTGACTCAATTTGTTTTTGGTTTCATA
GCTATATATTATTAGTGTAAAGTTTTTTAAACTACCAAACAATTAAAAGTTATCATTAATAA
CACTTTTAATTATTATAAAAATTAATAAATTTATCATATATAATAATATGTGATTGAATAAT
ATTATAAAATTATTTTACACTAAAACTGATTTACCTAAATGTTAAAGAATTTTTTAACTAGT
AAAAAAAATATAAAAATTAACTTAAATGTACTGCCAAATAAGTTGGGCCATTTAATAACATT
CTCTCTTATTTTCTTATTGTTTTTACAAGAATAAGTTGGACCAAATATATTTCTTAAAAAAC
TTTAAGAAAGGAATATAAAAATAATGCATGGAGATTATATTTTTTTTTAATAAATAATATGT
GCATTTGATTGTATAAAGTTTTTCTATGTATTTTCATTCGGTCACAATTATTATATTTGCTA
TCTTTGTTGATTTTATAACAATTACCTTAAAAATCTTATGAAAGATACTTTTTATAAGTTGA
CAATGTAAGTATGTAACTGATTTGAGTTTTTTAAAGTGTCATCGAAATATCAGTTGTTAATA
TTATCCCTTACCTTACATTTTTTAATTTTAAACTTGTATGATTGACTAATTAAATGTCTAAA
AACTTTAGATTTTTCTTAATTTCATAATGAAATATTAATAACATTATATAATTTCAATTACT
AAAATATTAATAGTTAACTCTTCCAGTTCTAGTTTAACTTCCCCAAACCTTTGTAGACTCTA
GCTATTATTAAAAAAATGAAAAAACAAAAGTCATAACAAAACGCTGTAGCAGGAGTAGGATG
TGGACCTAATTATTGTTGTATTGTAACGAAGCTCAACGTTAGAGAGGCAGCTTTTGCTTTAT
AAAAACCAGAGATTGAACAACCAAAGAGC

(SEQ ID NO:4, Glyma.10G017000^{resistant}, Forrest) or a variant thereof having at least 90%, 95%, or more sequence identity to SEQ ID NO:4.

In other forms, the Glyma.10G017000^{resistant} gene has a sequence as set forth in SEQ ID NO:5, below:

TCAGTGTGGCAGTTTGGTCGTTGAGTGAGTGAGTGAGGCTGGTTTCACTTTCACATTGCCTC
TTCAAAATGACTTTCTCCTTTTCCTTCTTCACCATCACCACTCTGCTTTCTCTGTTCTCTCT
AATTCTGCTTCATGCCAATGCCTTCCCAGTCCCCATACATCGCCACCCTCGCTTTGCAGGGA
AGCTCCCTCCTAACCAGAGAATGTCTTGGAGGAGAGACTCTGGCCTCTCTGATGGCTCAGCC
ATGCACGTATGCATATACCAACTCTCACATTACCATGTTTTTTTGTTTTTGTTTTTGGTTTG
CTTATTTATGTGTGAATGTGCAATGCAGGTTGATTTGGTTGGAGGGTACTATGATGCTGGGG
ACAATGTGAAATTTGGTTTTCCCATGGCCTTCACCATCACCATGCTTTCATGGAGCGTTATT
GAGTTTGGTGGGCTAATGAAAGGTGAGTTGCAGAATGCCAGAGAGGCCATTCGCTGGGGCAC
TGATTATCTTCTCAAAGCCACTGCACATCCAAACACCATTTATGTTCAGGTCAGTTAAGACA
ACAACACTCTTGCTAAAATCAACTTATACCCTTTTGAAAGTAACTATGGAAAAGTTTTTATT
TTCAAATATGGGGTAAGTCTTCTTTAGATTCTGATATTTGTTTGTTTGTTTGTTATTTGGCT
TTGCAGGTGGGAGACGCTAAGAAGGACCATGCTTGTTGGGAGAGACCAGAGGACATGGACAC
ACTAAGAAGCGCGTTTAAAATAGATGCAAACACACCTGGTTCAGAAGTTGCCGCGGAAACTG
CTGCAGCTCTTGCAGCTGCTTCTCTTGTTTTTAGAAGAAGTGACCCCACATACTCCAAAGTT
TTAGTGAGGAGAGCCATCAGGGTAAGTAAACCTTTGGTTCAATTTTTGCTAATTATTATTTT
TAATAGACCAAACTGCTCCCAACTAAATCTCAAAAGACTATTTTGTTTCTGAGTGTAGGTCT
TCCAGTTTGCTGATAAGTACAGGGGATCCTACAGCAATGCCTTGAAACCTTATGTGTGCCCC
TTCTATTGCTCTTACTCTGGTTATCAGGTAAAGTTTCAACTGTTCCATATGAGTGATGTTGT
TCAGTTTAGTTGGATGAGACATAATCATTACTCAAATTCTTTTGATGAATAATTATTATGGT
AGGATGAGCTGTTGTGGGGTGCTGCCTGGCTGCACAAGGCTACCAGGAATCCAATGTACCTA
AACTACATCAAAGTTAATGGCCAGATCCTTGGGGCTGCAGAGTTTGACAACACCTTTGGGTG
GGATAACAAGCATGCTGGAGCAAGAATACTTCTTTCCAAGGTACAAATTACTCATCCATCAC
ATGTATTCTGCTGTATATATATTGCAAAACTAGATAGAAAGTAAAAACTAATAATGGTTTGA
AATTTACATTTTTGTGTTCCATGGTTTTAGGAATTCCTGGTTCAAAGGGTACAATCCCTCCA
TGACTACAAGGGTCATGCGGACAATTTCGTCTGTTCTCTAATTCCTGGAACCTCTTTTTCTT
CCACTCAATATACCCCAGGTGAAGTCCCTTTTCCTTCTTAATTCTTTTACTTATCCTCTCTC
CCAGTGGAAACTGCGCGTGTGTGTGGTCCAGTTTTTGTAACGGTTACTTGCTTGTTACTAGG
TGGGCTTCTTTTCAAGATGAGTGATAGCAACATGCAGTATGTCACATCCACCTCCTTCCTAC
TGCTAACATATGCCAAATACTTGACCCAATCCCATATGCTTGTTAACTGTGGTGGAATCACA
GTAACCCCAAGGAGACTCCGGACAATAGCTAAGAAACAGGTTACAAAATGTGTCTATTATAT
GTTGAATAAGTTTTAATCAATTAAAATACTTTCTATATGATTAGTTAAAAATTATTTAAAAT
AATTAATTTTGATAAGTTTTATTTCTTATTCAACATAACTAAATCAGGAGCAGAAAAGAGAA
AATTATTAGATGAGAAATAAAACAAACAAAAAAAAAATTCTAACCAATTATGAAGAACTTCT
TAAACAAAGAATGTAAGAGAGTGCCACCAGAAAAAGTGTTGTTAGATTTCCTATTCTTGTTA
GTTGAACTTGATTTTTTTTTTTAATTTTTGTAATTTTAGGTGGATTACTTGCTTGGAGACAA
CCCGTTGAAGATGTCGTACATGGTGGGGTATGGTCCACGGTACCCACGAAGGATACACCATA
GGGGATCATCTCTACCGTCGATTGCTGTGCACCCGGGAAAGATCCAGTGTTCCGCAGGGTTC
AGTGTGATGAATTCACAATCTCCCAACCCAAACATTCTAGTGGGGGCCATTGTTGGTGGACC
GGATGAGCATGATAGGTTCCCAGATCAACGGTCAGATTATGAGCAATCAGAGCCAGCTACAT
ACATTAACTCACCCCTTGTAGGAGCACTGGCCTATCTTGCACACTCATTCGGTCAACTCTAG
GAACCAACCATATCGTGTATCCAATGTTTTATTTTACTAGTTTCGTTAGTTACTTACTTGTG
TCTCCTAGAATTATGCACACACTCATTTGAGACCAGTACGGCAGTACCACACCACTAAGCCT
TATAAACTTGCTTACAAGCTTAACTTTACTACTATGTGTAACTTCATATTGCCGCCATGCTT
TTGTAAGTATTATGATTTCGGAAAGTGTTATATATGTATCTCCCGTGATGTAGATGTGTTTG
ATTTTGCATGTTATTTGGGTGTAAAAAATGGATTAAGTTTGGAATAGCTAGGGTATCGGTTC

(SEQ ID NO:5, Glyma.10G017000^{resistant}, Forrest) or a variant thereof having at least 90%, 95%, or more sequence identity to SEQ ID NO:5.

In other forms, the Glyma.10G017000^{resistant} gene has a CDS sequence as set forth in SEQ ID NO:6, below:

ATGACTTTCTCCTTTTCCTTCTTCACCATCACCACTCTGCTTTCTCTGTTCTCTCTAATTCT
GCTTCATGCCAATGCCTTCCCAGTCCCCATACATCGCCACCCTCGCTTTGCCACTCATAACT
ACAGAGATGCTCTCACTAAATCCATTCTCTTCTTTGAAGGCCAGAGGTCAGGGAAGCTCCCT
CCTAACCAGAGAATGTCTTGGAGGAGAGACTCTGGCCTCTCTGATGGCTCAGCCATGCACGT
TGATTTGGTTGGAGGGTACTATGATGCTGGGGACAATGTGAAATTTGGTTTTCCCATGGCCT
TCACCATCACCATGCTTTCATGGAGCGTTATTGAGTTTGGTGGGCTAATGAAAGGTGAGTTG
CAGAATGCCAGAGAGGCCATTCGCTGGGGCACTGATTATCTTCTCAAAGCCACTGCACATCC
AAACACCATTTATGTTCAGGTGGGAGACGCTAAGAAGGACCATGCTTGTTGGGAGAGACCAG
AGGACATGGACACACTAAGAAGCGCGTTTAAAATAGATGCAAACACACCTGGTTCAGAAGTT
GCCGCGGAAACTGCTGCAGCTCTTGCAGCTGCTTCTCTTGTTTTTAGAAGAAGTGACCCCAC
ATACTCCAAAGTTTTAGTGAGGAGAGCCATCAGGGTCTTCCAGTTTGCTGATAAGTACAGGG
GATCCTACAGCAATGCCTTGAAACCTTATGTGTGCCCCTTCTATTGCTCTTACTCTGGTTAT
CAGGATGAGCTGTTGTGGGGTGCTGCCTGGCTGCACAAGGCTACCAGGAATCCAATGTACCT
AAACTACATCAAAGTTAATGGCCAGATCCTTGGGGCTGCAGAGTTTGACAACACCTTTGGGT
GGGATAACAAGCATGCTGGAGCAAGAATACTTCTTTCCAAGGAATTCCTGGTTCAAAGGGTA
CAATCCCTCCATGACTACAAGGGTCATGCGGACAATTTCGTCTGTTCTCTAATTCCTGGAAC
CTCTTTTTCTTCCACTCAATATACCCCAGGTGGGCTTCTTTTCAAGATGAGTGATAGCAACA
TGCAGTATGTCACATCCACCTCCTTCCTACTGCTAACATATGCCAAATACTTGACCCAATCC
CATATGCTTGTTAACTGTGGTGGAATCACAGTAACCCCAAGGAGACTCCGGACAATAGCTAA
GAAACAGGTGGATTACTTGCTTGGAGACAACCCGTTGAAGATGTCGTACATGGTGGGGTATG
GTCCACGGTACCCACGAAGGATACACCATAGGGGATCATCTCTACCGTCGATTGCTGTGCAC
CCGGGAAAGATCCAGTGTTCCGCAGGGTTCAGTGTGATGAATTCACAATCTCCCAACCCAAA
CATTCTAGTGGGGGCCATTGTTGGTGGACCGGATGAGCATGATAGGTTCCCAGATCAACGGT
CAGATTATGAGCAATCAGAGCCAGCTACATACATTAACTCACCCCTTGTAGGAGCACTGGCC
TATCTTGCACACTCATTCGGTCAACTCTAG

(SEQ ID NO:6, Glyma.10G017000^{resistant}, Forrest) or a variant thereof having at least 90%, 95%, or more sequence identity to SEQ ID NO:6.

Therefore, a polynucleotide is disclosed having a nucleic acid sequence SEQ ID NO: 4, or 5, or 6, or a fragment or variant thereof. Also disclosed is a fragment or variant of Glyma.10G017000^{resistant} coding sequence having a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO:4, or 5, or 6. A fragment can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 50, 75, 100, or more nucleotides shorter than SEQ ID NO: 4, or 5, or 6.

b. Polypeptides
i. Glyma.10G017000^{susceptible}

An amino acid sequence encoded by a Glyma.10G017000^{susceptible} gene is also disclosed.

Thus disclosed is a polypeptide encoded by the nucleic acid sequence of SEQ ID NO:2, or 3, or a fragment or variant thereof. Also disclosed is a polypeptide encoded by a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO:2, or 3, or a fragment or variant thereof. Also disclosed is a polypeptide encoded by a polynucleotide that hybridizes under stringent conditions to a polynucleotide including the nucleic acid sequence SEQ ID NO:2, or 3, or a fragment or variant thereof.

A polypeptide that is a fragment or variant of a Glyma.10G017000^{susceptible} gene product is also disclosed. Thus, a polypeptide encoded by a polynucleotide having a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO:2, or 3 is disclosed. The fragment can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 50, 75, or more amino acids shorter than the polypeptide encoded by the nucleic acid sequence SEQ ID NO:2, or 3.

In some embodiments, the gene product of Glyma.10G017000^{susceptible} includes the amino acid sequence encoded by SEQ ID NO:7

In still other embodiments an amino acid sequence of a Glyma.10G017000^{susceptible} polypeptide is:

MTFSFSFFTITTLLSLFSLILLHANAFPVPMHRHPRFATHNYRDALTKSILFFEGQRSGK
LPPNORMSWRRDSGLSDGSAMHVDLVGGYYDAGDNVKFGFPMAFTTTMLSWSVIEFGGLM
KGELQNAREAIRWGTDYLLKATAHPNTIYVQVGDAKKDHACWERPEDMDTLRSVFKIDAN
TPGSEVAAETAAALAAASLVFRRSDPTYSKVLVRRAIRVFQFADKYRGSYSNALKPYVCP
FYCSYSGYQDELLWGAAWLHKATRNPMYLNYIKVNGQILGAAEFDNTFGWDNKHAGARIL
LSKEFLVQRVQSLHDYKGHADNFVCSLIPGTSFSSTQYTPGGLLFKMSDSNMQYVTSTSF
LLLTYAKYLTQSHMLVNCGGITVTPRRLRTIAKKQVDYLLGDNPLKMSYMVGYGPRYPRR
IHHRGSSLPSIAVHPGKIQCSAGFSVMNSQSPNPNILVGAIVGGPDEHDRFPDORSDYEQ
SEPATYINSPLVGALAYLAHSFGQL

(SEQ ID NO:7, Glyma10G017000^{susceptible}, Williams 82) or a variant thereof having at least 90%, 95%, or more sequence identity to SEQ ID NO:7.

Therefore, a polypeptide is disclosed having an amino acid sequence of SEQ ID NO:7, or a fragment or variant thereof. Also disclosed is a fragment or variant of a Glyma.10G017000^{susceptible} polypeptide having a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO:7. A fragment can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 50, 75, 100, or more amin acids shorter than SEQ ID NO:7.

Also disclosed are polynucleotides encoding the amino acid sequence SEQ ID NO:7, or fragments or variants thereof.

ii. Glyma.10G017000^{resistant}

An amino acid sequence encoded by a Glyma.10G017000^{resistant} gene is also disclosed.

Thus disclosed is a polypeptide encoded by the nucleic acid sequence of SEQ ID NO:4, or 5, or 6, or a fragment or variant thereof. Also disclosed is a polypeptide encoded by a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO:4, or 5, or 6, or a fragment or variant thereof. Also disclosed is a polypeptide encoded by a polynucleotide that hybridizes under stringent conditions to a polynucleotide including the nucleic acid sequence SEQ ID NO: 4, or 5, or 6, or a fragment or variant thereof.

A polypeptide that is a fragment or variant of a Glyma.10G017000^{resistant} gene product is also disclosed. Thus, a polypeptide encoded by a polynucleotide having a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO:4, or 5, or 6 is disclosed. The fragment can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 50, 75, or more amino acids shorter than the polypeptide encoded by the nucleic acid sequence SEQ ID NO:4, or 5, or 6.

In some embodiments, the gene product of Glyma.10G017000^{resistant} includes the amino acid sequence encoded by SEQ ID NO:8:

MTFSFSFFTITTLLSLFSLILLHANAFPVPIHRHPRFATHNYRDALTKSILFFEGORSGKLP
PNQRMSWRRDSGLSDGSAMHVDLVGGYYDAGDNVKFGFPMAFTITMLSWSVIEFGGLMKGEL
QNAREAIRWGTDYLLKATAHPNTIYVQVGDAKKDHACWERPEDMDTLRSAFKIDANTPGSEV
AAETAAALAAASLVFRRSDPTYSKVLVRRAIRVFQFADKYRGSYSNALKPYVCPFYCSYSGY
QDELLWGAAWLHKATRNPMYLNYIKVNGQILGAAEFDNTFGWDNKHAGARILLSKEFLVQRV
QSLHDYKGHADNFVCSLIPGTSFSSTQYTPGGLLFKMSDSNMQYVTSTSFLLLTYAKYLTQS
HMLVNCGGITVTPRRLRTIAKKQVDYLLGDNPLKMSYMVGYGPRYPRRIHHRGSSLPSIAVH
PGKIQCSAGFSVMNSQSPNPNILVGAIVGGPDEHDRFPDORSDYEQSEPATYINSPLVGALA
YLAHSFGQL

(SEQ ID NO:8) or a variant thereof having one or more conservative amino acid substitutions and at least 90%, 95%, or more sequence identity compared to SEQ ID NO:8.

A polypeptide is therefore disclosed having the amino acid sequence SEQ ID NO:8, or a fragment or variant thereof. A polypeptide having an amino acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO:8 is also disclosed.

A polypeptide that is a fragment or variant of Glyma10G017000^{resistant} protein including the amino acid sequence SEQ ID NO:8 is also disclosed. A polypeptide having an amino acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to a fragment of SEQ ID NO:8 is disclosed. The fragment can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 50, or 75 amino acids shorter than SEQ ID NO:8.

Also disclosed are polynucleotides encoding the amino acid sequence SEQ ID NO:8, or fragments or variants thereof.

### 2. Homologs and Orthologs of Glyma.10G017000

The Glyma.10G017000 gene and/or protein is homologous or orthologous to the β-1,4-endoglucanse of other plants, such as plants other than soybean plants.

Typically, genes that are homologous or orthologous share at least 75% sequence identity with Glyma.10G017000 and share at least part of the biological function of Glyma.10G017000. Exemplary genes that are homologous or orthologous to the described Glyma.10G017000 gene include: Gossypium hirsutum LOC107912359 (NCBI Accession: NP_001313998.1); Gossypium hirsutum LOC107924647 (NCBI Accession: XP_016710672.1); Capsicum annum LOC107864053 (NCBI Accession: XP_016565793.2); Nicotiana tabacum LOC107778715 (NCBI Accession: XP_016454485.1); Nicotiana tabacum LOC107795934 (NCBI Accession: XP_016474128.1); Solanum lycopersicum LOC543999 (NCBI Accession: NP_001234882.1); Nicotiana tabacum LOC107788548 (NCBI Accession: XP_016465715.1); Nicotiana tabacum LOC107807087 (NCBI Accession: XP_016486862.1); and Nicotiana tabacum LOC107795934 (NCBI Accession: XP_016474138.1), the NCBI sequence data of all of which are hereby incorporated by reference herein in their entirety.

### B. Pectin Methylesterase Inhibitor

In some forms, gene(s) associated with susceptibility to parasites, such as nematodes, include genetic material associated with or encoding a pectin methylesterase enzyme inhibitor (also known as “PME1”). PME1 encodes a type I pectin methylesterase inhibitor, containing a PME catalytic domain and a PME inhibitor domain. The inhibitor domain likely acts as an intramolecular chaperone for the PME domain (Micheli 2001). An Arabidopsis type I PME was found to directly interact with a cellulose binding protein (CBP) secreted by Heterodera schachtii to promote feeding site initiation (Hewezi et al. 2008). M. incognita infective J2s also secrete a CBP effector (Mi-CBP) while migrating intercellularly through the pectin-rich middle lamella of the root (Ding et al. 1998). PME1 is expressed at nearly undetectable levels, making it more difficult to accurately quantify its expression. However, the ability to amplify PME1 cDNA does verify that it is expressed at some level in root tissue.

In plants, PMEs typically demethylesterify (DM) pectin in a linear fashion, acting on chains of homogalacturonan (HG), the most abundant component of pectin (Wolf et al. 2009). This releases methyl groups from the HG, leaving a binding site for Ca²⁺ ions. The connection of HG chains through Ca²⁺ binding leads to “egg-box” structures that rigidify the cell wall (Micheli 2001, Wormit and Usadel 2018, Molina et al. 2024). If Ca²⁺ binding does not occur, this leaves the HG chains open to pectin-degrading enzymes, like polygalacturonases (Pelloux et al. 2007). HG degradation by polygalacturonases releases oligogalacturonides that can function as damage-associated molecular patterns (DAMPs) that initiate DAMP-triggered immunity (DPI) (Lionetti et al. 2012, Molina et al. 2024). The level of pectin esterification has been implicated in resistance against wheat fungus (Wietholter, et al. 2003) and liquorice rot in carrots (Le Cam et al. 1994). There has also been a direct interaction found between an Arabidopsis type I PME and a cellulose binding protein (CBP) secreted by Heterodera schachtii during feeding site formation (Hewezi et al. 2008). The specific binding sites in the interaction were not determined. M. incognita infective J2s also secrete a CBP effector (Ding et al. 1998). Mi-CBP could target the catalytic domain in PME1 that is missing from the resistant allele, thus leading to a loss of a susceptibility factor in resistant lines. While establishment of a giant cell requires precise manipulation of the cell wall in order to rapidly increase the cell size while maintaining wall integrity, J2s travel intercellularly through the middle lamella, a primarily pectin layer, while migrating through the root before selecting an initial feeding cell. RKN secrete other pectin-modifying enzymes while migrating (Jaubert et al. 2002, Huang et al. 2005).

### 1. Glyma.10G017100

In some forms, the pectin methylesterase inhibitor is a soybean Glyma.10G017100. Polynucleotides having a Glyma.10G017100 gene from a soybean plant are disclosed. The term “Glyma.10G017100^{susceptible}” refers to a gene that encodes a functional pectin methylesterase inhibitor in soybean. The term “Glyma.10G017100^{resistant}” refers to a gene that encodes altered pectin methylesterase inhibitor, such as a gene that includes one or more missense mutation(s), or which encodes a truncated or alternative splice variant pectin methylesterase inhibitor protein, in soybean. Plants that express a functional pectin methylesterase inhibitor, such as the Glyma.10G017100^{susceptible} disclosed herein, are susceptible to herbivory, while plants that express an altered gene that encodes an altered pectin methylesterase inhibitor, such as a gene that includes one or more missense mutation(s), or which encodes a truncated or alternative splice variant pectin methylesterase inhibitor protein, such as the Glyma.10G017100^{resistant} disclosed herein, are resistant to herbivory.

It is understood that where coding sequences for a Glyma.10G017100 gene is provided, also provided are the non-coding sequences that are known or can be identified to correspond to the coding sequence that is provided. For example, where a Glyma.10G017100 gene is provided, also provided for use in the disclosed compositions and methods is the 5′ untranslated region (UTR), which contains the endogenous promoter for the Glyma.10G017100 gene. It is understood that the skilled artisan can identify these sequences with routine skill and experimentation based on the sequences that are provided.

i. Nucleic Acids
a. Glyma.10G017100^{susceptible}

In some forms, the Glyma.10G017100^{susceptible} gene has a promoter sequence as set forth in SEQ ID NO:9, below:

GCACCCAACCCTTTATGAACAAAGCCAATGTGACATTGTGAAGCTTCAAGTCATCAGAGAAA
GATGAGGACAACTCATTCTTCGCTCTTGCGTCTGAACTCTGTAACCCATTTAAACATGTTTG
TTGGTTAGTCAAAACGGCACTGAGATAGGTTTCAAAATCTTTAGCTTCAGAAGTGTGAAGAA
AACCACTAACTTGGTTAGCAGTGGCATAGATGTTTGATAAGTATTCTAGACTTTGTTCAACA
ACGAATTGCCAATCTTCAAGAGCACCAAGTGAGTATTGAGACAAAGACAAGCTACCTTGAAG
ATATGAGTCCACTAAGTTCAAGAACTTACGAGCTTGGGACAAGGACTTTCGAAAAGAAATAC
GACCATAGTCGAAGATGTTTCCATTTTGATTAGCAAGCATGGTTTTGCAATAAGTAGGGTCT
AGGGTGGACTCACAAATAGTTTCGGGTGGAACAATGGAGGATAAACTGGTATATGCTATGGA
CAATGAGGCCAAGACCGAGAAGGAAAGCATTTAGGAAACAAACAACTTAAGGTCAAAGATAT
ACTTGACAGTCATTGGTTTTTTTTTTTTATGAAGGATTTAGGGTTGTTGTTACTACGGATGA
ACAAATAAGGTGCATGACTAGTTTGTCTTATATAGCGGACGAAGATTTTGTGTTATTATAAG
GATTAGTGGGAATGAACTATGGGAGCTTTGTCAGTTAGAAGTCAACATCCAAAAGGAATTTG
TCTTGGTGTGTTTGAAGTTGTTGCCATGGCTACCACGAATGCAGTCCCAACCGGCCATTTTG
AAATTTTTTATTAGGAGCTTAGGAGGAACATACATTAAGTTTACTATAAACGTCCATGTATG
CAAATATTCCTTGGGATTATATTTATTCGTTTGAATTGATATTAAGGTTTGTTTTGGTACAT
GGTTTATTACCTTGTGACATATATTTAATTTAATTTTAGTTTGTCAACGCGGGATTGGTATC
CAACAAAAAAACATGTATGTATATATTGCTGGTCATGCTTCTATTTCTTATATTAATAATAT
TTCTCTTGGATATTGTATTGGCTCAGGAGGCCCTGCCCCGCTATTGAGGAAATTAAGATATA
TTCAGAATATTATCAAGTCAGACGTAACACATAGTCACATAACTGAAATTGAAGATCAAATA
TTCATATATGCTTTGGGAGCAAAGCTTTTAGATTTTTATGTTACTTGATTTTACACATCAAT
TTCACATTTCAGGTTAATTTTGTATATATCTTTCTATAAAACTAGCCCTTCCAAAAGAACAA
GAAATAAACTGCAAAAGGAATTACCATTTCAGCCAAACAGAATTAGCCTGAAGCAATATATT
TAAAGCTTTCCTACCAGAAAGGCAGGTATGAAATAATTAAGAGAGTGGAAAAGGGAATTGAA
GCTCCAGTACTTGATGCAGGAGCTAAAATGATTAAGGATCTCACGCATAACAACACGTTGCA
TACTCGAGAGTTCAATTAATCGAGGTGGCATATTTAACCTAGTCAGTATGAGGTCCATGTCA
CATTATGACTAAAGCTAGCTCATTTTTTTTTCAAAAAATAAAAATAAAAATAAAAAAATAAA
AAATAAAATATATATATATATATATATATATATATATATATATATATATATATATATAGTTT
CACAGTGTCAACGTGAAACACCCCACTCCATGTTACAAT

(SEQ ID NO:9; Glyma.10G017100^{susceptible}, Williams 82) or a variant thereof having at least 90%, 95%, or more sequence identity to SEQ ID NO:9.

In other forms, the Glyma.10G017100^{susceptible} gene has a sequence as set forth in SEQ ID NO:10, below:

CCAACCACTCTATTTATCCACAGCACGTACCCTGCTAGCTAGTTACACATACACAGTAATCC
TAACCCACCTCCTAAGTCCTATCACATCATGAACAACCTCACACTTGCATCCATTCTGACCG
TGATTTCTTCTCTACTATTCTTTGGAACAACTCATTTAACCAACACCCAAACAACAAGAGTC
CCAGATCAAAAACATAAACACCTCCATTTCCAAAAGCACATACAAGTAGTAGCCCAATCCAC
ATGCGAAGGAACACTCTACCCAGACCTATGTGTCTTAACACTAGCCACATTCCCAGATCTCA
CAACAAAATCTGTCCCACAAGTGATATCCTCAGTGGTCAACCATACCATGTACGAGGTAAGA
TCAACGTCCTACAACTGCAGCGGCCTCAAAAAGATGCTCAAAAACCTCAACCCACTCGACCA
GAGAGCCCTCGACGACTGTCTCAAACTGTTTGAAGACACCAGCGTCGAGCTCAAAGCCACCA
TCGACGATCTCTCCATCAAGAGCACCATAGGGTCTAAACTGCACCATGACTTGCAGACTCTG
CTGAGTGGAGCAATGACCAACTTGTACACGTGCCTCGATGGCTTTGCGTACAGCAAAGGGCG
CGTGGGGGACAGAATCGAGAAGAAGCTGCTTCAAATATCGCATCACGTGAGCAACTCGTTGG
CCATGCTGAACAAAGTGCCTGGAGTTGAGAAATTAACAACTTCTTCGGAATCTGATGAGGTG
TTTCCAGAATATGGAAAGATGCAAAAGGGGTTCCCTTCGTGGGTGTCCTCTAAAGACCGAAA
GCTTCTTCAAGCTAAAGTGAATGAGACCAAGTTCAATCTTGTTGTTGCCAAAGATGGCACTG
GCAACTTCACCACTATAGGGGAAGCACTGTCTGTGGCTCCCAACTCAAGCACAACTAGGTAC
ATAACAAAATACTTTATTTCAGACCAATGTGATTGATTATAATTTACACTAGTATAAATGAA
TTACATAGTAAAAACGTAGTACCAATGGGATACAAATTAAGAGAAAAGAGAAAGGTGTTAGT
AGAGTGTCAATTTTTTTTTAAAGATTTTTAATAATGCCATGTATTCAATTGGAAATGTATAC
ACTATATACAGTGAGCTTTTTTTTTTTTTATGTATAAGTGAAAAGAATAAATATTAAGCATT
AGTCAAAATAAAATACAGAAGTCATATGAGTTATTTAAAAATCACGTTAAGAAACTCATTCT
CACATTATATCTATGTATAATTATATATGACTTAGATGTACTGTTTTCAATCTGATGTAAGA
AACTCATTCTTACATTATACCTCTCCTATTTGATTGTAAGAAATATTTCTTCATTGGGTTGT
AAAATTAAATGAGTGTAAGTGATAACAGGTTTGTGATACACGTAACGGCGGGGGCATACTTC
GAGAATGTGGAAGTGATAAGGAAGAAGACGAATCTAATGTTTGTTGGAGACGGTATTGGAAA
GACAGTAGTGAAAGGCAGTAGGAATGTTGAGGACGGGTGGACCATTTTTCAATCTGCTACTG
TTGGTAAGTATTAAATTTAGAGTGCAGCAATGCATTCGGAAGGTCCCCTTAAAAATCGGTTC
GTCAGCATTCATCGGGCAGAATATGGCTCTGATACCATATTAAATTTATAGTGTAGCAAAGA
CATTCGAAAAATTCCCTTAAAAACCGGTTTATAAGGGGTGGCCTACCGAACTATATAAGTAC
TTATCAAAATTTGCTAAACATCCAATGTAGGACTATTTTCAACAGTAAATATGTCTTTCTTC
TTCCCACCTTCTGCTACTATTTTTAATTAGTTATGTTTTTTTCTTGGTTTGATATTCCAACT
TTGAAAAATAAAAATAGGTATGATATATAGAATGGTCATAAGTGAGATTTGATTAGTAGCTC
GGTCACTTAATAAAGTTTTATAAATGCTCTACTTTGAGGCTGGCGTTGTGATTGTTAAGTCA
AAGACAGAGAAAAAAAATCATTAGAAAAAGTTAGAACAAGACAACGAAAATGGCAACTTGAT
GCACAGCCCACATGTTGGTTTAAAAATTATTAATAAACTAATTTTGCTAGGTGATTTTGTAG
TTAAAATACATAAAGTAGATGTGGGAAGATCATTTTAAATGGTTTGGACAGACATGGACTAC
TTGGGTTTGTCAAGACATCGTGCAGTTCCTGCATTGCATTGAAAAATAGCGTTGTAGAGGTT
GATGCATGTAATTATCTTTTGTTTTGTTTTGTGTGTGTACACTAGTAAAATCCTACCTCTTG
TGAGAATAAGCACAACTTAGTTTTGAATGTTTGGAATAAAACCAAGGAAAGTTCAAAAGATA
AAAGGCTTTGTCATTTTAAAATTTTTGAGGATTCTGCTTTCCGTGATTGAACATAATGCTGA
AAAAAGATAGGTGATGAGTGCATGTATATATGTTTAAAGACTATTTTCAAAGGCATCACTGC
AAATTTAAACTACCCCAGCTATCAATGACGTATGCATGAAATATAAATAGTGTCAAAGTGAT
GCTAGTATTATTGGTTACAATGCTGACTAGCTAGATTCCAAAATGCTTGTTTTTTTTTTGTG
ATCATTGCCCGTCGTCAACTATTCCTGTTATGGGAATAAATTTTGAAGACTCTAATTTTGTG
TTTTCAGTGTTCTGTTTGGTTTTTCCAGTTATATTGCAATCCAGTAATATCAATTGGACTTC
TCCATCAGACAACGGGTCTCAATCACAATGACATTAGCAAATATGTATTGTAAATAAATCAA
ATTTCTTATTTCTTGTTAAAGGTAGCAGTAATAAACCTAGATTTGGGATCTCCTGCCCGAAA
GCATGTTTTCTTTATTAATATTTTCCGAGCTATGAGTGGTGACACCATTTTTTGGAAAGAAA
ATGACGTGAGTAATCTTGGTGAAAGATAGAAAAAGAACAGAGAAAAAAAACATAACCGGTGT
GGTCTAATGAGAAGAAATAAAAATAGAATTGGAAACTCTTTTATAAGACAAAAAAAAAAAAA
AATACATGCAGGCAGAGGTAGGACTATCTTTGGCCTTAGATACATGATAATTTTATTTATAT
TTTCGCGAAAAGTAGAGTTACCTCTATGGTTGGTCAAAGTAAAGGGTCCACATGCCTATCCA
CGCCCACCTAAAGATCTGCATAATTCTCATCACAACCAAACTACTTTTTCAGTGCCATTAGG
TCCACTTTGCTTGTTTTTTCTTCTATGCTTCCCCTTTTACTTTTCTGCTTTTGCTCTTAACA
AATAAAAATGCTTTAATTTCCTCTTTTGGGATAACATGGATAAAAGAAAGTGAATTTTATGA
TCTTTATTATTATTCATGTGACAAATAATGTGCAGTACAAAGTTAACAGACTTTTAGTTCTC
TTTCTTCGTTTCAATGATACCTTTATGTTGAGTTTGTGTCTCCCAGCAAAGTTTGCTCTTTA
TTGTTCATTTATGAATAATGATCCAAAATCTAATCCTAACAGCGTTGACAATAATGGTATGT
TGCAACTATTCAGTTGTAACTCAGTTGGCAACACATCTTAAGTTTATGAAAAAAAGATCTGT
GTTCAATCCTTACAAGAAAAAGGATGAATAATTTTAAATGTAATTAAGTCTCGAACAGAATG
ATTATGATTGTTGTTAATAAAGGAAAATGGTATGTTGCAGCTGTTGTAGGAGCAGGATTCAT
AGCAAAGGGTATAACATTTGAGAAGTCAGCAGGACCCGACAAACACCAAGCTGTGGCACTGA
GAAGTGGTGTGCTGACTTCTCAGCTTTCTACCAATGCAGTTTCGTTGGCTACCAGGACACTC
TCTACGTCCATTCCCTGCGCCAATTCTACCGTGAACGTGACATTTATGGCACTGTAGACTTC
ATTTTCGGCAATGCAGCTGTGGTATTCCAAAACTGCAACTTATACGCACGCAAGCCAAACGA
AAATCAGAAGAACTTGTTCATGGCACAAGGCAGAGAGGACCCTAACCAAAACACTGGCATAT
CCATCTTGAACTGCAAGATTGCAGCTGCTGCAGATTTGATCCCTGTGAAATCCTCGTTCAAG
AGCTACCTAGGACGTCCTTGGAAAATGTACTCTATGACTGTTGTGTTAAAATCTTACGTGGA
TATAGACCCAGCAGGGTGGTTGGAATGGAATGAAACATTTGCATTGGATACGTTGTATTATG
GGGAGTACATGAATAGGGGTCCATGTTCAAACACAAGTGGTAGGGTTACGTGGCCAGGTTAT
AGGGTCATTAACAGCTCCATTGAGGCAAGCCAATTCACAGTTGGACAGTTTATTCAAGACAA
TGATTGGTTGAACAACACTGGCATCCCATTCTTCTCTGGTTTGAGTTGAGAATATTTTTAGG
TTGAGCTGAACCTAGGAGATCACGTGATTTGTAAGGTTACATTACATTACATTCCTGTGTAT
TACTTCATTGGAGTTCCTATAGTATAGGGCTATTGTGGCTATAGCAGTCACGTGATGACTAT
ACCCAAAAATTAGTCTTGTGAGGGATTATATATTTTAGTGCTCTAATAAAAGCAACTAACCC
CACCAGAGATAT

(SEQ ID NO:10, Glyma.10G017100^{susceptible}, Williams 82) or a variant thereof having at least 90%, 95%, or more sequence identity to SEQ ID NO: 10.

In other forms, the Glyma.10G017100^{susceptible} has a CDS sequence as set forth in SEQ ID NO:11, below:

ATGAACAACCTCACACTTGCATCCATTCTGACCGTGATTTCTTCTCTACTATTCTTTGGAAC
AACTCATTTAACCAACACCCAAACAACAAGAGTCCCAGATCAAAAACATAAACACCTCCATT
TCCAAAAGCACATACAAGTAGTAGCCCAATCCACATGCGAAGGAACACTCTACCCAGACCTA
TGTGTCTTAACACTAGCCACATTCCCAGATCTCACAACAAAATCTGTCCCACAAGTGATATC
CTCAGTGGTCAACCATACCATGTACGAGGTAAGATCAACGTCCTACAACTGCAGCGGCCTCA
AAAAGATGCTCAAAAACCTCAACCCACTCGACCAGAGAGCCCTCGACGACTGTCTCAAACTG
TTTGAAGACACCAGCGTCGAGCTCAAAGCCACCATCGACGATCTCTCCATCAAGAGCACCAT
AGGGTCTAAACTGCACCATGACTTGCAGACTCTGCTGAGTGGAGCAATGACCAACTTGTACA
CGTGCCTCGATGGCTTTGCGTACAGCAAAGGGCGCGTGGGGGACAGAATCGAGAAGAAGCTG
CTTCAAATATCGCATCACGTGAGCAACTCGTTGGCCATGCTGAACAAAGTGCCTGGAGTTGA
GAAATTAACAACTTCTTCGGAATCTGATGAGGTGTTTCCAGAATATGGAAAGATGCAAAAGG
GGTTCCCTTCGTGGGTGTCCTCTAAAGACCGAAAGCTTCTTCAAGCTAAAGTGAATGAGACC
AAGTTCAATCTTGTTGTTGCCAAAGATGGCACTGGCAACTTCACCACTATAGGGGAAGCACT
GTCTGTGGCTCCCAACTCAAGCACAACTAGGTTTGTGATACACGTAACGGCGGGGGCATACT
TCGAGAATGTGGAAGTGATAAGGAAGAAGACGAATCTAATGTTTGTTGGAGACGGTATTGGA
AAGACAGTAGTGAAAGGCAGTAGGAATGTTGAGGACGGGTGGACCATTTTTCAATCTGCTAC
TGTTGCTGTTGTAGGAGCAGGATTCATAGCAAAGGGTATAACATTTGAGAAGTCAGCAGGAC
CCGACAAACACCAAGCTGTGGCACTGAGAAGTGGTGTGCTGACTTCTCAGCTTTCTACCAAT
GCAGTTTCGTTGGCTACCAGGACACTCTCTACGTCCATTCCCTGCGCCAATTCTACCGTGAA
CGTGACATTTATGGCACTGTAG

(SEQ ID NO:11, Glyma.10G017100^{susceptible}, Williams 82) or a variant thereof having at least 90%, 95%, or more sequence identity to SEQ ID NO: 11.

Therefore, a polynucleotide is disclosed having a nucleic acid sequence SEQ ID NO: 9, or 10, or 11, or a fragment or variant thereof. Also disclosed is a fragment or variant of Glyma.10G017100^{susceptible} coding sequence having a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO:9, or 11, or 12. A fragment can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 50, 75, 100, or more nucleotides shorter than SEQ ID NO:9, or 10, or 11.

Also disclosed is a polynucleotide that hybridizes under stringent conditions to a polynucleotide having the nucleic acid sequence SEQ ID NO:9, or 10, or 11, or a fragment or variant thereof.

ii. Glyma.10G017100^{resistant}

In some forms, the resistant form of Glyma.10G017100 is derived from a QTL derived from the Forrest soybean line. In some forms, a resistant version of PME1 has a premature stop codon before the PME catalytic domain. If a direct interaction between Mi-CBP and the catalytic domain of PME1 is required, the resistant allele would lack the binding site, resulting in the loss of a susceptibility factor.

Soybean plants containing a major QTL, which express Glyma.10G017100^{resistant}, do not express a functional Glyma.10G017100 protein. Glyma.10G017100^{resistant} contains a SNP unique to nematode-resistant soybeans, which produces an altered Glyma.10G017100 protein that may be non-expressed, or a truncated or alternative splice variant of the wildtype protein. The protein encoded by Glyma.10G017100^{resistant} may have altered function or be non-functional.

It has also been discovered that expression of Glyma10G017100 in soybean is induced by herbivory. Accordingly, the control/promoter sequences that control expression of Glyma10G017100 can be used to promote herbivory-induced expression of a gene of interest. In some embodiments, the control sequences include genomic untranslated regions 5′ of Glyma10G017100, 3′ of Glyma10G017100, or combinations thereof.

In other embodiments, the control/promoter sequence that controls expression of Glyma10G017100 includes 50, 100, 150, 250, 500, 750, 1,000, 1,250, 1,500, 2,500, 5,000 nucleotides 5′ of Glyma10G017100. In some embodiments, the control sequence ends immediately before the ATG start codon of Glyma10G017100. For example, the control sequence that controls expression of Glyma10G017100 can include 50, 100, 150, 250, 500, 750, 1,000, 1,250, 1,500, 2,500, 5,000 nucleotides of the nucleic acid sequence

In some forms, the Glyma.10G017100^{resistant} has a promoter sequence as set forth in SEQ ID NO:12 below:

GCACCCAACCCTTTATGAACAAAGCCAATGTGACATTGTGAAGCTTCAAGTCATCAGAGAAA
GATGAGGACAACTCATTCTTCGCTCTTGCGTCTGAACTCTGTAACCCATTTAAACATGTTTG
TTGGTTAGTCAAAACGGCACTGAGATAGGTTTCAAAATCTTTAGCTTCTGAAGTGTGAAGAA
AACCACTAACTCGGTTAGCAGTGGCATAGATGTTTGATAAGTATTCTAGACTTTGTTCAACA
ACGAATTGCCAATCTTCAAGAGCACCAAGTGAGTATTGAGACAAAGACAAGCTACCTTGAAG
ATATGAGTCCACTAAGTTCAAGAACTTACGAGCTTGGGACAAGGACTTTCGAAAAGAAATAC
GACCATAGTCGAAGATGTTTCCATTTTGATTAGCAAGCATGGTTTTGCAATAAGTAGGGTCT
AGGGTGGACTCACAAATAGTTTCGGGTGGAACAATGGAGGATAAACTGGTATATGCTATGGA
CAATGAGACCAAGACCGAGAAGGAAAGCATTTAGGAAAAAAACAACTTAAGGTCAAAGATAT
ACTTGACAGTCATTAGTTTTCTCTTTTGTTTTTCTTCGTATGCTTGTAAGGAACCCATGAGT
TGCTCAATAGTCATGGTCTTTAAATCCTTGTTTTCTTCAATGTTGGTAACAATGAAGTCAAA
ACTTGGATTTAAAGTTCGAAGTATTTTTTCCATGACCTTCACCTCATCAACATTTTCACCAT
TTCTTTTAAGTTGATTGACTACGGCCAATACTCGAGAAAAATAATCAGAAATTGACTCGGAC
TCCTCCATAAACAAACGCTCAAAGTCACCTCTAAGAGTTTGAAGACGAATCTTTTTTACCTG
CTCAACTCCTTTGTTGCAAGTTTGAAGCTTATCCCATGCTTCTTTGGCCGTCGTTGCGTTGG
ATATCTTCTCAAATGTATCTTCATCCACCGATTGATAAATGAGAAAGAGAGCTTTCTTGTCT
CTCTTTCTTGACTCCTTCAACGTCTCCTTTACACCTTGGCTTAGCGAGACTTCATCTTGCTC
CTCGAAGCCATTCTCTACGATATCCCACACATCTTGAGCTCCTAGTAGCGCCTTCATCTTGA
TACTCCAATTATCATAGTTGTTCTTTGTGAGCATCGGCATTTGGAAATGAAAACCTCCATTC
GCCATCTTTTGAGGATCTTGAAGCTCTGATACCACTTTGTTGGAAATAAGGCTTTTTATGTT
TAGGAAAAGTGTTTAGGAATATTGGAGACTTTGAATAGAAACTTGATAGGAAGGAGAATTCT
TTATGGAGGAGAGAACTTTGTATTTTTGCTTGATACAAATGTGTAGGATTACATCTCTATTT
ATACTACTCTAAGGAGAACTCTAGACACACTAATTCTAGAGAGTTCTCAACTCTAGAGATCC
AAAGAGTATTCTAGAGAATATTAAAACCATAAGAAATATCTAGACATTCCAAACACTACAAG
AATTCTCTAGAAACATGACCCATAATTACTTAAGCCCAAAATAACTAAGTCCAAGAAACCAA
ATAATTAATTTGGGCCCAAATCAAGTTTATATTTCAACACAAATAAGGTGCATGACTAGTTT
GTCTTATATAGCGGACGAAGATTTTGTGTTATTATAAGGATTAGTGGGAATGAACTATGGGA
GCTTTGTCAGTTAGAAGTCAACATCCAAAAGGAATTTGTCTTGTTTGTTTGAAGTTGTTGCC
ATGGCTACCACGAATGCAGTCCCAACCGGCCATTTTGAAATTTTTTATTAGGAGCTTAGGAG
GAACATACATTAAGTTTACTATAAACGTCCATGTATGCAAATATTCCTTGGGATTATATTTA
TTCGTTTGAATTGATATTAAGGTTTGTTTTGGTACATGGTTTATTACCTTGTGACATATATT
TAATTTAATTTTAGTTTGTCAACGCGGGATTGGTATCCAACAAAAAAACATGTATGTATATA
TTGCTGGTCATGCTTCTATTTCTTATATTAATAATATTTCTCTTGGATATTGTATTGGCTCA
GGAGGCCCTGCCCCGCTATTGAGGAAATTAAGATATATTCAGAATATTATCAAGTCAGACGT
AACACATAGTCACATAACTGAAATTGAAGATCAAATATTCATATATGCTTTGGGAGCAAAGC
TTTTAGATTTTTATGTTACTTGATTTTACACATCAATTTCACATTTCAGGTTAATTTTGTAT
ATATCTTTCTATAAAACTAGCCCTTCCAAAAGAACAAGAAATAAACTGCAAAAGGAATTACC
ATTTCAGCCAAACAGAATTAGCCTGAAGCAATATATTTAAAGCTTTCCTACCAGAAAGGCAG
GTATGAAATAATTAAGAGAGTGGAAAAGGGAATTGAAGCTCCAGTACTTGATGCAGGAGCTA
AAATGATTAAGGATCTCACGCATAACAACACGTTGCATACTCGAGAGTTCAATTAATCGAGG
TGGCATATTTAACCTAGTCAGTATGAGGTCCATGTCACATTATGACTAAAGCTAGCTCATTT
TTTTTCAAAAAAAAAAAAATATATATATATATATATATATATATATATATATAGTTTCACAG
TGTCAACGTGAAACACCCCACTCCATGTTACAAT

(SEQ ID NO:12, Glyma.10G017100^{resistant}, Forrest) or a variant thereof having at least 90%, 95%, or more sequence identity to SEQ ID NO: 12.

In other forms, the Glyma.10G017100^{resistant} has a gene sequence as set forth in SEQ ID NO:13, below:

CCAACCACTCTATTTATCCACAGCACGTACCCTGCTAGCTAGTTACACATACACAGTAATCC
TAACCCACCTCCTAAGTCCTATCACATCATGAACAACCTCACACTTGCATCCATTCTGACCG
TGATTTCTTCTCTACTATTCTTTGGAACAACTCATTTAACCAACACCCAAACAACAAGAGTC
CCAGATCAAAAACATAAACACCTCCATTTCCAAAAGCATATACAAGTAGTAGCCCAATCCAC
ATGCGAAGGAACACTCTACCCAGACCTATGTGTCTTAACACTAGCCACATTCCCAGATCTCA
CAACAAAATCTGTCCCACAAGTGATATCCTCAGTGGTCAACCATACCATGTACGAGATAAGA
TCAACGTCCTACAACTGCAGCGGCCTCAAAAAGATGCTCAAAAACCTCAACCCACTCGACCA
GAGAGCCCTCGACTACTGTCTCAAACTGTTTGAAGACACCAGCGTCGAGCTCAAAGCCACCA
TCGACGATCTCTCCATCAAGAGCACCATAGGGTCGAAACGGCATCATGACTTGCAGACTCTG
CTGAGTGGAGCAATGACCAACTTGTACACGTACCTCGATGGCTTTGCGTACAGCAAAGGGCG
CGTGGGGGACAGAATCGAGAAGAAGCTGCTTCAAATATCGCATCACGTGAGCAACTCGTTGG
CCATGCTGAACAAAGTGCCTGGAGTTGAGAAATTAACAACTTCTTCGGATTCTGATGAGGTG
TTTCCAGAATATGGAAAGATGCAAAAGGGGTTCCCTTCGTGGGTGTCCTCCAAAGACCGAAA
GCTTCTTTAAGCTAAAGTGAATGAGACCAAGTTCAATCTTGTTGTTGCCAAAGATGGCACTG
GCAACTTCACCACTATAGGGGAAGCACTGTCTGTGGCTCCCAACTCAAGCACAACTAGGTAC
ATAACAAAATACTTTATTTCAGACCAATGTGATTGATTATAATTTACTCTAGTATAAATGAA
TTACATAGTAAAAACGTAGTACCAATGGGATACAAATTAAGAGAAAAGAGAAAGGTGTTAGT
AGAGTGTCAATTTTTTTTTAAAGATTTTTAATAATGCCATGTATTCAATTGGAAATGTATAC
ACTATATACAGTGAGCTTTTTTTTTTTTTTTTTATGTATAAGTGAAAAGAATAAATATTAAG
CATTAGTCAAAATAAAATACAGAAGTCATATGAGTTATTTAAAAATCACGTTAAGAAACTCA
TTCTCACATTATATCTATGTATAATTATATATGACTTAGATGTACTGTTTTCAATCCGATGT
AAGAAACTCATTCTTACATTATACCTCTCCTATTTGATTGTAAGAAATATTTCTTCATTGGG
TTGTAAAATTAAATGAGTGTAAGTGATAACAGGTTTGTGATACACGTAACGGCGGGGGCATA
CTTCGAGAATGTGGAAGTGATAAGGAAGAAGACGAATCTAATGTTTGTTGGAGACGGTATTG
GAAAGACAGTAGTGAAAGGCAGTAGGAATGTTGAGGACGGGTGGACAATTTTTCAATCTGCT
ACTGTTGGTAAGTATGTCTTTCTTCTTCCCACCTTCTGCTACTATTTTTAATTAGTTATGTT
TTTTTCTTGGTTTGATATTCCAACTTTGAAAAATAAAAATAGGTATGATATATAGAATGGTC
ATAAGTGAGATTTGATTAGTAGCTCGGTCACTTAATAAAGTTTTATAAATGCTCTACTTTGA
GGCTGGCGTTGTGATTGTTAAGTCAAAGACAGAAAAAAAATCATTAGAAAAAGTTAGAACAA
GACAACGAAAATGGCAACTTGATGCACAGCCCACATGTTGGTTTAAAAATTATTAATAAACT
AATTTTGCTAGGTGATTTTGTAGTTAAAATACATAATGTAGATGTGGGAAGATCATTTTAAA
TGGTTTGGACAGACATGGACTACTTGGGTTTGTCAAGACATCGTGCAGTTCCTGCATTGCAT
TGAAAAATAGTGTTGTAGAGGTTGATGCATGTAATTATTTTTTTTTTGTTTTGTGTGTGTAC
ACTAGTAAAATCCTACCTCTGGTGAGAATAAGCACAACTTAGTTTTGAATGTTTGGAATAAA
ACCAAGGAAAGTTCAAAAGATAAAAGGCTTTGCCATTTTAAAGACTAGGTGATGAGTACATG
TATATATGTTTAAAGACTATTTTCAAAGGCATCACTGCAAATTTAAACTACCCCAGCTATCA
ATGACGTATGCATGAAATATAAATAGTGTCAAAGTGATGCTAGTATTATTGGTTACAATGCT
GACTAGCTAGATTCCAAAATGGTTGTTTTTTTTTTTTATCATTGCCCGTTGTCAACTATTCC
TGTTATGGGAATAAATTTTGAGGACTCTAATTTTGTGTTTTCAGTGTTCTGTTTGGTTTTTC
CAGTTATATTGCAATCCAGTAATATCAATTGGACTTCTCCATCAGACAACGCGTCTCAATCA
CAATGACATTAGCAAATATATATTGTAAATAAATCAAATTTCTTATTTCTTGTTAAAGTTAG
CAGTAATAAACCTAGATTTGGGATCTCCTGCCCGAAAGCATGTTTTCTTTATTAATATTTTC
CGAGCTATGAGTGGTGACACCATTTTTTGGAAAGAAAATGACGTGAGTAATCTTGGTGAAAG
ATAGAAAAAGAACAGAGAAAAAAAACATAACCGGTGTGGTCTAATGAGAAGAAATAAAAATA
GAATTAGAAACTCTTTTATAAGACCAAAAAAAAAAAAAATACATGCAGGCAGGCAGAGGAGG
TAGGACTATCTCTGGCCTTAGATACATGATAATTTTATTTATATTTTCGCGAAAAGTAGAGT
TACCTCTATGGTTGGTCAAAGTAAAGGGTCCACATGCCTATCCACGCCCACCTAAAGATCTG
CATAATTCTCATCACAACCAAACTACTTTTTCAGTGCCATTAGGTCCACTTTGCTTGTTTTT
TCTTCTATGCTTCCCCTTTTACTTTTCTGCTTTTGCTTTTAACAAATAAAAATGCTTTAATT
TCCTCTTTTGGGATAACATGGATAAAAGAAAGTGAATTTTATGATCTTTATTATTCATGTGA
CAAATAATGTGCAGTACAAAGTTAACAGACTTTTAGTTTTCTTTCTTCGTTTCAATGATACC
TTTATGTTGAGTTTGTGTCTCTCAGCAAAGTTTGCTCTTTATTGTTCATTTATGAATAATGA
TCCAAAATCTAATCCTAACAGCGTTGACAATAATGGTATGTTGCAACTATTCAGTTGTAACT
CAGTTGGCAACAGATCTTAAGTTTATGGAAAAAAAATCTGTGTTCAATCCTTACAAGAAAAA
GGATGAATAATTTAAAATGTAATTAAGTCTCGAACAGAATGATTATGATTGTTGTTAATAAA
AGAAAATGGTATGTTGCAGCTGTTGTAGGAGCAGGATTCATAGCAAAGGGTATAACATTTGA
GAAGTCAGCAGGACCCGACAAACACCAAGCTGTGGCACTGAGAAGTGGTGTGCTGACTTCTC
AGCTTTCTACCAATGCAGTTTCGTTGGCTACCAGGACACTCTCTACGTCCATTCCCTGCGTC
AATTCTACCGTGAACGTGACATTTATGGCACTGTAGACTTCATTTTCGGCAATGCAGCTGTG
GTATTCCAAAACTGCAACTTATACGCACGCAAGCCAAACGAAAATCAGAAGAACTTGTTCAT
GGCACAAGGCAGAGAGGACCCTAACCAAAACACTGGCATATCCATCTTGAACTGCAAGATTG
CAGCTGCTGCAGATTTGATCCCTGTGAAATCCTCGTTCAAGAGCTACCTAGGACGTCCTTGG
AAAATGTACTCTATGACTGTTGTGTTAAAATCTTACGTGGATATAGACCCAGCAGGGTGGTT
GGAATGGAATGAAACATTTGCATTGGATACGTTGTATTATGGGGAGTACATGAATAGGGGTC
CATGTTCAAACACAAGTGGTAGGGTTACGTGGCCAGGTTATAGGGTCATTAACAGCTCCATT
GAGGCAAGCCAATTCACAGTTGGACAGTTTATTCAAGACAATGATTGGTTGAACAACACTGG
CATCCCATTCTTCTCTGGTTTGAGTTGAGAATATTTTTAGGTTGAGCTGAACCTAGGAGATC
ACGTGATTTGTAAGGTTACATTACATTACATTCCTGTGTATTACTTCATTGGAGTTCCTATA
GTATAGGGCTATTGTGGCTATAGCAGTCACGTGATGACTATACCCAAAAATTAGTCTTGTGA
GGGATTATATATTTTAGTGCTCTAATAAAAGCAACTAACCCCACCAGAGATAT

(SEQ ID NO:13, Glyma.10G017100^{resistant} Forrest) or a variant thereof having at least 90%, 95%, or more sequence identity to SEQ ID NO: 13.

In other forms, the Glyma.10G017100^{resistant} has a CDS sequence as set forth in SEQ ID NO: 14, below:

ATGAACAACCTCACACTTGCATCCATTCTGACCGTGATTTCTTCTCTACTATTCTTTGGAAC
AACTCATTTAACCAACACCCAAACAACAAGAGTCCCAGATCAAAAACATAAACACCTCCATT
TCCAAAAGCATATACAAGTAGTAGCCCAATCCACATGCGAAGGAACACTCTACCCAGACCTA
TGTGTCTTAACACTAGCCACATTCCCAGATCTCACAACAAAATCTGTCCCACAAGTGATATC
CTCAGTGGTCAACCATACCATGTACGAGATAAGATCAACGTCCTACAACTGCAGCGGCCTCA
AAAAGATGCTCAAAAACCTCAACCCACTCGACCAGAGAGCCCTCGACTACTGTCTCAAACTG
TTTGAAGACACCAGCGTCGAGCTCAAAGCCACCATCGACGATCTCTCCATCAAGAGCACCAT
AGGGTCGAAACGGCATCATGACTTGCAGACTCTGCTGAGTGGAGCAATGACCAACTTGTACA
CGTACCTCGATGGCTTTGCGTACAGCAAAGGGCGCGTGGGGGACAGAATCGAGAAGAAGCTG
CTTCAAATATCGCATCACGTGAGCAACTCGTTGGCCATGCTGAACAAAGTGCCTGGAGTTGA
GAAATTAACAACTTCTTCGGATTCTGATGAGGTGTTTCCAGAATATGGAAAGATGCAAAAGG
GGTTCCCTTCGTGGGTGTCCTCCAAAGACCGAAAGCTTCTTTAAGCTAAAGTGAATGAGACC
AAGTTCAATCTTGTTGTTGCCAAAGATGGCACTGGCAACTTCACCACTATAGGGGAAGCACT
GTCTGTGGCTCCCAACTCAAGCACAACTAGGTTTGTGATACACGTAACGGCGGGGGCATACT
TCGAGAATGTGGAAGTGATAAGGAAGAAGACGAATCTAATGTTTGTTGGAGACGGTATTGGA
AAGACAGTAGTGAAAGGCAGTAGGAATGTTGAGGACGGGTGGACAATTTTTCAATCTGCTAC
TGTTGCTGTTGTAGGAGCAGGATTCATAGCAAAGGGTATAACATTTGAGAAGTCAGCAGGAC
CCGACAAACACCAAGCTGTGGCACTGAGAAGTGGTGTGCTGACTTCTCAGCTTTCTACCAAT
GCAGTTTCGTTGGCTACCAGGACACTCTCTACGTCCATTCCCTGCGTCAATTCTACCGTGAA
CGTGACATTTATGGCACTGTAG

(SEQ ID NO:14, Glyma.10G017100^{resistant} Forrest) or a variant thereof having at least 90%, 95%, or more sequence identity to SEQ ID NO: 14.

Therefore, a polynucleotide is disclosed having a nucleic acid sequence SEQ ID NO: 12, or 13, or 14, or a fragment or variant thereof. Also disclosed is a fragment or variant of Glyma.10G017100^{resistant} coding sequence having a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO:12, or 13, or 14. A fragment can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 50, 75, 100, or more nucleotides shorter than SEQ ID NO: 12, or 13, or 14.

b. Polypeptides
i. Glyma.10G017100^{susceptible}

An amino acid sequence encoded by a Glyma.10G017100^{susceptible} gene is also disclosed.

Thus disclosed is a polypeptide encoded by the nucleic acid sequence of SEQ ID NO:10, or 11, or a fragment or variant thereof. Also disclosed is a polypeptide encoded by a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO:10, or 11 or a fragment or variant thereof. Also disclosed is a polypeptide encoded by a polynucleotide that hybridizes under stringent conditions to a polynucleotide including the nucleic acid sequence SEQ ID NO: 10, or 11, or a fragment or variant thereof.

A polypeptide that is a fragment or variant of a Glyma.10G017100^{susceptible} gene product is also disclosed. Thus, a polypeptide encoded by a polynucleotide having a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 10, or 11 is disclosed. The fragment can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 50, 75, or more amino acids shorter than the polypeptide encoded by the nucleic acid sequence SEQ ID NO:10, or 11.

In some embodiments, the gene product of Glyma.10G017100^{susceptible} long isoform includes the amino acid sequence encoded by SEQ ID NO: 15.

In still other embodiments an amino acid sequence of a Glyma.10G017100^{susceptible} polypeptide is:

MNNLTLASILTVISSLLFFGTTHLINTOTTRVPDQKHKHLHFQKHIQVVAQSTCEGTLYPDL
CVLTLATFPDLTTKSVPQVISSVVNHTMYEVRSTSYNCSGLKKMLKNLNPLDORALDDCLKL
FEDTSVELKATIDDLSIKSTIGSKLHHDLQTLLSGAMINLYTCLDGFAYSKGRVGDRIEKKL
LQISHHVSNSLAMLNKVPGVEKLTTSSESDEVFPEYGKMQKGFPSWVSSKDRKLLQAKVNET
KFNLVVAKDGTGNFTTIGEALSVAPNSSTTRFVIHVTAGAYFENVEVIRKKTNLMFVGDGIG
KTVVKGSRNVEDGWTIFQSATVAVVGAGFIAKGITFEKSAGPDKHOAVALRSGVLTSQLSTN
AVSLATRTLSTSIPCANSTVNVTFMAL

(SEQ ID NO:15, Glyma.10G017100^{susceptible}, Williams 82) or a variant thereof having at least 90%, 95%, or more sequence identity to SEQ ID NO: 15.

Therefore, a polypeptide is disclosed having an amino acid sequence of SEQ ID NO:15, or a fragment or variant thereof. Also disclosed is a fragment or variant of a Glyma.10G017100^{susceptible} polypeptide having a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO:15. A fragment can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 50, 75, 100, or more amin acids shorter than SEQ ID NO:15.

Also disclosed are polynucleotides encoding the amino acid sequence SEQ ID NO: 15, or fragments or variants thereof.

ii. Glyma.10G017100^{resistant}

An amino acid sequence encoded by a Glyma.10G017100^{resistant} gene is also disclosed.

Thus disclosed is a polypeptide encoded by the nucleic acid sequence of SEQ ID NO:13, or 14, or a fragment or variant thereof. Also disclosed is a polypeptide encoded by a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO:13, or 14, or a fragment or variant thereof. Also disclosed is a polypeptide encoded by a polynucleotide that hybridizes under stringent conditions to a polynucleotide including the nucleic acid sequence SEQ ID NO: 13, or 14, or a fragment or variant thereof.

A polypeptide that is a fragment or variant of a Glyma.10G017100^{resistant} gene product is also disclosed. Thus, a polypeptide encoded by a polynucleotide having a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 13, or 14 is disclosed. The fragment can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 50, 75, or more amino acids shorter than the polypeptide encoded by the nucleic acid sequence SEQ ID NO:13, or 14.

In some embodiments, the gene product of Glyma.10G017100^{resistant} altered long isoform includes the amino acid sequence encoded by SEQ ID NO: 16:

MNNLTLASILTVISSLLFFGTTHLINTOTTRVPDQKHKHLHFQKHIQVVAQSTCEGTLYP
DLCVLTLATFPDLTTKSVPQVISSVVNHTMYEIRSTSYNCSGLKKMLKNLNPLDQRALDY
CLKLFEDTSVELKATIDDLSIKSTIGSKRHHDLQTLLSGAMINLYTYLDGFAYSKGRVGD
RIEKKLLQISHHVSNSLAMLNKVPGVEKLTTSSDSDEVFPEYGKMQKGFPSWVSSKDRKLL

(SEQ ID NO:16) or a variant thereof having one or more conservative amino acid substitutions and at least 90%, 95%, or more sequence identity compared to SEQ ID NO: 16.

A polypeptide is therefore disclosed having the amino acid sequence SEQ ID NO:16, or a fragment or variant thereof. A polypeptide having an amino acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO:16 is also disclosed.

A polypeptide that is a fragment or variant of Glyma10G017100^{resistant} protein including the amino acid sequence SEQ ID NO: 16 is also disclosed. A polypeptide having an amino acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to a fragment of SEQ ID NO:16 is disclosed. The fragment can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 50, or 75 amino acids shorter than SEQ ID NO: 16.

Also disclosed are polynucleotides encoding the amino acid sequence SEQ ID NO: 16, or fragments or variants thereof.

### 2. Homologs and Orthologs of Glyma.10G017100

The Glyma.10G017100 protein is homologous or orthologous to the pectin methylesterase inhibitor of soybean plants.

Exemplary genes that are homologous or orthologous to the described Glyma.10G017100 gene include:

Typically, genes that are homologous or orthologous share at least 75% sequence identity with Glyma.10G017100 and share at least part of the biological function of Glyma.10G017100. Exemplary genes that are homologous or orthologous to the described Glyma.10G017100 gene include: Gossypium hirsutum LOC107912360 (NCBI Accession: XP_016695983.2); Gossypium hirsutum LOC107924376 (NCBI Accession: XP_016710281.2); Gossypium hirsutum LOC107954268 (NCBI Accession: XP_016745265.2); Gossypium hirsutum (NCBI Accession: KAG4191929.1 and KAL1161596.1); Solanum lycopersicum LOC101260941 (NCBI Accession: XP_010321856.2); Nicotiana tabacum LOC107789276 (NCBI Accession: XP_016466534.1); Gossypium hirsutum LOC107925603 (NCBI Accession: XP_040938871.1); Capsicum annum LOC107855110 (NCBI Accession: PHT77615.1); Gossypium hirsutum LOC107946060 (NCBI Accession: XP_016735737.2); Capsicum annum LOC107864052 (NCBI Accession: LOC107864052); Gossypium hirsutum (NCBI Accession: KAG4191930.1 and KAL1161597.1); Nicotiana tabacum LOC107818898 (NCBI Accession: XP_016486864); Nicotiana tabacum LOC107782372 (NCBI Accession: XP_016458739.1); Solanum lycopersicum LOC101266668 (NCBI Accession: XP_004247400.2); Capsicum annum (NCBI Accession: KAF3671491.1); Nicotiana tabacum LOC107759512 (NCBI Accession: XP_016432961.1); Capsicum annum (NCBI Accession: PHT86133.1, KAF3622130.1, and KAF3634052.1); and Nicotiana tabacum LOC107763142 (NCBI Accession: XP_016437071.1), the NCBI sequence data of all of which are hereby incorporated by reference herein in their entirety.

### III. Methods for Increasing Pest Resistance

Methods of modulating pest resistance in plants are also disclosed. The methods can be used, for example, to increase a plant's resistance to pests such as parasites, such as nematodes. The methods can include, for example, altering, reducing or inhibiting the expression of a functional the β-1,4-endoglucancase (EG) and/or pectin methylesterase inhibitor (PME1) genes in a plant, expressing a pest resistance gene or QTL in a plant, or combinations thereof. In some embodiments, the pest resistance gene is placed under the control of an herbivory inducible expression control sequence, or sequences.

### A. Methods of Modulating Functional Expression of Susceptibility Genes

Methods of modulating pest resistance can include modulating the expression of a susceptibility gene, such as Glyma.10G017000, or a fragment, variant, ortholog, or homolog thereof, and/or Glyma.10G017100, or a fragment, variant, ortholog, or homolog thereof.

In some embodiments, pest resistance is increased in a plant by altering or decreasing the expression of a susceptibility gene or gene product, for example, a functional Glyma.10G017000 protein or a functional fragment, variant, ortholog or homolog thereof, and/or a functional Glyma.10G017100 protein or a functional fragment, variant, ortholog or homolog thereof.

In other embodiments, the method involves inhibiting functional β-1,4-endoglucancase (EG) and/or pectin methylesterase inhibitor (PME1) activity in a plant. In still other embodiments, the method involves engineering a transgenic plant to alter, reduce or inhibit expression of a functional β-1,4-endoglucancase (EG) and/or pectin methylesterase inhibitor (PME1) gene or gene product, or translation of a functional β-1,4-endoglucancase (EG) protein and/or translation of a functional pectin methylesterase inhibitor (PME1) protein. For example, the method can involve introducing to the plant a composition that alters or silences gene expression. The composition can include an antisense nucleic acid that encodes RNAi, dsRNA, miRNA, or siRNA that targets the β-1,4-endoglucancase (EG) and/or pectin methylesterase inhibitor (PME1) in the plant and prevents or inhibits translation of the encoded protein(s), or alters expression of the protein(s), for example by producing an alternative splice variant. In some embodiments, the compositions mediate production by the plant of transacting small-interfering RNAs (tasiRNA) against the β-1,4-endoglucancase (EG) and/or pectin methylesterase inhibitor (PME1). In still other embodiments, the method involves introducing into the plant a composition that binds to the protein encoded by the β-1,4-endoglucancase (EG) and/or pectin methylesterase inhibitor (PME1), and inhibits one or more of the physiological activities of β-1,4-endoglucancase (EG) and/or pectin methylesterase inhibitor (PME1).

In some forms, expression of one or more genes is reduced or inhibited using known techniques, such as Zing fingers, TALENs, and CRISPR RNAs. Typically, when the methods include gene editing to disrupt, reduce or prevent expression of a specific gene, the methods include administering into a target plant one or more gene editing compounds in an amount effective to inhibit the expression of one or more genes in the plant. For example, in some forms, expression of β-1,4-endoglucanse is reduced or inhibited using known techniques, such as Zing fingers, TALENs, and CRISPR RNAs designed to specifically recognize and cleave the promoter and/or gene associated with expression of a functional β-1,4-endoglucanse. In other forms, expression of the a pectin methylesterase inhibitor is reduced or inhibited using known techniques, such as Zing fingers, TALENs, and CRISPR guide RNAs (gRNA) designed to specifically recognize and cleave the promoter and/or gene associated with expression of a pectin methylesterase inhibitor.

### 1. CRISPR-Based Methods for Gene Editing

Vectors configured for CRISPR-based gene editing using one or more of the sgRNA sequences configured to cleave one or more of the promoter or gene of Glyma.10G017000^{susceptible}, and/or the promoter or gene of Glyma.10G017100^{susceptible} are described.

CRISPR (Clustered Regularly Interspaced Short Palindromic Repeats) is an acronym for DNA loci that contain multiple, short, direct repetitions of base sequences. The prokaryotic CRISPR/Cas system has been adapted for use as gene editing (silencing, enhancing or changing specific genes) for use in eukaryotes (see, for example, Cong, Science, 15:339(6121):819-823 (2013) and Jinek, et al., Science, 337(6096):816-21 (2012)). Methods of preparing compositions for use in genome editing using the CRISPR/Cas systems are described in detail in WO 2013/176772 and WO 2014/018423, which are specifically incorporated by reference herein in their entireties.

As used herein, the term “Cas” generally refers to an effector protein of a CRISPR Cas system or complex. The term “Cas” may be used interchangeably with the terms “CRISPR” protein, “CRISPR Cas protein,” “CRISPR effector,” “CRISPR Cas effector,” “CRISPR enzyme,” “CRISPR Cas enzyme” and the like, unless otherwise apparent. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. One or more tracr mate sequences operably linked to a guide sequence (e.g., direct repeat-spacer-direct repeat) can also be referred to as pre-crRNA (pre-CRISPR RNA) before processing or crRNA after processing by a nuclease. Typically, the described vectors include an sgRNA from the described libraries, together with a Crispr-Cas effector protein.

sgRNA Structures

In some forms, the described sgRNA and libraries thereof include a tracrRNA and crRNA that are linked and form a chimeric crRNA-tracrRNA hybrid where a mature crRNA is fused to a partial tracrRNA via a synthetic stem loop to mimic the natural crRNA:tracrRNA duplex as described in Cong, Science, 15:339(6121):819-823 (2013) and Jinek, et al., Science, 337(6096):816-21 (2012)). A single fused crRNA-tracrRNA construct can also be referred to as a guide RNA or gRNA (or single-guide RNA (sgRNA)). Within an sgRNA, the crRNA portion can be identified as the ‘target sequence’ and the tracrRNA is often referred to as the ‘scaffold’.

### Crispr-Cas Effector Protein

The Crispr-Cas effector protein may be without limitation a type II, type V, or type VI Cas effector protein.

Non-limiting examples of Crispr-Cas effector proteins include Casl, CaslB, Cas2, Cas3, Cas4, Cas5, Cas6, Cas7, Cas8, Cas9 (also known as Csnl and Csxl2), CaslO, Csyl, Csy2, Csy3, Csel, Cse2, Cscl, Csc2, Csa5, Csn2, Csm2, Csm3, Csm4, Csm5, Csm6, Cmrl, Cmr3, Cmr4, Cmr5, Cmr6, Csbl, Csb2, Csb3, Csxl7, Csxl4, CsxlO, Csxl6, CsaX, Csx3, Csxl, Csxl5, Csfl, Csf2, Csf3, Csf4, homologues thereof, or modified versions thereof. In some forms, the unmodified CRISPR enzyme has DNA cleavage activity. Preferably, the Crispr-Cas effector protein is mutated with respect to a corresponding wild-type enzyme such that the mutated CRISPR enzyme lacks the ability to cleave one or both strands of a target polynucleotide containing a target sequence.

### Cas9

In some forms, the Type II CRISPR enzyme is a Cas9 enzyme such as disclosed in International Patent Application Publication No. WO/2014/093595. In some forms, the Cas9 enzyme is S. pneumoniae, S. pyogenes or S. thermophilus Cas9, and may include mutated Cas9 derived from these organisms. The enzyme may be a Cas9 homolog or ortholog. Additional orthologs include, for example, Cas9 enzymes from Corynebacter diptheriae, Eubacterium ventriosum, Streptococcus pasteurianus, Lactobacillus farciminis, Sphaeroachaeta globus, Azospirillum B510, Gluconacetobacter diazotrophicus, Neisseria cinereal, Roseburia intestinalis, Parvibaculum lavamentivorans, Staphylococcus aureus, Nitratifractor salsuginis DSM 16511, Camplyobacter lari CF89 12, and Streptococcus thermophilus LMD 9.

In some forms, the Cas9 effector protein and orthologs thereof may be modified for enhanced function. For example, improved target specificity of a CRISPR Cas9 system may be accomplished by approaches that include, but are not limited to, designing and preparing guide RNAs having optimal activity, selecting Cas9 enzymes of a specific length, truncating the Cas9 enzyme making it smaller in length than the corresponding wild-type Cas9 enzyme by truncating the nucleic acid molecules coding therefor and generating chimeric Cas9 enzymes wherein different parts of the enzyme are swapped or exchanged between different orthologs to arrive at chimeric enzymes having tailored specificity.

### Crispr-Cas Cleavage of Glyma.10G017000^{susceptible}

In some forms, one or more gRNA is designed to selectively bind and cleave nucleotide bonds at one or more positions within the sequence of the promoter for Glyma.10G017000, or to selectively bind and cleave at one or more region within the sequence of the Glyma.10G017000 gene. For example, in some forms, one or more gRNA is designed to selectively bind and cleave nucleotide bonds at one or more positions within SEQ ID NO:1, or within SEQ ID NO:2. Preferably, the gRNAs induce cleavage of the genome that abrogates expression of the Glyma.10G017000^{susceptible} gene.

As set forth in the Examples, below, it has been established that nematode resistant plants include a Glyma.10G017000 promoter that varies from that of the Glyma.10G017000^{susceptible} by having 23 SNPs, a 10 bp insertion, a 13 bp deletion, and a 15 bp insertion in the promoter of Glyma.10G017000. Therefore, in some forms, methods of gene editing a plant to induce nematode resistance in the plant include introducing one or more of 23 SNPs, a 10 bp insertion, a 13 bp deletion, and a 15 bp insertion in the promoter of Glyma.10G017000.

As set forth in the Examples, below, it has been established that nematode resistant plants include a Glyma.10G017000 that varies from the Glyma.10G017000^{susceptible} genome by having three nonsynonymous SNPs (M31I, T106I, V174A), two silent exonic SNPs, five intronic SNPs, a 1-bp intronic deletion, and a 3′ UTR SNP in Glyma.10G017000. Therefore, in some forms, methods of gene editing a plant to induce nematode resistance in the plant include introducing one or more of three nonsynonymous SNPs (M31I, T106I, V174A), two silent exonic SNPs, five intronic SNPs, a 1-bp intronic deletion, and a 3′ UTR SNP in Glyma.10G017000.

Crispr-Cas Cleavage of Glyma.10G017100^{susceptible}

In some forms, one or more gRNA is designed to selectively bind and cleave nucleotide bonds at one or more positions within the sequence of the promoter for Glyma.10G017100, or to selectively bind and cleave at one or more region within the sequence of the Glyma.10G017100 gene. For example, in some forms, one or more crRNA is designed to selectively bind and cleave nucleotide bonds at one or more positions within SEQ ID NO:9, or within SEQ ID NO:10. Preferably, the gRNAs induce cleavage of the genome that abrogates expression of the Glyma.10G017100^{susceptible} gene.

As set forth in the Examples, below, it has been established that nematode resistant plants include a Glyma.10G017100 promoter that varies from the Glyma.10G017100^{susceptible} promoter by having an insertion of 968 bp approximately 1.1 kb upstream of the 5′ UTR of Glyma.10G017100, as well as two single bp deletions after the 3′ end of this insertion and multiple SNPs before the 5′ end of the insertion. Therefore, in some forms, methods of gene editing a plant to induce nematode resistance in the plant include introducing into the Glyma.10G017100^{susceptible} promoter one or more of an insertion of 968 bp approximately 1.1 kb upstream of the 5′ UTR of Glyma.10G017100, as well as two single bp deletions after the 3′ end of this insertion and multiple SNPs before the 5′ end of the insertion.

As set forth in the Examples, below, it has been established that nematode resistant plants include a Glyma.10G017100 gene that varies from the Glyma.10G017100^{susceptible} gene by having five SNPs resulting in amino acid changes (V93I, D120Y, L149R, C167Y, E214D, one resulting in a premature stop codon (Q242*), and five silent exonic SNPs of Glyma.10G017100^{susceptible} Therefore, in some forms, methods of gene editing a plant to induce nematode resistance in the plant include introducing into the Glyma.10G017100^{susceptible} gene one or more of five SNPs resulting in amino acid changes (V93I, D120Y, L149R, C167Y, E214D, one resulting in a premature stop codon (Q242*), and five silent exonic SNPs.

2. Other Methods of Modifying Genes or Gene Expression In still other embodiments, the method involves introducing into the plant or plant cell a nucleic acid sequence that alters or silences expression of a β-1,4-endoglucancase (EG) in the plant. In a preferred embodiment, the β-1,4-endoglucancase (EG) is Glyma.10G01700, or an ortholog or homolog thereof. In still other embodiments, the method involves introducing into the plant or plant cell a nucleic acid sequence that alters or silences expression of a pectin methylesterase inhibitor (PME1) in the plant. In a preferred embodiment, the pectin methylesterase inhibitor (PME1) is Glyma.10G017100, or an ortholog or homolog thereof.

Preferably, the nucleic acid is operably linked to an expression control sequence. The expression control sequence can be an herbivory inducible control sequence, for example the endogenous control sequence of Glyma.10G017000, or Glyma.10G017100. The expression control sequence can be a heterologous control sequence. Selection of this control sequence can be used to select the amount of gene-silencing nucleic acid expressed and therefore control expression of the a β-1,4-endoglucanse in the plant. As a result of this method, the transgenic plant preferably has lower β-1,4-endoglucancase (EG) and/or pectin methylesterase inhibitor (PME1) activity compared to a control (e.g., wild-type) plant of the same species or cultivar. In some embodiments, the nucleic acid can silence a polynucleotide having the nucleic acid sequence SEQ ID NO:1, 2, or 3, or a nucleic acid encoding the polypeptide of SEQ ID NO: 7 or 8 or fragments, variants, orthologs, or homologs thereof. In some embodiments, the nucleic acid can silence a polynucleotide having the nucleic acid sequence SEQ ID NO: 9, 10, or 11, or a nucleic acid encoding the polypeptide of SEQ ID NO:15 or 16 or fragments, variants, orthologs, or homologs thereof.

In one embodiment, the method of reducing expression of a β-1,4-endoglucanse in a plant includes transfecting the plant with compositions that induce production of tasiRNA that mediate alteration or silencing of the β-1,4-endoglucancase (EG) expression and/or pectin methylesterase inhibitor (PME1) expression. The method can include introducing a polynucleotide including an miRNA target sequence operably linked to a sequence encoding the β-1,4-endoglucancase (EG) and/or pectin methylesterase inhibitor (PME1) into a cell. The miRNA target sequence operably linked to a sequence encoding the β-1,4-endoglucancase (EG) and/or pectin methylesterase inhibitor (PME1) can be transcribed in vitro and transiently transfected into the cell. Such methods are known in the art, see for example U.S. Published Application No. 2011/0165133 which is incorporated by reference in its entirety.

The miRNA target sequence can be operably linked to the β-1,4-endoglucancase (EG) and/or pectin methylesterase inhibitor (PME1) are expressed from an expression construct. In preferred embodiments, the miRNA target sequence operably linked to a sequence encoding the β-1,4-endoglucancase (EG) and/or pectin methylesterase inhibitor (PME1) is inserted into a plant vector, which can be transformed into the plant cell. The miRNA target sequence can also be operably linked to a sequence encoding a polynucleotide of interest is integrated into the nuclear or an organelle genome of the plant. In some embodiments the construct is expressed extra-chromosomally.

To induce gene silencing in a plant, the miRNA target sequence operably linked to a sequence encoding the β-1,4-endoglucancase (EG) and/or pectin methylesterase inhibitor (PME1) is typically co-expressed with an miRNA specific for the target miRNA sequence. Similar to the construct containing the miRNA target sequence operably linked to the pectin methylesterase inhibitor, the miRNA can be transiently transfected into the cell, or expressed from a vector. The miRNA can be integrated into the nuclear genome or an organelle genome of the plant, or expressed extra-chromosomally. In some embodiments, the miRNA is an endogenous miRNA that is expressed, or can be induced to be expressed by the plant cell. The miRNA can also be a heterologous miRNA.

As discussed above, when co-expressed, the miRNA binds to the miRNA target sequence and induce generation of tasiRNA which can mediate gene silencing of the β-1,4-endoglucancase (EG) and/or pectin methylesterase inhibitor (PME1). tasiRNA can be used to create transgenic plants with inducible or stable silencing the β-1,4-endoglucancase (EG) and/or pectin methylesterase inhibitor (PME1) expression.

Methods of interfering with the non-coding segments of a the β-1,4-endoglucancase (EG) such as Glyma.10G017000 and/or pectin methylesterase inhibitor (PME1) such as Glyma.10G017100 can be used to modulate the pest resistance. Deleting or altering some or all of the non-coding segments or inserting additional nucleotides into the non-coding segments can be effective to increase resistance to pests. Deleting, mutating, or inserting nucleotides in one or more of the β-1,4-endoglucancase (EG) expression control sequences, for example, the Glyma.10G017000 control sequences disclosed herein can decrease the expression of the β-1,4-endoglucancase (EG). Deleting, mutating, or inserting nucleotides in one or more of the pectin methylesterase inhibitor (PME1) such as Glyma.10G017100 expression control sequences, for example, the Glyma.10G017100 control sequences disclosed herein can decrease the expression of the pectin methylesterase inhibitor (PME1). Therefore, in some embodiments deleting or mutating nucleotides in β-1,4-endoglucancase (EG) and/or pectin methylesterase inhibitor (PME1) expression control sequence can shift the plant from pest susceptible to pest resistant. For example, in some embodiments insertions, mutations, or deletions are introduced into a polynucleotide having SEQ ID NO:1 or a functional fragment, variant, or complement thereof to reduce the herbivory sensitivity of the expression control sequence, or deletions are introduced into a polynucleotide having SEQ ID NO:9 or a functional fragment, variant, or complement thereof to reduce the herbivory sensitivity of the expression control sequence.

Inhibiting the regulatory function of the non-coding segments can also be used to modulate a β-1,4-endoglucancase (EG) and/or pectin methylesterase inhibitor (PME1). For instance, inhibiting or preventing the interaction of one or more of the non-coding segments with another nucleic acid sequence or protein. The additional nucleotides can be dependent on or independent of a functional copy of the β-1,4-endoglucancase (EG) and/or pectin methylesterase inhibitor (PME1) gene.

In some forms, methods of modifying the pest resistance of a plant can include replacing or supplementing the endogenous control sequences of a β-1,4-endoglucancase (EG) and/or pectin methylesterase inhibitor (PME1) with heterologous control sequences. The expression control sequences of the β-1,4-endoglucancase (EG) and/or pectin methylesterase inhibitor (PME1) can be altered or replaced with an expression control sequence that reduces induction during herbivory, but wherein expression of the β-1,4-endoglucancase (EG) and/or pectin methylesterase inhibitor (PME1) can be activated or induced during other periods, for example in the absence of nematode infestation.

### B. Methods of Using Herbivory Inducible Control Sequences

Compositions and methods of using the disclosed promoters are also provided. For example, Glyma.10G017000^{susceptible} and Glyma.10G017100^{susceptible} promoters can be used to drive any gene that is desired to be activated by herbivory. In some embodiments, the gene is a Glyma.10G017000 or Glyma.10G017100 resistance gene, such as those disclosed herein. In other embodiments, the gene is heterologous transgene. Exemplary heterologous transgenes include those that induce pest resistance, such as resistance to RKN.

Thus, the methods of modulating pest resistance in plants disclosed herein can include inducing or increasing expression of pest resistance gene in a plant. In some embodiments, the pest resistance gene is placed under the control of an herbivory inducible expression control sequence, or sequences. Herbivory inducible expression control sequences include, but are not limited to the control sequences of Glyma.10G017100, or a fragment thereof, or the control sequences of Glyma.10G017000, or a fragment thereof. For example, the expression control sequence can include 50, 100, 150, 250, 500, 750, 1,000, 1,250, 1,500, 2,000, or 2,500 nucleotides of the nucleic acid sequence SEQ ID NO: 1, and/or the nucleic acid sequence SEQ ID NO:9.

The pest resistance gene, for example a nematode resistance gene, can be an endogenous or heterologous gene. The pest resistance gene can impart resistance through reduction of nematode colonization, reduction of nematode reproduction, reduction of syncytium formation, or tolerance, or combinations thereof.

In some forms, a nematode-resistance gene is Rhg1. Rhg1 is located on chromosome 18, and has two alleles, including Rhg1-a and Rhg1-b. Rhg1-a is found in Peking-type soybeans, while Rhg1-b is found in PI 88788-type soybeans.

In some forms, a nematode-resistance gene is Rhg4 GmSNAP18. Along with Rhg1, Rhg4 gene is one of the most important QTLs for nematode resistance. The Rhg4 GmSNAP18 gene is involved in both Peking-type and PI 88788-type resistance. The Peking-type GmSNAP18 differs from the PI 88788-type GmSNAP18 by only five amino acids.

In some forms, a nematode-resistance gene is GmERF71. The GmERF71 gene may regulate nematode resistance by activating defense genes and phytohormone pathways.

In some forms, a nematode-resistance gene is GmLOX1. The GmLOX1 gene is involved in jasmonic acid biosynthesis, which may enhance resistance to nematode.

In some forms, the promoters drive another nematode-resistance gene, and/or expression of dsRNA that targets one or more specific gene sequences of a nematode, for example, for use as an RNAi-based host-induced gene silencing of one or more target genes within a nematode parasite.

### C. Methods of Modulating Nematode Resistance Using the Described Major QTL on Chromosome 10

It has also been discovered that plants with the described Major QTL on Chromosome 10, alone, or in combination with other pest resistance imparting genes and QTLs, exhibit increased nematode resistance to some sucking nematodes. In particular, it has been discovered that the described Major QTL on Chromosome 10, imparts resistance to nematodes, such as Root-knot nematodes (RKN), and plants that harbor the QTL exhibit reduced nematode infestation and increased crop yield compared to control plants without the described QTL. The resistance can be imparted by the described Major QTL on Chromosome 10 in presence or absence of other nematode resistance genes, constructs that modify gene product expression, and QTLs. Therefore, compositions and transgenic and hybrid plants including the described Major QTL on Chromosome 10, and methods of use thereof for reducing nematode infection and increasing crop yield are also disclosed. In some embodiments, the plants include additional nematode resistance genes, constructs that modify gene product expression, QTLs, or combinations thereof. In other embodiments, the plants do not include additional nematode resistance genes, constructs that modify gene product expression, QTLs, or combinations thereof. For example, in some embodiments, the plant is resistant to nematodes without the described Major QTL on Chromosome 10.

For example, the data presented in Examples 1 and 2 show that the described Major QTL on Chromosome 10, can be effective to reduce feeding by nematodes. To identify spatial expression of EG, promoter:GUS constructs were created with both the resistant and susceptible versions of the promoter. GFP+ composite plants were inoculated with 1,200 J2s, then stained with GUS substrate. If GUS expression was observed in a root, it was stained with acid fuchsin to visualize nematode presence in relation to GUS expression. GUS expression was not quantified, merely evaluated for the presence/absence of GUS in response to infection. Five to eight independent transgenic roots were evaluated for each construct. GUS expression was observed in feeding sites under both promoters, however the occurrence of the expression was less frequently observed under the resistant promoter, while it was observed at the feeding sites of all swollen J2 under the susceptible promoter. When nematodes were not present in a root section, GUS expression was observed at low levels in vascular tissue under both the resistant and susceptible promoters. To functionally evaluate the length of the promoter required to induce expression in giant cells, 1.3 kb, approximately the first half) of the susceptible EG promoter was tested in ExF67 roots. GUS expression was still observed at feeding sites, indicating that the necessary promoter elements for expression in giant cells are located within 1.3 kb of EG promoter.

Therefore, transgenic and hybrid plants including the described Major QTL on Chromosome 10 are disclosed. In some embodiments, the plants are used in method of increasing resistance against root knot nematodes. The plant can be a soybean plant. Preferably the plant is also a transgenic plant where the plant exhibits altered, reduced or inhibited expression or activity of the β-1,4-endoglucancase (EG) and/or pectin methylesterase inhibitor (PME1) protein as discussed above. The plant can also contain the described Major QTL on Chromosome 10 are disclosed. The plants can be used to increase nematode resistance or decrease feeding or infestation of nematodes, and/or increase crop yield.

### IV. Methods for Making Pest Resistant Plants

Non-naturally occurring plants employing one or more of the above disclosed compositions or methods of modulating pest resistance are also disclosed.

In some embodiments, the plants are genetically modified (e.g., by CRISPR/Cas) to, for example, disrupt susceptibility gene(s). Targeted mutagenesis (also known as gene editing) is a very important technology to crop breeding. There are numerous methods to edit specific gene targets now, including CRISPR, TALEN, meganucleases, and zinc fingers. The endonuclease can be designed to target nearly any sequence. The endonuclease(s) can be constructed using methods such as, but not limited to, those described by Svitashev, et al., Plant Physiology, 169: 931-945 (2015), Lee, et al., Plant Biotechnology, 17(2):362-372 (2019)), Sander et al., Nature Met, 8(1):67-69, (2011), Cermak et al., Nucl Acids Res, 39(17):7879 (2011); with correction at Nucl Acids Res, 39:e82. doi: 10.1093/nar/gkr218, 2011), and Liang et al., et al., J Genet Genom, 41(2):63-68, (2014). The promoter used to drive expression of the endonuclease can be one expressed throughout development or specifically in egg cells or during early embryo development, and can be endogenous or exogenous. Examples of promoters 35S (CaMV d35S) or derivatives (e.g., double 35S) ZmUb1 (maize) APX (rice) OsCc1 (rice) EIF5 (rice) R1G1B (rice) PGD1 (rice) Act1 (rice) SCP1 (rice).

The gene editing machinery construct(s) may include a selectable marker (e.g., herbicide resistance) to assist with recovery of the transgene during whole plant transformation and subsequent backcrossing. In some cases, one or more (e.g., two or more, or three or more) endonucleases and/or CRISPR guide RNAs are combined into a single construct to target one or sequences of DNA.

One method to introduce editing machinery into plants is to use an Agrobacterium-based method (such as the method described by Ishida et al., Nature Biotechnol, 146:745-750 (1996)) or particle bombardment (such as the method described by Gordon-Kamm et al., Plant Cell Online, 2(7):603-618 (1990)) on plant tissue. Newer methods that incorporate developmental regulator genes have been devised that make it possible to transform plants without extensive tissue culture. See, e.g., Lowe, et al., The Plant Cell, 28: 1998-2015 (2016). In transformation, DNA coding for the editing machinery (e.g., CAS9 and guide RNA) is introduced into plant callus, seed or embryonic tissue. Stably-transformed plants (events) are then recovered, optionally with the help of a selectable marker. See, e.g., U.S. Pat. No. 11,584,936 and WO 2021/252619, each of which is specifically incorporated by reference herein in its entirety.

Additionally or alternatively, plants can be modified to include or express heterologous sequences including genes. In some embodiments, the heterologous sequence is from another plant of the same or different species. Thus, in some forms the plants include sequences from another plant and in some embodiments they do not.

Thus, in some embodiments the plants are transgenic plants, for example a transgenic plant expressing an antisense oligonucleotide that prevents, alters, reduces or inhibits expression of a β-1,4-endoglucancase, such as Glyma.10G017000, or a functional fragment, variant, ortholog or homolog thereof. In some embodiments the plants are transgenic plants, for example a transgenic plant expressing an antisense oligonucleotide that prevents, alters, reduces or inhibits expression of a pectin methylesterase inhibitor, such as Glyma.10G017100, or a functional fragment, variant, ortholog or homolog thereof. In other embodiments, the transgenic plant includes a promoter associated with nematode resistance, such as promoter for Glyma.10G017000 having a nucleotide sequence of SEQ ID NO:4, and/or a promoter for Glyma.10G017100 having a nucleotide sequence of SEQ ID NO: 12. In still other embodiments, the transgenic plants are characterized by a reduction or inhibition of a β-1,4-endoglucancase, such as Glyma.10G017000, and/or a reduction or inhibition of pectin methylesterase inhibitor, such as Glyma.10G017100.

The transgenic plant can include one or more nematode resistance transgenes or nematode resistant QTLs. For example, in some embodiments, the transgenic plant includes one or more alleles of a quantitative trait locus (QTL) on chromosome 10.

The disclosed pest resistant plants typically have increased resistance to one or more plant eating animals, increased resistance to one or more plant eating nematode, or combinations thereof relative to naturally occurring plants. In a preferred embodiment, the transgenic plants have increased resistance to nematodes. The nematode resistance can be reduction of nematode colonization, reduction of nematode reproduction, reduction of syncytium formation, or combinations thereof. In a preferred embodiment, the disclosed plants have an increased resistance to one or more nematodes including, but not limited to nematodes of the genus Heterodera, or Meloidogyne, such as Heterodera glycines, Heterodera schachtii, and Meloidogyne incognita.

### A. Constructs and Vectors

### 1. Recombinant Expression

In some forms, vectors and constructs contain a mutant or disrupted β-1,4-endoglucancase gene or coding sequence, such as Glyma.10G017000, and/or a pectin methylesterase inhibitor, such as Glyma.10G017100, or both Glyma.10G017000 and Glyma.10G017100, or fragments, variants, orthologs or homologs thereof can be operably linked to an endogenous or heterologous expression control sequence. The constructs can include an expression cassette containing a mutant or disrupted Glyma.10G017000 promoter, gene or coding sequence, or a mutant or disrupted Glyma.10G017100 promoter, gene and/or coding sequence(s) such as, those found on the described major QTL on chromosome 10, for example as set forth in any of SEQ ID NOs:4, 5, 6, 12, 13 or 14 or a nucleic acid encoding the amino acid sequence of SEQ ID NO:8, or 16.

### 2. Antisense

Antisense oligonucleotides that target a β-1,4-endoglucancase such as Glyma.10G017000, or and ortholog or a homolog thereof are disclosed. Antisense oligonucleotides that target a pectin methylesterase inhibitor, such as Glyma.10G017100, or and ortholog or a homolog thereof are also disclosed.

In some forms, the antisense oligonucleotides include, but are not limited to, RNAi, dsRNA, miRNA, siRNA, or transacting small-interfering RNAs (tasiRNA) that target the β-1,4-endoglucancase mRNA in a plant, and delay, inhibit, or prevent expression of the β-1,4-endoglucancase gene or gene product in plants. In some forms, the antisense oligonucleotides include, but are not limited to, RNAi, dsRNA, miRNA, siRNA, or transacting small-interfering RNAs (tasiRNA) that target the pectin methylesterase inhibitor mRNA in a plant, and delay, inhibit, or prevent expression of the pectin methylesterase inhibitor gene or gene product in plants.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively, the antisense molecule is designed to interrupt a processing function that normally would take place on the target molecule, such as transcription or replication.

In some forms, antisense molecules are designed based on the sequence of the target molecule, for example Glyma.10G017000 coding sequences including, but not limited to, SEQ ID NOs:1, 2, 3, or a nucleic acid encoding the amino acid sequence of SEQ ID NO:7. In some forms, antisense molecules are designed based on the sequence of the target molecule, for example Glyma.10G017100 coding sequences including, but not limited to, SEQ ID NOs:9, 10, 11, or a nucleic acid encoding the amino acid sequence of SEQ ID NO:15. Methods of designing antisense molecules directed to a target sequence, for example SEQ ID NOs:1, 2, 3, 9, 10 or 11, or a nucleic acid encoding the amino acid sequence of SEQ ID NO:7, or 15 are well also well known in the art. See for example, Elbashir, et al., Methods, 26:199-213 (2002).

The production of siRNA from a vector is more commonly done through the transcription of a short hairpin RNAs (shRNAs). Accordingly, vectors and constructs containing a nucleic acid sequence that silences Glyma.10G017000 gene expression (e.g., siRNA, RNAi, shRNA, tasiRNA) operably linked to a heterologous expression control sequence, as well as vectors and constructs containing a nucleic acid sequence that silences Glyma.10G017100 gene expression (e.g., siRNA, RNAi, shRNA, tasiRNA) operably linked to a heterologous expression control sequence, are also disclosed

### 3. Genes of Interest

Methods of modifying a plant gene, polynucleotide, or coding sequence to be pest resistant (i.e., nematode resistant) are also disclosed. The method generally involves operably linking a Glyma.10G017000 and/or Glyma.10G017100 inducible control sequence to a polynucleotide of interest. The polynucleotide of interest can be a coding sequence, for example a sequence encoding a polypeptide (with or without introns), or non-coding sequence such as an antisense or inhibitory nucleic acid. In some embodiments the polynucleotide includes a cDNA of a polypeptide of interest, for example a nematode resistance gene.

### 4. Transformation Constructs

Transformation constructs including the disclosed nucleic acids are also disclosed. Constructs can be engineered such that transformation of the nuclear genome and expression of transgenes from the nuclear genome occurs. Alternatively, transformation constructs can be engineered such that transformation of the plastid genome and expression of the plastid genome occurs. Transformation constructs can be used, for example, to express an antisense oligonucleotide that reduces or silences gene expression of a β-1,4-endoglucancase gene or coding sequence, such as Glyma.10G017000, gene or gene product in plants, or to express an antisense oligonucleotide that reduces or silences gene expression of pectin methylesterase inhibitor gene or coding sequence, such as Glyma.10G017100, gene or gene product in plants.

Generally, the nucleic acid sequences disclosed are operably linked to a suitable promoter expressible in plants, and used to modulate nematode resistance in a plant. Expression cassettes containing the disclosed nucleic acids may also include any further sequences required or selected for the expression of the transgene. Such sequences include, but are not restricted to, transcription terminators, extraneous sequences to enhance expression such as introns, vital sequences, and sequences intended for the targeting of the gene product to specific organelles and cell compartments. These expression cassettes can then be easily transferred to the plant transformation vectors. Representative plant transformation vectors are described in plant transformation vector options available (Gene Transfer to Plants (1995), Potrykus, et al., G. eds. Springer-Verlag Berlin Heidelberg New York; “Transgenic Plants: A Production System for Industrial and Pharmaceutical Proteins” (1996), Owen, M. R. L. and Pen, J. eds. John Wiley & Sons Ltd. England and Methods in Plant Molecular biology—a laboratory course manual (1995), Maliga, P., Klessig, D. F., Cashmore, A. R., Gruissem, W. and Varner, J. E. eds. Cold Spring Laboratory Press, New York).

An additional approach is to use a vector to specifically transform the plant plastid chromosome by homologous recombination (U.S. Pat. No. 5,545,818 to McBride, et al.), in which case it is possible to take advantage of the prokaryotic nature of the plastid genome and insert a number of transgenes as an operon.

The following is a description of various components of typical expression cassettes.

### 5. Promoters

Plant promoters can be selected to control the expression of the transgene in different plant tissues or organelles, for all of which methods are known to those skilled in the art (Gasser, et al., Science, 244:1293-99 (1989)). In a preferred embodiment, promoters are selected from those of plant or prokaryotic origin that are known to yield high expression in plastids. In certain embodiments the promoters are inducible. Inducible plant promoters are known in the art.

The transgenes can be inserted into an existing transcription unit (such as, but not limited to, psbA) to generate an operon. However, other insertion sites can be used to add additional expression units as well, such as existing transcription units and existing operons (e.g., atpE, accD). Such methods are described in, for example, U.S. Pat. App. Pub. 2004/0137631, which is incorporated herein by reference in its entirety. For an overview of other insertion sites used for integration of transgenes into the tobacco plastome, see Staub (Staub, J. M., “Expression of Recombinant Proteins via the Plastid Genome,” in: Vinci V A, Parekh S R (eds.) Handbook of Industrial Cell Culture: Mammalian, and Plant Cells, pp. 259-278, Humana Press Inc., Totowa, NJ (2002)).

In general, the promoter can be from any class I, II or III gene. For example, any of the following plastidial promoters and/or transcription regulation elements can be used for expression in plastids. Sequences can be derived from the same species as that used for transformation. Alternatively, sequences can be derived from other species to decrease homology and to prevent homologous recombination with endogenous sequences.

For instance, the following plastidial promoters can be used for expression in plastids.
PrbcL promoter (Allison, et al., EMBO J. 15:2802-2809 (1996); Shiina, et al., Plant Cell 10:1713-1722 (1998));
PpsbA promoter (Agrawal, et al., Nucleic Acids Research 29:1835-1843 (2001));
Prrn 16 promoter (Svab, et al., Proc. Natl. Acad. Sci. USA 90:913-917 (1993); Allison, et al., EMBO J. 15:2802-2809 (1996));
PaccD promoter (Hajdukiewicz P T J, Allison L A, Maliga P, EMBO J. 16:4041-4048 (1997); WO 97/06250);
PclpP promoter (Hajdukiewicz, et al., EMBO J. 16:4041-4048 (1997); WO 99/46394);
PatpB, PatpI, PpsbB promoters (Hajdukiewicz, et al., EMBO J. 16:4041-4048 (1997));
PrpoB promoter (Liere K, Maliga P, EMBO J. 18:249-257 (1999);
PatpB/E promoter (Kapoor, et al., Plant J. 11:327-337 (1997)).

In addition, prokaryotic promoters (such as those from, e.g., E. coli or Synechocystis) or synthetic promoters can also be used.

Promoters vary in their strength, i.e., ability to promote transcription. Depending upon the host cell system utilized, any one of a number of suitable promoters known in the art may be used. For example, for constitutive expression, the CaMV 35S promoter, the rice actin promoter, or the ubiquitin promoter may be used. For example, for regulatable expression, the chemically inducible PR-1 promoter from tobacco or Arabidopsis may be used (see, e.g., U.S. Pat. No. 5,689,044 to Ryals, et al.).

A suitable category of promoters is that which is wound inducible. Numerous promoters have been described which are expressed at wound sites. Preferred promoters of this kind include those described by Stanford, et al., Mol. Gen. Genet. 215: 200-208 (1989), Xu et al. Plant Molec. Biol. 22:573-588 (1993), Logemann, et al., Plant Cell 1:151-158 (1989), Rohrmeier, et al., Plant Molec. Biol. 22:783-792 (1993), Firek, et al., Plant Molec. Biol. 22:129-142 (1993), and Warner, et al., Plant J., 3:191-201 (1993).

Suitable tissue specific expression patterns include green tissue specific, root specific, stem specific, and flower specific. Promoters suitable for expression in green tissue include many which regulate genes involved in photosynthesis, and many of these have been cloned from both monocotyledons and dicotyledons. A suitable promoter is the maize PEPC promoter from the phosphoenol carboxylase gene (Hudspeth, et al., Plant Molec. Biol. 12:579-589 (1989)). A suitable promoter for root specific expression is that described by de Framond, FEBS, 290: 103-106 (1991); EP 0 452 269 to de Framond and a root-specific promoter is that from the T-1 gene. A suitable stem specific promoter is that described in U.S. Pat. No. 5,625,136 and which drives expression of the maize trpA gene.

The promoter can be a relatively weak plant expressible promoter. Thus, the promoter can in some embodiments initiate and control transcription of the operably linked nucleic acids about 10 to about 100 times less efficient that an optimal CaMV35S promoter. Relatively weak plant expressible promoters include the promoters or promoter regions from the opine synthase genes of Agrobacterium spp. such as the promoter or promoter region of the nopaline synthase, the promoter or promoter region of the octopine synthase, the promoter or promoter region of the mannopine synthase, the promoter or promoter region of the agropine synthase and any plant expressible promoter with comparably activity in transcription initiation. Other relatively weak plant expressible promoters may be dehiscence zone selective promoters, or promoters expressed predominantly or selectively in dehiscence zone and/or valve margins of fruits, such as the promoters described in WO97/13865.

Cis-regulatory elements from the promoter of photoperiod-responsive genes, coordinated motifs integrating hormones and stresses to photoperiod responses, and the promoters of photo-responsive genes such as those described in Mongkolsiriwatana, Katsetsart J. (Nat. Sci.) 43: 164-177 (2009), can also be used.

### 6. Transcriptional Terminators

A variety of transcriptional terminators are available for use in expression cassettes. These are responsible for the termination of transcription beyond the transgene and its correct polyadenylation. Appropriate transcriptional terminators are those that are known to function in plants and include the CaMV 35S terminator, the tm1 terminator, the nopaline synthase terminator and the pea rbcS E9 terminator. These are used in both monocotyledonous and dicotyledonous plants.

At the extreme 3′ end of the transcript, a polyadenylation signal can be engineered. A polyadenylation signal refers to any sequence that can result in polyadenylation of the mRNA in the nucleus prior to export of the mRNA to the cytosol, such as the 3′ region of nopaline synthase (Bevan, et al., Nucleic Acids Res., 11:369-385 (1983)).

### 7. Sequences for Expression Enhancement or Regulation

Numerous sequences have been found to enhance gene expression from within the transcriptional unit and these sequences can be used in conjunction with the genes to increase their expression in transgenic plants. For example, various intron sequences such as introns of the maize AdhI gene have been shown to enhance expression, particularly in monocotyledonous cells. In addition, a number of non-translated leader sequences derived from viruses are also known to enhance expression, and these are particularly effective in dicotyledonous cells.

### 8. Coding Sequence Optimization

The coding sequence of the disclosed genes can be genetically engineered by altering the coding sequence for optimal expression (also referred to herein as “codon optimized”) in the crop species of interest. Methods for modifying coding sequences to achieve optimal expression in a particular crop species are well known (see, e.g. Perlak, et al., Proc. Natl. Acad. Sci. USA 88: 3324 (1991); and Koziel, et al, Biotechnol. 11: 194 (1993)). Therefore, in some embodiments, the disclosed nucleic acids sequences, or fragments or variants thereof, are genetically engineered for optimal expression in the crop species of interest.

### 9. Selectable Markers

Genetic constructs may encode a selectable marker to enable selection of plastid transformation events. There are many methods that have been described for the selection of transformed plants [for review see (Miki et al., Journal of Biotechnology, 107:193-232 (2004) and references incorporated within]. Selectable marker genes that have been used extensively in plants include the neomycin phosphotransferase gene nptII (U.S. Pat. Nos. 5,034,322, 5,530,196), hygromycin resistance gene (U.S. Pat. No. 5,668,298), the bar gene encoding resistance to phosphinothricin (U.S. Pat. No. 5,276,268), the expression of aminoglycoside 3″-adenyltransferase (aadA) to confer spectinomycin resistance (U.S. Pat. No. 5,073,675), the use of inhibition resistant 5-enolpyruvyl-3-phosphoshikimate synthetase (U.S. Pat. No. 4,535,060) and methods for producing glyphosate tolerant plants (U.S. Pat. Nos. 5,463,175; 7,045,684). Methods of plant selection that do not use antibiotics or herbicides as a selective agent have been previously described and include expression of glucosamine-6-phosphate deaminase to inactive glucosamine in plant selection medium (U.S. Pat. No. 6,444,878) and a positive/negative system that utilizes D-amino acids (Erikson, et al., Nat Biotechnol, 22:455-8 (2004). European Patent Publication No. EP 0 530 129 A1 describes a positive selection system which enables the transformed plants to outgrow the non-transformed lines by expressing a transgene encoding an enzyme that activates an inactive compound added to the growth media. U.S. Pat. No. 5,767,378 describes the use of mannose or xylose for the positive selection of transgenic plants. Methods for positive selection using sorbitol dehydrogenase to convert sorbitol to fructose for plant growth have also been described (WO 2010/102293). Screenable marker genes include the beta-glucuronidase gene (Jefferson, et al., EMBO J. 6:3901-3907 (1987); U.S. Pat. No. 5,268,463) and native or modified green fluorescent protein gene (Cubitt, et al., Trends Biochem. Sci. 20:448-455 (1995); Pan, et al., Plant Physiol. 112:893-900 (1996)).

Transformation events can also be selected through visualization of fluorescent proteins such as the fluorescent proteins from the nonbioluminescent Anthozoa species which include DsRed, a red fluorescent protein from the Discosoma genus of coral (Matz, et al., Nat Biotechnol, 17:969-73 (1999)). An improved version of the DsRed protein has been developed (Bevis, et al., Nat Biotech, 20:83-87 (2002)) for reducing aggregation of the protein. Visual selection can also be performed with the yellow fluorescent proteins (YFP) including the variant with accelerated maturation of the signal (Nagai, et al., Nat Biotech, 20:87-90 (2002), the blue fluorescent protein, the cyan fluorescent protein, and the green fluorescent protein (Sheen, et al., Plant J, 8:777-84 (1995); Davis, et al., Plant Molecular Biology, 36:521-528 (1998)). A summary of fluorescent proteins can be found in Tzfira, et al., Plant Molecular Biology, 57:503-516 (2005) and Verkhusha, et al., Nat Biotech, 22:289-296 (2004) whose references are incorporated in entirety. Improved versions of many of the fluorescent proteins have been made for various applications. Use of the improved versions of these proteins or the use of combinations of these proteins for selection of transformants will be obvious to those skilled in the art. It is also practical to simply analyze progeny from transformation events for the presence of the PHB thereby avoiding the use of any selectable marker.

For plastid transformation constructs, a preferred selectable marker is the spectinomycin-resistant allele of the plastid 16S ribosomal RNA gene (Staub, et al., Plant Cell 4:39-45 (1992); Svab, et al., Proc. Natl. Acad. Sci. USA 87:8526-8530 (1990)). Selectable markers that have since been successfully used in plastid transformation include the bacterial aadA gene that encodes aminoglycoside 3′-adenyltransferase (AadA) conferring spectinomycin and streptomycin resistance (Svab, et al., Proc. Natl. Acad. Sci. USA, 90:913-917 (1993), nptII that encodes aminoglycoside phosphotransferase for selection on kanamycin (Carrer, et al., Mol. Gen. Genet. 241:49-56 (1993); Lutz, et al., Plant J., 37: 906-913 (2004); Lutz K A, et al., Plant Physiol. 145: 1201-1210 (2007)), aphA6, another aminoglycoside phosphotransferase (Huang F-C, et al, Mol. Genet. Genomics 268: 19-27 (2002)), and chloramphenicol acetyltransferase (Li, et al., Plant Mol Biol, DOI 10.1007/s11103-010-9678-4 (2010)). Another selection scheme has been reported that uses a chimeric betaine aldehyde dehydrogenase gene (BADH) capable of converting toxic betaine aldehyde to nontoxic glycine betaine (Daniell, et al., Curr. Genet., 39:109-116 (2001)).

### 10. Targeting Sequences

The disclosed vectors and constructs may further include, within the region that encodes the protein to be expressed, one or more nucleotide sequences encoding a targeting sequence. A “targeting” sequence is a nucleotide sequence that encodes an amino acid sequence or motif that directs the encoded protein to a particular cellular compartment, resulting in localization or compartmentalization of the protein. Presence of a targeting amino acid sequence in a protein typically results in translocation of all or part of the targeted protein across an organelle membrane and into the organelle interior. Alternatively, the targeting peptide may direct the targeted protein to remain embedded in the organelle membrane. The “targeting” sequence or region of a targeted protein may contain a string of contiguous amino acids or a group of noncontiguous amino acids. The targeting sequence can be selected to direct the targeted protein to a plant organelle such as a nucleus, a microbody (e.g., a peroxisome, or a specialized version thereof, such as a glyoxysome) an endoplasmic reticulum, an endosome, a vacuole, a plasma membrane, a cell wall, a mitochondria, a chloroplast or a plastid. A chloroplast targeting sequence is any peptide sequence that can target a protein to the chloroplasts or plastids, such as the transit peptide of the small subunit of the alfalfa ribulose-biphosphate carboxylase (Khoudi, et al., Gene, 197:343-351 (1997)). A peroxisomal targeting sequence refers to any peptide sequence, either N-terminal, internal, or C-terminal, that can target a protein to the peroxisomes, such as the plant C-terminal targeting tripeptide SKL (Banjoko, et al., Plant Physiol., 107:1201-1208 (1995); Wallace et al., Plant Organellular Targeting Sequences, in Plant Molecular Biology, Ed. R. Croy, BIOS Scientific Publishers Limited 287-288 (1993), and peroxisomal targeting in plant is shown in M. Volokita, The Plant J., 361-366 (1991)).

Plastid targeting sequences are known in the art and include the chloroplast small subunit of ribulose-1,5-bisphosphate carboxylase (Rubisco) (de Castro Silva Filho et al., Plant Mol. Biol. 30:769-780 (1996); Schnell, et al. J. Biol. Chem. 266(5):3335-3342 (1991)); 5-(enolpyruvyl)shikimate-3-phosphate synthase (EPSPS) (Archer, et al., J. Bioenerg. Biomemb. 22(6):789-810 (1990)); tryptophan synthase (Zhao, et al., J. Biol. Chem. 270(11):6081-6087 (1995)); plastocyanin (Lawrence, et al., J. Biol. Chem. 272(33):20357-20363 (1997)); chorismate synthase (Schmidt, et al., J. Biol. Chem. 268(36):27447-27457 (1993)); and the light harvesting chlorophyll a/b binding protein (LHBP) (Lamppa, et al., J. Biol. Chem. 263:14996-14999 (1988)). See also Von Heijne, et al., Plant Mol. Biol. Rep. 9:104-126 (1991); Clark, et al., J. Biol. Chem. 264:17544-17550 (1989); Della-Cioppa, et al., Plant Physiol. 84:965-968 (1987); Romer, et al., Biochem. Biophys. Res. Commun. 196:1414-1421 (1993); and Shah, et al., Science 233:478-481 (1986). Alternative plastid targeting signals have also been described in the following: US 2008/0263728; Miras, et al., J Biol Chem, 277:49 (2002): 47770-8; Miras, et al., J Biol Chem, 282:29482-29492 (2007).

### 11. Plants and Tissues for Transfection, Introgression, and Breeding

Both dicotyledons (“dicots”) and monocotyledons (“monocots”) can be used in the disclosed positive selection system. Monocot seedlings typically have one cotyledon (seed-leaf), in contrast to the two cotyledons typical of dicots. Eudicots are dicots whose pollen has three apertures (i.e. triaperturate pollen), through one of which the pollen tube emerges during pollination. Eudicots contrast with the so-called ‘primitive’ dicots, such as the magnolia family, which have uniaperturate pollen (i.e. with a single aperture).

Monocots include one of the large divisions of Angiosperm plants (flowering plants with seeds protected within a vessel). They are herbaceous plants with parallel veined leaves and have an embryo with a single cotyledon, as opposed to dicot plants (dicotyledonous), which have an embryo with two cotyledons. Most of the important staple crops of the world, the so-called cereals, such as wheat, barley, rice, maize, sorghum, oats, rye and millet, are monocots. Thus, the plant can be a grass, such as wheat, barley, rice, maize, sorghum, oats, rye and millet.

The plant can therefore be a cereal crop such as wheat, oat, barley, or rice; a forage such as bahiagrass, dallisgrass, kleingrass, guineagrass, reed canarygrass, orchardgrass, ricegrass, foxtail, or vetch; a legume such as soybean, lentil, or chickpea; an oilseed such as canola; a vegetable such as onion or carrot; or a specialty crop such as caraway, hemp, or sesame.

In some embodiments, the plant is a soybean, such as a Glycine max (L.) Merril. For example, the soybean plant can be any soybean cultivar known in the art. Exemplary soybean cultivars include, but are not limited to the Glycine max cultivars Jack, Resnik, Williams 82, Corsoy, Crawford, Hutcheson, Kunitz, Champ, Benning, Maria, Nattoking-K88, Gaillard, Mivak, Nattoking-K87, Mario, Arkadija, Danica, Rige, Odesskaja Balkan, Ugo, Maple Ridge, Schladming, Quito, McCall, Krajina, Dorota, OAC, Scorpio, Odell, Emerson, Sibley, Maverick, Bristol, Sturdy, Accord, Belmont, Simpson, AC Glengarry, Crystal, Lokon, Guntur, Tidar, Orba, Wilis, Kerinci, Dempo, Galunggung, SJ4, SJ5, Sukothai 1, Ankur, Alankar, Shilajeet, Gaurav, Durga, KHSb-2, PB-1 Nakhonsawan 1, KS No 10, KS No 1, UFUS Xavante, or Woodruff Additional suitable soybean varieties are available from both academic and commercial institutions, such as—for example—the University of Guelph (Ontario Agricultural College; e.g. soybean varieties RCAT Staples, Westag 97, RCAT Bobcat, OAC Prudence, OAC Woodstock, OAC 9908), or soybean varieties from Daryland or Soygenetics. Additional suitable varieties are P1548402 (Peking), P1437654 (Er-hejjan), P1438489 (Chiquita), P1507354 (Tokei 421), P1548655 (Forrest), P1548988 (Pickett), P188788, P1404198 (Sun Huan Do), P1404166 (Krasnoaarmejkaja), Hartwig, Manokin, Doles, Dyer, and Custer.

In some embodiments, the plant is a miscanthus. Thus, the plant can be of the species Miscanthus floridulus, Miscanthus x. giganteus, Miscanthus sacchariflorus (Amur silver-grass), Miscanthus sinensis, Miscanthus tinctorius, or Miscanthus transmorrisonensis.

Additional representative plants useful in the compositions and methods disclosed herein include the Brassica family including sp. napus, rapa, oleracea, nigra, carinata and juncea; industrial oilseeds such as Camelina sativa, Crambe, Jatropha, castor; Arabidopsis thaliana; soybean; cottonseed; sunflower; palm; coconut; rice; safflower; peanut; mustards including Sinapis alba; sugarcane and flax.

Crops harvested as biomass, such as silage corn, alfalfa, switchgrass, or tobacco, also are useful with the methods disclosed herein. Representative tissues for transformation using these vectors include protoplasts, cells, callus tissue, leaf discs, pollen, and meristems.

### B. Plant Transformation Techniques

The transformation of suitable agronomic plant hosts using vectors expressing transgenes can be accomplished with a variety of methods and plant tissues. Representative transformation procedures include Agrobacterium-mediated transformation, biolistics, microinjection, electroporation, polyethylene glycol-mediated protoplast transformation, liposome-mediated transformation, and silicon fiber-mediated transformation (U.S. Pat. No. 5,464,765 to Coffee, et al.; “Gene Transfer to Plants” (Potrykus, et al., eds.) Springer-Verlag Berlin Heidelberg New York (1995); “Transgenic Plants: A Production System for Industrial and Pharmaceutical Proteins” (Owen, et al., eds.) John Wiley & Sons Ltd. England (1996); and “Methods in Plant Molecular Biology: A Laboratory Course Manual” (Maliga et al. eds.) Cold Spring Laboratory Press, New York (1995)).

Plants can be transformed by a number of reported procedures (U.S. Pat. No. 5,015,580 to Christou, et al.; U.S. Pat. No. 5,015,944 to Bubash; U.S. Pat. No. 5,024,944 to Collins, et al.; U.S. Pat. No. 5,322,783 to Tomes et al.; U.S. Pat. No. 5,416,011 to Hinchee et al.; U.S. Pat. No. 5,169,770 to Chee et al.). A number of transformation procedures have been reported for the production of transgenic maize plants including pollen transformation (U.S. Pat. No. 5,629,183 to Saunders et al.), silicon fiber-mediated transformation (U.S. Pat. No. 5,464,765 to Coffee et al.), electroporation of protoplasts (U.S. Pat. No. 5,231,019 Paszkowski et al.; U.S. Pat. No. 5,472,869 to Krzyzek et al.; U.S. Pat. No. 5,384,253 to Krzyzek et al.), gene gun (U.S. Pat. No. 5,538,877 to Lundquist et al. and U.S. Pat. No. 5,538,880 to Lundquist et al.), and Agrobacterium-mediated transformation (EP 0 604 662 A1 and WO 94/00977 both to Hiei Yukou et al.). The Agrobacterium-mediated procedure is particularly preferred as single integration events of the transgene constructs are more readily obtained using this procedure which greatly facilitates subsequent plant breeding. Plants can be transformed by particle bombardment (U.S. Pat. No. 5,004,863 to Umbeck and 5,159,135 to Umbeck). Sunflower can be transformed using a combination of particle bombardment and Agrobacterium infection (EP 0 486 233 A2 to Bidney, Dennis; U.S. Pat. No. 5,030,572 to Power et al.). Flax can be transformed by either particle bombardment or Agrobacterium-mediated transformation. Switchgrass can be transformed using either biolistic or Agrobacterium mediated methods (Richards, et al., Plant Cell Rep., 20:48-54 (2001); Somleva, et al., Crop Science, 42:2080-2087 (2002)). Methods for sugarcane transformation have also been described (Franks, et al., Aust. J Plant Physiol. 18:471-480 (1991); WO 2002/037951 to Elliott, et al., et al.).

Recombinase technologies which are useful in practicing the current invention include the cre-lox, FLP/FRT and Gin systems. Methods by which these technologies can be used for the purpose described herein are described for example in (U.S. Pat. No. 5,527,695 to Hodges et al.; Dale, et al., Proc. Natl. Acad. Sci. USA, 88:10558-10562 (1991); Medberry, et al., Nucleic Acids Res., 23: 485-490 (1995)).

Engineered minichromosomes can also be used to express one or more genes in plant cells. Cloned telomeric repeats introduced into cells may truncate the distal portion of a chromosome by the formation of a new telomere at the integration site. Using this method, a vector for gene transfer can be prepared by trimming off the arms of a natural plant chromosome and adding an insertion site for large inserts (Yu, et al., Proc Natl Acad Sci US A, 103:17331-6 (2006); Yu, et al., Proc Natl Acad Sci USA, 104:8924-9 (2007)). The utility of engineered minichromosome platforms has been shown using Cre/lox and FRT/FLP site-specific recombination systems on a maize minichromosome where the ability to undergo recombination was demonstrated (Yu, et al., Proc Natl Acad Sci USA, 103:17331-6 (2006); Yu, et al., Proc Natl Acad Sci USA, 104:8924-9 (2007)). Such technologies could be applied to minichromosomes, for example, to add genes to an engineered plant. Site specific recombination systems have also been demonstrated to be valuable tools for marker gene removal (Kerbach, et al., Theor. Appl. Genet. 111:1608-1616 (2005)), gene targeting Chawla, et al., Plant Biotechnol J., 4:209-218 (2006); Choi, et al., Nucleic Acids Res., 28: E19 (2000); Srivastava, et al., Plant Mol Biol. 46:561-566 (2001); Lyznik, et al., Nucleic Acids Res., 21:969-975 (1993)) and gene conversion (Djukanovic, et al., Plant Biotechnol J., 4:345-357 (2006)).

An alternative approach to chromosome engineering in plants involves in vivo assembly of autonomous plant minichromosomes (Carlson, et al., PLoS Genet., 3:1965-74 (2007)). Plant cells can be transformed with centromeric sequences and screened for plants that have assembled autonomous chromosomes de novo. Useful constructs combine a selectable marker gene with genomic DNA fragments containing centromeric satellite and retroelement sequences and/or other repeats.

Another approach useful to the described invention is Engineered Trait Loci (“ETL”) technology (U.S. Pat. No. 6,077,697; US Patent Application 2006/0143732). This system targets DNA to a heterochromatic region of plant chromosomes, such as the pericentric heterochromatin, in the short arm of acrocentric chromosomes. Targeting sequences may include ribosomal DNA (rDNA) or lambda phage DNA. The pericentric rDNA region supports stable insertion, low recombination, and high levels of gene expression. This technology is also useful for stacking of multiple traits in a plant (US Patent Application 2006/0246586).

Zinc-finger nucleases (ZFNs) are also useful for practicing the invention in that they allow double strand DNA cleavage at specific sites in plant chromosomes such that targeted gene insertion or deletion can be performed (Shukla, et al., Nature, 459(7245):437-41 (2009)); Townsend et al., Nature, 459(7245):442-5 (2009)).

Following transformation by any one of the methods described above, the following procedures can, for example, be used to obtain a transformed plant expressing the transgenes: select the plant cells that have been transformed on a selective medium, regenerate the plant cells that have been transformed to produce differentiated plants, select transformed plants expressing the transgene producing the desired level of desired polypeptide(s) in the desired tissue and cellular location.

Transformation techniques for dicotyledons are well known in the art and include Agrobacterium-based techniques and techniques that do not require Agrobacterium. Non-Agrobacterium techniques involve the uptake of heterologous genetic material directly by protoplasts or cells. This is accomplished by PEG or electroporation mediated uptake, particle bombardment-mediated delivery, or microinjection. In each case the transformed cells may be regenerated to whole plants using standard techniques known in the art.

Transformation of most monocotyledon species has now become somewhat routine. Preferred techniques include direct gene transfer into protoplasts using PEG or electroporation techniques, particle bombardment into callus tissue or organized structures, as well as Agrobacterium-mediated transformation.

Plants from transformation events are grown, propagated and bred to yield progeny with the desired trait, and seeds are obtained with the desired trait, using processes well known in the art.

### C. Plastid Transformation

In another embodiment the transgene is directly transformed into the plastid genome. Plastid transformation technology is extensively described in U.S. Pat. No. 5,451,513 to Maliga et al., U.S. Pat. No. 5,545,817 to McBride et al., and U.S. Pat. No. 5,545,818 to McBride et al., in PCT application no. WO 95/16783 to McBride et al., and in McBride, et al., Proc. Natl. Acad. Sci. USA 91:7301-7305 (1994). The basic technique for chloroplast transformation involves introducing regions of cloned plastid DNA flanking a selectable marker together with the gene of interest into a suitable target tissue, e.g., using biolistics or protoplast transformation (e.g., calcium chloride or PEG mediated transformation). The 1 to 1.5 kb flanking regions, termed targeting sequences, facilitate homologous recombination with the plastid genome and thus allow the replacement or modification of specific regions of the plastome. Suitable plastids that can be transfected include, but are not limited to, chloroplasts, etioplasts, chromoplasts, leucoplasts, amyloplasts, proplastids, statoliths, elaioplasts, proteinoplasts and combinations thereof.

### D. Methods for Reproducing Plants

Following transformation by any one of the methods described above, the following procedures can be used to obtain a transformed plant expressing the transgenes: select the plant cells that have been transformed on a selective medium; regenerate the plant cells that have been transformed to produce differentiated plants; select transformed plants expressing the transgene producing the desired level of desired polypeptide(s) in the desired tissue and cellular location.

In plastid transformation procedures, further rounds of regeneration of plants from explants of a transformed plant or tissue can be performed to increase the number of transgenic plastids such that the transformed plant reaches a state of homoplasmy (all plastids contain uniform plastomes containing transgene insert).

The cells that have been transformed may be grown into plants in accordance with conventional techniques. See, for example, McCormick et al. Plant Cell Reports, 5:81-84 (1986). These plants may then be grown, and either pollinated with the same transformed variety or different varieties, and the resulting hybrid having constitutive expression of the desired phenotypic characteristic identified. Two or more generations may be grown to ensure that constitutive expression of the desired phenotypic characteristic is stably maintained and inherited and then seeds harvested to ensure constitutive expression of the desired phenotypic characteristic has been achieved.

In some scenarios, it may be advantageous to insert a multi-gene pathway into the plant by crossing of lines containing portions of the pathway to produce hybrid plants in which the entire pathway has been reconstructed. This is especially the case when high levels of product in a seed compromises the ability of the seed to germinate or the resulting seedling to survive under normal soil growth conditions. Hybrid lines can be created by crossing a line containing one or more the transgene miRNA targeting sequence constructs disclosed herein with a line containing the miRNA. Use of lines that possess cytoplasmic male sterility (Esser, et al., Progress in Botany, Springer Berlin Heidelberg. 67:31-52 (2006)) with the appropriate maintainer and restorer lines allows these hybrid lines to be produced efficiently.

Cytoplasmic male sterility systems are already available for some Brassicaceae species (Esser, et al., Progress in Botany, Springer Berlin Heidelberg. 67:31-52 (2006)).

### E. Breeding

### 1. Methods of Breeding Hybrid Plants

Field crops are bred through techniques that take advantage of the plant's method of pollination. A plant is self-pollinated if pollen from one flower is transferred to the same or another flower of the same plant. A plant is cross-pollinated if the pollen comes from a flower on a different plant. Plants that have been self-pollinated and selected for type for many generations become homozygous at almost all gene loci and produce a uniform population of true breeding progeny. A cross between two different homozygous lines produces a uniform population of hybrid plants that may be heterozygous for many gene loci. A cross of two plants each heterozygous at a number of gene loci will produce a population of hybrid plants that differ genetically and may not be uniform.

The plants disclosed herein include hybrid plants which can be produced using any known breeding techniques. Hybrids are the product of a cross between genetically different parents. The development of hybrids in a plant breeding program often involves the development of homozygous inbred lines, the crossing of these lines, and the evaluation of the crosses. Most plant breeding programs combine the genetic backgrounds from two or more inbred lines or various other broad-based sources into breeding pools from which new inbred lines are developed by selfing and selection of desired phenotypes. Hybrids can also be used as a source of plant breeding material or as source populations from which to develop or derive new plant lines. The expression of a trait in a hybrid may exceed the midpoint of the amount expressed by the two parents, which is known as hybrid vigor.

Plant breeding techniques known in the art include, but are not limited to, recurrent selection, pedigree breeding, DNA marker enhanced selection, genetic marker enhanced selection and transformation. Inbred lines may, for instance, be derived from hybrids by using said methods as pedigree breeding and recurrent selection breeding. Newly developed inbreds are crossed with other inbred lines and the hybrids from these crosses are evaluated to determine which of those have commercial potential.

Pedigree breeding is a system of breeding in which individual plants are selected in the segregating generations from a cross on the basis of their desirability judged individually and on the basis of a pedigree record.

Recurrent selection is a breeding method based upon intercrossing selected individuals followed by continuing cycles of selection and intercrossing to increase the frequency of desired alleles in the population.

Recurrent selection may, for instance, be performed by backcross breeding, which involves a system of breeding whereby recurrent backcrosses are made to one of the parents of a hybrid, accompanied by selection for a specific character or characters. The backcross is the cross of a hybrid to either of its parents. Backcrossing can for instance be used to transfer a specific desirable trait that is present in a donor plant line to another, superior plant line (e.g. an inbred line) that lacks that trait.

The first step of this process involves crossing the superior plant line (recurrent parent) to a donor plant line (non-recurrent parent), that carries the appropriate gene(s) for the trait in question. The progeny of this cross is then mated back to the superior recurrent parent followed by selection in the resultant progeny for the desired trait to be transferred from the non-recurrent parent. After five or more backcross generations with selection for the desired trait and for the germplasm inherited from the recurrent parent, the progeny will be homozygous for loci controlling the characteristic being transferred, but will be like the superior parent for nearly all other genes. The last backcross generation is then selfed to give pure breeding progeny for the gene(s) being transferred. A hybrid developed from inbreds containing the transferred gene(s) is practically the same as a hybrid developed from the same inbreds without the transferred gene(s).

Introgression, also known as introgressive hybridization, is another hybrid breeding technique. Introgressive hybridization results in the movement of one or more genes (gene flow) from one species into the gene pool of another by repeated backcrossing of an interspecific hybrid with one of its parent species. Introgression is a long-term process; it may take many hybrid generations before the backcrossing is completed.

One example of introgressive hybridization is known as advanced backcross-self breeding. The AB method includes of crossing one parental line (donor parent) with another parental line (recurrent parent) to produce F₁ progeny. The F₁ progeny can be optionally self-crossed to generate F₂ progeny. The F₁ or F₂ progeny is then crossed with the recurrent parent to produce a backcross progeny (BC₁). BC₁ are selected and crossed again with the recurrent parent resulting in a second generation of backcross progeny (BC₂). BC₂ can be optionally backcrossed with the recurrent parent to generate a third generation of backcross progeny (BC₃). Plants from the BC₂ and/or the BC₃ generation are then allowed to self-pollinate for one or more generations, followed by evaluation for presence of the characteristics transferred from the donor parent. Methods for evaluating the presence of donor characteristics can be accomplished using any technique known in the art. Specific methods for evaluating the presence of donor characteristics, are described in detail in the examples below.

The disclosed QTLs, transgenes, or combinations thereof can be used to establish a breeding program to cultivate hybrid plants with one or more of desired phenotypic characteristics. Because quantitative traits are phenotypic characteristics that vary in degree and may include environmental influence, breeders may also take into consideration the breeding environment and or breeding location when cultivating the plants disclosed herein.

### 2. Genotyping Hybrid Plants

Quantitative traits are phenotypic characteristics that vary in degree and are typically attributed to the interactions between two or more genes and their environment. Quantitative trait loci (QTLs) are stretches of DNA that are closely linked to the genes that underlie the trait in question. A QTL may encompass 0, 1, or typically more than one gene. QTLs can be associated with any quantitative trait. In the most preferred embodiments, QTLs are associated with commercially valuable traits, for example antixenosis and/or anibiosis (such as reduction of nematode colonization, reduction of nematode reproduction, reduction of syncytium formation), or tolerance, or other traits that improve resistance to pests such as nematodes or increase crop yields.

Desirable QTL, transgenes, or combinations thereof, such as those disclosed herein, can be analyzed according to any method known in the art. For example, phenotypes can be assessed for improvement of one or more desirable traits using phenotype analysis techniques or genotyping.

For example, the presence of QTLs in a plant or plant cell can be associated or linked to regions of the genome that are contributing to variation in a trait of interest. Once the trait is associated or linked with one or more genetic markers, the genetic markers can be used to determine if a particular plant has the desirable QTL (i.e. a genetic region or chromosomal segment including the desirable QTL) or not.

Genomic regions can be analyzed using any method known in the art. For example, hybrid plants can be genotyped with restriction fragment length polymorphism (RFLP) markers. An RFLP is a difference in homologous DNA sequences that can be detected by the presence of fragments of different lengths after digestion of the DNA samples in question with specific restriction endonucleases. An RFLP probe is a labeled oligonucleotide sequence that hybridizes with one or more fragments of the digested DNA sample after they are separated by gel electrophoresis, thus revealing a unique blotting pattern characteristic to a specific genotype at a specific locus. Short, single- or low-copy genomic DNA or cDNA clones are typically used as RFLP probes.

Alternatively, QTLs can be identified by amplified fragment length polymorphism (AFLP). AFLP uses restriction enzymes to digest genomic DNA, followed by ligation of adaptors to the sticky ends of the restriction fragments. Restriction fragments are selected, and amplified by PCR using primers complementary to the adaptor sequence, the restriction site sequence and a few nucleotides inside the restriction site fragments. The amplified fragments are visualized (i.e. detection of a specific genotype at a specific locus) on denaturing polyacrylamide gels using, for example, autoradiography or fluorescence methodologies.

Other methods useful in QTL genotyping may include analysis of randomly amplified polymorphic DNA (RAPD), highly polymorphic short tandem repeat (STR) or simple sequence repeat (SSR) markers also referred to as microsatellites, or polymorphic single nucleotide polymorphisms (SNPs), or sequencing fragments of the genome (i.e., genomic sequencing). In some embodiments, the plants disclosed herein are genotyped according to random fragment length polymorphisms (RFLP) markers from a known genetic map. In this way, the presence of the desired QTL or QTLs can be monitored or tracked in the progeny of each successive round of a breeding program.

In some embodiment, protoplast fusion is used transfer of nucleic acids from a donor plant to a recipient plant. Protoplast fusion is an induced or spontaneous union, such as a somatic hybridization, between two or more protoplasts (cells of which the cell walls are removed by enzymatic treatment) to produce a single bi- or multi-nucleate cell. The fused cell, that may even be obtained from plant species that cannot be interbred in nature, is tissue cultured into a hybrid plant exhibiting the desirable combination of traits. The protoplasts are then fused using traditional protoplast fusion procedures, which are known in the art.

In another embodiment, embryo rescue is employed to transfer a nucleic acid including one or more superior QTLs from a donor plant to a recipient plant. Embryo rescue can be used as a procedure to isolate embryos from crosses wherein plants fail to produce viable seed. In this process, the fertilized ovary or immature seed of a plant is tissue cultured to create new plants.

### V. Screening Methods

Methods are also provided for identifying treatments, such as chemical treatments, that can modify expression or bioavailability of a β-1,4-endoglucancase, such as Glyma.10G017000, and or a pectin methylesterase inhibitor, such as Glyma.10G017000, in a plant.

In some embodiments, the method involves administering a candidate agent to a transgenic plant disclosed herein and comparing the effect of the administration on β-1,4-endoglucancase, and/or pectin methylesterase inhibitor activity in the plant, relative to a control, such as a wild-type plant of the same species. For example, the purpose of the method can be to identify an agent that causes the transgenic plant to exhibit increased pest resistance.

In some embodiments, the method involves contacting cells expressing a β-1,4-endoglucancase gene, and/or pectin methylesterase inhibitor gene or an ortholog or homolog thereof with a candidate agent, monitoring the effect of the candidate agent on β-1,4-endoglucancase gene, and/or pectin methylesterase inhibitor gene expression, and comparing the effect of the candidate agent on β-1,4-endoglucancase gene, and/or pectin methylesterase inhibitor gene expression to a control. For example, the purpose of the method can be to identify an agent that promotes or reduces β-1,4-endoglucancase gene, and/or pectin methylesterase inhibitor gene expression. In these embodiments, a decrease in β-1,4-endoglucancase gene, and/or pectin methylesterase inhibitor gene expression would identify an agent that could be used to increase nematode resistance. Likewise, the purpose of the method can be to identify an agent that increases β-1,4-endoglucancase gene, and/or pectin methylesterase inhibitor gene expression. In these embodiments, an increase in β-1,4-endoglucancase gene, and/or pectin methylesterase inhibitor gene expression would identify an agent that could be used to increase nematode susceptibility.

Expression of a β-1,4-endoglucancase gene, such as a Glyma.10G017000 gene, and/or a pectin methylesterase inhibitor gene, such as a Glyma.10G017100 gene, can be detected using routine methods, such as immunodetection methods. The methods can be cell-based or cell-free assays. The steps of various useful immunodetection methods have been described in the scientific literature, such as, e.g., Maggio, et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods. Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

In some embodiments, a reporter construct, such as a fluorochrome or enzyme, is operably linked to a Glyma.10G017000 expression control sequence. In some embodiments, a reporter construct, such as a fluorochrome or enzyme, is operably linked to a Glyma.10G017100 expression control sequence. In these embodiments, the purpose of the method can be to identify an agent that modulates activation of the Glyma.10G017000, and/or Glyma.10G017100 expression control sequence by detecting the effect of a candidate agent on reporter expression.

In general, candidate agents can be identified from large libraries of natural products or synthetic (or semi-synthetic) extracts or chemical libraries according to methods known in the art. Those skilled in the field of drug discovery and development will understand that the precise source of test extracts or compounds is not critical to the disclosed screening procedure. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds.

Synthetic compound libraries are commercially available, e.g., from Brandon Associates (Merrimack, NH) and Aldrich Chemical (Milwaukee, WI). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

When a crude extract is found to have a desired activity, further fractionation of the positive lead can be used to isolate chemical constituents responsible for the observed effect. Thus, the goal of the extraction, fractionation, and purification process is the careful characterization and identification of a chemical entity within the crude extract having the activity. The same assays described herein for the detection of activities in mixtures of compounds can be used to purify the active component and to test derivatives thereof. Methods of fractionation and purification of such heterogenous extracts are known in the art. If desired, compounds shown to be useful agents for treatment are chemically modified according to methods known in the art. Compounds identified as being of therapeutic value may be subsequently analyzed using animal models for diseases or conditions, such as those disclosed herein.

Candidate agents encompass numerous chemical classes, but are most often organic molecules, e.g., small organic compounds having a molecular weight of more than 100 and less than about 2,500 Daltons. Candidate agents include functional groups necessary for structural interaction with proteins, particularly hydrogen bonding, and typically include at least an amine, carbonyl, hydroxyl or carboxyl group, for example, at least two of the functional chemical groups. The candidate agents often include cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. In a further embodiment, candidate agents are peptides.

### VI. Methods of Identifying Pest Resistance Genes in Related Plants

Methods are also provided for identifying genes that control nematode resistance in other plants. Therefore, methods for identifying homologs and/or orthologs of Glyma.10G017000, and/or Glyma.10G017100 in plants are provided. The methods generally involve using the gene sequences for Glyma.10G017000 and/or Glyma.10G017100 disclosed herein.

In preferred embodiments, the plant is closely related to soybean. In some embodiments, the method involves scanning the genetic sequences of a plant for genes that are orthologous or homologous to Glyma.10G017000 or to Glyma.10G017100.

In other embodiments, the method involves conducting a BLAST search of plant genomes for genes having the highest nucleic acid sequence identity to that of Glyma.10G017000. For example, the orthologous gene can have 10%, 15%, 20%, 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the nucleic acid sequence SEQ ID NO: 1, 2, 3, 4, 5, or 6, or a nucleic acid encoding the amino acid sequence of SEQ ID NO:7, or 8, or a fragment or variant thereof.

In other embodiments, the method involves conducting a BLAST search of plant genomes for genes having the highest nucleic acid sequence identity to that of Glyma.10G017100. For example, the orthologous gene can have 10%, 15%, 20%, 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the nucleic acid sequence SEQ ID NO:9, 10, 11, 12, 13, or 14, or a nucleic acid encoding the amino acid sequence of SEQ ID NO:15, or 16, or a fragment or variant thereof.

### VII. Methods of Genotyping Plants for Pest Resistance

### A. Haplotypes

The sequences disclosed herein can be used to screen for pest resistance in plants. For example, the genotype of one or more insertions, deletions, and polymorphisms in or around Glyma.10G017000 and/or Glyma.10G017100 that reduce or inhibit expression of the Glyma.10G017000 and/or Glyma.10G017100 gene product, or lead to an altered gene product, such as a truncated protein or alternative splice variant, or a gene product with reduced function can be used to genotype a plant as nematode resistant relative to a plant encoding a functional, full-length protein. For example, deletions, insertions, and polymorphisms can be determined by comparing the Glyma.10G017000 and/or Glyma.10G017100 sequence from a test plant to SEQ ID NOs:1-6; or 9-14, respectively, using global sequence alignment tools.

As set forth in the Examples, below, it has been established that nematode resistant plants include a Glyma.10G017000A having 23 SNPs, a 10 bp insertion, a 13 bp deletion, and a 15 bp insertion in the promoter of Glyma.10G017000, as well as three nonsynonymous SNPs (M31I, T106I, V174A), two silent exonic SNPs, five intronic SNPs, a 1-bp intronic deletion, and a 3′ UTR SNP in Glyma.10G017000. It has also been established that nematode resistant plants include a Glyma.10G017100A having an insertion of 968 bp approximately 1.1 kb upstream of the 5′ UTR of Glyma.10G017100, as well as two single bp deletions after the 3′ end of this insertion and multiple SNPs before the 5′ end of the insertion; and five SNPs resulting in amino acid changes (V93I, D120Y, L149R, C167Y, E214D, one resulting in a premature stop codon (Q242*), and five silent exonic SNPs of Glyma.10G017100.

Therefore, in some forms, a plant can also be determined to be pest resistant by detecting one or more of the 23 SNPs, a 10 bp insertion, a 13 bp deletion, and a 15 bp insertion in the promoter of Glyma.10G017000. A plant can also be determined to be pest resistant by detecting one or more of the three nonsynonymous SNPs (M31I, T106I, V174A), two silent exonic SNPs, five intronic SNPs, a 1-bp intronic deletion, and a 3′ UTR SNP of Glyma.10G017000. A plant can also be determined to be pest resistant by detecting the insertion of 968 bp approximately 1.1 kb upstream of the 5′ UTR of Glyma.10G017100. A plant can also be determined to be pest resistant by detecting the two single bp deletions after the 3′ end of this insertion and multiple SNPs before the 5′ end of the insertion. A plant can also be determined to be pest resistant by detecting the five SNPs resulting in amino acid changes (V93I, D120Y, L149R, C167Y, E214D, one resulting in a premature stop codon (Q242*), and five silent exonic SNPs of Glyma.10G017100.

The process of determining which specific nucleotide (i.e., allele) is present at each of one or more SNP positions, such as a disclosed SNP position in the Glyma.10G017000 and/or Glyma.10G017100 gene locus, is referred to as SNP genotyping. Methods for SNP genotyping are generally known in the art (Chen et al., Pharmacogenomics J., 3(2):77-96 (2003)); Kwok, et al., Curr. Issues Mol. Biol., 5(2):43-60 (2003)); Shi, Am. J. Pharmacogenomics, 2(3):197-205 (2002)); and Kwok, Annu. Rev. Genomics Hum. Genet., 2:235-58 (2001)).

SNP genotyping can include the steps of collecting a biological sample from a plant, isolating genomic DNA from the cells of the sample, contacting the nucleic acids with one or more primers which specifically hybridize to a region of the isolated nucleic acid containing a target SNP under conditions such that hybridization and amplification of the target nucleic acid region occurs, and determining the nucleotide present at the SNP position of interest, or, in some assays, detecting the presence or absence of an amplification product (assays can be designed so that hybridization and/or amplification will only occur if a particular SNP allele is present or absent). In some assays, the size of the amplification product is detected and compared to the length of a control sample; for example, deletions and insertions can be detected by a change in size of the amplified product compared to a normal genotype.

The neighboring sequence can be used to design SNP detection reagents such as oligonucleotide probes and primers. In some embodiments, probe or primers are designed based on the nucleic acid sequence disclosed herein, i.e., SEQ ID NO:1-6, or 9-14.

Common SNP genotyping methods include, but are not limited to, TaqMan assays, molecular beacon assays, nucleic acid arrays, allele-specific primer extension, allele-specific PCR, arrayed primer extension, homogeneous primer extension assays, primer extension with detection by mass spectrometry, pyrosequencing, multiplex primer extension sorted on genetic arrays, ligation with rolling circle amplification, homogeneous ligation, multiplex ligation reaction sorted on genetic arrays, restriction-fragment length polymorphism, single base extension-tag assays, and the Invader assay. Such methods may be used in combination with detection mechanisms such as, for example, luminescence or chemiluminescence detection, fluorescence detection, time-resolved fluorescence detection, fluorescence resonance energy transfer, fluorescence polarization, mass spectrometry, and electrical detection.

The disclosed invention can be further understood by the following numbered paragraphs:

1. A plant or plant cell including a genetic modification that reduces, inhibits or silences expression or translation of a target polynucleotide having a nucleic acid sequence of
(i) any one of SEQ ID NOs:1-3, or a complement thereof, and/or a nucleic acid encoding the polypeptide of SEQ ID NO:7 or 8, or a complement thereof, and/or
(ii) any one of SEQ ID NOs:9-11, or a complement thereof, and/or a nucleic acid encoding the polypeptide of SEQ ID NO:15 or 16, or a complement thereof, wherein the plant is a transgenic or non-transgenic plant.

2. The plant or plant cell wherein the genetic modification was induced by gene editing technology optionally selected from CRISPR/Cas, TALENs, and Zinc Finger Nucleases.

3. A transgenic plant or transgenic plant cell, including a polynucleotide including an expression control sequence operably linked to a nucleic acid sequence encoding an antisense nucleic acid that reduces, inhibits or silences expression or translation of a target polynucleotide having a nucleic acid sequence of
(i) any one of SEQ ID NOs:1-3, or a complement thereof, and/or a nucleic acid encoding the polypeptide of SEQ ID NO:7 or 8, or a complement thereof; and/or
(ii) any one of SEQ ID NOs:9-11, or a complement thereof, and/or a nucleic acid encoding the polypeptide of SEQ ID NO:15 or 16, or a complement thereof.

4. The plant or plant cell of paragraph 3, wherein the nucleic acid alters, reduces, inhibits, or silences expression or translation of the target polynucleotide by RNAi, dsRNA, miRNA, siRNA, or transacting small-interfering RNAs (tasiRNA).

5. The plant or plant cell of any one of paragraphs 1-4, further including a second polynucleotide including a promoter sequence including SEQ ID NO:4, or a fragment thereof including 50, 100, 150, 250, 500, 750, 1,000, 1,250, 1,500, or 2,000 or more nucleotides of SEQ ID NO:4 operably linked to one or more nucleic acid sequence(s) encoding one or more nematode susceptibility genes.

6. A plant or plant cell including a polynucleotide including a promoter sequence of SEQ ID NO:4, or a fragment thereof including 50, 100, 150, 250, 500, 750, 1,000, 1,250, 1,500, or 2,000 or more nucleotides of SEQ ID NO:4 operably linked to one or more nucleic acid sequence(s) encoding one or more nematode susceptibility genes.

7. A plant or plant cell including a polynucleotide including a nucleic acid sequence of SEQ ID NO:5 or 6,
optionally wherein the plant or cell expresses a polypeptide having an amino acid sequence of SEQ ID NO:8.

8. A plant or plant cell including a polynucleotide including a promoter sequence of SEQ ID NO:4, or a fragment thereof including 50, 100, 150, 250, 500, 750, 1,000, 1,250, 1,500, or 2,000 or more nucleotides of SEQ ID NO:4 operably linked to a polynucleotide including a nucleic acid sequence of SEQ ID NO:5.

9. The plant or cell of any one of paragraphs 5-8, wherein the plant or cell exhibits reduced expression of one or more nematode susceptibility genes, or has a reduced amount of one or more proteins encoded by one or more nematode susceptibility genes, relative to a wild-type control plant.

10. The plant or cell of any one of paragraphs 5-9, wherein the plant or cell does not express one or more nematode susceptibility genes, or does not express one or more proteins encoded by one or more nematode susceptibility genes, relative to a wild-type control plant.

11. The plant or cell of any one of paragraphs 5-10, wherein one nematode susceptibility gene includes a gene encoding a β-1,4-endoglucancase enzyme,
optionally wherein the optionally wherein the β-1,4-endoglucancase enzyme includes an amino acid sequence of SEQ ID NO:7.

12. The plant or cell of any one of paragraphs 5-11, wherein one nematode susceptibility gene includes Glyma.10G017000,
optionally wherein the nematode susceptibility gene includes SEQ ID NO:2.

13. The plant or plant cell of any one of paragraphs 1-12, further including a polynucleotide including a promoter sequence including SEQ ID NO:12, or a fragment thereof including 50, 100, 150, 250, 500, 750, 1,000, 1,250, 1,500, or 2,000 or more nucleotides of SEQ ID NO:12 operably linked to one or more nucleic acid sequence(s) encoding one or more nematode susceptibility genes.

14. A plant or plant cell including a polynucleotide including a promoter sequence of SEQ ID NO:12, or a fragment thereof including 50, 100, 150, 250, 500, 750, 1,000, 1,250, 1,500, or 2,000 or more nucleotides of SEQ ID NO:12 operably linked to one or more nucleic acid sequence(s) encoding one or more nematode susceptibility genes.

15. A plant or plant cell including a polynucleotide including a nucleic acid sequence of SEQ ID NO:13 or 14,
optionally wherein the plant or cell expresses a polypeptide having an amino acid sequence of SEQ ID NO:16.

16. A plant or plant cell including a polynucleotide including a promoter sequence of SEQ ID NO:12, or a fragment thereof including 50, 100, 150, 250, 500, 750, 1,000, 1,250, 1,500, or 2,000 or more nucleotides of SEQ ID NO:12 operably linked to a polynucleotide including a nucleic acid sequence of SEQ ID NO:13.

17. The plant or cell of any one of paragraphs 13-16, wherein the plant or cell exhibit reduced expression of one or more nematode susceptibility genes, or has a reduced amount of one or more proteins encoded by one or more nematode susceptibility genes, relative to a wild-type control plant.

18. The plant or cell of any one of paragraphs 13-17, wherein the plant or cell does not express of one or more nematode susceptibility genes, or does not express one or more proteins encoded by one or more nematode susceptibility genes, relative to a wild-type control plant.

19. The plant or cell of any one of paragraphs 5-18, wherein one nematode susceptibility gene includes a gene encoding a pectin methylesterase inhibitor,
optionally wherein the optionally wherein the pectin methylesterase inhibitor includes an amino acid sequence of SEQ ID NO:15.

20. The plant or cell of any one of paragraphs 5-19, wherein one nematode susceptibility gene includes Glyma.10G017100,
optionally wherein the nematode susceptibility gene includes SEQ ID NO:10.

21. A plant or plant cell including
(i) a polynucleotide including a promoter sequence of SEQ ID NO:1, or a fragment thereof including 50, 100, 150, 250, 500, 750, 1,000, 1,250, 1,500, or 2,000 or more nucleotides of SEQ ID NO: 1; and/or
(ii) a polynucleotide including a promoter sequence of SEQ ID NO:9, or a fragment thereof including 50, 100, 150, 250, 500, 750, 1,000, 1,250, 1,500, or 2,000 or more nucleotides of SEQ ID NO:9;
operably linked to one or more nucleic acid sequence(s) encoding one or more heterogenous transgenes, optionally wherein the transgene(s) includes pest resistance gene optionally a nematode resistance gene.

22. The plant or cell of any one of paragraphs 1-21, wherein the plant includes a soybean plant optionally, wherein the soybean plant includes a Glycine max plant,
optionally wherein the Glycine max is a cultivar selected from the group consisting of Jack, Resnik, Williams 82, Corsoy, Crawford, Hutcheson, Benning, Woodruff, Kunitz and Champ.

23. The plant of any one of paragraphs 1-21, wherein the plant includes a cereal crop plant, such as wheat, oat, barley, or rice; or a forage plant such as bahiagrass, dallisgrass, kleingrass, guineagrass, reed canarygrass, orchardgrass, ricegrass, foxtail, or vetch; or a legume such as lentil, or chickpea; or an oilseed such as canola; or a vegetable such as onion or carrot; or a specialty crop such as caraway, hemp, or sesame.

24. The plant or cell of any one of paragraphs 1-23, wherein the plant is resistant to one or more species of nematode relative to a non-plant of the same species.

25. The plant or cell of paragraph 24, wherein the nematode genus is selected from the group consisting of Heterodera, and Meloidogyne.

26. The plant or cell of paragraph 24 or 25, wherein the nematode is selected from Heterodera glycines, Heterodera schachtii, and Meloidogyne incognita.

27. The plant or cell of any one of paragraphs 24-26, wherein the nematode is Meloidogyne incognita.

28. The plant of any one of paragraphs 24-27, wherein the resistance is conferred through reduction of nematode colonization, reduction of nematode reproduction, reduction of syncytium formation, tolerance, or combinations thereof.

29. The plant of any one of paragraphs 1-28, further including a pest resistance QTL.

30. A seed from any of the plants according to any one of paragraphs 1-29.

31. A foodstuff including a plant part from the plant according to any one of paragraphs 1-30.

32. A method for increasing pest resistance in a plant including altering or reducing expression of Glyma.10G017000 β-1,4-endoglucancase, or its ortholog in another plant species by an amount effective to increase pest resistance in the plant.

33. The method of paragraph 32, wherein expression of the Glyma.10G017000 β-1,4-endoglucancase is altered or reduced by reducing expression or translation of a target polynucleotide having a nucleic acid sequence according to SEQ ID NO:2 or 3, or a complement thereof, or a nucleic acid encoding the polypeptide of SEQ ID NO:7, or a complement thereof.

34. A method for increasing pest resistance in a plant including altering or reducing expression of Glyma.10G017100 pectin methylesterase inhibitor, or its ortholog in another plant species by an amount effective to increase pest resistance in the plant.

35. The method of paragraph 34, wherein expression of the Glyma.10G017100 pectin methylesterase inhibitor is altered or reduced by reducing expression or translation of a target polynucleotide having a nucleic acid sequence according to SEQ ID NO: 10 or 11, or a complement thereof, or a nucleic acid encoding the polypeptide of SEQ ID NO:16, or a complement thereof.

36. The method of any one of paragraphs 32-35, wherein the pest includes a nematode,
optionally wherein the nematode genus is selected from the group consisting of Heterodera, and Meloidogyne.
optionally wherein the nematode is selected from Heterodera glycines, Heterodera schachtii, and Meloidogyne incognita.

37. A method of increasing nematode resistance including reducing expression of Glyma.10G017000 β-1,4-endoglucancase or its ortholog in another plant species by an amount effective to increase nematode resistance in the plant.

38. The method of paragraph 37, wherein expression of the Glyma.10G017000 β-1,4-endoglucancase, or its ortholog in another plant species, is reduced by reducing expression or inhibiting translation of a target polynucleotide having a nucleic acid sequence according to any one of SEQ ID NO:1, 2, 3, or a complement thereof, or a nucleic acid encoding the polypeptide of SEQ ID NO:7 or 8, or a complement thereof.

39. A method of increasing nematode resistance including reducing expression of Glyma.10G017100 pectin methylesterase inhibitor or its ortholog in another plant species by an amount effective to increase nematode resistance in the plant.

40. The method of paragraph 39, wherein expression of the Glyma.10G017000 β-1,4-endoglucancase, or its ortholog in another plant species, is reduced by reducing expression or inhibiting translation of a target polynucleotide having a nucleic acid sequence according to any one of SEQ ID NO:9, 10, 11, or a complement thereof, or a nucleic acid encoding the polypeptide of SEQ ID NO:15 or 16, or a complement thereof.

41. The method of any one of paragraphs 37-40, wherein the plant includes a polynucleotide including any one of SEQ ID NOs:4-6 and/or 12-14.

42. The method of any one of paragraphs 32-41, wherein the plant is a soybean plant,
optionally wherein the soybean plant includes a Glycine max plant,
optionally wherein the Glycine max is a cultivar selected from the group consisting of Jack, Resnik, Williams 82, Corsoy, Crawford, Hutcheson, Benning, Woodruff, Kunitz and Champ.

43. A plant, composition, method, or other material or process described herein in the text, drawings, or combination thereof.

### EXAMPLES

### Example 1: Fine Mapping Resistance to M. incognita in Soybean Cultivar Forrest

### Methods

### Plant and Nematode Materials

A cross was made between the soybean cultivars Bossier and Forrest. Forrest, a maturity group (MG) V cultivar (Hartwig and Epps 1973), exhibits a high level of resistance to M. incognita (Luzzi et al. 1994), while Bossier, an MG VIII cultivar, is highly susceptible to this nematode (Luzzi et al. 1994). The F₁ plants were grown in a winter nursery during the subsequent winter. Subsequently, F₂ plants were the grown. In the subsequent fall, pods were taken from each plant for advancement through a single seed descent method. Seeds from the F₂ generation were then advanced for two generations during the following season. The resulting F₅ seeds were planted the following summer. In the fall, 960 F₅ plants were harvested and threshed individually to form F_{5:6} recombinant inbred lines (RILs).

Lines ExF63 (resistant) and ExF67 (susceptible) were derived from a cross between Forrest and Essex (Lightfoot et al. 2005). The RILs shared 80% of their genomes while differing at Rmi1 (Gamage et al., unpublished).

The M. incognita race 3 population used is maintained primarily on eggplant cv. ‘Black Beauty’ with regular passage on susceptible soybean cv. ‘Williams 82’ to preserve virulence. Eggs were extracted for inoculation according to Hussey and Barker (1973). For inoculation with J2s, eggs were surface-sterilized in 0.02% sodium azide for 20 minutes, washed thoroughly with water, then placed on a hatching sieve in water supplemented with antibiotics (1.5 mg/ml gentamycin and 0.05 mg/ml nystatin) at 27° C. for three days.

Evaluation of M. incognita Resistance

Seeds were germinated in CYG germination pouches (Mega International, Roseville, MN) for three days at 25° C. in the dark. After three days, three seedlings of each F_{2:3} line, along with parental lines, were planted into cone-tainers containing a 1:1 sand:soil mixture. Plants were grown in a greenhouse in Athens, GA kept at 75-87° C. with 16/8 day/night light cycle supplemental lighting. Each plant was inoculated with 2,500 M. incognita eggs eleven days after planting. Six weeks after inoculation, the root systems were rinsed of soil and gall severity was scored from 1-5, with 1 being the galling level of the resistant parent Forrest and 5 being the galling level of the susceptible parent Bossier. The susceptible Williams 82 was also included as a susceptible check.

An additional screen of F_{2:3} plants was conducted using 21 randomly selected lines found to be segregating at GSM039, the marker currently used for marker-assisted selection at the University of Georgia (Pham et al. 2013). This screen was conducted as described above, with the modification of screening 12 replicates of each line.

Greenhouse screening of F₅ lines was conducted as described above with the following modifications—the plants were inoculated with 3,000 M. incognita eggs three days after planting and roots were phenotyped eight weeks after inoculation.

For comparison of the response of different cultivars and RILs to M. incognita, eggs were extracted from each root system seven weeks after inoculation.

### RIL Genotyping

A penny-sized section of a young leaf was taken from each plant two weeks after planting, collected in 1.4 ml tube in a 96-well latch rack, dried, then ground using a Genogrinder 2010 (SPEX SamplePrep, Metuchen, NJ) and a single bead per tube. DNA was extracted using a modified CTAB method. Ground tissue was incubated in 800 μl extraction buffer (2% CTAB, 1.4 M NaCl, 100 mM Tris-HCl pH 8, 20 mM EDTA, 0.1% β-mercaptoethanol, 0.34% PVP-40) at 65° C. for 2 hours, then centrifuged at 3,600 rpm for 10 min. The supernatant was mixed 1:1 with isopropanol, incubated at −20° C. for 2 hours, then centrifuged at 3,600 rpm for 15 min. The pellet was washed in 70% ethanol and resuspended in H₂O. Kompetitive allele-specific PCR (KASP) was performed using low ROX KASP-TF Master Mix (LGC Biosearch Technologies) in 4 μl reactions, with ˜20 ng genomic DNA using a Roche Lightcycler 480 II instrument (Roche). Fluorescence of the plates was read with a Tecan Infinite M1000 Pro (Tecan Trading AG, Switzerland). Allele calls were made using KlusterCaller (LGC Biosearch Technologies). The KASP marker GSM039 was used to genotype the chromosome 10 QTL (Pham et al. 2013).

Additional KASP primers were designed for high density near the overlapping region of prior mapping studies using Geneious Prime 2022 (Table 1, Table 2). Of the 235 kb QTL mapped by (Pham et al. 2013), 90 kb was covered by markers. After Glyma.10G017200, no SNPs associated with resistance were found. Markers in this region do not correspond to resistance but solely to parental lineage.

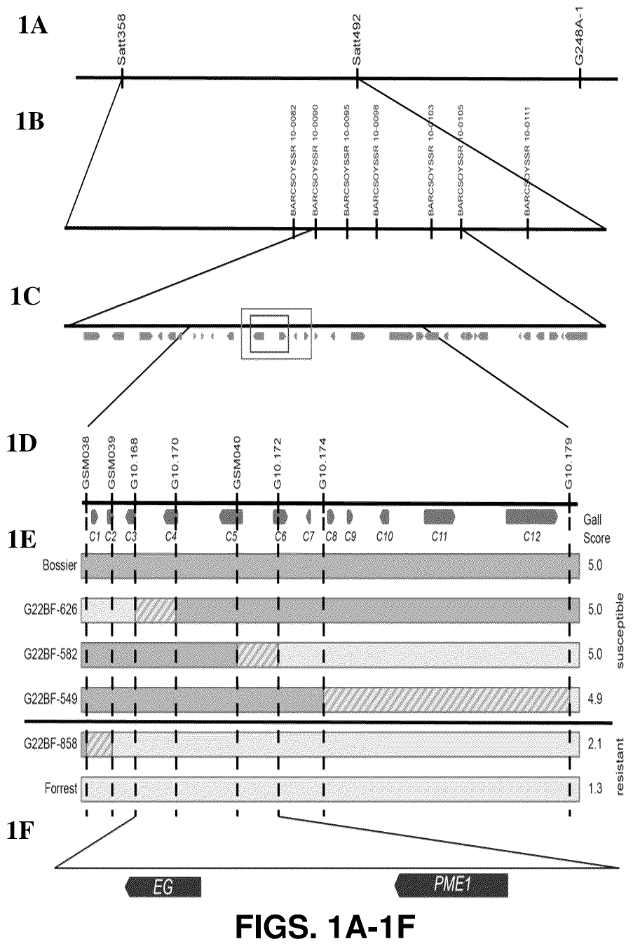

For initial genotypic screening of 884 F₅ Bossier x Forrest plants, DNA was extracted from 5 pooled seeds of each line (Edwards et al. 1991). Briefly, 5 seeds were incubated in 2 ml Edwards extraction buffer (200 nM Tris pH 7.5, 250 nM NaCl, 25 mM EDTA, 0.5% SDS) at 65° C. for 4 hours, then overnight at 4° C. After incubation, NaCl was added to final concentration 250 mM. DNA was precipitated with isopropanol, washed with ethanol, and suspended in H₂O. Genotyping was conducted using a series of KASP markers along the Rmi1 candidate region as described above (Table 1; FIGS. 1A-1F).

TABLE 1
Primers used for KASP genotyping amplification.

| | Marker | | | |
|---|---|---|---|---|
| Gene locus | name | Primer | Primer Sequence | Source |
| Glyma.10G016600 | GSM038 | FAM | GAAGGTGACCAAGTTCATGCTTTGTCTTCAGATCCGAGTCGATT | Pham et |
| | | HEX | GAAGGTCGGAGTCAACGGATTTGTCTTCCAGATCCGAGTCGATA | al. 2013 |
| | | common | GATGAAATTATACACCGTGGGGCCTT (SEQ ID NO: 17) | |
| Glyma.10G016700 | GSM039 | FAM | GAAGGTGACCAAGTTCATGCTGGTGTCGGTGATGCGGTGAATT | Pham et |
| | | HEX | GAAGGTCGGAGTCAACGGATTGGTGTCGGTGATGCGGTGAATA | al. 2013 |
| | | common | GATGAAATTATACACCGTGGGGCCTT (SEQ ID NO: 18) | |
| Glyma.10G016832 | G10.168 | FAM | GAAGGTGACCAAGTTCATGCTTAAGTGGTGTCGTTAATAATGATTTTC | this |
| | | HEX | GAAGGTCGGAGTCAACGGATTTAAGTGGTGTCGTTAATAATGATTTTT | study |
| | | common | ACCATCGTCAACCAACTAATGTT (SEQ ID NO: 19) | |
| Glyma.10G017000 | G10.170 | FAM | GAAGGTGACCAAGTTCATGCTGCTCCATGAAAGCATGGTGG | this |
| | | HEX | GAAGGTCGGAGTCAACGGATTGCTCCATGAAAGCATGGTGA | study |
| | | common | CGTATGCATATACCAACTCTCACATTAC (SEQ ID NO: 20) | |
| Glyma.10G017100 | GSM040 | FAM | GAAGGTGACCAAGTTCATGCTGAACTTGGTCTCATTCACTTTAGCTTA | Pham et |
| | | HEX | GAAGGTCGGAGTCAACGGATTAACTTGGTCTCATTCACTTTAGCTTG | al. 2013 |
| | | common | TGTCCTCYAAAGACCGAAAGCTTCTT (SEQ ID NO: 21) | |
| Glyma.10G017200 | G10.172 | FAM | GAAGGTGACCAAGTTCATGCTCGCTCAAGATGTCGAGCAG | this |
| | | HEX | GAAGGTCGGAGTCAACGGATTCGCTCAAGATGTCGAGCAC | study |
| | | common | GATGGTGGTGAAGTTCCCGT (SEQ ID NO: 22) | |
| Glyma.10G017400 | G10.174 | FAM | GAAGGTGACCAAGTTCATGCTTATCATTGGCGGACCTACATGT | this |
| | | HEX | GAAGGTCGGAGTCAACGGATTTATCATTGGCGGACCTACATGC | study |
| | | common | AATTAGTAACGGACCCAAGATCC (SEQ ID NO: 23) | |
| Glyma.10G017900 | G10.179 | FAM | GAAGGTGACCAAGTTCATGCTCCAACCAACCTTATATCTCCGT | this |
| | | HEX | GAAGGTCGGAGTCAACGGATTCCAACCAACCTTATATCTCCGC | study |
| | | common | TTGGCACCAAACTTCCCATC (SEQ ID NO: 24) | |

TABLE 2
Genes present in the region included in the new KASP marker design. The ID column refers to the label the gene has FIG. 1D.
The Gene column has the Wm82.a6.v1 gene name. The NCBI ID contains the Gene ID number used by NCBI to identify the gene.
The annotation columns contain the predictions from the Phytozome Gene Report and NCBI RefSeq annotation predictions.

| ID | Gene | NCBI ID | Phytozome Annotation | NCBI RefSeq Annotation |
|---|---|---|---|---|
| C1 | Glyma.10G016600 | 100527907 | pollen ole e 1 allergen and extensin family | uncharacterized LOC100527907 |
| | | | protein | |
| C2 | Glyma.10G016700 | 100785053 | pollen ole e 1 allergen and extensin family | protein SEED AND ROOT HAIR |
| | | | protein | PROTECTIVE PROTEIN |
| C3 | Glyma.10G016832 | 106794947 | no functional annotation available | uncharacterized LOC106794947, ncRNA |
| C4 | Glyma.10G017000 | 100789144 | endo-1,4-β-glucanase | endoglucanase 17 |
| C5 | Glyma.10G017100 | 100789666 | pectin methylesterase/pectin methylesterase | pectinesterase-like |
| | | | inhibitor | |
| C6 | Glyma.10G017200 | 100790729 | pectin methylesterase/pectin methylesterase | probable pectinesterase/pectinesterase |
| | | | inhibitor | inhibitor 41 |
| C7 | Glyma.10G017300 | 100791255 | plant invertase/pectin methylesterase inhibitor | pectinesterase inhibitor 3 |
| C8 | Glyma.10G017400 | 100500182 | unknown function; At5g01610-like | uncharacterized LOC100500182 |
| C9 | Glyma.10G017500 | 100808207 | unknown function; At5g01610-like | uncharacterized protein At5g01610 |
| C10 | Glyma.10G017600 | 100306369 | late embryogenesis abundant protein, LEA-5 | uncharacterized LOC100306369 |
| C11 | Glyma.10G017700 | 100814110 | poly(ADP-ribose) polymerase | poly [ADP-ribose] polymerase 2-A |
| C12 | Glyma.10G017800 | 100819794 | gamma-tubulin complex component protein | gamma-tubulin complex component 4 |
| | | | | homolog |

### Statistical Analysis

Statistical analysis was conducted using analysis of variance (ANOVA) in R (R Core Team 2024), with genotype treated as a fixed effect.

### Expression Analysis

To verify that mapped genes are expressed in root tissue, RNAseq reads mapped to the candidate region were extracted using samtools v1.17 (Danecek et al. 2021) and visualized using Geneious Prime 2024.

Soybean seeds were surface-sterilized by placing in glass Petri dishes inside a glass bell jar desiccator with a beaker containing 100 ml bleach (8.25% NaOCl) and 3.5 ml concentrated HCl for 16 hours. Seeds were then rolled in germination paper and placed upright in a glass beaker with a thin layer of dH₂O on the bottom, covered in plastic wrap with small holes poked through, and incubated in the dark at 25° C. After three days, seedlings of equivalent lengths were moved to fresh germination paper in Hoagland's solution. These germination rolls were kept in a 27° C. growth chamber on a 16/8 light (100 μmol m·⁻²·s⁻¹)/dark cycle. After two days, the bottom 2 cm of the primary root tip was collected from ten seedlings of each genotype. Collected tissue was ground using a liquid nitrogen-chilled porcelain mortar and pestle, then extracted using the QIAGEN Plant RNeasy Mini Kit (QIAGEN, Hilden, Germany). Total RNA (500 ng) from each sample was used for cDNA synthesis using PrimeScript 1st Strand cDNA Synthesis Kit (Takara Bio USA, Mountain View, CA). Quantitative RT-PCR was performed on a Bio-Rad CFX Real-Time System using PowerUp SYBR Master Mix (Applied Biosystems) according to the manufacturer's recommendation. The primers used for qPCR are listed in Table 3. The reference genes GmELF1A and Gmcons7 were used to normalize expression (Libault et al. 2008, Miranda et al. 2013). This assay was repeated three times. Another assay was conducted as above, using Williams 82, Essex, Forrest, ExF67, ExF63, and PI 96354 seeds.

To confirm that annotated genes are expressed in root tissue, cDNA from infected and non-infected ExF63 and ExF67 roots was amplified with ExTaq (Promega) using primers specific to mRNA for each fine-mapped gene (Table 3).

### Candidate Gene Sequence Evaluation

DNA from Bossier, Essex, PI 96354, and Forrest was extracted from leaf tissue punches using a CTAB/CHCl₃/Isopropanol method. Finely ground leaf tissue was incubated in CTAB extraction buffer (0.1 M Tris-HCl pH 9, 1.4 M NaCl, 20 mM EDTA, 2% CTAB) and 0.3 mg/μl RNAse A at 65° C. for 30 minutes. An equal amount of isoamyl chloroform was added before a 5 min incubation at 25° C. incubation and centrifuging at 17,900×g for 4 min. The supernatant was mixed 1:1 with isopropanol and incubated at −20° C. for 20 minutes, then centrifuged at 17,900×g for 15 min. The pellet was washed in 70% ethanol and resuspended in dH₂O. Both candidate genes were amplified from gDNA using Q5 PCR using primers listed in Table 3. The coding regions were amplified from cDNA using primers in Table 3.

TABLE 3
Primers used for mRNA and gDNA amplification for sequencing and qPCR.
Primers used for mRNA sequencing from cDNA

| Gene | Forward primer | Reverse primer |
|---|---|---|
| Glyma.10G017000 | ATGACTTTCTCCTTTTCCTTC | CTAGAGTTGACCGAATGAG |
| | (SEQ ID NO: 25) | (SEQ ID NO: 26) |
| Glyma.10G017100 | ATGAACAACCTCACACTTGCAT | ATAAATGTCACGTTCACGGTAGAA |
| | (SEQ ID NO: 27) | (SEQ ID NO: 28) |

Primers used for gPCR

| Gene | Forward primer | Reverse primer | Source |
|---|---|---|---|
| Glyma.02G016400 | CCCGTTCATTTCAATCACCCC | CCCTCCAACCAAATCAACATGC | this study |
| | (SEQ ID NO: 29) | (SEQ ID NO: 30) | |
| Glyma.10G017000 | AATGCCTTCCCAGTCCCCA | CCCTCCAACTAAATCAACGTGC | this study |
| | (SEQ ID NO: 31) | (SEQ ID NO: 32) | |
| Glyma.10G017100 | TCGGAATCTGATGAGGTGTTT | TTGGCAACAACAAGATTGAACT | this study |
| | (SEQ ID NO: 33) | (SEQ ID NO: 34) | |
| Gmcons7 | ATGAATGACGGTTCCCATGTA | GGCATTAAGGCAGCTCACTCT | Libault et |
| | (SEQ ID NO: 35) | (SEQ ID NO: 36) | al. 2008 |
| GmELF1A | GACCTTCTTCGTTTCTCGCA | CGAACCTCTCAATCACACGC | Miranda et |
| | (SEQ ID NO: 37) | (SEQ ID NO: 38) | al. 2013 |

Primers used for sequencing gDNA

| Gene | Forward primer | Reverse primer |
|---|---|---|
| Glyma.10G017000 | GTCTTTCTCCATCGTCCTCAC | TGACCCAATCCCATATGCTTG |
| | (SEQ ID NO: 39) | (SEQ ID NO: 40) |
| Glyma.10G017000 | AAGATAGGCCAGTGCTCCTAC | TGTAGGTCTTCCAGTTTGCTG |
| | (SEQ ID NO: 41) | (SEQ ID NO: 42) |
| Glyma.10G017000 | GAGAACAGACGAAATTGTCCG | AAAACGCTGTAGCAGGAGTAG |
| | (SEQ ID NO: 43) | (SEQ ID NO: 44) |
| Glyma.10G017100 | TTCATCCTGACTAACAACCG | TGTGAGATCTGGGAATGTGG |
| | (SEQ ID NO: 45) | (SEQ ID NO: 46) |
| Glyma.10G017100 | CACAGCACGTACCCTGCTAG | GCAGAAGGTGGGAAGAAGAAAG |
| | (SEQ ID NO: 47) | (SEQ ID NO: 48) |
| Glyma.10G017100 | CGGTTCGTCAGCATTCATCG | GTCTGTTAACTTTGTACTGCAC |
| | (SEQ ID NO: 49) | (SEQ ID NO: 50) |

### Results

### Mapping Rmi1 to Chromosome 10

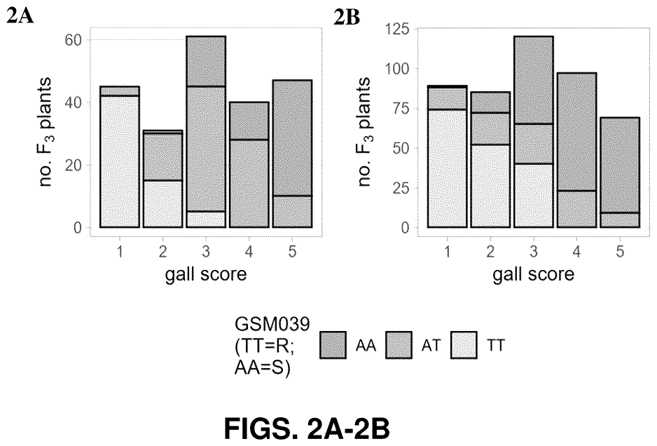

A population of F_{2:3} plants was generated from a cross between the susceptible Bossier and the resistant Forrest. Three replicates each of 173 lines were planted in the greenhouse for evaluation of M. incognita resistance. 472 plants representing 171 of the 173 F_{2:3} lines were able to be phenotyped. All reps of two lines died before the screen concluded. 89 plants showed a similar level of resistance to Forrest, 69 lines showed a similar level of susceptibility as Bossier, and the remaining 302 plants had intermediate phenotypes (FIG. 2A). 203 plants were homozygous for the susceptible allele and 166 plants were homozygous for the resistant allele. Lines genotyped as resistant had a mean gall score of 1.8. Lines genotyped as susceptible had a mean gall score of 3.9. Lines genotyped as heterozygous had a mean gall score of 3.0. A significant (p<0.001) correlation was found between the GSM039 marker and M. incognita response, with R²=0.5.

Twenty F_{2:3} lines were selected for additional screening, accounting for lines found to be segregating at GSM039 in the initial screen and randomly selected additional lines. A total of 246 plants were screened. Ninety-six genotyped as heterozygous, 77 genotyped as susceptible, and 73 genotyped as resistance. Lines genotyped as heterozygous had a mean gall score of 3.3. Lines genotyped as resistant had a mean a gall score of 1.3. Lines genotyped as susceptible had a mean gall score of 4.4. The correlation between M. incognita response and GSM039 was again significant (p<0.001) with R²=0.6 (FIG. 2B).

### Fine Mapping Rmi1

KASP markers were designed to span a 93 kb region. Between Glyma.10G016600 and Glyma.10G017200 there was one marker per gene. Two additional markers were designed 9 kb and 65 kb to the right of Glyma.10G017200 to ensure capture of recombinants. 884 F₅ lines were genotyped using DNA extracted from 5 seeds per line. Lines with putative recombination events were selected for a greenhouse screen. Seventy-two F₅ lines were phenotyped over four screens. Four RILs reliably genotyped as recombinant. Lines G22BF-858, G22BF-626, G22BF-582, G22BF-549 possessed breakpoints between Glyma.10G016600 and Glyma.10G06700, Glyma.10G016832 and EG, between PME1 and Glyma.10G017200, and between Glyma.10G017400 and Glyma.10G017900, respectively (FIG. 1E). All four RILs phenotyped as susceptible and genotyped as Bossier at G10.170 and GSM040. This narrowed the Rmi1 interval to a 27.5 kb region containing only two genes—Glyma.10G017000 (β-1,4-endoglucanase; EG) and Glyma.10G017100 (pectin methylesterase inhibitor; PME1) (FIG. 1F).

### Candidate Expression Confirmation

Expression of the mapped candidates was evaluated based on previously acquired RNAseq reads. 238 reads aligned to EG at either time point from ExF63 infected roots. 610 reads mapped to EG at either time point from ExF67 infected roots. 295 reads aligned to EG at either time point from ExF63 mock-infected roots. 606 reads mapped to EG at either time point from ExF67 mock-infected roots. All mapped reads confirmed the predicted splice sites.

Thirty-two reads aligned to PME1 at either time point from ExF63 infected roots. 31 reads mapped to PME1 at either time point from ExF67 infected roots. 13 reads aligned to PME1 at either time point from ExF63 mock-infected roots. 6 reads mapped to PME1 at either time point from ExF67 mock-infected roots. Despite the low number of mapped reads, cDNA of PME1 was able to be amplified from infected tissue (Appendix 1). qPCR was conducted on the RNAseq samples to verify gene expression results and to achieve greater sensitivity for these low expression genes.

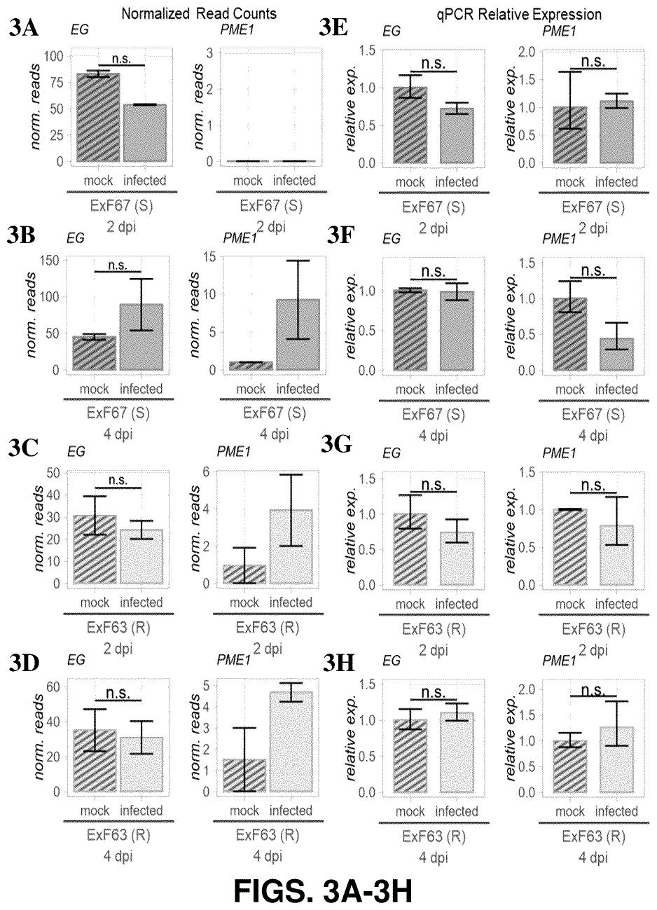

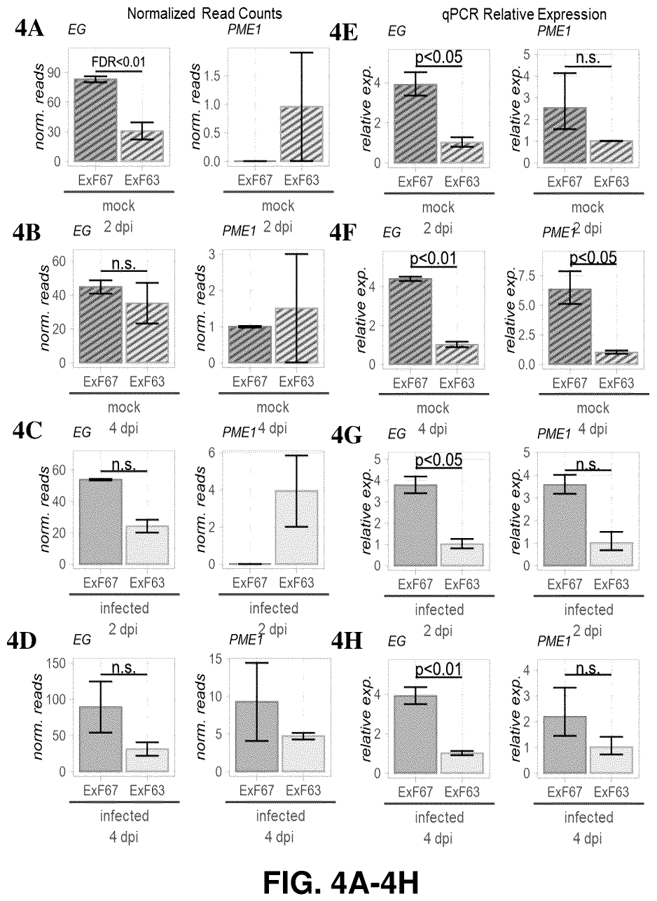

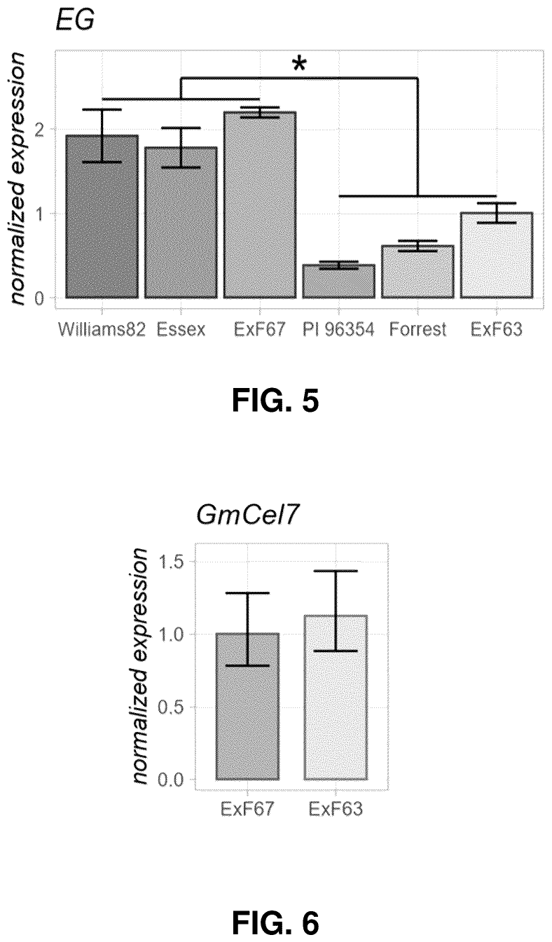

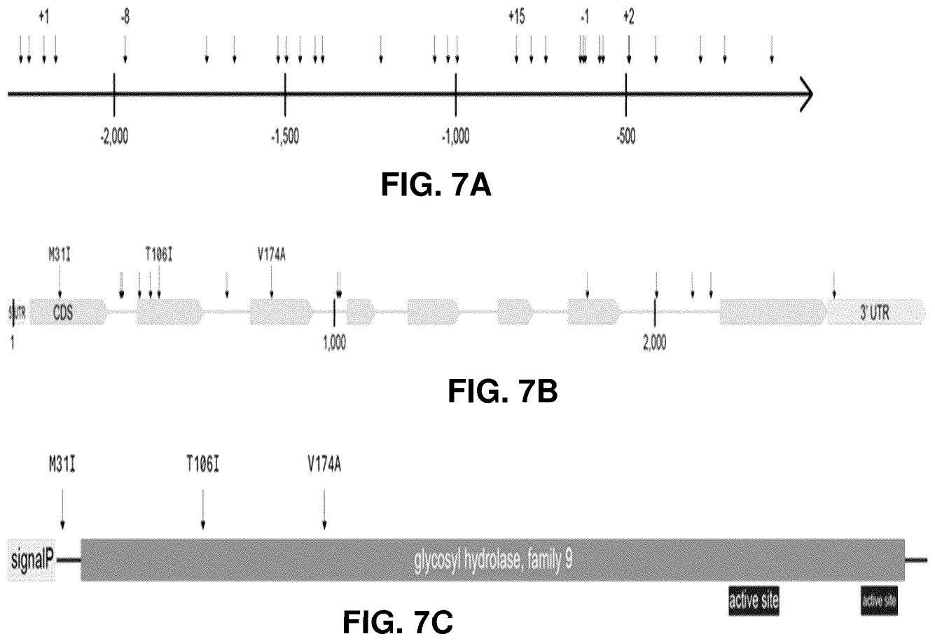

Neither EG nor PME1 was differentially expressed in response to infection when using either edgeR calculation based on read mapping or qPCR (FIGS. 3A-H). However, there is a significant difference in EG expression between genotypes between mock roots at 2 dpi, with ExF67 showing 3× higher expression than ExF63 (FIG. 4A). When using the more sensitive qPCR, at 2 dpi, EG had 4× higher expression in susceptible mock roots than in resistant mock roots; expression was also 4× higher in susceptible infected roots when compared to resistant infected roots (FIGS. 4E, 4G). At 4 dpi, EG expression was 5× higher in susceptible mock roots than in resistant mock roots; expression was 4× higher in susceptible infected roots than in resistant infected roots (FIGS. 4F, 4H). To determine if this phenotype is common among resistant genotypes, root tips were collected from uninfected susceptible (Williams 82, Essex, ExF67) and resistant (PI 96354, Forrest, and ExF63) lines. qPCR of cDNA of these root tips showed 3× increase in expression of EG in susceptible lines when compared to resistant lines (FIG. 5). Since EG shares 95% identity with its close ortholog, Glyma.02G016400 (GmCel7), expression of GmCel7 was also checked to investigate a possible interaction or functional redundancy. GmCel7 was not significantly differentially expressed between resistant or susceptible roots (FIG. 6).

Expression of PME1 trended higher in susceptible roots when compared to resistant roots, though the only statistically significant change was between mock susceptible and mock resistant roots at 4 dpi, where expression was 7× higher in the susceptible roots (FIG. 4G). However, these results may be unreliable due to the very low expression of PME1 affecting the accuracy of qPCR and edgeR results.

### Candidate Gene Sequences

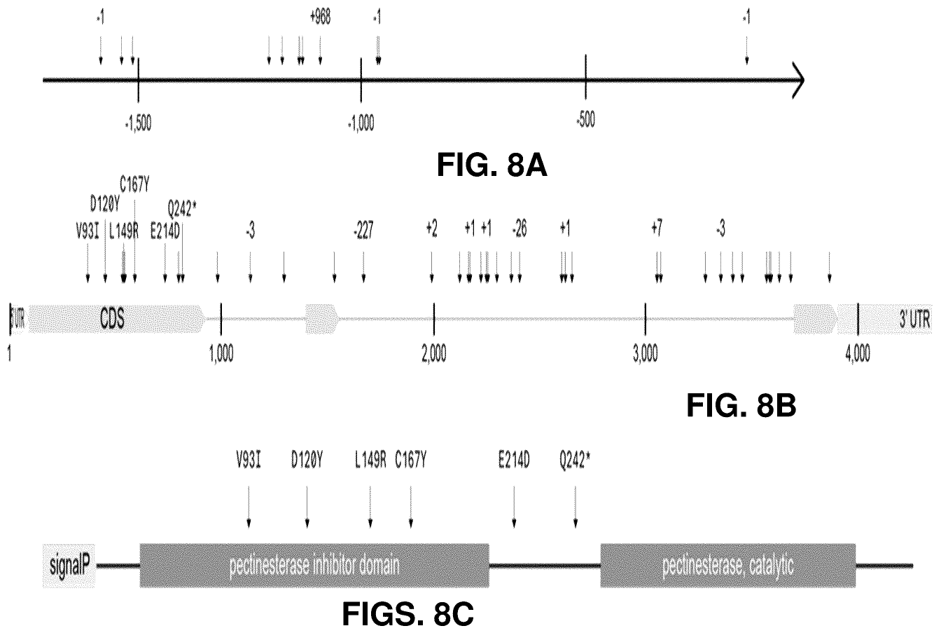

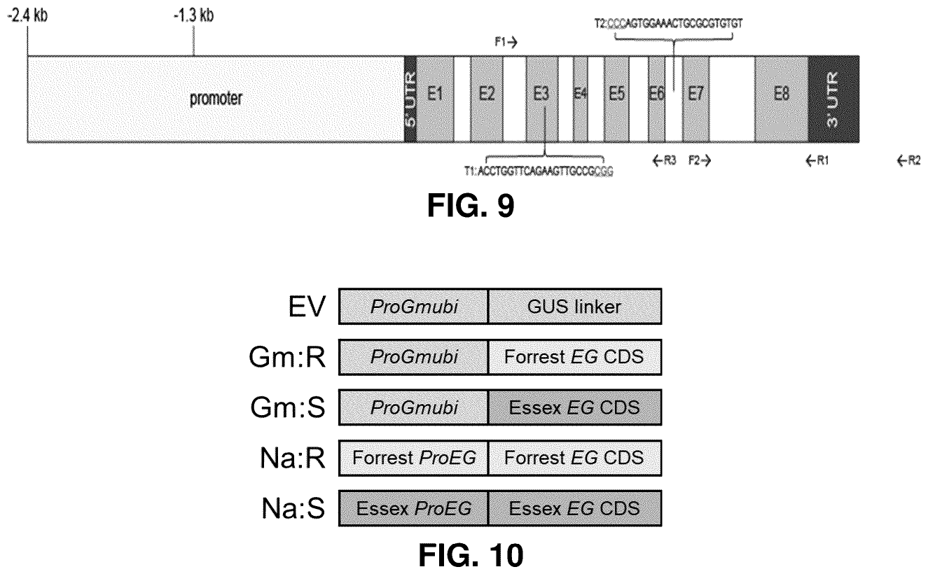

To determine the significance of a polymorphism, the sequences of the resistant Forrest and PI 96354 were compared to the susceptible Williams 82, Essex, and Bossier sequences. If a polymorphism was found to be shared in Forrest and PI 96354, but not present in Williams 82, Essex, or Bossier, it was considered conserved. In both genes, there are many differences between resistant and susceptible lines. The EG promoter has 23 SNPs, a 10 bp insertion, a 13 bp deletion, and a 15 bp insertion in the resistant line compared to the Wm82.a4.v1 reference (FIG. 7A). The resistant gene sequence contains three nonsynonymous SNPs (M31I, T106I, V174A), two silent exonic SNPs, five intronic SNPs, a 1-bp intronic deletion, and a 3′ UTR SNP (FIG. 7B-7C). The PME1 promoter has an insertion of 968 bp approximately 1.1 kb upstream of the 5′ UTR in the resistant lines (FIG. 8A). There are also two single bp deletions after the 3′ end of this insertion and multiple SNPs before the 5′ end of the insertion. In PME1, there are five SNPs resulting in amino acid changes (V93I, D120Y, L149R, C167Y, E214D, one resulting in a premature stop codon (Q242*), and five silent exonic SNPs (FIG. 8B-8C). In the introns there are 227, 24, 22, 3, and 2 bp deletions, along with three single bp deletions, 3 and 7 bp insertions, and twenty SNPs between the resistant and susceptible lines (FIG. 8B). The predicted splicing patterns of each gene were confirmed by sequencing cDNA (Appendix 1).

### Discussion

Genetic resistance is the preferred method of controlling damage due to RKN. The first RKN resistance gene, Mi-1.2, was mapped in tomato (Milligan et al. 1998). Mi-1.2 is a canonical R gene, encoding a protein containing a nucleotide binding site (NBS) and leucine-rich repeat (LRR) domains. Since then, several other R genes for RKN have been cloned and even more R loci mapped (Goode and Mitchum 2022). A major QTL on chromosome 10 has been identified in every published mapping study of M. incognita resistance in soybean, regardless of methods or resistance sources (Fourie et al. 2008, Pham et al. 2013, Xu et al. 2013, Jiao et al. 2015, Passianotto et al. 2017). Unlike other RKN resistance QTL, this region contains no canonical R genes. There are a variety of genes in this region, including some cell wall-related genes while others are not characterized or have unknown functions.

The use of Forrest for identifying the causal gene(s) in this region is ideal because its resistance is derived from a single gene. This study confirmed that the causal gene(s) Rmi1 resistance is in the previously identified QTL on chromosome 10. Confirmation of this location allows linkage of the J2 emigration phenotype demonstrated by Herman et al. (1991) with the chromosome 10 QTL.

When comparing the sequences of the resistant Forrest and PI 96354 to the susceptible Williams 82 and Bossier, there are hundreds of conserved polymorphisms in the genomic region between Glyma.10G016600 and Glyma.10G017300, the region encompassing genes of interest identified by Pham et al. (2013) and Xu et al. (2013). However, this shared region of polymorphisms stops abruptly just before Glyma.10G017400. This lack of resistance-correlated SNPs necessitated the use of markers not correlated with resistance further down the region, as the only SNPs available were just between the parental Bossier and Forrest lines.

The Rmi1 interval fine-mapped contains two predicted genes in the Wm82.a4.v1 reference genome—EG and PME1. A conserved SNP results in a premature stop codon before the predicted PME catalytic domain. PME1 is a type I PME, possessing both a catalytic PME domain and a PME inhibitor domain. The PME inhibitor domain is cleaved before a PME is found in the cell wall and likely functions as an intramolecular chaperone of the PME catalytic domain (Micheli 2001). The NCBI RefSeq database predicts PME1 (LOC102665079) to be a pseudogene. Few global soybean RNA transcriptome studies determining expression patterns throughout the plant have identified PME1 expression and when it has been identified it is at very low levels. Libault et al. (2010) identified PME1 expression in the shoot apical meristem (SAM). Danzer et al. (2015) identified expression in the seed cotyledon abaxial epidermis. Pelletier et al. (2017) identified expression in the hilum. The maximum level of expression in any of these experiments was 0.33 FPMK or RPMK (Danzer et al. 2015). In the same set of experiments, EG was identified in SAM, embryo vasculature, seed cotyledon vasculature, embryo, and hilum with expression levels ranging from 28-100 FPMK or RPMK. Severin et al. (2010) also identified EG expression in young leaves, a study which found no PME1 expression.

Only three other expression studies used in SoyBase's gene expression profiling identified expression of PME1 above 1 RPMK or FPMK. Expression was identified at up to 5 FPKM in response to aphid infestation (Brechenmacher et al. 2015), in glaborus and wild-type shoot tips of cv. Clark at up to 2 RPMK (Hunt et al. 2011), and up to 6.6 RPMK in a dehydration and salinity response experiment (Belamkar et al. 2014). In contrast, only two datasets did not identify EG expression above 1 RPMK or FPMK. These were experiments looking at gene expression in the seed coat (Kour et al. 2014) and gene expression in leaves during wilting (Devi et al. 2015). Since PME1 expression has been identified in some circumstances and was able to be amplified through cDNA in this study, it is likely not a pseudogene. Rather, it is a gene that has very low expression and is likely only expressed in specific circumstances and cell types, making it difficult for broader gene prediction programs to identify.

In plants, PMEs typically demethylesterify (DM) pectin in a linear fashion, acting on chains of homogalacturonan (HG), the most abundant component of pectin (Wolf et al. 2009). This releases methyl groups from the HG, leaving a binding site for Ca²⁺ ions. The connection of HG chains through Ca²⁺ binding leads to “egg-box” structures that rigidify the cell wall (Micheli 2001, Wormit and Usadel 2018, Molina et al. 2024). If Ca²⁺ binding does not occur, this leaves the HG chains open to pectin-degrading enzymes, like polygalacturonases (Pelloux et al. 2007). HG degradation by polygalacturonases releases oligogalacturonides that can function as damage-associated molecular patterns (DAMPs) that initiate DAMP-triggered immunity (DPI) (Lionetti et al. 2012, Molina et al. 2024). The level of pectin esterification has been implicated in resistance against wheat fungus (Wietholter et al. 2003) and liquorice rot in carrots (Le Cam et al. 1994). Arabidopsis cell walls were found to be DM by plant PMEs in response to infection by Alternaria brassicicola and Pseudomonas syringae (Bethke et al. 2014).

There has also been a direct interaction found between an Arabidopsis type I PME and a cellulose binding protein (CBP) secreted by Heterodera schachtii during feeding site formation (Hewezi et al. 2008). The specific binding sites in the interaction were not determined. M. incognita infective J2s also secrete a CBP effector (Ding et al. 1998). Mi-CBP could target the catalytic domain in PME1 that is missing from the resistant allele, thus leading to a loss of a susceptibility factor in resistant lines. While establishment of a giant cell requires precise manipulation of the cell wall in order to rapidly increase the cell size while maintaining wall integrity, J2s travel intercellularly through the middle lamella, a primarily pectin layer, while migrating through the root before selecting an initial feeding cell. RKN secrete other pectin-modifying enzymes while migrating (Jaubert et al. 2002, Huang et al. 2005). Since the exact reason for J2 emigration is unclear, it is not known how close a J2 comes to initiating a feeding site before emigrating. PME1 could function as a susceptibility factor for either nematode migration or feeding site initiation.

EG encodes an EGase belonging to glycosyl hydrolase family 9 (Urbanowicz et al. 2007). EGases hydrolyze bonds in the β-1,4-glucan backbone of cellulose, a compound accounting for 30% of cell wall mass (Ochoa-Villarreal et al. 2012). In other pathogen systems, EGases can act as susceptibility factors. SlCel1 and SlCel2 are needed for tomato susceptibility to Botrytis cinerea (Flors et al. 2007). In Arabidopsis, plants lacking the EGase KOR1 were more susceptible to P. syringae (López-Cruz et al. 2014). Expression of several plant EGases has been identified in giant cells, including the tobacco NtCel7 and NtCel8 and the Arabidopsis AtCel1 (Goellner et al. 2001, Sukno et al. 2006). Knockouts of an Arabidopsis EGase (At4g16260) increased susceptibility to H. schachtii, while overexpression of a different Arabidopsis EGase (AtCel6) in soybean decreased susceptibility to both Heterodera glycines (soybean cyst nematode; SCN) and M. incognita (Hamamouch et al. 2012, Woo et al. 2014). RNAi knockouts of GmCel7 decreased susceptibility of soybean to SCN (Woo et al. 2014). Due to the high sequence similarity between EG and GmCel7, it is likely that EG was also silenced in this study. EG expression could, like other GH9 EGases, be induced in giant cells. If EG is required for successful infection, the J2 might be able to sense the lack of the susceptible EG allele and emigrate before a feeding site is established. Since the resistance phenotype occurs through emigration of J2s, it is necessary for the nematode to sense the lack of the susceptibility factor early enough in the infection process to emigrate. It is unknown how far the J2 can get in initiating a feeding site without becoming sedentary. It is also unknown what exactly a J2 is sensing when selecting a feeding site. The lack of EG due to lower expression in resistant roots before infection could be sensed as the J2 is trying to select an initial feeding cell. The amino acid changes in the resistant EG could also prevent the J2 from determining a cell to be a suitable feeding cell. If a compatible cell with the susceptible EG cannot be located, the nematode would emigrate to find another root that could have suitable cells.

EG was overlooked in prior studies as a candidate. Pham et al. (2013) set certain criteria when narrowing down the thirty identified genes in the QTL for sequencing. Genes that were sequenced to identify polymorphisms needed to either have a root-specific expression pattern or have been shown to be involved in RKN response previously. Based on the available data at the time, EG was not selected for further study. Passianotto et al. (2017) performed a GWAS in which they sequenced the nearly 200 lines used to create a unique SNP panel. Five SNPs in the genes adjacent to EG were identified. It is unclear whether SNPs located more closely to EG were used in the evaluation. Xu et al. (2013) utilized a bin-mapping strategy to fine map a 30 kb region. The exact borders of this bin are unclear, but it does border EG.

While neither of these candidates belongs to a class of canonical R genes, there is precedent for nematode resistance in soybean to be controlled by a non-canonical R gene. Neither of the two primary resistance genes against SCN, Rhg1 and Rhg4, are canonical R genes. Rhg1 is controlled by copy number variation of a block of three genes—an amino acid transporter, α-soluble N-ethylmaleimide sensitive factor attachment protein (SNAP18), and a wound-inducible domain protein (Cook et al. 2012). Rhg4 encodes a serine hydroxymethyltransferase (Liu et al. 2012). Another member of the soybean α-SNAP family, GmSNAP11, also functions in SCN resistance (Shaibu et al. 2022). GmSNAPO2 functions as a susceptibility gene for SCN infection (Usovsky et al. 2023).

The narrowing of the Rmi1 window to two candidate genes greatly decreases the number of genes to be further evaluated for a role in resistance. Both candidates have the potential to be involved in the resistance response, since they both possess amino acids changes, including a premature stop in PME1, and show higher expression in susceptible roots than in resistant roots.

### Example 2: Fine Mapping Resistance to M. incognita in Soybean Cultivar Forrest

### Methods

Rmi1 was fine mapped to two genes, Glyma.10G017000 (EG) and Glyma.10G017100 (PME1). Both genes belong to classes involved in cell wall modification, a tightly regulated process in giant cell formation as the nematode should rapidly increase cell size while maintaining wall integrity (Abad et al. 2009). EG encodes a β-1,4-endoglucanase (EGase). EGases hydrolyze the β-1,4-D-glucose bonds in cellulose, which accounts for 30% of cell wall mass (Ochoa-Villarreal et al. 2012). RKN can induce expression of plant EGases during giant cell formation. Expression of the EGases NtCel2, NtCel4, NtCel7 and NtCel8 was found in tobacco roots after M. incognita infection; NtCel7 and NtCel8 expression was further localized to giant cells (Goellner et al. 2001). The Arabidopsis AtCel1 was also found to be expressed in giant cells (Goellner et al. 2000). EG is more highly expressed in the roots of susceptible plants than in resistant plants before infection occurs. This creates a basal difference that could be detectable by a nematode, either directly through recognition of the EGase itself, or through differences in the cell walls of resistant plants caused by the lower expression of EG. Another possible cause for emigration could be the changed EG protein resulting from three amino acid changes. Once again, these changes could result in the nematode being unable to detect the presence of EG protein in resistant plants. If the amino acid changes result in a loss-of-function, then the resulting cell wall changes could be the impetus for emigration.

PME1 encodes a type I pectin methylesterase inhibitor, containing a PME catalytic domain and a PME inhibitor domain. The inhibitor domain likely acts as an intramolecular chaperone for the PME domain (Micheli 2001). An Arabidopsis type I PME was found to directly interact with a cellulose binding protein (CBP) secreted by Heterodera schachtii to promote feeding site initiation (Hewezi et al. 2008). M. incognita infective J2s also secrete a CBP effector (Mi-CBP) while migrating intercellularly through the pectin-rich middle lamella of the root (Ding et al. 1998). The resistant version of PME1 has a premature stop codon before the PME catalytic domain. If a direct interaction between Mi-CBP and the catalytic domain of PME1 is required, the resistant allele would lack the binding site, resulting in the loss of a susceptibility factor. PME1 is expressed at nearly undetectable levels, making it more difficult to accurately quantify its expression. However, the ability to amplify PME1 cDNA does verify that it is expressed at some level in root tissue.

Both candidate genes merit further investigation through functional studies. The following assays aim to functionally characterize the role of EG, the candidate with highest expression levels and a basal increase (3-4×) in expression in the roots of susceptible plants, in resistance against M. incognita.

### Methods

### Sequence Analysis

Candidate genes were amplified from five soybean lines, including the susceptible Bossier and Essex and the resistant Forrest, PI 96354, and Lee 74 using Q5 DNA polymerase (New England Biolabs) and sequenced by Sanger sequencing (Azenta Life Sciences, South Plainfield, NJ). The primers used are shown in Table 3. For comparison of genes outside the candidate region, publicly available Illumina reads were used after confirmation that the resulting candidate gene sequences matched the Sanger reads generated (Table 4). The resulting sequences were compared to the susceptible Wm82.a4.v1 reference genome (Valliyodan et al. 2019). To identify promoter elements altered between resistant and susceptible genotypes, the promoter sequences were analyzed using PlantPAN4.0 (Chow et al. 2024) and PLACE (Higo et al. 1999). Sequence for analysis of endoglucanases from Arabidopsis, tobacco, tomato, and soybean were collected from NCBI (Table 5). A relationship tree was created using Geneious Prime 2024.0.5.

TABLE 4
Publicly available Illumina accessions used for sequence
comparisons of resistant and susceptible soybean lines.
The stated genotype of each accession was verified with
sanger reads generated from lines used in this research

| Line | Accession |
|---|---|
| Essex | SRR12191283 |
| Essex | SRR12191295 |
| Bossier | SRR14424420 |
| Forrest | SRR13567764 |
| Forrest | SRR19134685 |
| Lee 74 | SRR12335866 |
| Lee 74 | SRR12335877 |
| PI 96354 | SRR9714646 |

### Expression Analysis

The genotypes used in the following assays are ExF63 (resistant) and ExF67 (susceptible), RILs generated from a cross between the susceptible Essex and the Rmi1-containing Forrest (Lightfoot et al. 2005). These RILs shared 80% of their genome, while differing in the sequences for the Rmi1 candidates (Gamage et al., unpublished). ExF67 shares the candidate sequence with Essex; ExF63 shares the candidate sequence with Forrest. An infection assay was conducted using ExF63 and ExF67 seeds. Seeds were germinated and inoculated as described previously (Ithal et al. 2007). Twenty seedlings of each genotype were inoculated with either 250 J2s in 100 μl 0.1% agarose or 100 μl 0.1% agarose only. Trays were prepared by coating one layer of 3 mm Whatman paper, then two layers of wet germination paper. Excess water was removed by compressing the layers with a clean glass test tube. Roots were placed on the trays and marked with a fresh sharpie ˜1 cm above the root tip. 100 μl of inoculum were pipetted onto the mark. The inoculation point was covered with a single layer of damp germination paper. Trays were covered in plastic wrap with holes poked throughout, then an additional tray was placed on top to ensure darkness. Trays were left in a 25° C. growth chamber. After 24 hours, roots were fully rinsed of inoculum to synchronize the infection. Seedlings were rolled in germination paper supplemented with Hoagland's solution and placed in a 25° C. growth chamber on a 16 h day/8 h night light cycle. Galls or comparable root tissue were excised six days after inoculation. Collected tissue was ground using a liquid nitrogen-chilled porcelain mortar and pestle, then extracted using the QIAGEN Plant RNeasy Mini Kit (QIAGEN, Hilden, Germany). Total RNA (500 ng) from each sample was used for cDNA synthesis using PrimeScript 1st Strand cDNA Synthesis Kit (Takara Bio USA, Mountain View, CA). Quantitative RT-PCR was performed on a Bio-Rad CFX Real-Time System using PowerUp SYBR Master Mix (Applied Biosystems) according to the manufacturers recommendation. The primers used for qPCR are listed in Table 4. The reference genes GmELF1A and Gmcons7 were used to normalize expression (Libault et al. 2008, Miranda et al. 2013).

TABLE 5
NCBI accessions used for relationship tree of EGases from Arabidopsis, tobacco, tomato, and soybean

| Sequence Name | Organism | Accession | Sequence Description |
|---|---|---|---|
| Arabidopsis Cel1 | Arabidopsis thaliana | NP_177228.1 | glycosyl hydrolase 9B1 [Arabidopsis thaliana] |
| Arabidopsis Cel2 | Arabidopsis thaliana | NP_171779.1 | Cellulase 2 [Arabidopsis thaliana] |
| Arabidopsis Cel6 | Arabidopsis thaliana | NP_175323.1 | glycosyl hydrolase 9C1 [Arabidopsis thaliana] |
| Glyma.04G111300 | Glycine max | XP_003522825.1 | endoglucanase 9 [Glycine max] |
| Glyma.05G236400 | Glycine max | XP_003525241.1 | endoglucanase 8 [Glycine max] |
| Glyma.06G323100 | Glycine max | XP_003527576.1 | endoglucanase 9 [Glycine max] |
| Glyma.08G043600 | Glycine max | XP_003530757.1 | endoglucanase 8 [Glycine max] |
| Glyma.10G017000 | Glycine max | XP_003536878.1 | endoglucanase 17 [Glycine max] |
| Glyma.11G101300 | Glycine max | XP_006590804.1 | endoglucanase 5 isoform X1 [Glycine max] |
| Glyma.126027200 | Glycine max | XP_014619917.1 | endoglucanase 5 isoform X2 [Glycine max] |
| Soybean Cel2 | Glycine max | XP_003537838.1 | endoglucanase 24 [Glycine max] (GLYMA_11G111500v4) |
| Soybean Cel7 | Glycine max | NP_001340344.1 | alpha family endo-beta-1,4-glucanase precursor [Glycine max] |
| | | | (GLYMA_02G016400v4) |
| Soybean Cel8.1 | Glycine max | XP_003525345.1 | endoglucanase 6 [Glycine max] (GLYMA_05G216400v4) |
| Soybean Cel8.2 | Glycine max | NP_001341798.1 | endo-1,4-beta glucanase precursor [Glycine max] (GLYMA_08G022300v4) |
| Soybean Cel9 | Glycine max | XP_003552557.1 | endoglucanase 1 [Glycine max] (GLYMA_18G030700v4) |
| Tobacco Cel2 | Nicotiana tabacum | NP_001312102.1 | endoglucanase 8-like precursor [Nicotiana tabacum] |
| Tobacco Cel4 | Nicotiana tabacum | XP_016465715.1 | PREDICTED: endoglucanase 17-like [Nicotiana tabacum] |
| Tobacco Cel5 | Nicotiana tabacum | XP_016505399.1 | PREDICTED: endoglucanase 1-like [Nicotiana tabacum] |
| Tobacco Cel7 | Nicotiana tabacum | XP_016467406.1 | PREDICTED: endoglucanase 9-like [Nicotiana tabacum] |
| Tobacco Cel8 | Nicotiana tabacum | NP_001312729.1 | endoglucanase 6-like precursor [Nicotiana tabacum] |
| Tomato Cel2 | Solanum lycopersicum | NP_001234867.1 | endo-1,4-beta-glucanase precursor [Solanum lycopersicum] |
| Tomato Cel4 | Solanum lycopersicum | NP_001234882.1 | endo-1,4-beta-glucanase precursor [Solanum lycopersicum] |
| Tomato Cel5 | Solanum lycopersicum | NP_001234090.2 | endo-1,4-beta-glucanase precursor [Solanum lycopersicum] |
| Tomato Cel7 | Solanum lycopersicum | NP_001234323.1 | endo-1,4-beta-D-glucanase precursor [Solanum lycopersicum] |
| Tomato Cel8 | Solanum lycopersicum | NP_001234172.1 | endo-beta-1,4-D-glucanase precursor [Solanum lycopersicum] |

### Promoter:GUS Vector Construction

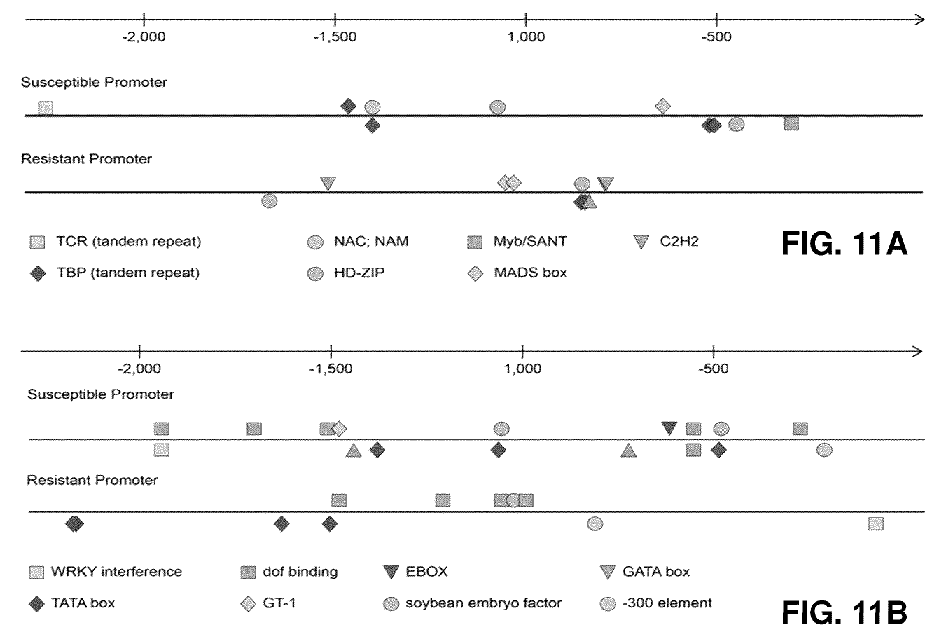

The promoter region (˜2.4 kb) for EG was amplified from Essex and Forrest using ExTaq (TaKaRa Bio) and cloned into the donor vector pDONR/Zeo (Invitrogen) using Gateway BP Clonase II according to the manufacturer's protocol (FIG. 9). Sequence-confirmed donor vectors were then cloned into the destination vector pcamGFP-GW:GUS (Damodaran et al. 2019) using Gateway LR Clonase II according to the manufacturer's protocol to make the constructs R_ProEG:GUS (Forrest) and S_ProEG:GUS (Essex), which were transformed into HST08 Stellar™ Competent Cells (TaKaRa Bio), then K599 Rhizobium rhizogenes. To validate promoter regions necessary for expression, a 1.3 kb fragment was amplified from Essex, then cloned into the pcamGFP-GW:GUS vector as described above to create the vector S_ProEG1.3:GUS, which was transformed into HST08, then K599. All primers used are listed in Table 4.

### CRISPR Vector Construction

The CRISPR vector was constructed according to Kang (2016). Briefly, dual gRNAs for the candidate gene were selected using Geneious Prime 2023.1 (Table 4, FIG. 9). Oligos for the gRNAs were ligated into AtU6-26SK vector before ligation into the pCAMGFP-CvMV:GWox (Invitrogen) backbone with Cas9 from the 35S-Cas9-SK vector (Feng et al. 2013). The final CRISPR construct, containing dual AtU6:gRNAs, 35S:Cas9, kanamycin resistance, and CvMV:GFP was transformed into HST08, then K599. An empty vector (EV) containing the BbsI restriction site was also used.

### Overexpression Vector Construction

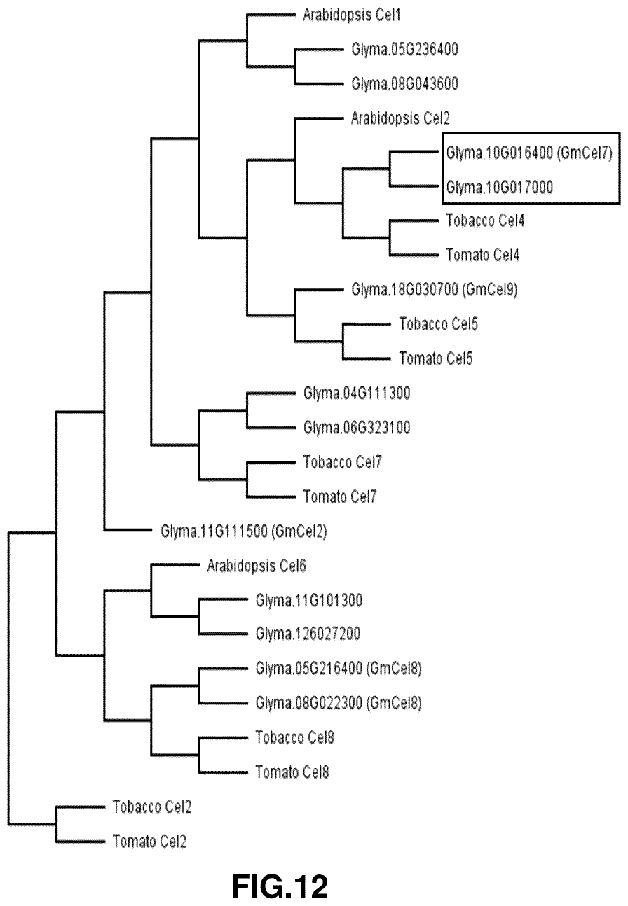

The coding sequence of EG was amplified from cDNA of both genotypes using ExTaq. AscI and AvrII restriction sites added to allow cloning into the pG2RNAi2 vector (Mazarei et al. 2023) to create the overexpression constructs Gm:R (Forrest) and Gm:S (Essex), placing the coding sequence under the Gmubi promoter, a soybean ubiquitin promoter with constitutive expression in roots (Hernandez-Garcia et al. 2009) (FIG. 10). For expression under the 2.4 kb native promoter, the promoter was amplified from gDNA using ExTaq. AscI and ApaI restriction sites were added to allow cloning to replace the Gmubi promoter in the previously confirmed overexpression constructs, creating the constructs Na:R (Forrest) and Na:S (Essex) (FIG. 10). All constructs were transformed into HST08, then K599. All primers used are listed in Table 6.

TABLE 6
Primers used for construction of vectors for promoter: GUS, overexpression vector,
and CRISPR, and for confirmation of CRISPR edits
Primers used for PmpEG:GUS vector construction

| Primer use | Primer sequence |
|---|---|
| 2.4 kb EG promoter | GGGGACAAGTTTGTACAAAAAAGCAGGCTTAGACAAGTAGCAAACTTTTAGGCAA |
| (forward) with attB1 site | (SEQ ID NO: 51) |
| 1.3 kb EG promoter | GGGGACAAGTTTGTACAAAAAAGCAGGCTTAACCTAACATGCACCGAATCAC |
| (forward) with attB1 site | (SEQ ID NO: 52) |
| EG promoter (reverse) | GGGGACCACTTTGTACAAGAAAGCTGGGTAGCTCTTTGGTTGTTCAATCTCTGG |
| with attB2 site | (SEQ ID NO: 53) |

Primers used for overexpression vector construction

| Primer use | Primer sequence |
|---|---|
| EG promoter (forward) | TGCTTAGGGCCCGACAAGTAGCAAACTTTTAGGCAA (SEQ ID NO: 54) |
| with ApaI site | |
| EG promoter (reverse) | TGCTTAGGCGCGCCTTTGAAGAGGCAATGTGAAAGTGAAAC (SEQ ID NO: 55) |
| with AscI site | |
| EG CDS (forward) with | TGCTTAGGCGCGCCATGACTTTCTCCTTTTCCTTC (SEQ ID NO: 56) |
| AscI site | |
| EG CDS (reverse) with | TGCTTACCTAGGCTAGAGTTGACCGAATGAG (SEQ ID NO: 57) |
| AvrII site | |

Primers used for CRISPR vector construction

| Primer use | Primer sequence |
|---|---|
| EG gRNA target 1 (forward) | GATTGACACACGCGCAGTTTCCACT (SEQ ID NO: 58) |
| with BbsI overhang | |
| EG gRNA target 1 (reverse) | AAACAGTGGAAACTGCGCGTGTGTC (SEQ ID NO: 59) |
| with BbsI overhang | |
| EG gRNA target 2 (forward) | GATTGACCTGGTTCAGAAGTTGCCG (SEQ ID NO: 60) |
| with BbsI overhang | |
| EG gRNA target 2 (reverse) | AAACCGGCAACTTCTGAACCAGGTC (SEQ ID NO: 61) |
| with BbsI overhang | |

Primers used for CRISPR edit confirmation

| Primer name | Primer sequence |
|---|---|
| F1 | TGGGGTAAGTCTTCTTTAGATTCTG (SEQ ID NO: 62) |
| F2 | TGACCCAATCCCATATGCTTG (SEQ ID NO: 63) |
| R1 | AAGATAGGCCAGTGCTCCTAC (SEQ ID NO: 64) |
| R2 | GTCTTTCTCCATCGTCCTCAC (SEQ ID NO: 65) |
| R3 | GAGAACAGACGAAATTGTCCG (SEQ ID NO: 66) |

### Composite Plant Generation

Composite plants were generated using a modified protocol from Fan et al. (2020) and Gamage et al. (submitted) for infection assays involving RKN. Five-day-old seedlings of ExF63 or ExF67 were slant-cut ˜1 cm below the hypocotyl. The wound was thoroughly coated in K599 containing the construct of interest, then the seedling placed in moist fine vermiculite. 5 ml of ddH₂O was pipetted around the seedling. Two pots were sealed together in a plastic bag and left in a Conviron Gen1000 growth chamber set at 26° C. on a 16 h light (240 μmol·m⁻²·s⁻¹)/8 h dark cycle. After ten days, plants were removed from vermiculite briefly to trim adventitious roots, then placed back into the same vermiculite. Pots were placed in a domed germination flat with bottom watering in the growth chamber. After four days, the lids were vented slightly to acclimate plants to lower humidity over a two-day period. Plants were then moved to the greenhouse for three days of acclimatation. Non-GFP+ roots were trimmed from the root systems, then composite plants with comparable GFP+ roots were selected for transplant to 1:1 soil:sand mix in cone-tainers in the greenhouse. Three days after planting, each plant was inoculated with 1,000 M. incognita J2s. To verify overexpression constructs were indeed increasing EG expression, three whole roots of each overexpression construct/genotype condition were sampled at planting and frozen in liquid nitrogen. RNA was extracted and qPCR performed as described above.

### Composite Plant Phenotyping

Eleven days after inoculation, the root systems were thoroughly rinsed free of soil. To confirm edits, 1 cm sections cut from the tips of three lateral roots originating in the midsection of the primary GFP+ root were collected in 2 ml microcentrifuge tubes with two 2.3 mm zirconia beads and frozen in liquid nitrogen. The tissue was ground in a mini-beadbeater (BioSpec Products, Bartlesville, OK), then stored at −80° C. until extraction using a CTAB/CHCl₃/Isopropanol method. The ground tissue was incubated in CTAB extraction buffer (0.1 M Tris-HCl pH 9, 1.4 M NaCl, 20 mM EDTA, 2% CTAB) and 0.3 mg/μl RNAse A at 65° C. for 30 minutes. An equal amount of isoamyl chloroform was added before a 5 min incubation at 25° C. incubation and centrifuging at 17,900×g for 4 min. The supernatant was mixed 1:1 with isopropanol and incubated at −20° C. for 20 minutes, then centrifuged at 17,900×g for 15 min. The pellet was washed in 70% ethanol and resuspended in dH₂O. The region surrounding the gRNA target sites was amplified using ExTaq using primers listed in Table 4 and sequenced.

To evaluate infection phenotypes, root systems were shaken in 10% bleach for five minutes, rinsed, then stained with acid fuchsin by boiling roots in a 1:50 dilution of acid fuchsin staining solution (0.35 g acid fuchsin in 25 ml acetic acid and 75 ml H₂O) for one minute, allowing to cool for thirty minutes, then destaining in glycerin at 65° C. until nematodes were easily visible. The number of nematodes in each root system was counted under a stereomicroscope, with nematodes classified as either vermiform or swollen.

### GUS Staining

Twelve days after inoculation, roots were vacuum infiltrated with GUS substrate (100 mM Tris-HCl, 50 mM NaCl, 1 mM X-Gluc, 0.6 mM K+ ferricyanide, 0.06% Triton X-100) overnight at 37° C. After verification of GUS staining, roots were stained with acid fuchsin as described above. Images were taken under an Olympus SZH stereo microscope with a LABOMED camera or under an Olympus BH-2 with a Canon EOS M50 camera.

### Results

### EG Polymorphisms Between Susceptible and Resistant Lines

EG and its promoter were sequenced in five soybean lines—the susceptible Bossier and Essex and the resistant Forrest, PI 96354, and Lee 74. Bossier and Essex have identical sequences throughout the promoter and gene. They differ from the Williams 82 reference at 3 locations—a single bp deletion 677 bp upstream of the 5′ UTR, a transversion 1,076 bp upstream of the 5′ UTR, and an intronic transversion. Forrest and PI 96354 have identical sequences throughout the promoter and gene. Lee 74 differs in only one location, a deletion 2,039 bp upstream of the 5′ UTR. In Lee 74, this deletion is 8 bp long while in Forrest and PI 96354 the deletion is 12 bp.

Twelve SNPs were identified in the gene between resistant and susceptible genotypes; three SNPs resulted in an amino acid change—M31I, T106I, or V174A (FIG. 7B). Twenty-five SNPs are found within 2,364 bp of the 5′ UTR (FIG. 7A). This was chosen as the cutoff for promoter analysis due to the presence of a 10 bp insertion in the resistant lines. The resistant promoters also contain a 2 bp insertion, a 15 bp insertion, and a deletion of 8-13 bp.

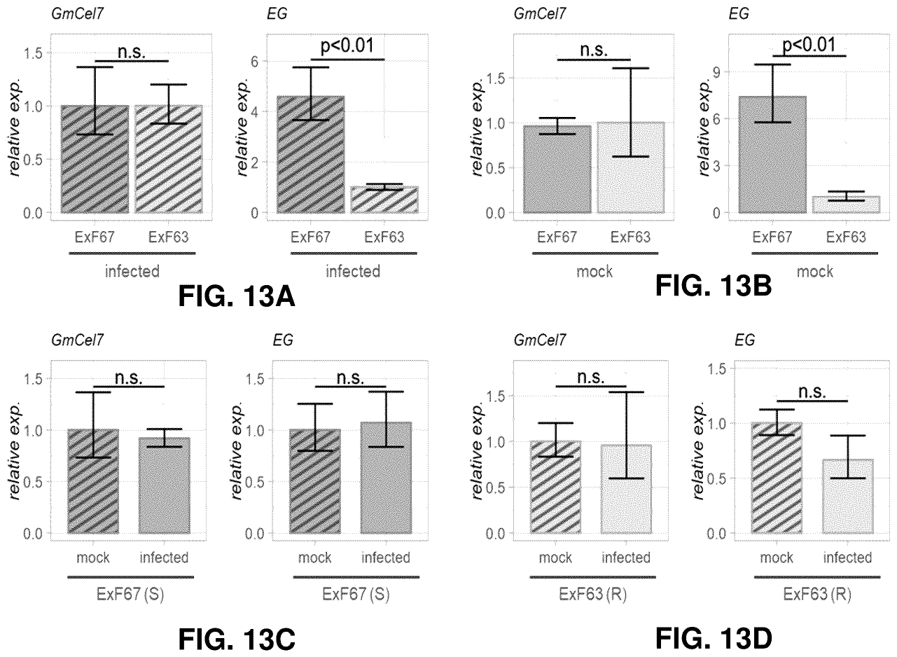

These polymorphisms result in the loss of several promoter elements (FIGS. 11A-11B), including a MADS box (tttgCCAAAtaagttggg) (SEQ ID NO:67), HD-Zip homeodomain (ttaTTAATttt) (SEQ ID NO:68), and a NAC (tTTACTtat), It also results in a gain of multiple C2H2 zinc finger binding sequences (aataACACTttta (SEQ ID NO:69), cACACTt, tAGTGTa), two MADS boxes (acaacaaaaaagtAGAAAgag (SEQ ID NO:70), tatttTTTTGg (SEQ ID NO:71)), and two HD-zip homeodomains (ttaATTAAtaa (SEQ ID NO:72), ctataTATTAtta (SEQ ID NO:73)). Two previously identified cis-acting elements identified in the promoter of the tobacco Cel7 EGase found to be upregulated in giant cells, included a pyrimidine box (CCTTTT) and a WBOX (TGACC) (Wang et al. 2007). In the EG promoter, a pyrimidine box located 122 bp upstream of the 5′ UTR is lost due to a SNP in the resistant lines and a WBOX (TGACC) located 2,030 bp upstream of the 5′ UTR is absent in the resistant lines. A WBOX interference element (TCAGY) is created 121 bp upstream of the 5′ UTR in the resistant lines, a motif associated with repression of ethylene signaling in response to wounding (Nishiuchi et al. 2004). This motif is also associated with repression of the gibberellin signaling pathway (Zhang et al. 2004). Neither version of the EG promoter contains the GH3 or DR5 motifs previously associated with auxin regulation (Karczmarek et al. 2004) and identified in the RKN-inducible tobacco Cel7 EGase promoter (Wang et al. 2007).

Five root-specific motifs found in the rolD promoter are found in the first 500 bp upstream of the 5′ UTR in all genotypes, supporting the root expression pattern (Elmayan and Tepfer 1995). However, the RKN-inducible TobRB7 promoter does not require the root-specific promoter elements to retain expression in giant cells (Opperman et al. 1994).

### Endoglucanases

The most closely related gene to EG is Glyma.02G016400 (GmCel7). To investigate the role of the close EG paralog, GmCel7, sequences of the susceptible and resistant lines were compared. The GmCel7 and EG protein sequences shared 95% pairwise identity. The differences are most concentrated in the signal peptide. The promoter and coding sequence of GmCel7 of the analyzed lines are nearly identical, with only a single intronic SNP occurring in the susceptible Bossier.

To identify similarity to other EGases that have been investigated for involvement in RKN infection, a genetic relationship tree using the Jukes-Cantor genetic distance model of endoglucanases from Arabidopsis, tobacco, tomato, and soybean was created using Geneious Prime 2024.0.5, showing that the EGases with the most similarity to EG are NtCel4 and SlCel4 (FIG. 12). EG and GmCel7 amino acid sequences share 80% identity with NtCel4, an EGase upregulated in tobacco during M. incognita infection (Goellner et al. 2001).

### EG Expression

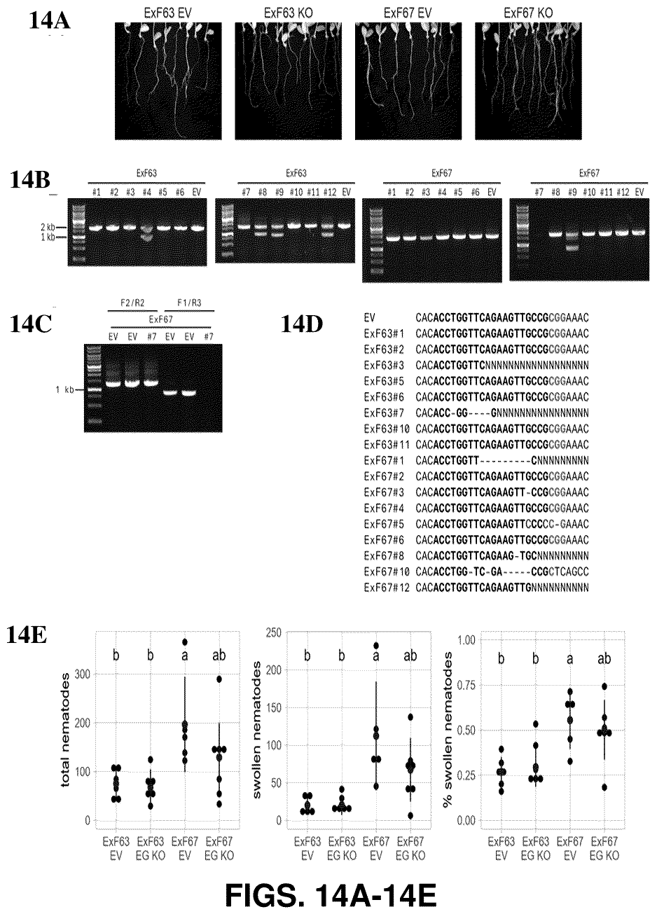

To further evaluate EG expression in soybean roots before and after nematode infection, cDNA collected from early galls of resistant and susceptible plants at 6 dpi, along with comparable tissue from uninfected plants, was used for qPCR. Expression of EG was 5× higher in uninfected susceptible root tissue than uninfected resistant root tissue and 7× higher in galls of susceptible roots than in galls collected from resistant roots (FIGS. 13A-13B). No change in expression was found in response to infection in either genotype (FIGS. 13A-13D). GmCel7 showed no change in expression, either in response to infection or between genotypes (FIGS. 13A-13D).

To identify spatial expression of EG, promoter:GUS constructs were created with both the resistant and susceptible versions of the promoter. GFP+ composite plants were inoculated with 1,200 J2s, then stained with GUS substrate. If GUS expression was observed in a root, it was stained with acid fuchsin to visualize nematode presence in relation to GUS expression. GUS expression was not quantified, merely evaluated for the presence/absence of GUS in response to infection. Five to eight independent transgenic roots were evaluated for each construct. GUS expression was observed in feeding sites under both promoters, however the occurrence of the expression was less frequently observed under the resistant promoter, while it was observed at the feeding sites of all swollen J2 under the susceptible promoter. When nematodes were not present in a root section, GUS expression was observed at low levels in vascular tissue under both the resistant and susceptible promoters. To functionally evaluate the length of the promoter required to induce expression in giant cells, 1.3 kb, approximately the first half) of the susceptible EG promoter was tested in ExF67 roots. GUS expression was still observed at feeding sites, indicating that the necessary promoter elements for expression in giant cells are located within 1.3 kb of EG promoter.

### CRISPR Knockout of EG

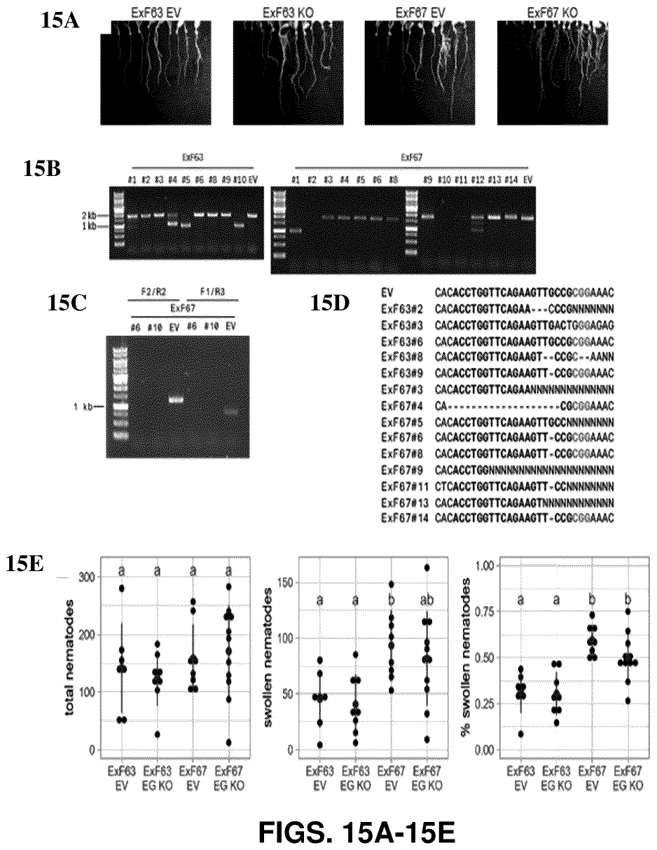

EG was knocked out using dual gRNAs to allow for identification of edits using PCR amplification. Composite plants with comparable GFP+ root systems were planted in cone-tainers in the greenhouse. Eleven days after inoculation, root systems were evaluated for RKN. Three lateral roots were collected from each plant for DNA extraction to confirm editing. If no knockout was observed after running the PCR product on a gel, the purified product was sequenced to check for edits within the coding region. Roughly 60-90% of GFP+ roots had edits, depending on the individual assay replication. Roots systems were stained with acid fuchsin to quantify the number of nematodes in each root system. Nematodes were categorized by development, either vermiform or swollen. Among EV root systems, ExF67 had significantly more total nematodes and swollen nematodes than ExF63 (FIGS. 14A-14E). ExF67 also had a significantly higher percentage of total nematodes being swollen (FIG. 14E). There was no significant difference in any of the three phenotypes measured in ExF63 after EG knockout, showing no decrease in resistance when EG is knocked out (FIGS. 14E, 15E). In edited ExF67 roots, a trend towards decreased susceptibility based on the number of swollen nematodes was observed when EG was knocked out in two biological replicates (FIGS. 14, 15E) though the difference was not statistically different from the ExF67 roots that were transformed with EV (FIGS. 14E, 15E). This may be attributed to the wide variation among the number of nematodes and the percentage of nematodes appearing swollen in the ExF67 roots. However, the trend towards decreased susceptibility was also evident in the lack of significant difference between ExF67 EG knockouts and ExF63 roots transformed with EV (FIGS. 15A-15E). The observed delay in nematode development in the ExF67 EG KO lines indicates that EG may play a role in feeding site establishment and therefore the reduced expression of EG in the resistant line may be contributing to RKN resistance.

### Overexpression of EG

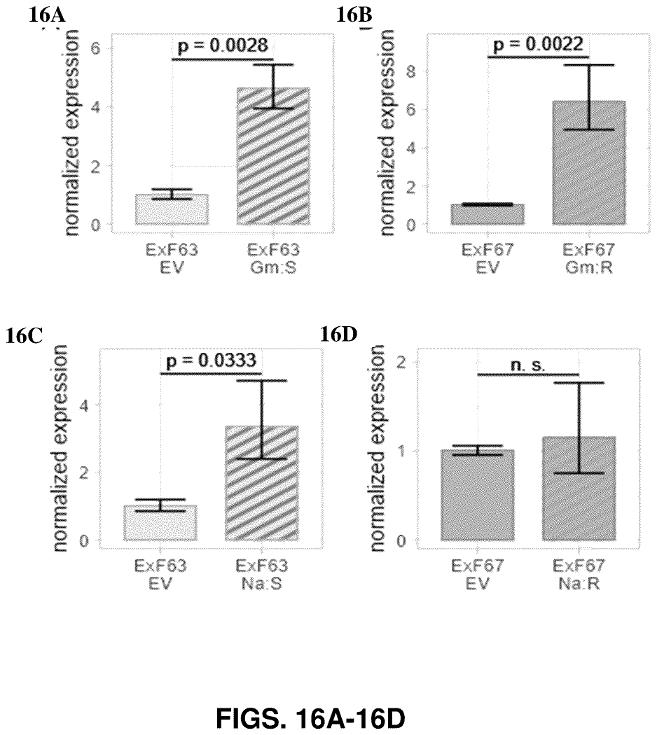

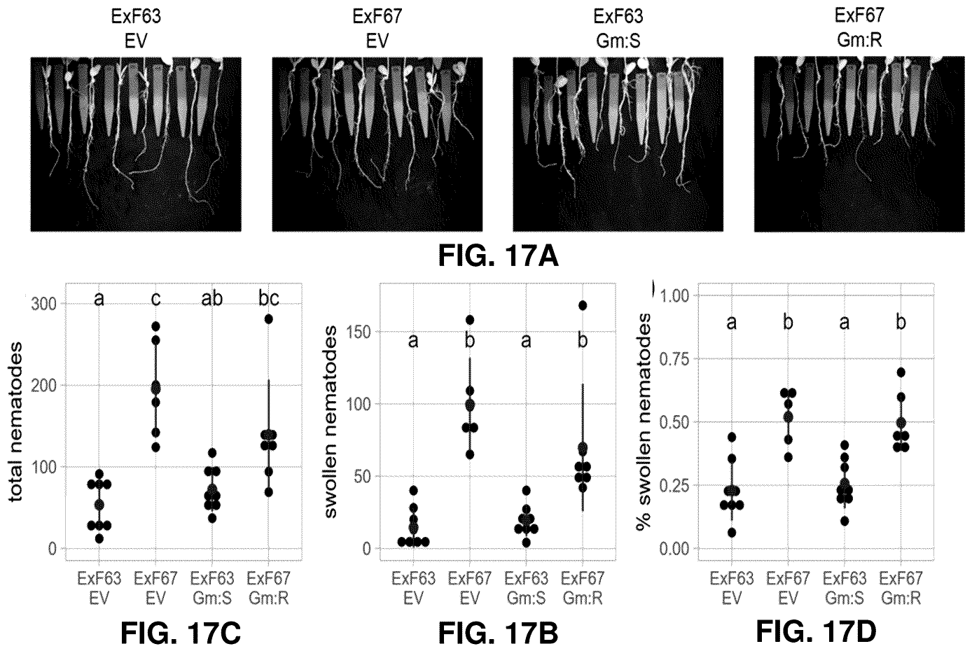

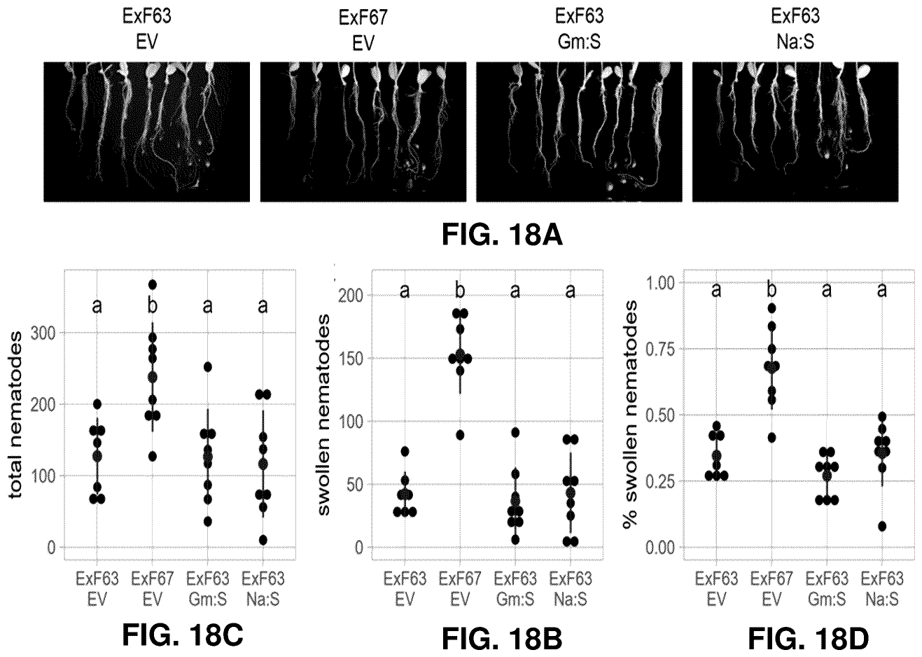

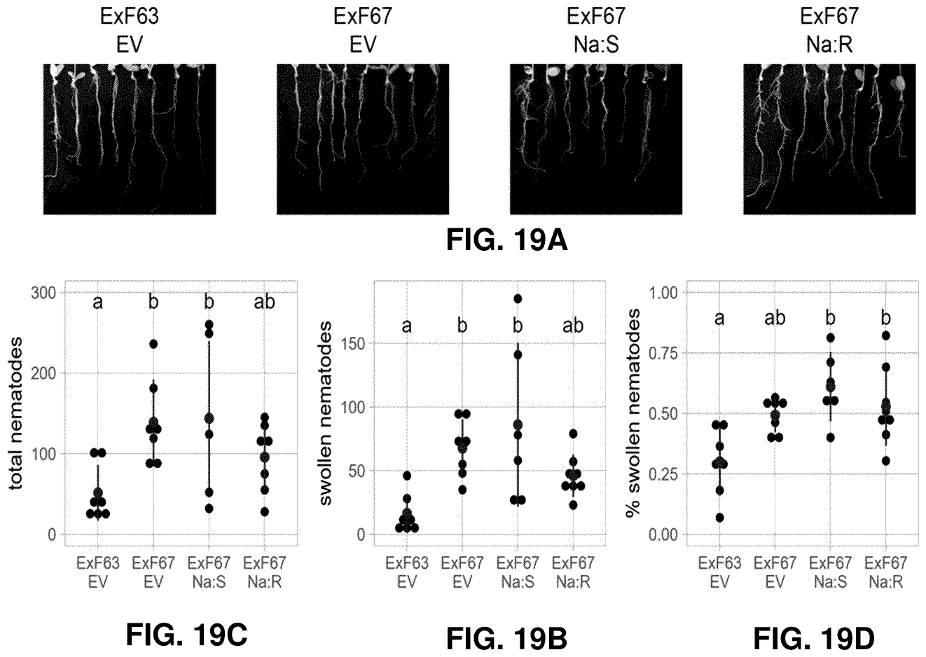

Expression of either susceptible or resistant EG CDS under the Gmubi promoter significantly increased the transcript abundance of EG (FIGS. 16A-16B). Under the corresponding native promoter, only the susceptible promoter showed significantly increased transcript abundance (FIG. 16C-16D). In all experiments, the total number of nematodes and number of swollen nematodes was significantly different between the resistant ExF67 and susceptible ExF63 EV composite plant roots, validating the bioassay. Introduction of the susceptible CDS under either its native promoter (Na:S) or the Gmubi promoter (Gm:S) into ExF63 did not result in a decrease in resistance in two biological replicates (FIGS. 17A-17D, 18A-18D). In addition, introduction of an additional copy of the susceptible EG CDS under its native promoter (Na:S) in the susceptible ExF67 did not result in hypersusceptibility in one biological replicate (FIGS. 19A-19D). In contrast, expression of the resistant EG CDS under its native promoter (Na:R) in the susceptible ExF67 did slightly decrease susceptibility. This difference was not significant for the total number of J2s or swollen nematodes between the Na:R and EV susceptible lines, but evident in the lack of significant difference between the Na:R and EV resistant lines (FIGS. 19B-19C). A similar trend was observed for expression of the resistant EG CDS under Gmubi promoter (Gm:R) in the susceptible ExF67 (FIG. 17B). These data indicate that introduction of the resistant version of EG into a susceptible background may enhance resistance to RKN.

### Discussion

The EG sequence similarities between unrelated resistant lines are compelling. The same amino acid changes occur in all three lines. Since PI 96354 had been heavily studied in its response to M. incognita, it was included in the sequence analysis. The use of a third, unrelated resistant soybean line added additional evidence to support identified polymorphisms. Lee 74 was selected because it was found to be unrelated to Forrest through a pedigree analysis (Ha et al. 2004). The promoters are nearly identical, with the only differences being in the length of an indel. In all tested genotypes, the susceptible allele is expressed more highly than in resistant genotypes. Expression of EG was observed in giant cells with both the susceptible and resistant promoter. The lack of significant expression differences identified between infected and mock tissue of the same genotype could be due to the larger portion of the root sampled at an early timepoint, diluting the ability to detect expression specific to giant cell formation. To detect this expression difference, a single-cell RNAseq study may be required. EG is expressed under both the susceptible and resistant promoters. The expression is less reliable under the resistant promoter. There are many differences between the sequenced susceptible and resistant promoters that could contribute to this difference.

Due to the diploidized autotetraploid nature of the soybean genome, most genes have a closely related paralog. The paralog of EG is GmCel7. The genomic sequences for these genes share 81% identity. EG and GmCel7 share 95% identity, with most of the differences occurring in the predicted signal peptide. GmCel7 contains no polymorphism between the susceptible and resistant lines analyzed in either the gene or promoter and does not exhibit differential expression either after infection or between susceptible and resistant lines. A prior study using RNAi to target GmCel7 in soybean found decreased susceptibility to Heterodera glycines (Woo et al. 2014). The methods used for RNAi silencing of GmCel7 almost certainly silenced EG as well due to the high level of sequence similarity (97% identity among the 249 bp amplified for RNAi). Similar host genes have been associated with RKN response in other host species. A tobacco endoglucanase, NtCel7, sharing 60% identity with EG was found to be expressed exclusively in giant cells (Goellner et al. 2001). The NtCel7 promoter was later confirmed to be nematode responsive (Wang et al. 2007). The Arabidopsis AtCel1 is expressed in elongation zones and giant cells (Mitchum et al. 2004). Among sequences used for phylogenetic analysis, EG is most closely related to NtCel4 and SlCel4. NtCel4, was shown to be upregulated in RKN-infected tobacco roots by Goellner et al. (2001). Knocking out EG in resistant roots did not increase susceptibility to M. incognita. While knocking out EG in susceptible roots did not significantly decrease the number of nematodes found in the root systems, it lost the significant difference between ExF63 EV and ExF67 EV roots. These data support EG playing an important role in soybean susceptibility to M. incognita. Overexpression of the susceptible EG in the resistant background did not increase susceptibility to M. incognita. It is the introduction of the resistant EG that had a trend toward decreased susceptibility. If EG is required for susceptible giant cell development, the loss of the susceptible allele could be what caused the observed decrease in susceptibility. It is important to note that the Rmi1 resistance response comes from nematode emigration before giant cell initiation. Knowledge of what the nematodes are sensing and how they are sensing it could inform the possible role of EG in resistance or susceptibility. Nematodes could be responding to the presence of the resistant allele, thus triggering emigration. They could also be responding to the lack of the susceptible EG in the root system, caused by the low basal level of expression present in the Rmi1-containing plants. The presence of GUS expression in the vascular cylinder indicates that the nematodes could be considering the presence or absence of EG when selecting an initial feeding cell. If a suitable cell cannot be found, the nematode will emigrate from the root. Since nematodes remaining in resistant roots are able to fully develop and produce eggs at the same level as resistant plants, the nematode is able to successfully create giant cells using the resistant EG (Moura et al. 1993). The decrease in susceptibility in ExF67 EG knockouts would result from the lack of EG present in those plants. However, this would not explain the decrease in susceptibility resulting from the introduction of the resistant EG into susceptible plants because the plant already possesses the alleles to generate enough EG for nematodes to not emigrate. This phenotype could result from the nematode not sensing the presence of EG, but the presence of the resistant allele, resulting in an unnecessary emigration. The expression of the resistant EG or the changes in the amino acid sequence could affect function to alter the cell wall in some way that is detectable by the nematode, leading to emigration.

The composite plant assay used for this study has some drawbacks. While it does offer a quicker method of generating CRISPR-edited plants, with usable roots being ready for inoculation in a matter of weeks versus more than six months for stable edited lines, the edits are not uniform throughout a treatment. This composite plant system is unable to determine the specific point in the root where the first CRISPR edits occurred in the gene and additional edits may occur further down the root. As a result, a single GFP+ root could contain root portions that are not edited, have heterozygous edits, or have homozygous edits. Each root is its own transformation event, making every plant a unique combination of the three editing possibilities. The additive action of Rmi1 compounds the incomplete nature of the edits in a single root system. This can lead to wide variation among replicates, as seen in the ExF67 knockout plants. However, the observed results in composite plant infection assays warrant further testing in stably edited plants.

Another source of variation is the growth of additional roots after planting in cones. When composite plants are transplanted, each treatment has a comparable set of roots, with the ideal roots being long with many lateral roots. The plants are left in the cones in the greenhouse for two days to allow for acclimation and settling in to the new soil. When the soil is rinsed from the plants eleven days after inoculation, there are frequently multiple new adventitious roots or non-GFP+ roots that have emerged from the callus. Sometimes these new, non-transgenic roots have become larger than the GFP+ root that was planted nearly two weeks prior. If the new roots had begun to grow soon after transplant into cones, the J2s inoculated into the soil would have an additional root to infect, decreasing the infection rate into the root that is stained for counting. This would result in an apparent decrease in susceptibility that was not due to the introduced construct and could also increase variation among a treatment.

Taken together, the evidence indicates that EG acts like a susceptibility gene (S gene) for M. incognita. There is precedent for S gene involvement in nematode resistance in soybean. GmSNAPO2 confers susceptibility to soybean cyst nematode (Usovsky et al. 2023). Susceptibility is lost through a premature stop codon in PI 437654 and a 6 kb insertion in PI 90763. Other characterized S genes include the Mlo gene in tomato that confers susceptibility to powdery mildew, the PsMLO1 gene that confers powdery mildew susceptibility in pea, and SWEET genes conferring susceptibility to bacterial blight in rice (Bai et al. 2008, Pavan et al. 2011, Eom et al. 2019).

While the three susceptible lines studied here had nearly identical sequences throughout the EG gene and promoter, the ubiquity of the loci among mapping studies indicates that there may have been more than one event leading to loss of susceptibility. If resistance arises from reduced expression or a loss of function, there are multiple mutations that could result in resistance, as demonstrated by GmSNAPO2.

### REFERENCES

Belamkar, V., Weeks, N. T., Bharti, A. K., Farmer, A. D., Graham, M. A., and Cannon, S. B. (2014). “Comprehensive characterization and RNA-Seq profiling of the HD-Zip transcription factor family in soybean (Glycine max) during dehydration and salt stress.” BMC Genomics 15: 1-25.
Bethke, G., Grundman, R. E., Sreekanta, S., Truman, W., Katagiri, F., and Glazebrook, J. (2014). “Arabidopsis PECTIN METHYLESTERASE INHIBITORs contribute to immunity against Pseudomonas syringae.” Plant Physiology 164(2): 1093-1107.
Brechenmacher, L., Nguyen, T. H. N., Zhang, N., Jun, T.-H., Xu, D., Mian, M. R., and Stacey, G. (2015). “Identification of soybean proteins and genes differentially regulated in near isogenic lines differing in resistance to aphid infestation.” Journal of Proteome Research 14(10): 4137-4146.
Cook, D. E., Lee, T. G., Guo, X., Melito, S., Wang, K., Bayless, A. M., Wang, J., Hughes, T. J., Willis, D. K., and Clemente, T. E. (2012). “Copy number variation of multiple genes at Rhg1 mediates nematode resistance in soybean.” Science 338(6111): 1206-1209.
Crop Protection Network (2024). Estimates of crop yield losses due to diseases and invertebrate pests: an online tool. https://loss.cropprotectionnetwork.org. DOI: 10.31274/cpn-20191121-0.
Danecek, P., Bonfield, J. K., Liddle, J., Marshall, J., Ohan, V., Pollard, M. O., Whitwham, A., Keane, T., McCarthy, S. A., and Davies, R. M. (2021). “Twelve years of SAMtools and BCFtools.” Gigascience 10(2): giab008.
Danzer, J., Mellott, E., Bui, A. Q., Le, B. H., Martin, P., Hashimoto, M., Perez-Lesher, J., Chen, M., Pelletier, J. M., and Somers, D. A. (2015). “Down-regulating the expression of 53 soybean transcription factor genes uncovers a role for SPEECHLESS in initiating stomatal cell lineages during embryo development.” Plant Physiology 168(3): 1025-1035.
Devi, M. J., Sinclair, T. R., and Taliercio, E. (2015). “Comparisons of the effects of elevated vapor pressure deficit on gene expression in leaves among two fast-wilting and a slow-wilting soybean.” PloS One 10(10): e0139134.
Ding, X., Shields, J., Allen, R., and Hussey, R. S. (1998). “A secretory cellulose-binding protein cDNA cloned from the root-knot nematode (Meloidogyne incognita).” Molecular Plant-Microbe Interactions 11(10): 952-959.
Edwards, K., Johnstone, C., and Thompson, C. (1991). “A simple and rapid method for the preparation of plant genomic DNA for PCR analysis.” Nucleic Acids Research 19(6): 1349.
Espina, M. J. C., Lovell, J. T., Jenkins, J., Shu, S., Sreedasyam, A., Jordan, B. D., Webber, J., Boston, L., Brůna, T., and Talag, J. (2024). “Assembly, comparative analysis, and utilization of a single haplotype reference genome for soybean.” The Plant Journal.
Flors, V., Leyva, M. d. l. O., Vicedo, B., Finiti, I., Real, M. D., Garcia-Agustin, P., Bennett, A. B., and González-Bosch, C. (2007). “Absence of the endo-β-1,4-glucanases Cell and Cel2 reduces susceptibility to Botrytis cinerea in tomato.” The Plant Journal 52(6): 1027-1040.
Fourie, H., Mienie, C. M., McDonald, A. H., and De Waele, D. (2008). “Identification and validation of genetic markers associated with Meloidogyne incognita race 2 resistance in soybean, Glycine max (L.) Merr.” Nematology 10(5): 651-661.
Goellner, M., Wang, X., and Davis, E. L. (2001). “Endo-β-1,4-glucanase expression in compatible plant-nematode interactions.” The Plant Cell 13(10): 2241-2255.
Goode, K., and Mitchum, M. G. (2022). “Pattern-triggered immunity against root-knot nematode infection: A minireview.” Physiologia Plantarum 174(2): e13680.
Ha, B. K., Bennett, J. B., Hussey, R. S., Finnerty, S. L., and Boerma, H. R. (2004). “Pedigree analysis of a major QTL conditioning soybean resistance to southern root-knot nematode.” Crop Science 44(3): 758-763.
Hamamouch, N., Li, C., Hewezi, T., Baum, T. J., Mitchum, M. G., Hussey, R. S., Vodkin, L. O., and Davis, E. L. (2012). “The interaction of the novel 30C02 cyst nematode effector protein with a plant β-1,3-endoglucanase may suppress host defence to promote parasitism.” Journal of Experimental Botany 63(10): 3683-3695.
Hartwig, E., and Epps, J. (1973). “Registration of ‘Forrest’ soybeans.” Crop Science 13(2): 287.
Herman, M., Hussey, R. S., and Boerma, H. R. (1990). “Response of resistant soybean plant introductions to Meloidogyne incognita in field microplots.” Journal of Nematology 22(2): 237.
Herman, M., Hussey, R. S., and Boerma, H. R. (1991). “Penetration and Development of Meloidogyne incognita on Roots of Resistant Soybean Genotypes.” Journal of Nematology 23(2): 155-161.
Hewezi, T., Howe, P., Maier, T. R., Hussey, R. S., Mitchum, M. G., Davis, E. L., and Baum, T. J. (2008). “Cellulose binding protein from the parasitic nematode Heterodera schachtii interacts with Arabidopsis pectin methylesterase inhibitor: cooperative cell wall modification during parasitism.” The Plant Cell 20(11): 3080-3093.
Huang, G., Dong, R., Allen, R., Davis, E. L., Baum, T. J., and Hussey, R. S. (2005). “Developmental expression and molecular analysis of two Meloidogyne incognita pectate lyase genes.” International Journal for Parasitology 35(6): 685-692.
Hunt, M., Kaur, N., Stromvik, M., and Vodkin, L. (2011). “Transcript profiling reveals expression differences in wild-type and glabrous soybean lines.” BMC Plant Biology 11: 1-16.
Hussey, R. S., and Barker, K. R. (1973). “Comparison of methods of collecting inocula of Meloidogyne spp., including a new technique.” Plant Disease Reporter.
Jaubert, S., Laffaire, J. B., Abad, P., and Rosso, M. N. (2002). “A polygalacturonase of animal origin isolated from the root-knot nematode Meloidogyne incognita.” FEBS Letters 522(1-3): 109-112.
Jiao, Y., Vuong, T. D., Liu, Y., Li, Z., Noe, J., Robbins, R. T., Joshi, T., Xu, D., Shannon, J. G., and Nguyen, H. T. (2015). “Identification of quantitative trait loci underlying resistance to southern root-knot and reniform nematodes in soybean accession PI 567516C.” Molecular Breeding 35: 1-10.
Kour, A., Boone, A. M., and Vodkin, L. O. (2014). “RNA-Seq profiling of a defective seed coat mutation in Glycine max reveals differential expression of proline-rich and other cell wall protein transcripts.” PLoS One 9(5): e96342.
Le Cam, B., Massiot, P., Campion, C., and Rouxel, F. (1994). “Susceptibility of carrot cultivars to Mycocentrospora acerina and the structure of cell wall polysaccharides.” Physiological and Molecular Plant Pathology 45(2): 139-151.
Li, Z., Jakkula, L., Hussey, R. S., Tamulonis, J. P., and Boerma, H. R. (2001). “SSR mapping and confirmation of the QTL from PI96354 conditioning soybean resistance to southern root-knot nematode.” Theoretical and Applied Genetics 103: 1167-1173.
Libault, M., Farmer, A., Brechenmacher, L., Drnevich, J., Langley, R. J., Bilgin, D. D., Radwan, O., Neece, D. J., Clough, S. J., and May, G. D. (2010). “Complete transcriptome of the soybean root hair cell, a single-cell model, and its alteration in response to Bradyrhizobium japonicum infection.” Plant Physiology 152(2): 541-552.
Libault, M., Thibivilliers, S., Bilgin, D., Radwan, O., Benitez, M., Clough, S., and Stacey, G. (2008). “Identification of four soybean reference genes for gene expression normalization.” The Plant Genome 1(1): 44-54.
Lightfoot, D. A., Njiti, V. N., Gibson, P. T., and Kassem, M. A. (2005). “Registration of the Essex×Forrest recombinant inbred line mapping population.” Crop Science 45(4): 1678.
Lionetti, V., Cervone, F., and Bellincampi, D. (2012). “Methyl esterification of pectin plays a role during plant-pathogen interactions and affects plant resistance to diseases.” Journal of Plant Physiology 169(16): 1623-1630.
Liu, S., Kandoth, P. K., Warren, S. D., Yeckel, G., Heinz, R., Alden, J., Yang, C., Jamai, A., El-Mellouki, T., and Juvale, P. S. (2012). “A soybean cyst nematode resistance gene points to a new mechanism of plant resistance to pathogens.” Nature 492(7428): 256-260.
López-Cruz, J., Finiti, I., Fernindez-Crespo, E., Crespo-Salvador, O., Garcia-Agustin, P., and González-Bosch, C. (2014). “Absence of endo-1,4-β-glucanase KOR1 alters the jasmonate-dependent defence response to Pseudomonas syringae in Arabidopsis.” Journal of Plant Physiology 171(16): 1524-1532.
Luzzi, B. M., Boerma, H. R., and Hussey, R. S. (1987). “Resistance to Three Species of Root-Knot Nematode in Soybean.” Crop Science 27(2): 258-262.
Luzzi, B. M., Boerma, H. R., and Hussey, R. S. (1994). “A gene for resistance to the southern root-knot nematode in soybean.” Journal of Heredity 85(6): 484-486.
Micheli, F. (2001). “Pectin methylesterase inhibitors: cell wall enzymes with important roles in plant physiology.” Trends in Plant Science 6(9): 414-419.
Milligan, S. B., Bodeau, J., Yaghoobi, J., Kaloshian, I., Zabel, P., and Williamson, V. M. (1998). “The root knot nematode resistance gene Mi from tomato is a member of the leucine zipper, nucleotide binding, leucine-rich repeat family of plant genes.” The Plant Cell 10(8): 1307-1319.
Miranda, V. d. J., Coelho, R. R., Viana, A. A. B., de Oliveira Neto, O. B., Carneiro, R. M. D. G., Rocha, T. L., Grossi de Sa, M. F., and Fragoso, R. R. (2013). “Validation of reference genes aiming accurate normalization of qPCR data in soybean upon nematode parasitism and insect attack.” BMC Research Notes 6: 1-10.
Mitchum, M. G., Averitt, B., Goode, K., Martin, K., Lance, K., Mitchell, M., Hussey, R. S., Jagdale, G., and Kemerait, R. C. (2023). “Frequency of Occurrence of Heterodera glycines Types, Meloidogyne spp., and Eight Other Nematode Taxa Associated with Soybeans in Georgia.” Plant Health Progress 24(2): 214-220.
Molina, A., Jordá, L., Torres, M. Á., Martin-Dacal, M., Berlanga, D. J., Fernández-Calvo, P., Gómez-Rubio, E., and Martin-Santamaría, S. (2024). “Plant cell wall-mediated disease resistance: Current understanding and future perspectives.” Molecular Plant 17(5): 699-724.
Ochoa-Villarreal, M., Aispuro-Hernández, E., Vargas-Arispuro, I., and Martinez-Téllez, M. Á. (2012). “Plant cell wall polymers: function, structure and biological activity of their derivatives.” Polymerization 4: 63-86.
Passianotto, A. L., Sonah, H., Dias, W. P., Marcelino-Guimarães, F. C., Belzile, F., and Abdelnoor, R. V. (2017). “Genome-wide association study for resistance to the southern root-knot nematode (Meloidogyne incognita) in soybean.” Molecular Breeding 37: 1-11.
Paysan-Lafosse, T., Blum, M., Chuguransky, S., Grego, T., Pinto, B. L., Salazar, G. A., Bileschi, M. L., Bork, P., Bridge, A., and Colwell, L. (2023). “InterPro in 2022.” Nucleic Acids Research 51(D1): D418-D427.
Pelletier, J. M., Kwong, R. W., Park, S., Le, B. H., Baden, R., Cagliari, A., Hashimoto, M., Munoz, M. D., Fischer, R. L., and Goldberg, R. B. (2017). “LEC1 sequentially regulates the transcription of genes involved in diverse developmental processes during seed development.” Proceedings of the National Academy of Sciences 114(32): E6710-E6719.
Pelloux, J., Rusterucci, C., and Mellerowicz, E. J. (2007). “New insights into pectin methylesterase inhibitor structure and function.” Trends in Plant Science 12(6): 267-277.
Pham, A. T., McNally, K., Abdel-Haleem, H., Boerma, H. R., and Li, Z. (2013). “Fine mapping and identification of candidate genes controlling the resistance to southern root-knot nematode in PI 96354.” Theoretical and Applied Genetics 126: 1825-1838.
R Core Team (2024). “R: A Language and Environment for Statistical Computing.” R Foundation for Statistical Computing.
Severin, A. J., Woody, J. L., Bolon, Y.-T., Joseph, B., Diers, B. W., Farmer, A. D., Muehlbauer, G. J., Nelson, R. T., Grant, D., and Specht, J. E. (2010). “RNA-Seq Atlas of Glycine max: a guide to the soybean transcriptome.” BMC Plant Biology 10: 1-16.
Shaibu, A. S., Zhang, S., Ma, J., Feng, Y., Huai, Y., Qi, J., Li, J., Abdelghany, A. M., Azam, M., and Htway, H. T. P. (2022). “The GmSNAP11 contributes to resistance to soybean cyst nematode race 4 in Glycine max.” Frontiers in Plant Science 13: 939763.
Sukno, S., Shimerling, O., McCuiston, J., Tsabary, G., Shani, Z., Shoseyov, O., and Davis, E. L. (2006). “Expression and regulation of the Arabidopsis thaliana Cell endo 1, 4 β glucanase gene during compatible plant-nematode interactions.” Journal of Nematology 38(3): 354.
Tamulonis, J. P., Luzzi, B. M., Hussey, R. S., Parrott, W. A., and Boerma, H. R. (1997). “RFLP mapping of resistance to southern root-knot nematode in soybean.” Crop Science 37(6): 1903-1909.
Urbanowicz, B. R., Bennett, A. B., Del Campillo, E., Catalá, C., Hayashi, T., Henrissat, B., Hofte, H., McQueen-Mason, S. J., Patterson, S. E., and Shoseyov, O. (2007). “Structural organization and a standardized nomenclature for plant endo-1,4-β-glucanases (cellulases) of glycosyl hydrolase family 9.” Plant Physiology 144(4): 1693-1696.
Usovsky, M., Gamage, V. A., Meinhardt, C. G., Dietz, N., Triller, M., Basnet, P., Gillman, J. D., Bilyeu, K. D., Song, Q., and Dhital, B. (2023). “Loss-of-function of an α-SNAP gene confers resistance to soybean cyst nematode.” Nature Communications 14(1): 7629.
Wietholter, N., Graeßner, B., Mierau, M., Mort, A. J., and Moerschbacher, B. M. (2003). “Differences in the methyl ester distribution of homogalacturonans from near-isogenic wheat lines resistant and susceptible to the wheat stem rust fungus.” Molecular Plant-Microbe Interactions 16(10): 945-952.
Wolf, S., Mouille, G., and Pelloux, J. (2009). “Homogalacturonan methyl-esterification and plant development.” Molecular Plant 2(5): 851-860.
Woo, M. O., Beard, H., MacDonald, M. H., Brewer, E. P., Youssef, R. M., Kim, H., and Matthews, B. F. (2014). “Manipulation of two α-endo-β-1,4-glucanase genes, AtCel6 and GmCel7, reduces susceptibility to Heterodera glycines in soybean roots.” Molecular Plant Pathology 15(9): 927-939.
Wormit, A., and Usadel, B. (2018). “The multifaceted role of pectin methylesterase inhibitor inhibitors (PMEIs).” International Journal of Molecular Sciences 19(10): 2878.
Xu, X., Zeng, L., Tao, Y., Vuong, T., Wan, J., Boerma, H. R., Noe, J., Li, Z., Finnerty, S., and Pathan, S. M. (2013). “Pinpointing genes underlying the quantitative trait loci for root-knot nematode resistance in palaeopolyploid soybean by whole gen
Abad, P., Castagnone-Sereno, P., Rosso, M. N., de Almeida Engler, J., and Favery, B. (2009). Invasion, feeding and development. Root-knot Nematodes. R. N. Perry, M. Moens and J. L. Starr, CABI International: 163-181.
Bai, Y., Pavan, S., Zheng, Z., Zappel, N. F., Reinstadler, A., Lotti, C., De Giovanni, C., Ricciardi, L., Lindhout, P., and Visser, R. (2008). “Naturally occurring broad-spectrum powdery mildew resistance in a Central American tomato accession is caused by loss of Mlo function.” Molecular Plant-Microbe Interactions 21(1): 30-39.
Chow, C. N., Yang, C. W., Wu, N.Y., Wang, H. T., Tseng, K. C., Chiu, Y. H., Lee, T. Y., and Chang, W. C. (2024). “PlantPAN 4.0: Updated database for identifying conserved non-coding sequences and exploring dynamic transcriptional regulation in plant promoters.” Nucleic Acids Research 52(D1): D1569-D1578.
Crop Protection Network (2024). Estimates of crop yield losses due to diseases and invertebrate pests: an online tool. https://loss.cropprotectionnetwork.org. DOI: 10.31274/cpn-20191121-0.
Damodaran, S., Dubois, A., Xie, J., Ma, Q., Hindie, V., and Subramanian, S. (2019). “GmZPR3d interacts with GmHD-ZIP III proteins and regulates soybean root and nodule vascular development.” International Journal of Molecular Sciences 20(4): 827.
Ding, X., Shields, J., Allen, R., and Hussey, R. S. (1998). “A secretory cellulose-binding protein cDNA cloned from the root-knot nematode (Meloidogyne incognita).” Molecular Plant-Microbe Interactions 11(10): 952-959.
Elmayan, T., and Tepfer, M. (1995). “Evaluation in tobacco of the organ specificity and strength of the rolD promoter, domain A of the 35S promoter and the 35S2 promoter.” Transgenic Research 4(6): 388-396.
Eom, J.-S., Luo, D., Atienza-Grande, G., Yang, J., Ji, C., Thi Luu, V., Huguet-Tapia, J. C., Char, S. N., Liu, B., and Nguyen, H. (2019). “Diagnostic kit for rice blight resistance.” Nature Biotechnology 37(11): 1372-1379.
Fan, Y. L., Zhang, X. H., Zhong, L. J., Wang, X. Y., Jin, L. S., and Lyu, S. H. (2020). “One-step generation of composite soybean plants with transgenic roots by Agrobacterium rhizogenes-mediated transformation.” BMC Plant Biology 20: 1-11.
Feng, Z., Zhang, B., Ding, W., Liu, X., Yang, D.-L., Wei, P., Cao, F., Zhu, S., Zhang, F., and Mao, Y. (2013). “Efficient genome editing in plants using a CRISPR/Cas system.” Cell Research 23(10): 1229-1232.
Fourie, H., Mienie, C. M., McDonald, A. H., and De Waele, D. (2008). “Identification and validation of genetic markers associated with Meloidogyne incognita race 2 resistance in soybean, Glycine max (L.) Merr.” Nematology 10(5): 651-661.
Goellner, M., Shani, Z., Shoseyov, O., and Davis, E. (2000). “Plant endoglucanase expression in cyst and root knot nematode-induced feeding cells.” Phytopathology 90: S28.
Goellner, M., Wang, X., and Davis, E. L. (2001). “Endo-β-1,4-glucanase expression in compatible plant-nematode interactions.” The Plant Cell 13(10): 2241-2255.
Ha, B. K., Bennett, J. B., Hussey, R. S., Finnerty, S. L., and Boerma, H. R. (2004). “Pedigree analysis of a major QTL conditioning soybean resistance to southern root-knot nematode.” Crop Science 44(3): 758-763.
Herman, M., Hussey, R. S., and Boerma, H. R. (1990). “Response of resistant soybean plant introductions to Meloidogyne incognita in field microplots.” Journal of Nematology 22(2): 237.
Herman, M., Hussey, R. S., and Boerma, H. R. (1991). “Penetration and development of Meloidogyne incognita on roots of resistant soybean genotypes.” Journal of Nematology 23(2): 155-161.
Hernandez-Garcia, C. M., Martinelli, A. P., Bouchard, R. A., and Finer, J. J. (2009). “A soybean (Glycine max) polyubiquitin promoter gives strong constitutive expression in transgenic soybean.” Plant Cell Reports 28: 837-849.
Hewezi, T., Howe, P., Maier, T. R., Hussey, R. S., Mitchum, M. G., Davis, E. L., and Baum, T. J. (2008). “Cellulose binding protein from the parasitic nematode Heterodera schachtii interacts with Arabidopsis pectin methylesterase inhibitor: cooperative cell wall modification during parasitism.” The Plant Cell 20(11): 3080-3093.
Higo, K., Ugawa, Y., Iwamoto, M., and Korenaga, T. (1999). “Plant cis-acting regulatory DNA elements (PLACE) database: 1999.” Nucleic Acids Research 27(1): 297-300.
Ithal, N., Recknor, J., Nettleton, D., Maier, T., Baum, T. J., and Mitchum, M. G. (2007). “Developmental transcript profiling of cyst nematode feeding cells in soybean roots.” Molecular Plant-Microbe Interactions 20(5): 510-525.
Jiao, Y., Vuong, T. D., Liu, Y., Li, Z., Noe, J., Robbins, R. T., Joshi, T., Xu, D., Shannon, J. G., and Nguyen, H. T. (2015). “Identification of quantitative trait loci underlying resistance to southern root-knot and reniform nematodes in soybean accession PI 567516C.” Molecular Breeding 35: 1-10.
Kang, J. (2016). Application of CRISPR/Cas9-mediated genome editing for studying soybean resistance to soybean cyst nematode. M. S. Thesis, University of Missouri.
Karczmarek, A., Overmars, H., Helder, J., and Goverse, A. (2004). “Feeding cell development by cyst and root-knot nematodes involves a similar early, local and transient activation of a specific auxin-inducible promoter element.” Molecular Plant Pathology 5(4): 343-346.
Li, Z., Jakkula, L., Hussey, R. S., Tamulonis, J. P., and Boerma, H. R. (2001). “SSR mapping and confirmation of the QTL from PI96354 conditioning soybean resistance to southern root-knot nematode.” Theoretical and Applied Genetics 103: 1167-1173.
Libault, M., Thibivilliers, S., Bilgin, D., Radwan, O., Benitez, M., Clough, S., and Stacey, G. (2008). “Identification of four soybean reference genes for gene expression normalization.” The Plant Genome 1(1): 44-54.
Lightfoot, D., Njiti, V., Gibson, P., Kassem, M., Iqbal, J., and Meksem, K. (2005). “Registration of the Essexx Forrest recombinant inbred line mapping population.” Crop science 45(4): 1678-1681.
Luzzi, B. M., Boerma, H. R., and Hussey, R. S. (1994). “A gene for resistance to the southern root-knot nematode in soybean.” Journal of Heredity 85(6): 484-486.
Mazarei, M., Routray, P., Piya, S., Stewart Jr, C. N., and Hewezi, T. (2023). “Overexpression of soybean GmNAC19 and GmGRAB1 enhances root growth and water-deficit stress tolerance in soybean.” Frontiers in Plant Science 14: 1186292.
Micheli, F. (2001). “Pectin methylesterase inhibitors: cell wall enzymes with important roles in plant physiology.” Trends in Plant Science 6(9): 414-419.
Miranda, V. d. J., Coelho, R. R., Viana, A. A. B., de Oliveira Neto, O. B., Carneiro, R. M. D. G., Rocha, T. L., Grossi de Sa, M. F., and Fragoso, R. R. (2013). “Validation of reference genes aiming accurate normalization of qPCR data in soybean upon nematode parasitism and insect attack.” BMC Research Notes 6: 1-10.
Mitchum, M. G., Averitt, B., Goode, K., Martin, K., Lance, K., Mitchell, M., Hussey, R. S., Jagdale, G., and Kemerait, R. C. (2023). “Frequency of Occurrence of Heterodera glycines Types, Meloidogyne spp., and Eight Other Nematode Taxa Associated with Soybeans in Georgia.” Plant Health Progress 24(2): 214-220.
Mitchum, M. G., Sukno, S., Wang, X., Shani, Z., Tsabary, G., Shoseyov, O., and Davis, E. L. (2004). “The promoter of the Arabidopsis thaliana Cell endo-1,4-β glucanase gene is differentially expressed in plant feeding cells induced by root-knot and cyst nematodes.” Molecular Plant Pathology 5(3): 175-181.
Moura, R., Davis, E. L., Luzzi, B. M., Boerma, H. R., and Hussey, R. S. (1993). “Post-infectional development of Meloidogyne incognita on susceptible and resistant soybean genotypes.” Nematropica: 7-13.
Nishiuchi, T., Shinshi, H., and Suzuki, K. (2004). “Rapid and transient activation of transcription of the ERF3 gene by wounding in tobacco leaves: possible involvement of NtWRKYs and autorepression.” Journal of Biological Chemistry 279(53): 55355-55361.
Ochoa-Villarreal, M., Aispuro-Hernindez, E., Vargas-Arispuro, I., and Martinez-Tellez, M. A. (2012). “Plant cell wall polymers: function, structure and biological activity of their derivatives.” Polymerization 4: 63-86.
Opperman, C. H., Taylor, C. G., and Conkling, M. A. (1994). “Root-knot nematode-directed expression of a plant root-specific gene.” Science 263(5144): 221-223.
Passianotto, A. L., Sonah, H., Dias, W. P., Marcelino-Guimaraes, F. C., Belzile, F., and Abdelnoor, R. V. (2017). “Genome-wide association study for resistance to the southern root-knot nematode (Meloidogyne incognita) in soybean.” Molecular Breeding 37: 1-11.
Pavan, S., Schiavulli, A., Appiano, M., Marcotrigiano, A. R., Cillo, F., Visser, R. G., Bai, Y., Lotti, C., and Ricciardi, L. (2011). “Pea powdery mildew erl resistance is associated to loss-of-function mutations at a MLO homologous locus.” Theoretical and Applied Genetics 123: 1425-1431.
Tamulonis, J. P., Luzzi, B. M., Hussey, R. S., Parrott, W. A., and Boerma, H. R. (1997). “RFLP mapping of resistance to southern root-knot nematode in soybean.” Crop Science 37(6): 1903-1909.
Usovsky, M., Gamage, V. A., Meinhardt, C. G., Dietz, N., Triller, M., Basnet, P., Gillman, J. D., Bilyeu, K. D., Song, Q., and Dhital, B. (2023). “Loss-of-function of an α-SNAP gene confers resistance to soybean cyst nematode.” Nature Communications 14(1): 7629.
Valliyodan, B., Cannon, S. B., Bayer, P. E., Shu, S., Brown, A. V., Ren, L., Jenkins, J., Chung, C. Y. L., Chan, T. F., and Daum, C. G. (2019). “Construction and comparison of three reference-quality genome assemblies for soybean.” The Plant Journal 100(5): 1066-1082.
Wang, X., Replogle, A., Davis, E. L., and Mitchum, M. G. (2007). “The tobacco Cel7 gene promoter is auxin-responsive and locally induced in nematode feeding sites of heterologous plants.” Molecular Plant Pathology 8(4): 423-436.
Woo, M. O., Beard, H., MacDonald, M. H., Brewer, E. P., Youssef, R. M., Kim, H., and Matthews, B. F. (2014). “Manipulation of two α-endo-β-1,4-glucanase genes, AtCel6 and GmCel7, reduces susceptibility to Heterodera glycines in soybean roots.” Molecular Plant Pathology 15(9): 927-939.
Xu, X., Zeng, L., Tao, Y., Vuong, T., Wan, J., Boerma, H. R., Noe, J., Li, Z., Finnerty, S., and Pathan, S. M. (2013). “Pinpointing genes underlying the quantitative trait loci for root-knot nematode resistance in palaeopolyploid soybean by whole genome resequencing.” Proceedings of the National Academy of Sciences 110(33): 13469-13474.
Zhang, Z. L., Xie, Z., Zou, X., Casaretto, J., Ho, T. H. D., and Shen, Q. J. (2004). “A rice WRKY gene encodes a transcriptional repressor of the gibberellin signaling pathway in aleurone cells.” Plant Physiology 134(4): 1500-1513.
U.S. Pat. No. 8,198,509.

We claim:

1. A plant or plant cell comprising a genetic modification that reduces, inhibits or silences expression or translation of a target polynucleotide having a nucleic acid sequence of
(i) any one of SEQ ID NOs:1-3, or a complement thereof, and/or a nucleic acid encoding the polypeptide of SEQ ID NO:7 or 8, or a complement thereof, and/or
(ii) any one of SEQ ID NOs:9-11, or a complement thereof, and/or a nucleic acid encoding the polypeptide of SEQ ID NO:15 or 16, or a complement thereof, wherein the plant is a transgenic or non-transgenic plant.

2. The plant or plant cell of claim 1 wherein the genetic modification was induced by gene editing technology optionally selected from CRISPR/Cas, TALENs, and Zinc Finger Nucleases.

3. A transgenic plant or transgenic plant cell, comprising a polynucleotide comprising an expression control sequence operably linked to a nucleic acid sequence encoding an antisense nucleic acid that reduces, inhibits or silences expression or translation of a target polynucleotide having a nucleic acid sequence of
(i) any one of SEQ ID NOs:1-3, or a complement thereof, and/or a nucleic acid encoding the polypeptide of SEQ ID NO:7 or 8, or a complement thereof; and/or
(ii) any one of SEQ ID NOs:9-11, or a complement thereof, and/or a nucleic acid encoding the polypeptide of SEQ ID NO:15 or 16, or a complement thereof.

4. The plant or plant cell of claim 3, wherein the nucleic acid alters, reduces, inhibits, or silences expression or translation of the target polynucleotide by RNAi, dsRNA, miRNA, siRNA, or transacting small-interfering RNAs (tasiRNA).

5. The plant or plant cell of claim 1, further comprising a second polynucleotide comprising a promoter sequence comprising SEQ ID NO:4, or a fragment thereof comprising 50, 100, 150, 250, 500, 750, 1,000, 1,250, 1,500, or 2,000 or more nucleotides of SEQ ID NO:4 operably linked to one or more nucleic acid sequence(s) encoding one or more nematode susceptibility genes.

6. The plant or plant cell of claim 3, further comprising a polynucleotide comprising a promoter sequence comprising SEQ ID NO:12, or a fragment thereof comprising 50, 100, 150, 250, 500, 750, 1,000, 1,250, 1,500, or 2,000 or more nucleotides of SEQ ID NO:12 operably linked to one or more nucleic acid sequence(s) encoding one or more nematode susceptibility genes.

7. The plant or cell of claim 1, wherein the plant comprises a soybean plant optionally, wherein the soybean plant comprises a Glycine max plant,
optionally wherein the Glycine max is a cultivar selected from the group consisting of Jack, Resnik, Williams 82, Corsoy, Crawford, Hutcheson, Benning, Woodruff, Kunitz and Champ.

8. The plant or cell of claim 1, wherein the plant comprises a cereal crop plant, such as wheat, oat, barley, or rice; or a forage plant such as bahiagrass, dallisgrass, kleingrass, guineagrass, reed canarygrass, orchardgrass, ricegrass, foxtail, or vetch; or a legume such as lentil, or chickpea; or an oilseed such as canola; or a vegetable such as onion or carrot; or a specialty crop such as caraway, hemp, or sesame.

9. The plant or cell of claim 1, wherein the plant is resistant to one or more species of nematode relative to a non-plant of the same species.

10. The plant or cell of claim 9, wherein the nematode genus is selected from the group consisting of Heterodera, and Meloidogyne.

11. The plant or cell of claim 9, wherein the nematode is selected from Heterodera glycines, Heterodera schachtii, and Meloidogyne incognita.

12. The plant or cell of claim 9, wherein the nematode is Meloidogyne incognita.

13. The plant of claim 1, wherein the resistance is conferred through reduction of nematode colonization, reduction of nematode reproduction, reduction of syncytium formation, tolerance, or combinations thereof.

14. The plant of claim 1, further comprising a pest resistance QTL.

15. A seed from the plant according to claim 1.

16. A foodstuff comprising a plant part from the plant according to claim 1.

17. A method for increasing pest resistance in a plant comprising altering or reducing expression of
(i) Glyma.10G017000 β-1,4-endoglucancase, or its ortholog in another plant species by an amount effective to increase pest resistance in the plant; and/or
(ii) Glyma.10G017100 pectin methylesterase inhibitor, or its ortholog in another plant species by an amount effective to increase pest resistance in the plant.

18. The method of claim 17,
wherein expression of the Glyma.10G017000 β-1,4-endoglucancase is altered or reduced by reducing expression or translation of a target polynucleotide having a nucleic acid sequence according to SEQ ID NO:2 or 3, or a complement thereof, or a nucleic acid encoding the polypeptide of SEQ ID NO:7, or a complement thereof and/or
wherein expression of the Glyma.10G017100 pectin methylesterase inhibitor is altered or reduced by reducing expression or translation of a target polynucleotide having a nucleic acid sequence according to SEQ ID NO:10 or 11, or a complement thereof, or a nucleic acid encoding the polypeptide of SEQ ID NO:16, or a complement thereof.

19. The method of claim 18, wherein the pest comprises a nematode,
optionally wherein the nematode genus is selected from the group consisting of Heterodera, and Meloidogyne.
optionally wherein the nematode is selected from Heterodera glycines, Heterodera schachtii, and Meloidogyne incognita.

20. The method of claim 17, wherein the plant is a soybean plant,
optionally wherein the soybean plant comprises a Glycine max plant,
optionally wherein the Glycine max is a cultivar selected from the group consisting of Jack, Resnik, Williams 82, Corsoy, Crawford, Hutcheson, Benning, Woodruff, Kunitz and Champ.
